SEQUENCE LISTING (68 sequences)

SEQ ID NO: 1 - DNA, 5422 bp, Homo sapiens
gaaacggcccgagaagctcgcccggagaacggggaggaatatgctgtggagctcctctgccatataaacaaaaagaggtaaggctgcttttctctttgatttattggaaagttccaaattgcatgctcttcttttaattcctagctgagaagaaaaagactgtttggcaagcttatgtcgataggataactgaatagcaacctgctgctcatcagccaatgtaaggacactacatatgtgtatatacatgatcgggtctatgctttatactacttgatgttggatatttactaaccagaatgttacttagataatcatataaaaaagagtgaaaaagttaaggattttagtgtcaatacacagattaatgtaaagttaaccaagcccaatatcttgtttctttccaaaatttattatttctgccactaagagccagatacgaagccaggggtataatttatatgtttaatattcttaacagtaggtttccaatgatacaaaggctgtcaagcatgttagtcataaaggcaacccattttggaactaggttttttttctttgatatttatcttacaagagctctgaattaactaaccacataaaattcttttctgaacttgtatatttgaaagagcttacattattatctcctaattttagttctaagatgttaacttaggattttcctgaacactggattcttcctttttccagggcaaaatattatcttgggttgtaatttggctgtgaccatgaattacagagaaatgatcattttgaatacatgatgaaaatactcataactttgtaaattgttattgttattgctataatttgatttaaagaggatgttcaatatgttgctaaagattttgtctgggacaagtatttcattttttctgtatcccaactagttgaattgcttaaattacatatagtcacctgcatgtgtcaatgctctagcctgaattcaacagaaaagacattttaacttgagagattttaatgatagctatcaggggaaaaataatgttctaattatttggatttcagaaaattcaattttaaatcaaaccccatatagaatagtagagctgttgaaacatctttactacttgaaagagctgctacctgggaaaagaatcctttgggtctcactcaaaggcaataactatgtcattcaaactgaagctgaatagttaagcaaagggtagcacttcttaggctgtggtttaaacaactgcaatgagtagtctgcttcaactttacagcaaagtgcagttccaagataatgattttaaagatgagcatataatccacatgttaagactatttcagtattgtggcttcaaaaaaaataagttcagtaaggtttttaagattattatttcaaatgccaatgttgaaagttgattaccaaagctacattaatcctgaaataatataaacaagagtgcataatgtatttttatttgaacctcattattttatgaataaaattacttatagtaaacatgtatttataataaatattttggaagtatcctctcaaatgcctttcaagatgtttctttcaataaattaagtactctggtaaacgtgaatcatttaaataggtatatacgtatgctaaaatgttttcataactaagccattattgggctctaaaaaactggaaacaaaatcatcaatatgatcttgtgaacaatggtattttctacaattgatgtctaagctaacagaaaattgtatacatataacttagcttatctatcacaaaaccatgatctggtaaagatatgaacaaacttgtaaactcctcttacattcattttgtttacaacaaaatagccaatattaaaatagttttttcaatgttagcaatattaaatgtaaaacacataaaaactaatcctcttcctaattcctatgtaagattttaaggcatcggaggaaataatgaatggagatttttaagtaggaagaattcagcacatcatctgtgtttatttgaattgggatttacatagtggttattttaagatttttctaaacagtatgagtgatttaagatttcacaggtccataccaaaatagacacagacagaagacaaactgaatctgaacactcacaagttcagaaatagactagctaaaaaaacttatgttctctctgtagcatctgttattatttactatacactctgaagggctgatgaaaagtataaccacaattgcattttgcaagacagtcataatataaattattagaagaaatatgaagcaaagagattttaaaacactttattggtttagcacgttcacacaaaaagaaacttcattggtttagtctactggctggaatctgagaagacccgtgttctggaggttatttatggatcattggattcggtatcagattgagaacaagttatttctgaaaaatatatctggataggactccccagaaggtaattcggtatgattgacatataaaagtattactttgtcatatatggtcatgtaactgcagctgtgtttcagcaacaaaattagatggacatttcaggaaataaaccacatgttttttgacacaactttaaggacataatttgtgggcgcgtatgtgtaaatgcatgttttaaattaagccaacacattattttgccaatagacttcaatatataaaataattaaaacatcttgggaagtatttgctgctttattatagagaaacaacatgtactaaacatcaaactacaagttctgtaaatgaaaaaataaaatttaagaaatctcatatcagtgtttttgctgttgttttaactaagagaatgctatgcaagtttttggtgaatgagtaataattttgctaaagattcctgtggtttgcttgtgggaatcatgtggaaaatattcatgaatttttaaaaagagttatcctaagcttaatgtgaaataatatctactatgttttttcctcacctcaatgatcaattatttcttttgctatagtgtcttctagtttgtttcatttgtgtaactcatattttttatgttttaaggaaatctttcaaacatggctgaagcaaagacccactggcttggagcagccctgtctcttatccctttaattttcctcatctctggggctgaagcagcttcatttcagagaaaccagctgcttcagaaagaaccagacctcaggttggaaaatgtccaaaagtttcccagtcctgaaatgatcagggctttggagtacatagaaaacctccgacaacaagctcataaggaagaaagcagcccagattataatccctaccaaggtgtctctgtcccccttcagcaaaaagaaaatggcgatgaaagccacttgcccgagagggattcactgagtgaagaagactggatgagaataatactcgaagctttgagacaggctgaaaatgagcctcagtctgcaccaaaagaaaataagccctatgccttgaattcagaaaagaactttccaatggacatgagtgatgattatgagacacagcagtggccagaaagaaagcttaagcacatgcaattccctcctatgtatgaagagaattccagggataacccctttaaacgcacaaatgaaatagtggaggaacaatatactcctcaaagccttgctacattggaatctgtcttccaagagctggggaaactgacaggaccaaacaaccagaaacgtgagaggatggatgaggagcaaaaactttatacggatgatgaagatgatatctacaaggctaataacattgcctatgaagatgtggtcgggggagaagactggaacccagtagaggagaaaatagagagtcaaacccaggaagaggtgagagacagcaaagagaatatagaaaaaaatgaacaaatcaacgatgagatgaaacgctcagggcagcttggcatccaggaagaagatcttcggaaagagagtaaagaccaactctcagatgatgtctccaaagtaattgcctatttgaaaaggttagtaaatgctgcaggaagtgggaggttacagaatgggcaaaatggggaaagggccaccaggctttttgagaaacctcttgattctcagtctatttatcagctgattgaaatctcaaggaatttacagatacccccagaagacttaattgagatgctcaaaactggggagaagccgaatggatcagtggaaccggagcgggagcttgaccttcctgttgacctagatgacatctcagaggctgacttagaccatccagacctgttccaaaataggatgctctccaagagtggctaccctaaaacacctggtcgtgctgggactgaggccctaccagacgggctcagtgttgaggatattttaaatcttttagggatggagagtgcagcaaatcagaaaacgtcgtattttcccaatccatataaccaggagaaagttctgccaaggctcccttatggtgctggaagatctagatcgaaccagcttcccaaagctgcctggattccacatgttgaaaacagacagatggcatatgaaaacctgaacgacaaggatcaagaattaggtgagtacttggccaggatgctagttaaataccctgagatcattaattcaaaccaagtgaagcgagttcctggtcaaggctcatctgaagatgacctgcaggaagaggaacaaattgagcaggccatcaaagagcatttgaatcaaggcagctctcaggagactgacaagctggccccggtgagcaaaaggttccctgtggggcccccgaagaatgatgataccccaaataggcagtactgggatgaagatctgttaatgaaagtgctggaatacctcaaccaagaaaaggcagaaaagggaagggagcatattgctaagagagcaatggaaaatatgtaagctgctttcattaattaccctactttcattcctcccaccccaagcaaatcccaacatttctcttcagtgtgttgacttctatcctgttaacactgtaatatctttaaatgatgtacaggcagatgaaaccaggtcactggggagtctgcttcatttcctctgagctgttatcttgtgtatggatatgtgtaaatgttatgactccttgataaaaaatttattatgtccattattcaagaaagatatctatgactgtgtttaatagtatatctaatggctgtggcattgttgatgctcacatatgataaaaaagtgtcctataattctattgaaagtttttaatatttattgaattattttgttactgtctgtagtgttttgtggagtactggaccaaaaaaataaagcattataaatatatagttttatttataaggccttttctattgtgtgttttactgttgattaataaatgttatttctggacaa

SEQ ID NO: 2 - DNA, 2434 bp, Homo sapiens
gaaacggcccgagaagctcgcccggagaacggggaggaatatgctgtggagctcctctgccatataaacaaaaagaggaaatctttcaaacatggctgaagcaaagacccactggcttggagcagccctgtctcttatccctttaattttcctcatctctggggctgaagcagcttcatttcagagaaaccagctgcttcagaaagaaccagacctcaggttggaaaatgtccaaaagtttcccagtcctgaaatgatcagggctttggagtacatagaaaacctccgacaacaagctcataaggaagaaagcagcccagattataatccctaccaaggtgtctctgtcccccttcagcaaaaagaaaatggcgatgaaagccacttgcccgagagggattcactgagtgaagaagactggatgagaataatactcgaagctttgagacaggctgaaaatgagcctcagtctgcaccaaaagaaaataagccctatgccttgaattcagaaaagaactttccaatggacatgagtgatgattatgagacacagcagtggccagaaagaaagcttaagcacatgcaattccctcctatgtatgaagagaattccagggataacccctttaaacgcacaaatgaaatagtggaggaacaatatactcctcaaagccttgctacattggaatctgtcttccaagagctggggaaactgacaggaccaaacaaccagaaacgtgagaggatggatgaggagcaaaaactttatacggatgatgaagatgatatctacaaggctaataacattgcctatgaagatgtggtcgggggagaagactggaacccagtagaggagaaaatagagagtcaaacccaggaagaggtgagagacagcaaagagaatatagaaaaaaatgaacaaatcaacgatgagatgaaacgctcagggcagcttggcatccaggaagaagatcttcggaaagagagtaaagaccaactctcagatgatgtctccaaagtaattgcctatttgaaaaggttagtaaatgctgcaggaagtgggaggttacagaatgggcaaaatggggaaagggccaccaggctttttgagaaacctcttgattctcagtctatttatcagctgattgaaatctcaaggaatttacagatacccccagaagacttaattgagatgctcaaaactggggagaagccgaatggatcagtggaaccggagcgggagcttgaccttcctgttgacctagatgacatctcagaggctgacttagaccatccagacctgttccaaaataggatgctctccaagagtggctaccctaaaacacctggtcgtgctgggactgaggccctaccagacgggctcagtgttgaggatattttaaatcttttagggatggagagtgcagcaaatcagaaaacgtcgtattttcccaatccatataaccaggagaaagttctgccaaggctcccttatggtgctggaagatctagatcgaaccagcttcccaaagctgcctggattccacatgttgaaaacagacagatggcatatgaaaacctgaacgacaaggatcaagaattaggtgagtacttggccaggatgctagttaaataccctgagatcattaattcaaaccaagtgaagcgagttcctggtcaaggctcatctgaagatgacctgcaggaagaggaacaaattgagcaggccatcaaagagcatttgaatcaaggcagctctcaggagactgacaagctggccccggtgagcaaaaggttccctgtggggcccccgaagaatgatgataccccaaataggcagtactgggatgaagatctgttaatgaaagtgctggaatacctcaaccaagaaaaggcagaaaagggaagggagcatattgctaagagagcaatggaaaatatgtaagctgctttcattaattaccctactttcattcctcccaccccaagcaaatcccaacatttctcttcagtgtgttgacttctatcctgttaacactgtaatatctttaaatgatgtacaggcagatgaaaccaggtcactggggagtctgcttcatttcctctgagctgttatcttgtgtatggatatgtgtaaatgttatgactccttgataaaaaatttattatgtccattattcaagaaagatatctatgactgtgtttaatagtatatctaatggctgtggcattgttgatgctcacatatgataaaaaagtgtcctataattctattgaaagtttttaatatttattgaattattttgttactgtctgtagtgttttgtggagtactggaccaaaaaaataaagcattataaatatatagttttatttataaggccttttctattgtgtgttttactgttgattaataaatgttatttctggacaa

SEQ ID NO: 3 - DNA, 1854 bp, Homo sapiens
atggctgaagcaaagacccactggcttggagcagccctgtctcttatccctttaattttcctcatctctggggctgaagcagcttcatttcagagaaaccagctgcttcagaaagaaccagacctcaggttggaaaatgtccaaaagtttcccagtcctgaaatgatcagggctttggagtacatagaaaacctccgacaacaagctcataaggaagaaagcagcccagattataatccctaccaaggtgtctctgtcccccttcagcaaaaagaaaatggcgatgaaagccacttgcccgagagggattcactgagtgaagaagactggatgagaataatactcgaagctttgagacaggctgaaaatgagcctcagtctgcaccaaaagaaaataagccctatgccttgaattcagaaaagaactttccaatggacatgagtgatgattatgagacacagcagtggccagaaagaaagcttaagcacatgcaattccctcctatgtatgaagagaattccagggataacccctttaaacgcacaaatgaaatagtggaggaacaatatactcctcaaagccttgctacattggaatctgtcttccaagagctggggaaactgacaggaccaaacaaccagaaacgtgagaggatggatgaggagcaaaaactttatacggatgatgaagatgatatctacaaggctaataacattgcctatgaagatgtggtcgggggagaagactggaacccagtagaggagaaaatagagagtcaaacccaggaagaggtgagagacagcaaagagaatatagaaaaaaatgaacaaatcaacgatgagatgaaacgctcagggcagcttggcatccaggaagaagatcttcggaaagagagtaaagaccaactctcagatgatgtctccaaagtaattgcctatttgaaaaggttagtaaatgctgcaggaagtgggaggttacagaatgggcaaaatggggaaagggccaccaggctttttgagaaacctcttgattctcagtctatttatcagctgattgaaatctcaaggaatttacagatacccccagaagacttaattgagatgctcaaaactggggagaagccgaatggatcagtggaaccggagcgggagcttgaccttcctgttgacctagatgacatctcagaggctgacttagaccatccagacctgttccaaaataggatgctctccaagagtggctaccctaaaacacctggtcgtgctgggactgaggccctaccagacgggctcagtgttgaggatattttaaatcttttagggatggagagtgcagcaaatcagaaaacgtcgtattttcccaatccatataaccaggagaaagttctgccaaggctcccttatggtgctggaagatctagatcgaaccagcttcccaaagctgcctggattccacatgttgaaaacagacagatggcatatgaaaacctgaacgacaaggatcaagaattaggtgagtacttggccaggatgctagttaaataccctgagatcattaattcaaaccaagtgaagcgagttcctggtcaaggctcatctgaagatgacctgcaggaagaggaacaaattgagcaggccatcaaagagcatttgaatcaaggcagctctcaggagactgacaagctggccccggtgagcaaaaggttccctgtggggcccccgaagaatgatgataccccaaataggcagtactgggatgaagatctgttaatgaaagtgctggaatacctcaaccaagaaaaggcagaaaagggaagggagcatattgctaagagagcaatggaaaatatgtaa

SEQ ID NO: 4 - protein, 617 aa, Homo sapiens
MAEAKTHWLGAALSLIPLIFLISGAEAASFQRNQLLQKEPDLRLENVQKFPSPEMIRALEYIENLRQQAHKEESSPDYNPYQGVSVPLQQKENGDESHLPERDSLSEEDWMRIILEALRQAENEPQSAPKENKPYALNSEKNFPMDMSDDYETQQWPERKLKHMQFPPMYEENSRDNPFKRTNEIVEEQYTPQSLATLESVFQELGKLTGPNNQKRERMDEEQKLYTDDEDDIYKANNIAYEDVVGGEDWNPVEEKIESQTQEEVRDSKENIEKNEQINDEMKRSGQLGIQEEDLRKESKDQLSDDVSKVIAYLKRLVNAAGSGRLQNGQNGERATRLFEKPLDSQSIYQLIEISRNLQIPPEDLIEMLKTGEKPNGSVEPERELDLPVDLDDISEADLDHPDLFQNRMLSKSGYPKTPGRAGTEALPDGLSVEDILNLLGMESAANQKTSYFPNPYNQEKVLPRLPYGAGRSRSNQLPKAAWIPHVENRQMAYENLNDKDQELGEYLARMLVKYPEIINSNQVKRVPGQGSSEDDLQEEEQIEQAIKEHLNQGSSQETDKLAPVSKRFPVGPPKNDDTPNRQYWDEDLLMKVLEYLNQEKAEKGREHIAKRAMENM

SEQ ID NO: 5 - protein, 33 aa, Homo sapiens
TNEIVEEQYTPQSLATLESVFQELGKLTGPNNQ

SEQ ID NO: 6 - protein, 66 aa, Homo sapiens
ERMDEEQKLYTDDEDDIYKANNIAYEDVVGGEDWNPVEEKIESQTQEEVRDSKENIEKNEQINDEM

SEQ ID NO: 7 - protein, 40 aa, Homo sapiens
VPGQGSSEDDLQEEEQIEQAIKEHLNQGSSQETDKLAPVS

SEQ ID NO: 8 - protein, 42 aa, Homo sapiens
FPVGPPKNDDTPNRQYWDEDLLMKVLEYLNQEKAEKGREHIA

SEQ ID NO: 9 - DNA, 99 bp, Homo sapiens
acaaatgaaatagtggaggaacaatatactcctcaaagccttgctacattggaatctgtcttccaagagctggggaaactgacaggaccaaacaaccag

SEQ ID NO: 10 - DNA, 198 bp, Homo sapiens
gagaggatggatgaggagcaaaaactttatacggatgatgaagatgatatctacaaggctaataacattgcctatgaagatgtggtcgggggagaagactggaacccagtagaggagaaaatagagagtcaaacccaggaagaggtgagagacagcaaagagaatatagaaaaaaatgaacaaatcaacgatgagatg

SEQ ID NO: 11 - DNA, 120 bp, Homo sapiens
gttcctggtcaaggctcatctgaagatgacctgcaggaagaggaacaaattgagcaggccatcaaagagcatttgaatcaaggcagctctcaggagactgacaagctggccccggtgagc

SEQ ID NO: 12 - DNA, 126 bp, Homo sapiens
ttccctgtggggcccccgaagaatgatgataccccaaataggcagtactgggatgaagatctgttaatgaaagtgctggaatacctcaaccaagaaaaggcagaaaagggaagggagcatattgct

SEQ ID NO: 13 - DNA, 19 bp, Artificial Sequence
  synthetic oligonucleotide
acttacgctgagtacttcg

SEQ ID NO: 14 - DNA, 20 bp, Artificial Sequence
  synthetic oligonucleotide
ccttccactcaacagtcatt

SEQ ID NO: 15 - DNA, 20 bp, Artificial Sequence
  synthetic oligonucleotide
gaattggctggcgattcata

SEQ ID NO: 16 - DNA, 20 bp, Artificial Sequence
  synthetic oligonucleotide
gataatgatcatgatccatt

SEQ ID NO: 17 - DNA, 20 bp, Artificial Sequence
  synthetic oligonucleotide
gaagacattgcctgagctgt

SEQ ID NO: 18 - DNA, 20 bp, Artificial Sequence
  synthetic oligonucleotide
gcagacaagcaccttatgaa

SEQ ID NO: 19 - DNA, 20 bp, Artificial Sequence
  synthetic oligonucleotide
cccttgattctcagtctatt

SEQ ID NO: 20 - DNA, 20 bp, Artificial Sequence
  synthetic oligonucleotide
gctcccaaagagataaacat

SEQ ID NO: 21 - DNA, 8 bp, Artificial Sequence
  synthetic polynucleotide
  misc_feature (1)..(1): n is a, c, g, or t
  misc_feature (5)..(5): n is a, c, g, or t
ntgantca

SEQ ID NO: 22 - DNA, 5 bp, Artificial Sequence
  synthetic polynucleotide
  misc_feature (1)..(1): r is G or A
  misc_feature (3)..(3): r is G or A
rgraa

SEQ ID NO: 23 - DNA, 7 bp, Artificial Sequence
  synthetic polynucleotide
  misc_feature (2)..(2): n is a, c, g, or t
cncccac

SEQ ID NO: 24 - protein, 380 aa, Homo sapiens
MMFSGFNADYEASSSRCSSASPAGDSLSYYHSPADSFSSMGSPVNAQDFCTDLAVSSANFIPTVTAISTSPDLQWLVQPALVSSVAPSQTRAPHPFGVPAPSAGAYSRAGVVKTMTGGRAQSIGRRGKVEQLSPEEEEKRRIRRERNKMAAAKCRNRRRELTDTLQAETDQLEDEKSALQTEIANLLKEKEKLEFILAAHRPACKIPDDLGFPEEMSVASLDLTGGLPEVATPESEEAFTLPLLNDPEPKPSVEPVKSISSMELKTEPFDDFLFPASSRPSGSETARSVPDMDLSGSFYAADWEPLHSGSLGMGPMATELEPLCTPVVTCTPSCTAYTSSFVFTYPEADSFPSCAAAHRKGSSSNEPSSDSLSSPTLLAL

SEQ ID NO: 25 - protein, 302 aa, Homo sapiens
MFQAFPGDYDSGSRCSSSPSAESQYLSSVDSFGSPPTAAASQECAGLGEMPGSFVPTVTAITTSQDLQWLVQPTLISSMAQSQGQPLASQPPVVDPYDMPGTSYSTPGMSGYSSGGASGSGGPSTSGTTSGPGPARPARARPRRPREETETDQLEEEKAELESEIAELQKEKERLEFVLVAHKPGCKIPYEEGPGPGPLAEVRDLPGSAPAKEDGFSWLLPPPPPPPLPFQTSQDAPPNLTASLFTHSEVQVLGDPFPVVNPSYTSSFVLTCPEVSAFAGAQRTSGSDQPSDPLNSPSLLAL

SEQ ID NO: 26 - protein, 347 aa, Homo sapiens
MCTKMEQPFYHDDSYTATGYGRAPGGLSLHDYKLLKPSLAVNLADPYRSLKAPGARGPGPEGGGGGSYFSGQGSDTGASLKLASSELERLIVPNSNGVITTTPTPPGQYFYPRGGGSGGGAGGAGGGVTEEQEGFADGFVKALDDLHKMNHVTPPNVSLGATGGPPAGPGGVYAGPEPPPVYTNLSSYSPASASSGGAGAAVGTGSSYPTTTISYLPHAPPFAGGHPAQLGLGRGASTFKEEPQTVPEARSRDATPPVSPINMEDQERIKVERKRLRNRLAATKCRKRKLERIARLEDKVKTLKAENAGLSSTAGLLREQVAQLKQKVMTHVSNGCQLLLGVKGHAF

SEQ ID NO: 27 - DNA, 2104 bp, Homo sapiens
aaccgcatctgcagcgagcatctgagaagccaagactgagccggcggccgcggcgcagcgaacgagcagtgaccgtgctcctacccagctctgctccacagcgcccacctgtctccgcccctcggcccctcgcccggctttgcctaaccgccacgatgatgttctcgggcttcaacgcagactacgaggcgtcatcctcccgctgcagcagcgcgtccccggccggggatagcctctcttactaccactcacccgcagactccttctccagcatgggctcgcctgtcaacgcgcaggacttctgcacggacctggccgtctccagtgccaacttcattcccacggtcactgccatctcgaccagtccggacctgcagtggctggtgcagcccgccctcgtctcctccgtggccccatcgcagaccagagcccctcaccctttcggagtccccgccccctccgctggggcttactccagggctggcgttgtgaagaccatgacaggaggccgagcgcagagcattggcaggaggggcaaggtggaacagttatctccagaagaagaagagaaaaggagaatccgaagggaaaggaataagatggctgcagccaaatgccgcaaccggaggagggagctgactgatacactccaagcggagacagaccaactagaagatgagaagtctgctttgcagaccgagattgccaacctgctgaaggagaaggaaaaactagagttcatcctggcagctcaccgacctgcctgcaagatccctgatgacctgggcttcccagaagagatgtctgtggcttcccttgatctgactgggggcctgccagaggttgccaccccggagtctgaggaggccttcaccctgcctctcctcaatgaccctgagcccaagccctcagtggaacctgtcaagagcatcagcagcatggagctgaagaccgagccctttgatgacttcctgttcccagcatcatccaggcccagtggctctgagacagcccgctccgtgccagacatggacctatctgggtccttctatgcagcagactgggagcctctgcacagtggctccctggggatggggcccatggccacagagctggagcccctgtgcactccggtggtcacctgtactcccagctgcactgcttacacgtcttccttcgtcttcacctaccccgaggctgactccttccccagctgtgcagctgcccaccgcaagggcagcagcagcaatgagccttcctctgactcgctcagctcacccacgctgctggccctgtgagggggcagggaaggggaggcagccggcacccacaagtgccactgcccgagctggtgcattacagagaggagaaacacatcttccctagagggttcctgtagacctagggaggaccttatctgtgcgtgaaacacaccaggctgtgggcctcaaggacttgaaagcatccatgtgtggactcaagtccttacctcttccggagatgtagcaaaacgcatggagtgtgtattgttcccagtgacacttcagagagctggtagttagtagcatgttgagccaggcctgggtctgtgtctcttttctctttctccttagtcttctcatagcattaactaatctattgggttcattattggaattaacctggtgctggatattttcaaattgtatctagtgcagctgattttaacaataactactgtgttcctggcaatagtgtgttctgattagaaatgaccaatattatactaagaaaagatacgactttattttctggtagatagaaataaatagctatatccatgtactgtagtttttcttcaacatcaatgttcattgtaatgttactgatcatgcattgttgaggtggtctgaatgttctgacattaacagttttccatgaaaacgttttattgtgtttttaatttatttattaagatggattctcagatatttatatttttattttatttttttctaccttgaggtcttttgacatgtggaaagtgaatttgaatgaaaaatttaagcattgtttgcttattgttccaagacattgtcaataaaagcatttaagttgaatgcga

SEQ ID NO: 28 - DNA, 1143 bp, Homo sapiens
atgatgttctcgggcttcaacgcagactacgaggcgtcatcctcccgctgcagcagcgcgtccccggccggggatagcctctcttactaccactcacccgcagactccttctccagcatgggctcgcctgtcaacgcgcaggacttctgcacggacctggccgtctccagtgccaacttcattcccacggtcactgccatctcgaccagtccggacctgcagtggctggtgcagcccgccctcgtctcctccgtggccccatcgcagaccagagcccctcaccctttcggagtccccgccccctccgctggggcttactccagggctggcgttgtgaagaccatgacaggaggccgagcgcagagcattggcaggaggggcaaggtggaacagttatctccagaagaagaagagaaaaggagaatccgaagggaaaggaataagatggctgcagccaaatgccgcaaccggaggagggagctgactgatacactccaagcggagacagaccaactagaagatgagaagtctgctttgcagaccgagattgccaacctgctgaaggagaaggaaaaactagagttcatcctggcagctcaccgacctgcctgcaagatccctgatgacctgggcttcccagaagagatgtctgtggcttcccttgatctgactgggggcctgccagaggttgccaccccggagtctgaggaggccttcaccctgcctctcctcaatgaccctgagcccaagccctcagtggaacctgtcaagagcatcagcagcatggagctgaagaccgagccctttgatgacttcctgttcccagcatcatccaggcccagtggctctgagacagcccgctccgtgccagacatggacctatctgggtccttctatgcagcagactgggagcctctgcacagtggctccctggggatggggcccatggccacagagctggagcccctgtgcactccggtggtcacctgtactcccagctgcactgcttacacgtcttccttcgtcttcacctaccccgaggctgactccttccccagctgtgcagctgcccaccgcaagggcagcagcagcaatgagccttcctctgactcgctcagctcacccacgctgctggccctgtga

SEQ ID NO: 29 - DNA, 3667 bp, Homo sapiens
attcataagactcagagctacggccacggcagggacacgcggaaccaagacttggaaacttgattgttgtggttcttcttgggggttatgaaatttcattaatctttttttttccggggagaaagtttttggaaagattcttccagatatttcttcattttcttttggaggaccgacttactttttttggtcttctttattactcccctccccccgtgggacccgccggacgcgtggaggagaccgtagctgaagctgattctgtacagcgggacagcgctttctgcccctgggggagcaacccctccctcgcccctgggtcctacggagcctgcactttcaagaggtacagcggcatcctgtgggggcctgggcaccgcaggaagactgcacagaaactttgccattgttggaacgggacgttgctccttccccgagcttccccggacagcgtactttgaggactcgctcagctcaccggggactcccacggctcaccccggacttgcaccttacttccccaacccggccatagccttggcttcccggcgacctcagcgtggtcacaggggcccccctgtgcccagggaaatgtttcaggctttccccggagactacgactccggctcccggtgcagctcctcaccctctgccgagtctcaatatctgtcttcggtggactccttcggcagtccacccaccgccgccgcctcccaggagtgcgccggtctcggggaaatgcccggttccttcgtgcccacggtcaccgcgatcacaaccagccaggacctccagtggcttgtgcaacccaccctcatctcttccatggcccagtcccaggggcagccactggcctcccagcccccggtcgtcgacccctacgacatgccgggaaccagctactccacaccaggcatgagtggctacagcagtggcggagcgagtggcagtggtgggccttccaccagcggaactaccagtgggcctgggcctgcccgcccagcccgagcccggcctaggagaccccgagaggagacggagacagatcagttggaggaagaaaaagcagagctggagtcggagatcgccgagctccaaaaggagaaggaacgtctggagtttgtgctggtggcccacaaaccgggctgcaagatcccctacgaagaggggcccgggccgggcccgctggcggaggtgagagatttgccgggctcagcaccggctaaggaagatggcttcagctggctgctgccgcccccgccaccaccgcccctgcccttccagaccagccaagacgcaccccccaacctgacggcttctctctttacacacagtgaagttcaagtcctcggcgaccccttccccgttgttaacccttcgtacacttcttcgtttgtcctcacctgcccggaggtctccgcgttcgccggcgcccaacgcaccagcggcagtgaccagccttccgatcccctgaactcgccctccctcctcgctctgtgaactctttagacacacaaaacaaacaaacacatgggggagagagacttggaagaggaggaggaggaggagaaggaggagagagaggggaagagacaaagtgggtgtgtggcctccctggctcctccgtctgaccctctgcggccactgcgccactgccatcggacaggaggattccttgtgttttgtcctgcctcttgtttctgtgccccggcgaggccggagagctggtgactttggggacagggggtgggaaggggatggacacccccagctgactgttggctctctgacgtcaacccaagctctggggatgggtggggaggggggcgggtgacgcccaccttcgggcagtcctgtgtgaggattaagggacgggggtgggaggtaggctgtggggtgggctggagtcctctccagagaggctcaacaaggaaaaatgccactccctacccaatgtctcccacacccaccctttttttggggtgcctaggttggtttcccctgcactcccgaccttagcttattgatcccacatttccatggtgtgagatcctctttactctgggcagaagtgagccccccccttaaagggaattcgatgcccccctagaataatctcatccccccacccgacttcttttgaaatgtgaacgtccttccttgactgtctagccactccctcccagaaaaactggctctgattggaatttctggcctcctaaggctccccaccccgaaatcagcccccagccttgtttctgatgacagtgttatcccaagaccctgccccctgccagccgaccctcctggccttcctcgttgggccgctctgatttcaggcagcaggggctgctgtgatgccgtcctgctggagtgatttatactgtgaaatgagttggccagattgtggggtgcagctgggtggggcagcacacctctggggggataatgtccccactcccgaaagcctttcctcggtctcccttccgtccatcccccttcttcctcccctcaacagtgagttagactcaagggggtgacagaaccgagaagggggtgacagtcctccatccacgtggcctctctctctctcctcaggaccctcagccctggcctttttctttaaggtcccccgaccaatccccagcctaggacgccaacttctcccaccccttggcccctcacatcctctccaggaagggagtgaggggctgtgacatttttccggagaagatttcagagctgaggctttggtacccccaaacccccaatatttttggactggcagactcaaggggctggaatctcatgattccatgcccgagtccgcccatccctgaccatggttttggctctcccaccccgccgttccctgcgcttcatctcatgaggatttctttatgaggcaaatttatattttttaatatcggggggtggaccacgccgccctccatccgtgctgcatgaaaaacattccacgtgccccttgtcgcgcgtctcccatcctgatcccagacccattccttagctatttatccctttcctggtttccgaaaggcaattatatctattatgtataagtaaatatattatatatggatgtgtgtgtgtgcgtgcgcgtgagtgtgtgagcgcttctgcagcctcggcctaggtcacgttggccctcaaagcgagccgttgaattggaaactgcttctagaaactctggctcagcctgtctcgggctgacccttttctgatcgtctcggcccctctgattgttcccgatggtctctctccctctgtcttttctcctccgcctgtgtccatctgaccgttttcacttgtctcctttctgactgtccctgccaatgctccagctgtcgtctgactctgggttcgttggggacatgagattttattttttgtgagtgagactgagggatcgtagatttttacaatctgtatctttgacaattctgggtgcgagtgtgagagtgtgagcagggcttgctcctgccaaccacaattcaatgaatccccgacccccctaccccatgctgtacttgtggttctctttttgtattttgcatctgaccccggggggctgggacagattggcaatgggccgtcccctctccccttggttctgcactgttgccaataaaaagctcttaaaaacgca

SEQ ID NO: 30 - DNA, 909 bp, Homo sapiens
atgtttcaggctttccccggagactacgactccggctcccggtgcagctcctcaccctctgccgagtctcaatatctgtcttcggtggactccttcggcagtccacccaccgccgccgcctcccaggagtgcgccggtctcggggaaatgcccggttccttcgtgcccacggtcaccgcgatcacaaccagccaggacctccagtggcttgtgcaacccaccctcatctcttccatggcccagtcccaggggcagccactggcctcccagcccccggtcgtcgacccctacgacatgccgggaaccagctactccacaccaggcatgagtggctacagcagtggcggagcgagtggcagtggtgggccttccaccagcggaactaccagtgggcctgggcctgcccgcccagcccgagcccggcctaggagaccccgagaggagacggagacagatcagttggaggaagaaaaagcagagctggagtcggagatcgccgagctccaaaaggagaaggaacgtctggagtttgtgctggtggcccacaaaccgggctgcaagatcccctacgaagaggggcccgggccgggcccgctggcggaggtgagagatttgccgggctcagcaccggctaaggaagatggcttcagctggctgctgccgcccccgccaccaccgcccctgcccttccagaccagccaagacgcaccccccaacctgacggcttctctctttacacacagtgaagttcaagtcctcggcgaccccttccccgttgttaacccttcgtacacttcttcgtttgtcctcacctgcccggaggtctccgcgttcgccggcgcccaacgcaccagcggcagtgaccagccttccgatcccctgaactcgccctccctcctcgctctgtga

SEQ ID NO: 31 - DNA, 1830 bp, Homo sapiens
gggaccttgagagcggccaggccagcctcggagccagcagggagctgggagctgggggaaacgacgccaggaaagctatcgcgccagagagggcgacgggggctcgggaagcctgacagggcttttgcgcacagctgccggctggctgctacccgcccgcgccagcccccgagaacgcgcgaccaggcacccagtccggtcaccgcagcggagagctcgccgctcgctgcagcgaggcccggagcggccccgcagggaccctccccagaccgcctgggccgcccggatgtgcactaaaatggaacagcccttctaccacgacgactcatacacagctacgggatacggccgggcccctggtggcctctctctacacgactacaaactcctgaaaccgagcctggcggtcaacctggccgacccctaccggagtctcaaagcgcctggggctcgcggacccggcccagagggcggcggtggcggcagctacttttctggtcagggctcggacaccggcgcgtctctcaagctcgcctcttcggagctggaacgcctgattgtccccaacagcaacggcgtgatcacgacgacgcctacacccccgggacagtacttttacccccgcgggggtggcagcggtggaggtgcagggggcgcagggggcggcgtcaccgaggagcaggagggcttcgccgacggctttgtcaaagccctggacgatctgcacaagatgaaccacgtgacaccccccaacgtgtccctgggcgctaccggggggcccccggctgggcccgggggcgtctacgccggcccggagccacctcccgtttacaccaacctcagcagctactccccagcctctgcgtcctcgggaggcgccggggctgccgtcgggaccgggagctcgtacccgacgaccaccatcagctacctcccacacgcgccgcccttcgccggtggccacccggcgcagctgggcttgggccgcggcgcctccaccttcaaggaggaaccgcagaccgtgccggaggcgcgcagccgggacgccacgccgccggtgtcccccatcaacatggaagaccaagagcgcatcaaagtggagcgcaagcggctgcggaaccggctggcggccaccaagtgccggaagcggaagctggagcgcatcgcgcgcctggaggacaaggtgaagacgctcaaggccgagaacgcggggctgtcgagtaccgccggcctcctccgggagcaggtggcccagctcaaacagaaggtcatgacccacgtcagcaacggctgtcagctgctgcttggggtcaagggacacgccttctgaacgtcccctgcccctttacggacaccccctcgcttggacggctgggcacacgcctcccactggggtccagggagcaggcggtgggcacccaccctgggacctaggggcgccgcaaaccacactggactccggccctcctaccctgcgcccagtccttccacctcgacgtttacaagcccccccttccacttttttttgtatgttttttttctgctggaaacagactcgattcatattgaatataatatatttgtgtatttaacagggaggggaagagggggcgatcgcggcggagctggccccgccgcctggtactcaagcccgcggggacattgggaaggggacccccgccccctgccctcccctctctgcaccgtactgtggaaaagaaacacgcacttagtctctaaagagtttattttaagacgtgtttgtgtttgtgtgtgtttgttctttttattgaatctatttaagtaaaaaaaaaattggttctttattaa

SEQ ID NO: 32 - DNA, 1044 bp, Homo sapiens
atgtgcactaaaatggaacagcccttctaccacgacgactcatacacagctacgggatacggccgggcccctggtggcctctctctacacgactacaaactcctgaaaccgagcctggcggtcaacctggccgacccctaccggagtctcaaagcgcctggggctcgcggacccggcccagagggcggcggtggcggcagctacttttctggtcagggctcggacaccggcgcgtctctcaagctcgcctcttcggagctggaacgcctgattgtccccaacagcaacggcgtgatcacgacgacgcctacacccccgggacagtacttttacccccgcgggggtggcagcggtggaggtgcagggggcgcagggggcggcgtcaccgaggagcaggagggcttcgccgacggctttgtcaaagccctggacgatctgcacaagatgaaccacgtgacaccccccaacgtgtccctgggcgctaccggggggcccccggctgggcccgggggcgtctacgccggcccggagccacctcccgtttacaccaacctcagcagctactccccagcctctgcgtcctcgggaggcgccggggctgccgtcgggaccgggagctcgtacccgacgaccaccatcagctacctcccacacgcgccgcccttcgccggtggccacccggcgcagctgggcttgggccgcggcgcctccaccttcaaggaggaaccgcagaccgtgccggaggcgcgcagccgggacgccacgccgccggtgtcccccatcaacatggaagaccaagagcgcatcaaagtggagcgcaagcggctgcggaaccggctggcggccaccaagtgccggaagcggaagctggagcgcatcgcgcgcctggaggacaaggtgaagacgctcaaggccgagaacgcggggctgtcgagtaccgccggcctcctccgggagcaggtggcccagctcaaacagaaggtcatgacccacgtcagcaacggctgtcagctgctgcttggggtcaagggacacgccttctga

SEQ ID NO: 33 - protein, 577 aa, Mus musculus
MAGAKAYRLGAVLLLIHLIFLISGAEAASFQRNQLLQKEPDLRLENVQKFPSPEMIRALEYIEKLRQQAHREESSPDYNPYQGVSVPLQLKENGEESHLAESSRDALSEDEWMRIILEALRQAENEPPSAPKENKPYALNLEKNFPVDTPDDYETQQWPERKLKHMRFPLMYEENSRENPFKRTNEIVEEQYTPQSLATLESVFQELGKLTGPSNQKRERVDEEQKLYTDDEDDVYKTNNIAYEDVVGGEDWSPIEEKIETQTQEEVRDSKENTEKNEQINEEMKRSGQLGLPDEENRRESKDQLSEDASKVITYLRRNLQIPPEDLIEMLKAGEKPNGLVEPEQDLELAVDLDDIPEADLDRPDMFQSKMLSKGGYPKAPGRGMVEALPDGLSVEDILNVLGMENVVNQKSPYFPNQYSQDKALMRLPYGPGKSRANQIPKVAWIPDVESRQAPYENLNDQELGEYLARMLVKYPELLNTNQLKRVPSPVSSEDDLQEEEQLEQAIKEHLGPGSSQEMERLAKVSKRIPVGSLKNEDTPNRQYLDEDMLLKVLEYLNQEQAEQGREHLAKRAMENM

SEQ ID NO: 34 - protein, 619 aa, Rattus norvegicus
MAESKAYRFGAVLLLIHLIFLVPGTEAASFQRNQLLQKEPDLRLENVQKFPSPEMIRALEYIEKLRQQAHREESSPDYNPYQGISVPLQLKENGEESHLAESSRDVLSEDEWMRIILEALRQAENEPPSALKENKPYALNLEKNFPVDTPDDYETQQWPERKLKHMRFPLMYEENSRENPFKRTNEIVEEQYTPQSLATLESVFQELGKLTGPSNQKRERVDEEQKLYTDDEDDVYKTNNIAYEDVVGGEDWSPMEEKIETQTQEEVRDSKENTEKNEQINEEMKRSGHLGLPDEGNRKESKDQLSEDASKVITYLRRLVNAVGSGRSQSGQNGDRAARLLERPLDSQSIYQLIEISRNLQIPPEDLIEMLKAGEKPNGLVEPEQDLELAVDLDDIPEADIDRPDMFQSKTLSKGGYPKAPGRGMMEALPDGLSVEDILNVLGMENVANQKSPYFPNQYSRDKALLRLPYGPGKSRANQIPKVAWIPDVESRQAPYDNLNDKDQELGEYLARMLVKYPELMNTNQLKRVPSPGSSEDDLQEEEQLEQAIKEHLGQGSSQEMEKLAKVSKRIPAGSLKNEDTPNRQYLDEDMLLKVLEYLNQEQAEQGREHLAKRAMENM

SEQ ID NO: 35 - protein, 616 aa, Pan troglodytes
MAEAKTHWLGAALSLIPLIFLISGAEAASFQRNQLLQKEPDLRLENVQKFPSPEMIRALEYIEKLRQQAHKEESSPDYNPYQGVSVPLQQKENGDESHLPERDSLSEEDWMRIILEALRQAENEPQSAPKENKPYALNSEKNFPMDMSDDYETQQWPERKLKHMQFPPMYEENSRDNPFKRTNEIVEEQYTPQSLATLESVFQELGKLTGPNNQKRERMDEEQKLYTDDDDIYKANNIAYEDVVGGEDWNPVEEKIESQTQEEVRDSKENIEKNEQINDEMKRSGQLGIQEEDLRKESKDQLSDDVSKVIAYLKRLVNAAGSGRLQNGQNGERATRLFEKPLDSQSIYQLIEISRNLQIPPEDLIEMLKTGEKPNGSVEPERELDLPVDLDDISEADLDHPDLFQNKMLSKSGYPKTPGRAGTEALPDGLSVEDILNLLGMESAANQKTSYFPNPYNQEKVLPRLPYGPGRSRSNQLPKAAWIPYVENRQMAYENLNDKDQELGEYLARMLVKYPEIINSNQVKRVPGQGSSEDDLQEEEQIEQAIKEHLNQGSSQETDKLAPVSKRFPVGPPKNDDTPNRQYLDEDLLMKVLEYLNQEKAEKGREHIAKRAMENM

SEQ ID NO: 36 - DNA, 2604 bp, Mus musculus
aaacggcccgagccctcactcagcggcagagaggagcatgcttggagccttccacataatataagacagaggaaatctttaagacatggctggagctaaggcgtaccgacttggagcagttctgcttcttatccacttaattttcctcatctctggagccgaagcagcttccttccagcgaaaccagctgcttcagaaagaaccagacctcagattggagaatgtccaaaagtttcctagtccagaaatgatcagggctttggagtacatagaaaagctcaggcagcaagctcacagagaagaaagcagcccagactacaatccctaccaaggcgtctctgttcctcttcaactcaaagaaaacggagaagaaagccacttggcagagagctcaagggatgcactgagtgaagacgagtggatgcggataatactcgaggctctgaggcaggctgaaaatgagccgccatctgcccccaaagagaacaagccctatgccttgaatctggagaagaacttcccagtggacacgcctgatgactatgagactcaacagtggcctgagaggaaactcaagcacatgcggttccctctcatgtatgaagagaattccagagaaaaccccttcaaacgcacaaatgaaatagtcgaggaacaatacacaccccaaagtcttgctaccctggagtctgtgttccaagagcttgggaaactgacagggccaagcaaccagaagcgtgagagggttgacgaggaacaaaagctgtacacagatgatgaagacgacgtgtacaagaccaacaacattgcctatgaagatgtcgtggggggagaagactggagccccatagaggagaaaatagagactcaaacccaggaagaggtgagagacagcaaagagaacacagaaaaaaatgaacaaatcaatgaagagatgaaacgttcagggcagttggggctcccagatgaagaaaaccggagagagagtaaagaccaactctcagaggatgcctccaaagttatcacctacctgagaaggaatttgcagataccccctgaagatttaattgagatgctcaaagctggagagaagccaaatgggttggtggagccagagcaggatctggagcttgctgttgacctagatgacatcccagaggctgacctagaccgtccagacatgtttcaaagtaagatgctctccaagggtgggtatcccaaggcacctggtcgtggtatggtagaggccttgcctgatgggctgagtgtcgaggacattttaaatgttttagggatggagaatgtagtaaatcagaagtccccatattttcccaaccaatatagccaagacaaggctctgatgaggctcccttatggtcctgggaaatctagagccaaccagattcccaaagtagcctggatccctgatgttgaaagcagacaagcaccttatgaaaatctgaatgaccaagaattgggagagtacttagccaggatgctagttaagtaccctgagctcctgaataccaaccagctgaagagagtgcccagtccagtctcctcagaggatgacctccaagaagaagagcagctcgagcaggccatcaaggaacatctggggccaggaagctcccaggaaatggagagactggccaaggtgagcaaaaggatccccgtaggatccctgaagaatgaggacaccccaaacagacagtacctggatgaagatatgctcctgaaagtgctggagtacctcaaccaagagcaggcagagcaggggagggagcatcttgccaagcgggccatggaaaacatgtaaacagctttaatgcccaatttcccttctttcccccaagtaagccccctacatttctcttaagtgtgttgatctctatcctgttgacagtgtaatatctttaaagtgatgtataggcagatgactccaggtcattttgggggatctgcttcacttattctgagctgttacgttgtgtgtggatgtgtgtaaatgttatgattcccagattgaaaaaaaatgttctttattcaagaaagatatctatgatagtgttggctaatgtatctaatggtcatggaattgatgatgctcacatatgataaagagtatcctataattatcttggaagtttttaacatttattgaattattttgttactgtctgtagtgttttgtggagttctggagcaaaaccaataaagcattataaatatatagttttacttataaggccttttctattgtgtgttttattgttgattaataaatgttatttctggatacctttggactttttattctggaaaccagagacaactggtatggatcaagcagcatggagccagaggagaaaattattactgtccacaggcaacccaggtaagagatgaatcttatatgtgatcatattttctgcctacaggatgttgtgaacattcccgaacagccttacatcttttcatgttttccatatacctcattaacaaaacgagactttgggtataattcttacacttcacattgattcatataagtaaaagatattaaactttccccactcatcacaatttgaaaatgaaagaaaa

SEQ ID NO: 37 - DNA, 2291 bp, Rattus norvegicus
acaatataagacagaggaaaattttaagacatggctgaatcgaaggcttaccgatttggagcagttctgcttcttatccacttaattttccttgtccctggaaccgaagcagcttccttccagcgaaaccagctgcttcagaaagaaccagacctcagattggagaatgtccagaagtttcctagtccagaaatgatcagggctttggagtacatagaaaagctcaggcagcaggcccacagagaagaaagcagcccagactacaatccctaccaaggcatctctgttccccttcaactcaaagaaaacggagaagaaagtcacttggcagagagctcaagggatgtcttgagtgaagacgagtggatgcggataatacttgaggctttgaggcaggctgaaaatgagccgccatctgccctcaaggagaacaagccctatgccttgaatctggagaagaacttccctgtggacacgcctgatgactatgagactcaacaatggcctgagaggaaactcaagcacatgcggttccctctcatgtatgaagagaattccagggaaaaccccttcaaacgcacaaacgaaatagtagaagaacagtacacaccccaaagtcttgctaccctggagtctgtgttccaagagcttgggaaactgacagggccaagcaaccagaagcgtgagagggttgacgaggaacagaagctctacacggacgatgaagatgacgtgtacaagaccaacaacattgcctatgaagatgtggtcgggggagaagactggagtcctatggaggagaaaatagagactcaaacccaggaagaggtgagagacagcaaagagaacacagaaaaaaacgaacaaatcaatgaagagatgaaacggtcagggcacttggggctcccagatgaaggtaaccggaaagagagcaaagaccagctctcagaggacgcctccaaggtcatcacctacttgagaaggttagtgaatgctgtgggcagtgggaggtcccagagtgggcaaaacggggacagggcagccaggcttcttgagaggccccttgattctcagtctatttatcagctgattgaaatctccaggaatttgcagataccccctgaagacttaattgagatgctcaaagctggggagaaaccaaatgggttggtggagcccgagcaggatctggagcttgctgttgacctagatgacatcccggaagctgacatagaccgcccagacatgtttcaaagtaagacgctctccaagggtgggtatcccaaggcacctggtcgaggtatgatggaggccttgccagatggcctcagtgttgaagacattttaaatgttttagggatggagaatgtagcaaatcagaagtccccatatttccccaaccaatacagccgagacaaggctctgctgaggcttccttatggtcctgggaaatctagagccaaccagattcccaaagtagcctggatcccagacgttgaaagcagacaagccccctatgacaatctgaatgataaggaccaagaattgggagagtacttagccaggatgctagttaagtaccctgagctcatgaataccaaccagctgaagagagtgcccagcccaggctcctcagaagatgacctccaagaagaagagcagctcgagcaggccatcaaggagcatctgggtcaaggaagctcccaggaaatggagaaactggccaaggtgagcaaaaggatccctgcaggatccctgaagaatgaggataccccaaatagacagtacctggatgaagatatgctcctgaaagtgctagagtatctcaatcaagaacaggcagagcagggaagggaacatcttgccaaacgggccatggaaaacatgtaaacagctttaatgcccaatttcccttcttttccccaagtgaatcccctccctttctcttaagtgtgttaatctctatcctgttaacactgtaatatctttaagtgatgtacaagcagatgactccagatagttttggggatctgctttacttattctgagctgttatgttgtgtatggatgtgtataaatgttatgactctcagatttaaaaaatatgtcctttattcaagaaagatatctatgatagtgttgactaatgtatccaatggtcatggtattgacaatgctcacatatgatgaagagtatcctataattatcttggaagtttttaacatttattgaattattttgttactgtctgtagtgttttgtggagttctggagcaaaatcaataaagca

SEQ ID NO: 38 - DNA, 2568 bp, Pan troglodytes
ataaagtgattattttctcttggttctttgaaaaacctcgcttgtgctggggtttgtggctgaacccggtgacgtcagtgtggcagtgcggagtcaggcgcagcggctccctataagcagaggagctgtccgtgtgctgaaacggcccgagaagctcgcccggagaacggggaggaatatgctgtggagctcctctgccatataaacaaaaagaggaaatctttcagacatggctgaagcaaagacccactggcttggagcagccctgtctcttatccctttaattttcctcatctctggggctgaagcagcttcatttcagagaaaccagctgcttcagaaagaaccagacctcaggttggaaaatgtccaaaagtttcccagtcctgaaatgatcagggctttggagtacatagaaaagctccgacaacaggctcataaggaagaaagcagcccagattataatccctaccaaggtgtctctgtcccccttcagcaaaaagaaaatggcgatgaaagtcacttgcccgagagggattcactgagtgaagaagactggatgagaataatactcgaagctttgagacaggctgaaaatgagcctcagtctgcaccaaaagaaaataagccctatgccttgaattcagaaaagaactttccaatggacatgagtgatgattatgagacacagcagtggccagaaagaaagcttaagcacatgcaattccctcctatgtatgaagagaattccagggataacccctttaaacgcacaaatgaaatagtggaggaacaatatactcctcaaagccttgctacattggaatctgtcttccaagagctggggaaactgacaggaccaaacaaccagaaacgtgagaggatggatgaggagcaaaaactttatacggatgatgatgatatctacaaggctaataacattgcctatgaagatgtggtggggggagaagattggaacccagtagaggagaaaatagagagtcaaacccaggaagaggtgagagacagcaaagagaatatagaaaaaaatgaacaaatcaatgatgagatgaaacgctcagggcagcttggcatccaggaagaagatcttcggaaagagagtaaagaccaactctcagatgatgtctccaaagtaattgcctatttgaaaaggttagtaaatgctgcaggaagtgggaggttacagaatgggcaaaatggggaaagggccaccaggctttttgagaaacctcttgattctcagtctatttatcagctgattgaaatctcaaggaatttacagatacccccagaagacttaattgagatgctcaaaactggggagaagccgaatggatcagtggaaccggagcgggagcttgaccttcctgttgacctagatgacatctcagaggctgacttagaccatccagacctgttccaaaataagatgctctccaagagtggctaccctaaaacacctggtcgtgctgggactgaggccctaccagacgggctcagtgttgaggatattttaaatcttttagggatggagagtgcagcaaatcagaaaacttcgtattttcccaatccatataaccaggagaaagttctgccaaggctcccttatggtcctggaagatctagatcgaaccagcttcccaaagctgcctggattccatatgttgaaaacagacagatggcatatgaaaacctgaacgacaaggatcaagaattaggtgagtacttggccaggatgctagttaaataccctgagatcattaattcaaaccaagtgaagcgagttcctggtcaaggctcatctgaagatgacctacaggaagaggaacaaattgagcaggccatcaaagagcatttgaatcaaggcagctctcaggagactgacaagctggccccggtgagcaaaaggttccctgtggggcccccgaagaatgatgataccccaaataggcagtacttggatgaagatctgttaatgaaagtgctggaatacctcaaccaagaaaaggcagaaaagggaagggagcatattgctaagagagcaatggaaaatatgtaagctgctttcattaattaccctactttcattcctcccaccccaagcaaatcccaacatttctctttagtgtgttgacttctatcctgttaacactgtaatatctttaaatgatgtacaggcagatgaaaccaggtcactggggagtctgcttcatttcctctgagctgttatcttgtgtatggatacgtgtaaatgttatgactccttgataaaaaatttattatgtccattattcaagaaagatatctatgactgtgtttaatagtgtatctaatggctgtggcattgttgatgctcacatatgataaaaagtgtcctataattctattgaaagtttttaatatttattgaattattttgttactgtctgtagtgttttgtggagtactggaccaaaaaaataaagcattataaatatatagttttatttataaggccttttctattgtgtgttttactgttgattaataaatgttatttctggacaa

SEQ ID NO: 39 - protein, 18 aa, Artificial Sequence
  synthetic polypeptide
LLIILRRRIRKQAHAHSK

SEQ ID NO: 40 - protein, 10 aa, Artificial Sequence
  synthetic polypeptide
RRLSYSRRRF

SEQ ID NO: 41 - protein, 11 aa, Artificial Sequence
  synthetic polypeptide
YGRKKRRQRRR

SEQ ID NO: 42 - protein, 21 aa, Artificial Sequence
  synthetic polypeptide
AGYLLGKINLKALAALAKKIL

SEQ ID NO: 43 - protein, 29 aa, Artificial Sequence
  synthetic polypeptide
YTIWMPENPRPGTPCDIFTNSRGKRASNG

SEQ ID NO: 44 - protein, 19 aa, Artificial Sequence
  synthetic polypeptide
TFFYGGSRGKRNNFKTEEY

SEQ ID NO: 45 - protein, 19 aa, Artificial Sequence
  synthetic polypeptide
YEETKFNNRKGRSGGYFFT

SEQ ID NO: 46 - protein, 10 aa, Artificial Sequence
  synthetic polypeptide
LRKLRKRLLR

SEQ ID NO: 47 - protein, 10 aa, Artificial Sequence
  synthetic polypeptide
CGHKAKGPRK

SEQ ID NO: 48 - protein, 5 aa, Artificial Sequence
  synthetic polypeptide
  misc_feature (1)..(5): cyclic peptide
RGDFK

SEQ ID NO: 49 - protein, 16 aa, Artificial Sequence
  synthetic polypeptide
FKESWREARGTRIERG

SEQ ID NO: 50 - protein, 16 aa, Artificial Sequence
  synthetic polypeptide
GREIRTGRAERWSEKF

SEQ ID NO: 51 - protein, 13 aa, Artificial Sequence
  synthetic polypeptide
  misc_feature (13)..(13): O-beta-glucoside
YGGFLGGYTGFLS

SEQ ID NO: 52 - protein, 7 aa, Artificial Sequence
  synthetic polypeptide
  misc_feature (7)..(7): monosaccharide
GFTGFLS

SEQ ID NO: 53 - protein, 23 aa, Artificial Sequence
  synthetic polypeptide
HGLASTLTRWAHYNALIRAFGGG

SEQ ID NO: 54 - protein, 3 aa, Artificial Sequence
  synthetic polypeptide
GSH

SEQ ID NO: 55 - protein, 5 aa, Artificial Sequence
  synthetic polypeptide
LPFFD

SEQ ID NO: 56 - protein, 8 aa, Artificial Sequence
  synthetic polypeptide
  misc_feature (1)..(8): cyclized peptide
  misc_feature (1)..(1): diaminopropionic acid
KAPETALD

SEQ ID NO: 57 - protein, 16 aa, Artificial Sequence
  synthetic polypeptide
RQIKIWFQNRRMKWKK

SEQ ID NO: 58 - protein, 39 aa, Artificial Sequence
  synthetic polypeptide
KSVRTWNEIIPSKGCLRVGGRCHPHVNGGGRRRRRRRRR

SEQ ID NO: 59 - protein, 34 aa, Artificial Sequence
  synthetic polypeptide
YTWMPENPRPGTPCDIFTNSRGKRASNGGGGGGC

SEQ ID NO: 60 - protein, 36 aa, Artificial Sequence
  synthetic polypeptide
MCMPCFTTDHQMARKCDDCCGGKGRGKCYGPQCLCR

SEQ ID NO: 61 - protein, 7 aa, Artificial Sequence
  synthetic polypeptide
HAIYPRH

SEQ ID NO: 62 - protein, 16 aa, Artificial Sequence
  synthetic polypeptide
GRKKRRQRRRPPQGWC

SEQ ID NO: 63 - protein, 11 aa, Artificial Sequence
  synthetic polypeptide
YGRKKRRQRRR

SEQ ID NO: 64 - protein, 11 aa, Artificial Sequence
  synthetic polypeptide
RRRQRRKKRGY

SEQ ID NO: 65 - protein, 12 aa, Artificial Sequence
  synthetic polypeptide
TGNYKALHPHNG

SEQ ID NO: 66 - protein, 12 aa, Artificial Sequence
  synthetic polypeptide
THRPPMWSPVWP

SEQ ID NO: 67 - protein, 12 aa, Artificial Sequence
  synthetic polypeptide
PWVPSWMPPRHT

SEQ ID NO: 68 - protein, 8 aa, Artificial Sequence
  synthetic polypeptide
  misc_feature (2)..(8): cyclized peptide
CMPRLRGC

(19) United States
(12) Patent Application Publication
(10) Pub. No.: US 2024/0034765 A1
(43) Pub. Date: Feb. 1, 2024

(54) SCG2 NEUROPEPTIDES AND USES THEREOF

(71) Applicant: PRESIDENT AND FELLOWS OF HARVARD COLLEGE, Cambridge, MA (US)

(72) Inventors: Michael E. GREENBERG, Cambridge, MA (US); Ee-Lynn YAP, Cambridge, MA (US)

(73) Assignee: PRESIDENT AND FELLOWS OF HARVARD COLLEGE, Cambridge, MA (US)

(21) Appl. No.: 18/265,523

(22) PCT Filed: Dec. 6, 2021
(86) PCT No.: PCT/US2021/061950

§ 371 Date: Jun. 6, 2023

Related U.S. Application Data
(60) Provisional application No. 63/122,156, filed on Dec. 7, 2020.

(51) Int. Cl. C07K 14/575; C12N 15/63; G01N 33/68; A61P 25/28

(52) U.S. Cl. CPC C07K 14/575; C12N 15/63; G01N 33/6896; A61P 25/28; G01N 2800/2814; G01N 2800/52

(57) ABSTRACT

The technology described herein is directed to pharmaceutical compositions comprising at least one Scg2 neuropeptide, as well as cell culture media or kits comprising such Scg2 neuropeptides. Also described herein are nucleic acids, vectors, or viral vectors encoding at least one Scg2 neuropeptide. In further aspects, described herein are methods of treating a memory-associated disorder, a learning disability, a neurodegenerative disease or disorder, or epilepsy with a pharmaceutical composition, nucleic acid, vector, or viral vector as described herein. In further aspects, described herein are detection methods of memory-associated analytes, such as Scg2 neuropeptides.

### CROSS-REFERENCE TO RELATED APPLICATIONS

This application claims benefit under 35 U.S.C. § 119(e) of U.S. Provisional Application No. 63/122,156 filed Dec. 7, 2020, the contents of which are incorporated herein by reference in their entirety.

### GOVERNMENT SUPPORT

This invention was made with government support under NS112455, NS028829, NS115965, NS072030, NS089521, and NS007473 awarded by the National Institutes of Health. The government has certain rights in the invention.

### SEQUENCE LISTING

The instant application contains a Sequence Listing which has been submitted in ASCII format via EFS-Web and is hereby incorporated by reference in its entirety. Said ASCII copy, created on Dec. 6, 2021, is named 002806-099210WOPT_SL.txt and is 81,274 bytes in size.

### TECHNICAL FIELD

The technology described herein relates to methods and compositions for modulating cognitive function, and uses thereof.

### BACKGROUND

Neurons convert new experiences into stable representations in the brain to inform future actions. Mounting evidence suggests that sparse populations of neurons distributed across multiple regions of the brain form the neural substrates for a variety of behaviors. A hallmark of these active neuronal ensembles is the transient expression of a set of genes, termed the immediate early genes (IEGs), one of which encodes the Fos transcription factor (TF). Once activated by salient stimuli, Fos-expressing neurons undergo modifications that facilitate the encoding of specific features of an experience, such that subsequent reactivation of even a subset of these neurons is sufficient to elicit recall of the initial experience. Yet whether these neuronal ensembles in fact become persistently modified, and if so, the nature of these changes and their underlying molecular mechanisms, has remained unclear. Moreover, whether Fos induction, beyond serving as a proxy for recent neural activity, plays a causal role in coordinating circuit modifications required to encode an experience remains unresolved. Complicating progress in this regard is the fact that the Fos family of TFs (also known as Activator protein 1 (AP-1)) comprises seven at least partially functionally redundant members (Fos, Fosb, Fosl1, Fosl2, Jun, Junb, and Jund). See e.g., Josselyn & Tonegawa, Science 367, (2020); Tanaka et al. Science 361, 392-397, (2018); Greenberg & Ziff, Nature 311, 433-438 (1984); Yap & Greenberg, Neuron 100, 330-348, (2018); the contents of each of which are incorporated herein by reference in their entireties.

Fos-activated neurons in the hippocampal CA1 region have been shown to stably encode contextual information as compared to their non-Fos-activated counterparts. As recurrent excitatory connectivity is weak within CA1, pyramidal cells (PCs) are known to be regulated in concert either via their common excitatory inputs or through a local network of inhibitory γ-aminobutyric acid-releasing (GABAergic) interneurons (INs). Perisomatic-targeting INs, by virtue of their extensive axonal arborizations, are uniquely positioned to control spike frequency and duration in populations of PCs. In this regard, two functionally distinct forms of perisomatic inhibition have been described, mediated by parvalbumin (PV)-expressing INs or cholecystokinin-expressing (CCK)-INs. Whereas PV-INs display fast, non-adapting firing patterns and are predominantly activated in a feedforward fashion, CCK-expressing INs fire regular, adapting trains of action potentials and provide predominantly feedback inhibition. Perisomatic inhibition has also been shown to coordinate behavioral state-dependent network oscillations. For example, PV-INs regulate gamma rhythms, which are critical for transient synchrony of PCs, and both PV-INs and CCK-INs fire preferentially at different phases of theta, which have been associated with memory encoding or retrieval. There is thus a need to consider how inputs of each IN subtype are selectively modified onto Fos-activated neurons, in order to gain mechanistic insights into how experience alters the temporal dynamics of network function to support long-term memories. See e.g., Tanaka et al. (2018), supra; Freund & Katona, Neuron 56, 33-42 (2007); Klausberger et al., J Neurosci 25, 9782-9793 (2005); Bartos & Elgueta, J Physiol 590, 669-681 (2012); Ryan et al., Science 348, 1007-1013 (2015); Glickfeld & Scanziani, Nat Neurosci 9, 807-815 (2006); Hefft & Jonas, Nat Neurosci 8, 1319-1328 (2005); Buzsaki, Neuron 33, 325-340 (2002); Buzsaki & Wang, Annu Rev Neurosci 35, 203-225 (2012); Hasselmo & Stern, Neuroimage 85 Pt 2, 656-666 (2014); the contents of each of which are incorporated herein by reference in their entireties.

Overall, memory and learning are complicated neural processes. Many diseases, such as memory-associated disorders, learning disabilities, neurodegenerative diseases or disorders, or epilepsy, are associated with improper functioning of such processes. There is thus great need for therapies that can specifically modulate the associated neural pathways and circuits in order to treat such diseases.

### SUMMARY

The technology described herein is associated with the following pathway elucidated herein: a novel environment was shown to lead to Fos (TF) activation in CA1 pyramidal cells, which led to Scg2 expression and subsequent cleavage and expression of four Scg2 neuropeptides, which are 33-66 amino acids long. Such Scg2 neuropeptides lead to modulation and re-wiring of interneurons, including increased parvalbumin (PV)-expressing interneuron inhibition of the pyramidal cells (PCs) and decreased cholecystokinin-expressing (CCK) interneuron inhibition of the PC. Such interneuron modulation and re-wiring subsequently led to modulation of hippocampal gamma rhythms as well as pyramidal cell coupling to theta phase, which were associated with consolidation and/or retention of memories. As such, Scg2 neuropeptides (see e.g., FIG. 4F) are associated with neural pathways that lead to unexpected beneficial neurological effects, such as memory consolidation, memory retention, and learning. For example, knockout of Scg2 led to the loss of the beneficial neurological effects as described herein, whereas rescue or overexpression restored such effects (see e.g., FIG. 4-6, FIG. 14-16). Furthermore, a cleavage-deficient Scg2, which cannot be cleaved into the Scg2 neuropeptides, did not have the same effects as WT Scg2, further demonstrating the beneficial effects of the Scg2 neuropeptides (see e.g., FIG. 5I-5K, FIG. 15A-15I).

Accordingly, the technology described herein is directed to pharmaceutical compositions comprising at least one Scg2 neuropeptide, as well as cell culture media or kits comprising such Scg2 neuropeptides. Also described herein are nucleic acids, vectors, or viral vectors encoding at least one Scg2 neuropeptide. In further aspects, described herein are methods of treating a memory-associated disorder, a learning disability, a neurodegenerative disease or disorder, or epilepsy with a pharmaceutical composition, nucleic acid, vector, or viral vector as described herein. In further aspects, described herein are detection methods of memory-associated analytes, such as Scg2 neuropeptides.

In one aspect, described herein is a pharmaceutical composition comprising at least one secretogranin II (scg2) neuropeptide and a pharmaceutically acceptable carrier.

In some embodiments of any of the aspects, the pharmaceutical composition is formulated for delivery to the central nervous system (CNS).

In some embodiments of any of the aspects, the pharmaceutical composition is formulated for delivery across the blood-brain barrier (BBB).

In some embodiments of any of the aspects, the pharmaceutical composition is formulated for delivery to the brain.

In some embodiments of any of the aspects, the pharmaceutical composition is formulated for delivery to the hippocampus.

In some embodiments of any of the aspects, the pharmaceutical composition is formulated for delivery to pyramidal cells.

In some embodiments of any of the aspects, the formulation of the pharmaceutical composition is selected from the group consisting of: direct injection or infusion into the CNS; formulation as a solution comprising a carrier protein; formulation as a nanoparticle; formulation as a liposome; formulation as a nucleic acid; formulation as a CNS-tropic viral vector; formulation with or linkage to an agent that is endogenously transported across the BBB; formulation with or linkage to a cell penetrating peptide (CPP); formulation with or linkage to a BBB-shuttle; formulation with or linkage to an agent that increases permeability of the BBB.

In some embodiments of any of the aspects, the scg2 neuropeptide is a cleavage product of secretogranin II (scg2) polypeptide.

In some embodiments of any of the aspects, the scg2 polypeptide comprises SEQ ID NO: 4.

In some embodiments of any of the aspects, the scg2 neuropeptide, when present in the scg2 polypeptide, is flanked at its N-terminus and at its C-terminus by a dibasic cleavage residue.

In some embodiments of any of the aspects, the dibasic cleavage residue is selected from the group consisting of: arginine-lysine (RK); lysine-arginine (KR); and arginine-arginine (RR).

In some embodiments of any of the aspects, the dibasic cleavage residue is lysine-arginine (KR).

In some embodiments of any of the aspects, the dibasic cleavage residue is a specific cleavage site for a Pcsk1/2 protease.

In some embodiments of any of the aspects, the at least one scg2 neuropeptide is selected from the group consisting of: secretoneurin; EM66; manserin; and SgII.

In some embodiments of any of the aspects, the scg2 neuropeptide is secretoneurin.

In some embodiments of any of the aspects, the scg2 neuropeptide is EM66.

In some embodiments of any of the aspects, the scg2 neuropeptide is manserin.

In some embodiments of any of the aspects, the scg2 neuropeptide is SgII.

In some embodiments of any of the aspects, secretoneurin comprises

(SEQ ID NO: 5)
TNEIVEEQYTPQSLATLESVFQELGKLTGPNNQ.

In some embodiments of any of the aspects, EM66 comprises

(SEQ ID NO: 6)
ERMDEEQKLYTDDEDDIYKANNIAYEDVVGGEDWNPVEEKIESQTQEEV
RDSKENIEKNEQINDEM.

In some embodiments of any of the aspects, manserin comprises

(SEQ ID NO: 7)
VPGQGSSEDDLQEEEQIEQAIKEHLNQGSSQETDKLAPVS.

In some embodiments of any of the aspects, SgII comprises

(SEQ ID NO: 8)
FPVGPPKNDDTPNRQYWDEDLLMKVLEYLNQEKAEKGREHIA.

In some embodiments of any of the aspects, the scg2 neuropeptide comprises a human, mouse, rat, or chimpanzee scg2 neuropeptide or a chimera thereof.

In some embodiments of any of the aspects, the scg2 neuropeptide comprises a peptidomimetic.

In one aspect, described herein is a nucleic acid comprising at least one nucleic acid sequence encoding a secretogranin II (scg2) neuropeptide.

In some embodiments of any of the aspects, the scg2 neuropeptide is selected from the group consisting of: secretoneurin; EM66; manserin; and SgII.

In some embodiments of any of the aspects, the nucleic acid sequence encodes secretoneurin.

In some embodiments of any of the aspects, the nucleic acid sequence encodes EM66.

In some embodiments of any of the aspects, the nucleic acid sequence encodes manserin.

In some embodiments of any of the aspects, the nucleic acid sequence encodes SgII.

In some embodiments of any of the aspects, the nucleic acid sequence encoding secretoneurin comprises

(SEQ ID NO: 9)
ACAAATGAAATAGTGGAGGAACAATATACTCCTCAAAGCCTTGCTACAT
TGGAATCTGTCTTCCAAGAGCTGGGGAAACTGACAGGACCAAACAACCA
G.

In some embodiments of any of the aspects, the nucleic acid sequence encoding EM66 comprises

(SEQ ID NO: 10)
GAGAGGATGGATGAGGAGCAAAAACTTTATACGGATGATGAAGATGATA
TCTACAAGGCTAATAACATTGCCTATGAAGATGTGGTCGGGGGAGAAGA
CTGGAACCCAGTAGAGGAGAAAATAGAGAGTCAAACCCAGGAAGAGGTG
AGAGACAGCAAAGAGAATATAGAAAAAAATGAACAAATCAACGATGAGA
TG.

In some embodiments of any of the aspects, the nucleic acid sequence encoding manserin comprises

(SEQ ID NO: 11)
GTTCCTGGTCAAGGCTCATCTGAAGATGACCTGCAGGAAGAGGAACAAA
TTGAGCAGGCCATCAAAGAGCATTTGAATCAAGGCAGCTCTCAGGAGAC
TGACAAGCTGGCCCCGGTGAGC.

In some embodiments of any of the aspects, the nucleic acid sequence encoding SgII comprises

(SEQ ID NO: 12)
TTCCCTGTGGGGCCCCCGAAGAATGATGATACCCCAAATAGGCAGTACT
GGGATGAAGATCTGTTAATGAAAGTGCTGGAATACCTCAACCAAGAAAA
GGCAGAAAAGGGAAGGGAGCATATTGCT.

In one aspect, described herein is a vector comprising a nucleic acid as described herein.

In some embodiments of any of the aspects, the vector further comprises a promoter that is operatively linked to the nucleic acid sequence encoding the scg2 neuropeptide.

In some embodiments of any of the aspects, the promoter comprises an Activator protein 1 (AP-1) family driven promoter.

In some embodiments of any of the aspects, the promoter comprises a constitutive promoter.

In some embodiments of any of the aspects, the promoter comprises a nervous tissue-specific promoter.

In one aspect, described herein is a viral vector comprising a nucleic acid as described herein or a vector as described herein.

In some embodiments of any of the aspects, the viral vector is an adenovirus-associated virus (AAV).

In some embodiments of any of the aspects, the AAV is serotype AAV2/1.

In one aspect, described herein is a cell comprising a pharmaceutical composition as described herein, a nucleic acid as described herein, a vector as described herein, or a viral vector as described herein.

In some embodiments of any of the aspects, the cell is a neuronal cell.

In some embodiments of any of the aspects, the cell is a hippocampal cell.

In some embodiments of any of the aspects, the cell is a pyramidal cell.

In some embodiments of any of the aspects, the cell is a CA1 pyramidal cell.

In one aspect, described herein is a composition comprising a nucleic acid as described herein, a vector as described herein, a viral vector as described herein, or a cell as described herein, and a pharmaceutically acceptable carrier.

In one aspect, described herein is a method of increasing memory consolidation and/or memory retention, comprising administering an effective amount of a pharmaceutical composition as described herein, a nucleic acid as described herein, a vector as described herein, or a viral vector as described herein to a subject in need thereof.

In one aspect, described herein is a method of treating a memory-associated disorder, comprising administering an effective amount of a pharmaceutical composition as described herein, a nucleic acid as described herein, a vector as described herein, or a viral vector as described herein to a subject in need thereof.

In one aspect, described herein is a method of treating a learning disability, comprising administering an effective amount of a pharmaceutical composition as described herein, a nucleic acid as described herein, a vector as described herein, or a viral vector as described herein to a subject in need thereof.

In one aspect, described herein is a method of treating a neurodegenerative disease or disorder, comprising administering an effective amount of a pharmaceutical composition as described herein, a nucleic acid as described herein, a vector as described herein, or a viral vector as described herein to a subject in need thereof.

In one aspect, described herein is a method of treating epilepsy, comprising administering an effective amount of a pharmaceutical composition as described herein, a nucleic acid as described herein, a vector as described herein, or a viral vector as described herein.

In some embodiments of any of the aspects, the pharmaceutical composition, nucleic acid, vector, or viral vector is administered intracranially, epidurally, intrathecally, intraparenchymally, intraventricularly, or subarachnoidly.

In some embodiments of any of the aspects, the pharmaceutical composition, nucleic acid, vector, or viral vector is administered in a formulation that crosses the blood-brain barrier.

In some embodiments of any of the aspects, the scg2 neuropeptide binds to a G-protein coupled receptor (GPCR).

In some embodiments of any of the aspects, the administration modulates activity of interneurons in the central nervous system of the subject.

In some embodiments of any of the aspects, the administration modulates activity of interneurons in the hippocampus of the subject.

In some embodiments of any of the aspects, the administration modulates activity of γ-aminobutyric acid-releasing (GABAergic) interneurons in the CA1 region of the hippocampus of the subject.

In some embodiments of any of the aspects, the interneurons are parvalbumin-expressing interneurons (PV-IN) or cholecystokinin-expressing interneurons (CCK-IN).

In some embodiments of any of the aspects, the administration increases PV-IN perisomatic inhibitory activity on an associated pyramidal cell.

In some embodiments of any of the aspects, the administration decreases CCK-IN perisomatic inhibitory activity on an associated pyramidal cell.

In some embodiments of any of the aspects, the administration increases the power of fast gamma waves (60 Hz-90 Hz) in the CA1 region of the hippocampus.

In some embodiments of any of the aspects, the administration increases firing of pyramidal cells in the CA1 region of the hippocampus during the descending phase of the theta_{pyr} cycle.

In some embodiments of any of the aspects, the administration increases spatial learning of the subject by at least 10% compared to a subject that is not administered the pharmaceutical composition, nucleic acid, vector, or viral vector.

In some embodiments of any of the aspects, memory consolidation and/or memory retention is increased by at least 10% compared to a subject that is not administered the pharmaceutical composition, nucleic acid, vector, or viral vector.

In some embodiments of any of the aspects, the memory-associated disorder is a learning disability or a neurodegenerative disease or disorder.

In some embodiments of any of the aspects, the memory-associated disorder is selected from the group consisting of amnesia, dementia, Alzheimer's disease, mild cognitive impairment, vascular cognitive impairment, and hydrocephalus.

In some embodiments of any of the aspects, the learning disability is selected from the group consisting of dyscalculia, dysgraphia, dyslexia, a non-verbal leaning disability, an oral and/or written language disorder and specific reading comprehension deficit, attention deficit hyperactivity disorder (ADHD), attention deficit disorder (ADD), dyspraxia, an executive mal-functioning, an auditory processing disorder, a language processing disorder, and a visual perceptual/visual motor deficit.

In some embodiments of any of the aspects, the neurodegenerative disease or disorder is selected from the group consisting of Alzheimer's disease, Parkinson's disease, Huntington's disease, amyotrophic lateral sclerosis (ALS), frontotemporal dementia, chronic traumatic encephalopathy (CTE), multiple sclerosis, and neuroinflammation.

In some embodiments of any of the aspects, the epilepsy is selected from the group consisting of focal seizures without loss of consciousness (simple partial seizures); focal seizures with impaired awareness (complex partial seizures); absence seizures (petit mal seizures); tonic seizures; atonic seizures; clonic seizures; myoclonic seizures; and tonic-clonic seizures.

In one aspect, described herein is a method of diagnosing a memory-associated disorder, learning disability, neurodegenerative disease or disorder, or epilepsy in a subject; comprising: (a) obtaining a sample from the subject; (b) detecting the level of a memory-associated analyte in the sample; and (c) determining that the subject: (i) has or is at risk of developing a memory-associated disorder, learning disability neurodegenerative disease or disorder, or epilepsy if the analyte level is below a pre-determined level; or (ii) does not have or is not at risk of developing a memory-associated disorder, learning disability, neurodegenerative disease or disorder, or epilepsy if the analyte level is at or above a pre-determined level.

In some embodiments of any of the aspects, the method further comprises administering to the subject a pharmaceutical composition as described herein, a nucleic acid as described herein, a vector as described herein, or a viral vector as described herein, if the subject is determined to have or be at risk for developing a memory-associated disorder, learning disability, neurodegenerative disease or disorder, or epilepsy.

In one aspect, described herein is a method for detecting a memory-associated analyte in a sample from a subject comprising: (a) obtaining a sample from the subject; and (b) detecting the level of the memory-associated analyte in the sample.

In some embodiments of any of the aspects, the step of detecting the level of the memory-associated analyte comprises mRNA detection or polypeptide detection.

In some embodiments of any of the aspects, the mRNA detection comprises reverse transcription polymerase chain reaction (RT-PCR); quantitative RT-PCR; Northern blot analysis; differential gene expression; RNase protection assay; microarray based analysis; next-generation sequencing; or hybridization methods.

In some embodiments of any of the aspects, the polypeptide detection comprises immunoassays, Western blot; immunoprecipitation; enzyme-linked immunosorbent assay (ELISA); radioimmunological assay (RIA); sandwich assay; immunohistological staining; radioimmunometric assay; immunofluorescence assay; mass spectroscopy; or immunoelectrophoresis assay.

In one aspect, described herein is a method of increasing memory consolidation and/or memory retention in a subject in need thereof, comprising: (a) obtaining results detecting the level of a memory-associated analyte in a sample from the subject; and (b) administering to the subject: (i) a pharmaceutical composition as described herein, a nucleic acid as described herein, a vector as described herein, or a viral vector as described herein, if the analyte level is below a pre-determined level; or (ii) an alternative treatment, if the analyte level is at or above a pre-determined level.

In one aspect, described herein is a method of treating a memory-associated disorder in a subject in need thereof, comprising: (a) obtaining results detecting a memory-associated analyte in a sample from the subject; and (b) administering to the subject: (i) a pharmaceutical composition as described herein, a nucleic acid as described herein, a vector as described herein, or a viral vector as described herein, if the analyte level is below a pre-determined level; or (ii) an alternative treatment, if the analyte level is at or above a pre-determined level.

In one aspect, described herein is a method of treating a learning disability in a subject in need thereof, comprising: (a) obtaining results detecting a memory-associated analyte in a sample from the subject; and (b) administering to the subject: (i) a pharmaceutical composition as described herein, a nucleic acid as described herein, a vector as described herein, or a viral vector as described herein, if the analyte level is below a pre-determined level; or (ii) an alternative treatment, if the analyte level is at or above a pre-determined level.

In one aspect, described herein is a method of treating a neurodegenerative disease or disorder in a subject in need thereof, comprising: (a) obtaining results detecting the level of a memory-associated analyte in a sample from the subject; and (b) administering to the subject: (i) a pharmaceutical composition as described herein, a nucleic acid as described herein, a vector as described herein, or a viral vector as described herein, if the analyte level is below a pre-determined level; or (ii) an alternative treatment, if the analyte level is at or above a pre-determined level.

In one aspect, described herein is a method of treating epilepsy in a subject in need thereof, comprising: (a) obtaining results detecting the level of a memory-associated analyte in a sample from the subject; and (b) administering to the subject: (i) a pharmaceutical composition as described herein, a nucleic acid as described herein, a vector as described herein, or a viral vector as described herein, if the analyte level is below a pre-determined level; or (ii) an alternative treatment, if the analyte level is at or above a pre-determined level.

In some embodiments of any of the aspects, the sample is a cerebrospinal fluid sample or a CNS sample.

In some embodiments of any of the aspects, the memory-associated analyte is Scg2 mRNA, polypeptide, or neuropeptide.

In some embodiments of any of the aspects, the memory-associated analyte is Fos, Fosb, or Junb mRNA or polypeptide.

In some embodiments of any of the aspects, the memory-associated analyte is Fos+, Fosb+, or Junb+ neurons.

In one aspect, described herein is a cell culture medium comprising at least one Scg2 neuropeptide.

In one aspect, described herein is a method for culturing a neuron, comprising contacting the neuron with a cell culture medium as described herein.

### BRIEF DESCRIPTION OF THE DRAWINGS

FIG. 1A-1O is a series of schematics, images, and graphs showing bidirectional perisomatic inhibitory plasticity. FIG. 1A is a schematic of standard housing (Strd) or novel environment (NE). FIG. 1B is a schematic showing the experimental timeline and configuration of AAV-based activity reporter; nuclear mKate2 labeling is achieved with a nuclear localization signal (NLS) and temporally controlled via doxycycline (Dox). IEG stands for immediate early gene; pRAM stands for promoter robust activity marking; d2tTA stands for destabilized tetracycline transactivator; TRE stands for tTA-responsive element. FIG. 1C shows representative images depicting Fos-activated neurons (which fluoresced red) and PV-IN-specific channelrhodopsin-2 (ChR2, which fluoresced green) in the hippocampal CA1 region in mice exposed to standard housing or 2-3 days of NE. The bar graph of FIG. 1C shows the number of mKate2+ cells/mm² in Strd (N=13 mice) and NE conditions (N=10 mice). Scale: 100 μm. ****p=2.6×10⁻¹⁰. FIG. 1D shows a schematic of a Fos-activated CA1 PC and its perisomatic-targeting inputs (indicated with question marks) from PV-INs or CCK-INs. The schematic shows activity-induced gene expression kinetics. In the early wave, immediate early genes such as Fos are expressed. Fos subsequently activates late-response genes. FIG. 1E and FIG. 1I are schematics of the genetic strategy to introduce ChR2 into PV-INs (see e.g., FIG. 1E) or CCK-INs (see e.g., FIG. 1I) and measure light-evoked IPSCs. WT stands for wildtype; s.p. stands for stratum pyramidale; s.r. stands for stratum radiatum. FIG. 1F shows scatter plots of recorded pairs of mKate2⁻ neurons in Strd (left; n=51/6) or mKate2⁺ and mKate2⁻ pairs after 2-3 days NE conditions (right; n=58/7). Representative traces from a pair of neurons are shown; vertical bars above the left-most-end of traces depict light onset. Scale: 100 pA; 40 ms. FIG. 1G is a bar graph showing the mean PV-IPSC amplitudes from FIG. 1F. ****p=3.2×10⁻⁶. FIG. 1H is a bar graph showing the normalized differences in PV-IPSC amplitudes between pairs of neurons in FIG. 1F (see e.g., Methods in Example 1), ***p=3.4×10⁻⁴. FIG. 1J-FIG. 1L show a series of graphs performed as in FIG. 1F-FIG. 1H, but for CCK-IPSCs. Strd, n=60/7; NE, n=48/8. Scale: 100 pA; 40 ms. FIG. 1K: **p=5.5×10⁻³. FIG. 1L: *p=0.014. FIG. 1M shows the IN-to-CA1 PC paired recording configuration, representative traces, and uIPSC amplitudes for PV-IN (left) and CCK-IN (right) to CA1 PC pairs. As used herein, KA stands for kainic acid; Veh stands for vehicle. PV-IN to CA1 PC: vehicle, n=13/6; KA, n=19/7; **p=0.003. CCK-to-CA1: Veh., n=16/9; KA, n=16/4; **p=9.6×10⁻³. Scale: 30 mV (IN response); 20 pA (PC response); 20 ms. Mann-Whitney test (two-sided). FIG. 1N is a series of dot plots showing normalized differences in amplitudes of PV-IPSCs (left) and CCK-IPSC (Right) of pairs of untransduced (WT) and hM3D_{Gq} (mCherry⁺) neurons after 24 h treatment with vehicle or clozapine N-oxide (CNO). PV (Veh., n=16/5; CNO, n=16/7; **p=0.006); CCK (Veh., n=22/5; CNO, n=21/7; *p=0.014). FIG. 1O show a series of graphs performed as in FIG. 1N but with Kir2.1. Control was a non-conducting mutant (KirMut). Mice were exposed to 7-10 days NE conditions, a period over which many CA1 PCs expressed Fos (see e.g., FIG. 7C, FIG. 7D). PV (KirMut, n=18/3; Kir2.1, n=19/5; **p=0.007); CCK (KirMut, n=25/3; Kir2.1, n=17/4; *p=0.023). In, FIG. 1F, FIG. 1H, FIG. 1J, FIG. 1L, FIG. 1M-FIG. 1O, each open circle represents a pair of simultaneously recorded neurons. FIG. 1C, FIG. 1F-FIG. 1H, FIG. 1J-FIG. 1O show mean±standard error of the mean (SEM). In FIG. 1F, FIG. 1J, FIG. 1M-FIG. 1O, n is expressed as number of pairs/number of mice. Data are mean±s.e.m. in FIG. 1C, FIG. 1F-FIG. 1H, FIG. 1J-FIG. 1O. FIG. 1C, FIG. 1K, FIG. 1L, FIG. 1N, FIG. 1O used the two-sided t-test. FIG. 1G, FIG. 1K used ordinary one-way ANOVA, corrected for multiple comparisons.

FIG. 2A-2M is a series of schematics, images, and graphs showing the causal role of Fos family TFs. FIG. 2A is a schematic depicting possible AP-1 homo-dimers and heterodimers. FIG. 2B is a bar graph showing the mean fold-induction of each AP-1 member upon KCl-mediated depolarization in hippocampal neurons (bulk RNA-sequencing; see e.g., Methods in Example 1) showing significantly more induction of Fos (****p=9.1×10⁻⁵), Fosb (***p=0.008), and Junb (****p=2.2×10⁻⁷) compared to the four other factors. n=2 biological replicates. FIG. 2C shows a schematic of Fos^{fl/fl}; Fosb^{fl/fl}; Junb^{fl/fl} (FFJ) mice transduced with AAV to sparsely express Cre (which fluoresced red). Representative CA1 image is shown. Scale: 100 μm. FIG. 2D is a schematic show the recording configuration with stimulus electrode placement in the stratum pyramidale, to measure perisomatic eIPSCs, or stratum radiatum, for Schaffer-collateral eEPSCs or proximal dendritic eIPSCs. FIG. 2E-FIG. 2G are dot plots showing the normalized differences in the indicated pharmacologically-isolated current amplitudes between pairs of FFJ-WT and KO PCs. FIG. 2E shows perisomatic eIPSCs. FIG. 2F shows Schaffer collateral eEPSCs. FIG. 2G shows proximal dendritic eIPSCs. FIG. 2E: Veh., n=26/6; KA, n=33/7; **p=0.005, FIG. 2F: Veh., n=18/5; KA, n=17/4, FIG. 2G: Veh., n=30/4; KA, n=30/6. FIG. 2H is a schematic of the strategy to introduce ChR2 into PV-INs and sparse Cre into the CA1 of PV^{Flp}; FFJ mice. FIG. 2I shows scatter plots of recorded pairs of FFJ-WT and FFJ-KO CA1 PCs, in Strd (left; n=16/3) or 7-10d NE (right; n=20/3). Representative traces from pairs of neurons are shown; vertical bars above the left-most-end of traces depict light onset. Scale: 50 pA (left) or 100 pA (right); 40 ms. FIG. 2J shows a dot plot, which was performed as in FIG. 2E-FIG. 2G, but for the pairs depicted in FIG. 2I and 24 hours post-kainic acid (KA) treatment (n=19/3). *p=0.014 (NE); **p=0.002 (KA). FIG. 2J used an ordinary one-way ANOVA, corrected for multiple comparisons. FIG. 2K is a line graph showing the fraction of time spent swimming in target quadrant for FFJ-WTs (N=11 mice) and FFJ-KOs (N=12 mice). *p=0.014 (Day 4); 0.016 (Day 5), where day 1 is defined as the start of training FIG. 2L shows Example probe trial swim traces (top images), and mean probe trial occupancy maps, 5 cm bins (bottom images). FIG. 2M shows box plots of mean trial (left) speed and (right) path length; mice were tested as in FIG. 2K. In box plots, the center line shows median, box edges indicate top and bottom quartiles, whiskers extend to minimum and maximum values and + indicates an outlier. In FIG. 2E-FIG. 2G, FIG. 2I, FIG. 2J, each open circle represents a pair of simultaneously recorded neurons; n is expressed as number of pairs/number of mice. In FIG. 2E-FIG. 2G, FIG. 2I-FIG. 2K, data are mean±SEM. FIG. 2B, FIG. 2E-FIG. 2G, FIG. 2K, FIG. 2M use the two-sided t-test.

FIG. 3A-3H is a series of schematics, graphs, visualizations, and diagrams showing Fos targets in CA1 pyramidal neurons. FIG. 3A, FIG. 3C, and FIG. 3F are schematics showing the workflow for RIBOTAG, FFJ snRNA-seq, and Fos CUT&RUN (see e.g., Methods in Example 1).

FIG. 3B is a scatter plot showing CaMK2a-specific ARGs after 6 hour kainic acid treatment compared with vehicle conditions. Significantly different genes (dark grey); FDR≤0.005. CaMK2a-enriched (immunoprecipitated (IP) over input) genes are additionally indicated (see e.g., gene labels or medium grey points. Points represent mean±SE. n=4 mice per biological replicate; 3 biological replicates per condition. FIG. 3D shows a Uniform Manifold Approximation and Projection (UMAP) visualization of nuclei from Cre⁺ and control FFJ snRNA-seq with (Left) cell type information or (Right) genotype assignments overlaid. “Control”: Cre⁻ in untransduced control hemispheres; “Cre-GFP”: Cre⁺ in injected hemispheres; “Other”: Cre or Cre⁺ in injected hemispheres or Cre⁺ cells in untransduced control hemispheres, respectively. n=58,536 cells/6 mice. FIG. 3E shows a volcano plot of genes in the CA1 excitatory cluster. The γ-axis shows −log 10 Bonferroni-corrected p-values (two-sided Wilcoxon rank-sum,). Fold changes are calculated from Cre⁺ compared with control cells. Each point represents a gene detected in ≥5% of untransduced cells, where light grey points and horizontal dashed line indicate p≥0.05 (n=3,429); darker grey dashed vertical lines indicate fold change ≤20% in either direction (n=42), dark grey points were p<0.05 and fold change >20% (n=3,514). FIG. 3G is an aggregate plot showing spike-in normalized Fos coverage per bin averaged across all Fos peaks for CaMK2a-SUN1 CUT&RUN (see e.g., Methods in Example 1). IgG serves as a specificity control. n=1 mouse per biological replicate, 3 biological replicates per condition. FIG. 3H is a Venn diagram showing the intersection of significant CA1 PC-specific genes from CaMK2a-RIBOTAG (fold change≥2), FFJ snRNA-seq (fold change>20% decreased expression in FFJ-KO cells), and CUT&RUN (Fos peaks within 10 kb of the TSS). For the schematic images in FIG. 3C, see e.g., Franklin & Paxinos, The Mouse Brain in Stereotaxic Coordinates 3rd ed. (Academic Press/Elsevier, 2007); the content of which is incorporated herein by reference in its entirety.

FIG. 4A-4L is a series of schematics, images, and graphs showing the Fos-dependent effector of inhibition. FIG. 4A and FIG. 4B are schematics of the FlpOFF shRNA AAV construct (FIG. 4A) used for the recordings as depicted in FIG. 4B. CAG stands for CAG promoter; FRT stands for flippase recognition target (FRT) cassette; U6 stands for U6 promoter. FIG. 4C is a dot plot showing normalized differences in PV-IPSC amplitudes between pairs of shRNA⁻ and shRNA⁺ PCs after 24 h kainic acid treatment. Control, n=17/9; Inhba, n=15/4; Rgs2, n=20/3; Bdnf, n=26/10; Nptx2, n=16/3; Pcsk1, n=17/6; Scg2#1, n=17/7 (**p=0.002); Scg2#2, n=17/6 (*p=0.016). Ordinary one-way ANOVA was used with multiple comparisons correction. FIG. 4D is a scatter plot of recorded PV-IPSC amplitudes for the Scg2#1 shRNA shown in FIG. 4C. Representative traces from a pair of neurons are also shown; the vertical bar above the left-most-end of each trace denotes light onset. n=17/7. Scale: 100 pA; 40 ms. FIG. 4E is a dot plot as in FIG. 4C showing Scg2#1 shRNA in Strd (n=14/5) or 7-10d NE (n=16/4). *p=0.048. FIG. 4F shows a schematic of the Scg2 protein, depicting the four Scg2-derived neuropeptides and nine dibasic (KR, RK, or RR) cleavage residues. FIG. 4G is a dot plot showing Scg2 expression from CaMK2a-RIBOTAG in FIG. 3B showing induction and enrichment (immuno-precipitated (IP) mRNA over total input RNA) after 6 h KA treatment. FIG. 4H shows violin plots depicting Scg2 expression in CA1 PCs in Cre or ΔCre pyramidal cells (PAs) compared with the respective controls from FFJ snRNA-seq in FIG. 3E. TPT: tags per ten thousand. **** represents p=9.4×10⁻³⁰² and >20% decrease. Data are mean±2 standard deviation (SD). FIG. 4I shows tracks displaying Fos-binding sites surrounding the Scg2 locus from CUT&RUN in FIG. 3G. Y-axis shows spike-in normalized coverage scaled to maximum value (in brackets) observed at the displayed locus. FIG. 4J shows representative smRNA-FISH images of CA1 in Strd and 6 h NE mice, probing for Fos (which fluoresced magenta), mature Scg2 (which fluoresced red), and intron-targeting Scg2 (which fluoresced green) transcripts (see e.g., lower magnification shown in FIG. 13H). DAPI staining (which fluoresced blue) for DNA is also shown. Strd, N=4; NE, N=6 mice. Scale: 20 μm. FIG. 4K shows violin plots of the number of puncta per cell for smRNA-FISH in FIG. 4J. Dashed lines: medians and quartiles. Each point represents a cell. Strd, n=909; NE, n=1,548 cells. ****p=1×10⁻¹⁵. The top of FIG. 4L is a schematic showing the workflow of NE snRNA-seq. Mice were exposed to NE briefly (5 min), returned to Strd for 1 h or 6 h prior to CA1 dissection. The bottom of FIG. 4L shows violin plots of normalized gene expression in CA1 PCs (n=1,659 cells after downsampling). Strd, N=2 mice; NE (1 h, 6 h), N=4 mice each. Fos (****p=4.2×10⁻⁹; *p=0.025), Scg2 (****p=2.2×10⁻¹⁶; *p=0.032), Actb (*p=0.014). For FIG. 4C-FIG. 4E, each open circle represents a pair of simultaneously recorded neurons; n is expressed as number of pairs/number of mice. FIG. 4C-FIG. 4E, and FIG. 4G show Mean±SEM. FIG. 4E and FIG. 4K used a two-sided t-test. FIG. 4H and FIG. 4L used a Wilcoxon rank-sum (two-sided).

FIG. 5A-5K is a series of schematics, images, and graphs showing that Scg2 mediates bidirectional perisomatic inhibitory plasticity. FIG. 5A is a schematic depicting the strategy for generation of a Scg2^{fl/fl} mouse line using CRISPR/Cas9. CDS stands for coding sequence; UTR stands for untranslated region. FIG. 5B is a series of images showing smRNA-FISH validation of Scg2^{fl/fl} crossed to Emx1^{Cre}, in order to excise Scg2 in all excitatory cells. N=2 mice/line. Scale: 20 μm. FIG. 5C shows a schematic of the strategy to introduce ChR2 into PV-INs in PV^{Flp}; Scg2^{fl/fl} mice, in order to mark recently active cells with the viral activity reporter mKate2, and sparsely transduce Cre into CA1 PCs. FIG. 5D shows scatter plots of recorded mKate2⁻ (left; n=21/4) or mKate2⁺ (right; n=22/9) pairs of Scg2-WT and Scg2-KO neurons after 2-3d NE. Representative traces from pairs of neurons are also shown; the vertical bar above the left-most-end of each trace denotes light onset. Scale: 50 pA; 40 ms. FIG. 5E is a dot plot showing the normalized differences in PV-IPSC amplitudes between pairs of neurons in FIG. 5D and mKate2⁻ pairs from Strd (n=22/5). **p=0.004, ***p=1.4×10⁻⁴. Ordinary one-way ANOVA was used, with multiple comparisons correction. FIG. 5F shows a schematic of the pharmacological strategy used to isolate CCK-IPSCs in Scg2^{fl/fl} mice. The following treatments were used: NBQX, which stands for 2,3-dihydroxy-6-nitro-7-sulphamoyl-benzo(F)quinoxaline an is an antagonist of the α-amino-3-hydroxy-5-methyl-4-isoxazole-propionate (AMPA receptor); (R)-CPP which stands for 3-((R)-2-Carboxypiperazin-4-yl)-propyl-1-phosphonic acid and is an N-methyl-d-aspartate (NMDA) antagonist; and ω-agatoxin subtype IVA (a selective antagonist for P/Q-type calcium channels; to block PV-IPSCs). FIG. 5G and FIG. 5H, are as in FIG. 5D and FIG. 5E, respectively, but for CCK-IPSCs, rather than PV-INs. mKate2⁻, n=22/6; mKate2⁺, n=26/6. Scale: 100 pA; 40 ms. **p=0.001. FIG. 5I-FIG. 5K show testing of a cleavage-resistant form of Scg2 in which the nine dibasic sequences were mutated to alanine (9AA-Mut). As in FIG. 5E and FIG. 5H, FIG. 5I-FIG. 5K show normalized differences in PV-IPSC or CCK-IPSC amplitudes for pairs of neurons, as depicted in the schematics shown in FIG. 5I-FIG. 5J. FIG. 5I shows rescue with WT-Scg2 protein or 9AA-Mut-Scg2 protein after Scg2^{fl/fl} knockout; PV (WT, n=22/5; 9AA, n=23/4; ****p=1.2×10⁻⁵); CCK (WT, n=27/3; 9AA, n=23/4, **p=0.005). FIG. 5J-5K show overexpression of WT-Scg2 protein (FIG. 5J) or 9AA-Mut-Scg2 protein (FIG. 5K). For FIG. 5J, PV (n=20/5; **p=0.001); CCK (n=25/3; **p=0.004). For FIG. 5K, PV (n=19/4); CCK (n=16/3). In FIG. 5D, FIG. 5E, and FIG. 5G-FIG. 5K, each open circle represents a pair of simultaneously recorded neurons; mean±SEM is shown; n=number of pairs/mice. FIG. 5H and FIG. 5I used two-sided t-test. FIG. 5J and FIG. 5K used a one-sample t-test (two-sided) with hypothetical mean of 0.

FIG. 6A-6E is a series of schematics and graphs showing that Scg2 is required to maintain network rhythms in vivo. The left panel of FIG. 6A shows a schematic of silicon probe placement in CA1 pyramidal layer; and the right panel of FIG. 6A shows a schematic of the head-fixed awake-behaving setup. After AAV injections, mice were exposed to NE daily for 1-2 weeks before recordings. FIG. 6B is a line graph showing the normalized power spectra of network oscillations in running Scg2-WT (N=4 mice; AAV-ΔCre-GFP; black) or Scg2-KO (N=5 mice; AAV-Cre-GFP; grey); one session per mouse. AU stands for arbitrary units. FIG. 6C is a series of bar graphs showing the mean of the normalized power spectra within theta, slow gamma, and fast gamma bands during running as in FIG. 6B; Scg2-WT shown in dark grey and Scg2-KO shown in light grey. *p=0.009, using a two-sided t-test. FIG. 6D is a series of graphs showing the theta phase modulation of putative CA1 PCs. Two cycles of theta are shown (see e.g., bottom-most line graph of FIG. 6D). The top histograms of FIG. 6D show the mean spike-triggered theta phase distributions for Scg2-WT (dark grey, n=67 units) and Scg2-KO (light grey, n=103 units) units. ***p<0.001, using bootstrap significance test of difference between circular means of the two distributions; 1000 shuffles. The middle dot plots of FIG. 6D show the mean theta phase and mean resultant length for each unit (Scg2-WT in black; Scg2-KO in grey). The bottom line graphs of FIG. 6D show the fraction of spikes in each theta phase bin (10° bins) (Scg2-WT in black; Scg2-KO in grey). FIG. 6E shows a schematic model depicting the experience-dependent reorganization of perisomatic inhibitory networks upon Fos activation in CA1 PCs, where weights of PV-IN and CCK-IN synaptic inputs are bidirectionally modulated. FIG. 6B-FIG. 6D show mean±SEM. For the schematic image in FIG. 6A, see e.g., Franklin & Paxinos (2007), supra.

FIG. 7A-7G is a series of schematics, images, and graphs showing characterization of the novel environment paradigm, the AAV-based activity reporter mKate2, and the intersectional genetic strategy for CCK-INs. The top panels of FIG. 7A show representative immunostaining images of Fos and Npas4 in hippocampus sections obtained from mice housed under standard (Strd) conditions or exposed to novel environment (NE) for 6 h. Scale: 400 μm. The bottom panels show higher magnification of the insets indicated by the dotted line box. Scale: 100 μm. To immunostain for both Fos and Npas4 proteins in the same sections, mice where Fos or Npas4 had been endogenously-tagged with a FLAG-HA tag (Fos-FLAGHA and Npas4-FLAGHA) were used with a rat anti-hemagglutinin (HA) antibody, while the reciprocal protein was probed with a rabbit polyclonal antibody (see e.g., Methods in Example 1). FIG. 7B is a series of bar graphs. The left bar graph of FIG. 7B shows the number of Fos⁺ and Npas4⁺ nuclei in the CA1 of Strd or 6 h NE mice. Strd, N=6 mice; NE, N=6 mice. Note that within CAL significantly fewer Npas4⁺ cells were detected, indicating that the AAV-based activity reporter mKate2 mainly labels Fos-activated neurons. Two-sided t-test; ***p=1.6×10⁻⁴, *p=0.033. The right bar graph of FIG. 7B shows the quantification of number of Npas4⁺ cells that were also Fos+. FIG. 7C shows representative images of mKate2⁺ neurons across different timepoints and conditions as in FIG. 7D. An AAV encoding GFP was used as a control for the viral injections. Scale: 100 μm. FIG. 7D is a bar graph showing percentages of mKate2⁺ neurons over total number of DAPI cells (left γ-axis) or density of mKate2⁺ neurons (right γ-axis). The average percentages of mKate2⁺ neurons were 1%, 12%, 66% and 96% under Strd (N=13 mice), 2-3 d NE (N=10 mice, ***p=2.7×10⁻⁴), 7-10 d NE (N=15 mice, ****p<1×10⁻¹⁵), and 24 h post-KA injection (N=3 mice, ****p=7.3×10⁻¹⁰), respectively. Ordinary one-way ANOVA was used with multiple comparisons correction. Note that data for Strd and 2-3 d NE are replotted from FIG. 1C. FIG. 7E shows bar plots of additional electrophysiological parameters for mKate2⁻ and mKate2⁺ neurons. n=30 pairs/4 mice per group. Two-sided t-test was used; the tests were not significant (n.s.) for all parameters. FIG. 7F shows a schematic of the intersectional genetic strategy involving Dlx5/6^{Flp}; CCK^{Cre} mice transduced with a dual Cre/Flp recombinase-dependent ChR2^{EYFP} fusion protein necessary to target specifically CCK-INs. FIG. 7F also shows representative immunostaining for PV, which fluoresced in magenta, and ChR2^{EYFP}, which fluoresced green. The bar graph in FIG. 7F shows that the percentage of ChR2⁺ cells in the CA1 field showing overlap with PV expression is low, indicating that the Dlx5/6^{Flp}; CCK^{Cre} line is suited for genetic targeting of CCK-INs. N=4 mice. Scale: 40 μm. FIG. 7G shows a representative image of the CA1 region of CCK^{Cre} mice transduced with AAV encoding Cre-dependent EYFP, depicting widespread EYFP expression in the CA1 and underscoring the necessity of the intersectional strategy in FIG. 7F for targeting CCK-INs specifically. N=2 mice. Scale: 100 μm. FIG. 7B and FIG. 7D-FIG. 7F show mean±SEM. For the schematic images in FIG. 7F-7G, see e.g., Franklin & Paxinos (2007), supra.

FIG. 8A-8Q is a series of schematics, images, and graphs showing IN-to-CA1 PC paired recordings and cell health parameters in 24 h post-KA condition. FIG. 8A and FIG. 8G are schematics of the genetic strategy to label PV-INs (PV^{Cre}; Ai14; FIG. 8A) or CCK-INs (D1×5/6^{Flp}; CCK^{Cre}; Ai65; FIG. 8G). FIG. 8B and FIG. 8H are representative images of tdTomato fluorescence in the CA1 field. Scale: 100 μm. N=2 mice per line. FIG. 8C and FIG. 8I are bar graphs showing quantification of the fraction of (FIG. 8C) PV- or (FIG. 8I) CCK-to-CA1 PC synaptically-connected pairs from the overall number of pairs recorded in both vehicle (Veh.) and 24 h post-KA mice. In FIG. 8C, Veh., n=13/22; KA, n=19/30; in FIG. 8I, Veh., n=16/40; KA, n=16/3, where n=number of connections/total pairs. FIG. 8D and FIG. 8J are dot plots showing quantification of the maximum firing rate of (FIG. 8D) PV- or (FIG. 8J) CCK-INs from connected pairs. In FIG. 8D, Veh., n=10/6; KA, n=14/7; in FIG. 8J, Veh., n=15/9; KA, n=14/4, where n=number of cells/number of mice. FIG. 8E and FIG. 8K are dot plots showing quantification of spike adaptation ratio of (FIG. 8E) PV- or (FIG. 8J) CCK-INs from connected pairs as in FIG. 8D and FIG. 8J, respectively. FIG. 8F and FIG. 8L are line graphs showing quantification of paired pulse ratios (PPRs) of uIPSCs at the indicated interstimulus intervals (ISI) for (FIG. 8F) PV- (Veh., n=13/6; KA, n=19/7) or (FIG. 8L) CCK- (Veh., n=16/9; KA, n=16/4) to-CA1 PC connected pairs, where n=number of pairs/number of mice. Two-sided t-tests were performed at each ISI or for all ISIs comparing Veh. and 24 h post-KA conditions; *p=0.039, ****p=4.4×10⁻⁵. FIG. 8M and FIG. 8N show representative hippocampal images from (FIG. 8M) Veh. and (FIG. 8N) 24 h post-KA conditions. Sections were immunostained for NeuN (which fluoresced green) and cleaved-caspase 3 (which fluoresced red), and counterstained with Hoechst (which fluoresced blue). Scale: 200 μm (left); 100 μm (right, CA1 field). N=2 mice per condition. FIG. 8O-FIG. 8Q are bar graphs showing the quantification of (FIG. 8O) Hoechst⁺ nuclei, (FIG. 8P) NeuN⁺ nuclei, and (FIG. 8Q) Cleaved-caspase⁺ cells per 40-μm section in all layers of CAL Results indicate that KA injection did not induce cell death within 24 h. Veh. and KA, n=10 sections/2 mice, respectively. In FIG. 8O-FIG. 8Q, vehicle control is indicated by black bars, and 24-h post-KA is indicated by grey bars. FIG. 8D-FIG. 8F, FIG. 8J-FIG. 8L, and FIG. 8O-FIG. 8Q show mean±SEM.

FIG. 9A-9G is a series of schematics and graphs showing that the chemogenetic activation of CA1 PCs recapitulated bidirectional changes in perisomatic inhibition, while silencing of CA1 PCs led to inverse effects. The top panels of FIG. 9A-FIG. 9D, FIG. 9F, and FIG. 9G each show a schematic of the recording configuration. The bottom panels of FIG. 9A, FIG. 9C, FIG. 9D, and FIG. 9F show scatter plots of PV-IPSCs, and the bottom panels of FIG. 9B and FIG. 9G), show CCK-IPSCs; the bottom panels of FIG. 9A-FIG. 9D, FIG. 9F, and FIG. 9G were each recorded from untransduced WT and the indicated viral-transduced neighboring CA1 PCs. In FIG. 9A, Veh., n=16/5; CNO, n=16/7. In FIG. 9B, Veh., n=22/5; CNO, n=21/7. In FIG. 9C, CNO, n=16/4. In, FIG. 9D, CNO, n=8/3 (note that FIG. 9D shows pairs of untransduced cells). In FIG. 9F, KirMut, n=18/3; Kir2.1, n=19/5. In FIG. 9G, KirMut, n=25/3; Kir2.1, n=17/4. In FIG. 9A-FIG. 9D, FIG. 9F, and FIG. 9G, n=number of pairs/number of mice, and each open circle represents a pair of simultaneously recorded neurons, with closed circles representing mean±SEM. FIG. 9E shows a representative trace of spikes detected from a CA1 PC in cell-attached mode in a slice after bath application of CNO. As expected, addition of CNO led to firing rate increases in hM3D_{Gq}-expressing neurons, providing further data that CNO intraperitoneal injection in mice in vivo chemogenetically activates hM3D_{Gq}-expressing neurons in the CAL N=3 cells/3 mice. Scale: 50 pA, 60 s.

FIG. 10A-10O is a series of schematics, images, and graphs showing validation of Fos^{fl/fl}; Fosb^{fl/fl}; Junb^{fl/fl} (FFJ) mouse line and additional electrophysiological parameters in FFJ-WT and KO cells. FIG. 10A is a schematic representation of the AP-1 members conditionally deleted in FFJ line. FIG. 10B and FIG. 10C show representative images of smRNA-FISH, validating loss of Fos and Fosb (and Junb in FIG. 10C) upon Cre expression in the CA1 field of 1-1.5 h post-KA-injected FFJ mice (see e.g., asterisk-marked cells). Scale: 20 μm. N=4 mice. FIG. 10D is a bar graph showing the normalized pixel intensity for Cre-negative and Cre-positive cells. Each point represents the average for individual sections across N=4 mice. Fos, ***p=7 0.7×10⁻⁴; Fosb, *p=0.031; Junb, *p=0.047. FIG. 10E shows scatter plots of normalized pixel intensities of Cre signal against Fos, Fosb, or Junb signals for each cell. Pearson correlation coefficients (r) are shown. Fos, n=315; Fosb, n=86; Junb, n=229 cells from N=4 mice. FIG. 10F shows representative images of Cre-injected sections immunostained for Fos, Fosb, Junb, and Npas4 proteins in the CA1 field of 3 h post-KA-injected FFJ mice. Scale: 100 μm. N=3 mice. FIG. 10G, FIG. 10J, and FIG. 10M are schematics of stimulus electrode placement in the stratum pyramidale to stimulate perisomatic inhibitory axons (FIG. 10G), the stratum radiatum to stimulate Schaffer collaterals (FIG. 10J), or proximal dendritic inhibitory axons (FIG. 10M). FIG. 10H, FIG. 10K, FIG. 10N are scatterplots of recorded pairs of FFJ-WT and FFJ-KO CA1 PCs in 24 h post-vehicle (left graph) or -KA injected (right graph) mice. In FIG. 10H, Veh., n=26/6; KA, n=33/7. In FIG. 10K, Veh., n=18/5; KA, n=17/4. In FIG. 10N, Veh., n=30/4; KA, n=30/6. FIG. 10I, FIG. 10L, and FIG. 10O are line graphs showing quantification of PPRs for the indicated currents. In FIG. 10I, Veh., n=17/3; KA, n=18/4. In FIG. 10L, Veh., n=18/5; KA, n=17/4. In FIG. 10O, Veh., n=19/2; KA, n=26/5. In FIG. 10H, FIG. 10I, FIG. 10K, FIG. 10L, FIG. 10N, and FIG. 10O, n=number of pairs/mice. FIG. 10D, FIG. 10H, FIG. 10I, FIG. 10K, FIG. 10L, FIG. 10N, and FIG. 10O show mean±SEM.

FIG. 11A-11H is a series of graphs, visualizations, and heatmaps showing RNA-sequencing that was used to identify CA1 pyramidal neuron-specific Fos targets. FIG. 11A. is a scatter plot showing PV-specific activity-regulated genes (ARGs) identified by comparing 6 h post-KA to vehicle-injected conditions. Significantly different genes are shown in medium grey; FDR 0.005. PV-enriched (IP over input) genes are shown in dark grey. Points represent mean±SE. n=9-10 mice/biological replicate; 4 biological replicates per condition. FIG. 11B. is a uniform manifold approximation and projection (UMAP) visualization of IN subtypes using only Gad2-expressing (“Inhibitory”) cells from FIG. 3C. FIG. 11C is a UMAP visualization of ΔCre⁺ and respective control nuclei with (left panel) cell type information or (right panel) genotype assignments overlaid. “Control”: ΔCre⁻ in control hemispheres; “ΔCre-GFP”: ΔCre⁺ in injected hemispheres; “Other”: ΔCre⁻ or ΔCre⁺ in injected or control hemispheres, respectively. n=25,214 cells/4 mice. FIG. 11D shows violin plots of quality control metrics for each transcriptionally distinct cell type identified by snRNA-seq in both Cre⁺ and ΔCre⁺ (“Del”) samples as in FIG. 11C and FIG. 3D. The top panel of FIG. 11D shows the number of unique genes per cell; the middle panel shows the number of RNA molecules per cell; and the bottom panel shows the percentage of reads that map to mitochondrial genome. FIG. 11E shows violin plots depicting CA1 PC-specific expression of Fos (****p=9.7×10⁻¹²⁷), Fosb, Junb (****p=7.2×10⁻²⁶; *p=0.003), and viral-derived WPRE (****p=0). Note that the design of the FFJ line renders snRNA-seq validation of excision of Fosb and Junb suboptimal (see e.g., FIG. 10B-FIG. 10F and Methods of Example 1). TPT stands for tags per ten thousand. FIG. 11F is a strip plot displaying differential gene expression (DGE) between Cre and control samples for each transcriptionally distinct cell type. The lightest grey points represent non-significant genes; the darker grey points represent significant genes (Bonferroni-corrected p-value <0.05, with average natural log FC >20%). FIG. 11G is a heatmap depicting normalized gene expression values from 100 randomly selected cells from each indicated cell type identity. Genes are cell-type-enriched AP-1 targets downregulated by at least 20% with loss of AP-1, and whose expression is detected in at least 25% of untransduced cells. FIG. 11H is a volcano plot of shuffled data where Cre and control CA1 excitatory nuclei were randomly assigned between two groups, showing no significant gene expression differences (light grey; Bonferroni-corrected p-value >0.05), thus further indicating that the expression differences observed between Cre⁺ and control were due to presence of Cre. FIG. 11D and FIG. 11E show Mean±2 SD. FIG. 11E-FIG. 11H used Wilcoxon rank-sum (two-sided).

FIG. 12A-12K is a series of tables, graphs, schematics, and tracks showing that CaMK2a-Sun1 Fos CUT&RUN revealed Fos binding sites across genome. FIG. 12A is a series of tables showing the pairwise Pearson correlation between CaMK2a-Sun1 Fos CUT&RUN biological replicates for each antibody and stimulus condition. FIG. 12B shows a histogram plotting distribution of distances between CaMK2a-Sun1 Fos CUT&RUN peaks and the nearest REFSEQ transcription start site (TSS). Peaks with a distance of 0 overlap the TSS. ˜90% of Fos-bound sites were distal to the TSS; see e.g., Malik et al., Nat Neurosci 17, 1330-1339, (2014), the contents of which are incorporated herein by reference in their entirety. FIG. 12C-FIG. 12E are histograms plotting the distributions of distances between the TSS of (FIG. 12C) all REFSEQ genes, (FIG. 12D) CaMK2a-RIBOTAG ARGs, or (FIG. 12E) CA1 excitatory genes downregulated with AP-1 loss (FFJ snRNA-seq), and the nearest Fos binding site. A distance of 0 indicates overlap of a Fos peak with the TSS. Notably, both CaMK2a-specific ARGs (FIG. 12D) and putative AP-1 targets downregulated with AP-1 loss in FFJ snRNA-seq (FIG. 12E) were significantly enriched for Fos-bound sites, which were significantly closer to the TSS when compared to all genes (FIG. 12C) (p<2.2×10⁻¹⁶, Wilcoxon rank-sum, two-sided), providing further data that these genes are direct targets of Fos. FIG. 12F is a schematic showing the top three enriched motifs identified by MEME-ChIP from CaMK2a-Sun1 Fos CUT&RUN peaks. E-values and matching transcription factor motifs are displayed to the right of each enriched motif. Fos CUT&RUN peaks identified therefore showed significant enrichment for the AP-1 motif. SEQ ID NO: 21, nTGAnTCA, was identified as significantly similar to the motif for ATF3 and FOS/AP-1 family members JUNB, FOSL2, FOSL1, JUN, and FOSB. SEQ ID NO: 22, rGrAA, where “r” indicates G or A, was identified as significantly similar to the motif for STA5A, STA5B, and STAT2. SEQ ID NO: 23, CnCCCAC was identified as significantly similar to the motif for EGR2, KLF4, SALL4, GLI1, and KLF8. FIG. 12G-FIG. 12K show tracks displaying Fos or IgG binding under 2-3 h post-vehicle or KA conditions for genomic regions surrounding the (FIG. 12G) Bdnf, (FIG. 12H) Inhba, (FIG. 12I) Rgs2, (FIG. 12J) Nptx2, or (FIG. 12K) Pcsk1 genes (see e.g., FIG. 4I for Scg2). Y-axis shows spike-in normalized CUT&RUN coverage. Tracks are scaled to the maximum value observed for all samples for the displayed genomic locus, shown in brackets.

FIG. 13A-13H is a series of tables, graphs, blots, and images showing the analyses of AP-1-regulated candidate genes to identify molecular effector of bidirectional perisomatic inhibitory plasticity. FIG. 13A is a table of the high-confidence AP-1-regulated candidate genes analyzed and their known functions. FIG. 13B is a bar graph showing RT-qPCR validation of shRNA efficacy using cultured hippocampal neurons transduced with lentivirus encoding the indicated shRNA. n=3 biological replicates for each shRNA. Mean±SEM. FIG. 13C shows Western blot confirmation of the efficacy of the FlpOFF shRNA strategy, where Bdnf shRNA-containing plasmid was transfected in 293T cells along with Bdnf-myc, and excision of the shRNA expression cassette via introduction of Flp recombinase was confirmed. Loading controls (Gapdh) were run on a separate blot (see e.g., FIG. 18A for full scans). 100-ng or 500-ng transfections of indicated u6-plasmid were loaded side-by-side on blot. n=2 biological replicates. FIG. 13D-FIG. 13F are scatterplots of recorded PV-IPSC amplitudes from untransduced shRNA⁻ (“Control”) and neighboring shRNA⁺CA1 PCs from mice 24 h post-KA injection. The shRNA target is shown on the γ-axis. In FIG. 13D, scrambled control, n=17/9; Inhba, n=15/4; Rgs2, n=20/3; Bdnf, n=26/10; Nptx2, n=16/3; Pcsk1, n=17/6. In FIG. 13E, Scg2 shRNA#2, n=17/6; representative traces from a pair of neurons are also shown; the vertical bas above the left-most-end of each trace denotes light onset. Scale: 100 pA, 40 ms. In FIG. 13F, Scg2 shRNA#1, Strd, n=14/5; 7-10 d NE, n=16/4, where n=number of pairs/number of mice. In FIG. 13D-FIG. 13F, each open circle represents a pair of simultaneously recorded neurons; closed circles represent mean±SEM. FIG. 13G shows smRNA-FISH scatter plots as in FIG. 4K, depicting the correlation between Fos and (left plot) Scg2 intron or (right plot) Scg2 mRNA expression. Each point represents the mean number of Scg2 puncta/cell within a bin, with a bin width of 1 Fos punctum/cell. Pearson correlation coefficients (r) are shown. FIG. 13H shows lower magnification images of smRNA-FISH as in FIG. 4J. Scale: 100 lam.

FIG. 14A-14G is a series of schematics and graphs showing that Scg2 is a molecular effector of bidirectional perisomatic inhibitory plasticity. FIG. 14A is a series of bar graphs showing RT-qPCR validation of Scg2^{fl/fl} conditional knockout line, where normalized (left plot) Scg2 and (right plot) Fos RNA levels in cultured hippocampal neurons derived from Scg2^{fl/fl} mice are shown. Cultures were transduced with lentiviral Cre or ΔCre and the membrane was depolarized with KCl for h or 6 h. n=3 biological replicates. Mean±SEM. Two-sided t-test, **p=0.002. FIG. 14B is a schematic of the intersectional genetic strategy to introduce ChR2 into CCK-INs and sparsely introduce shRNAs specifically into CA1 PCs of Dlx5/6^{Flp}; CCK^{Cre} mice. FIG. 14C is a dot plot showing the normalized differences in CCK-IPSC amplitudes between pairs of Scg2 shRNA⁻ and shRNA⁺ PCs depicted in FIG. 14D-FIG. 14F. Strd, n=30/4; NE, n=24/3; KA, n=19/4. Ordinary one-way ANOVA was used, with multiple comparisons correction; NE, **p=0.005; KA, **p=0.002. FIG. 14D-FIG. 14F are scatter plots of CCK-IPSC amplitudes of pairs as in FIG. 14C. Representative traces from pairs of neurons shown; vertical bars above the left-most-end of traces depict light onset. Scale: 100 pA, 40 ms. The top panel of FIG. 14G is a schematic of the recording configuration. Scatter plots of PV-IPSC (bottom left graphs of FIG. 14G) or CCK-IPSC (bottom right graphs of FIG. 14G) amplitudes recorded from pairs of neurons, of which one was untransduced (WT) and the other expressed a Scg2 shRNA with an shRNA-resistant full-length Scg2 rescue construct. Normalized differences in IPSC amplitudes between pairs of neurons are shown to the right of each scatter plot. PV, n=19/6; CCK, n=19/4. One-sample t-test (two-sided) was used with hypothetical mean of 0, *p=0.011. In FIG. 14C-FIG. 14G, each open circle represents a pair of simultaneously recorded neurons; closed circles represent mean±SEM; n=number of pairs/number of mice.

FIG. 15A-15I is a series of schematics, graphs, and blots showing that a series of rescue and overexpression analyses indicate a critical role for the processing of Scg2. FIG. 15A and FIG. 15B are scatter plots of PV-IPSC (FIG. 15A) and CCK-IPSC (FIG. 15B) amplitudes recorded from mKate2⁺ pairs that are either Cre (WT) or Cre⁺ (KO). Scg2-KO neurons also expressed a Cre-dependent full-length Scg2 construct (Rescue WT) to rescue the loss of Scg2. PV, n=22/5; CCK, n=27/3. FIG. 15C and FIG. 15D were as in FIG. 15A and FIG. 15B, respectively, but using a Cre-dependent non-cleavable Scg2 mutant (Rescue 9AA) instead, which failed to rescue the loss of Scg2. PV, n=23/4; CCK, n=23/4. FIG. 15E and FIG. 15F are scatter plots of PV-IPSC (FIG. 15E) and CCK-IPSC (FIG. 15F) amplitudes recorded from untransduced (WT) and neighboring full-length Scg2-overexpressing CA1 PCs (OE WT), showing that gain-of-function of Scg2 was sufficient to induce bidirectional perisomatic inhibitory plasticity in the absence of neural activity. PV, n=20/5; CCK, n=25/3. FIG. 15G shows Western blot confirmation of stable expression of Scg2 and the non-cleavable Scg2 mutant (9AA-Mutant) constructs containing an HA-tag in 293T cells. Expression levels were measured by immunoblot analysis with HA antibody. Loading controls (Gapdh) were run on a separate blot (see e.g., FIG. 18B for full scans). n=2 biological replicates. FIG. 15H and FIG. 15I were as in FIG. 15E and FIG. 15F, respectively, but with overexpression of the non-cleavable Scg2 mutant (9AA Mutant) instead, which failed to induce changes in inhibition. PV, n=19/4; CCK, n=16/3. In FIG. 15A-FIG. 15F, FIG. 15H, and FIG. 15I, each open circle represents a pair of simultaneously recorded neurons; closed circles represent mean±SEM; n=number of pairs/number of mice.

FIG. 16A-16G is a series of schematics, images, and graphs showing the silicon probe recordings in Scg2-WT and Scg2-KO mice to assess effects on network oscillations. The left panel of FIG. 16A shows a schematic of the stereotaxic injection and recording site in CA1 pyramidal layer. The right panel of FIG. 16A shows a representative image of silicon probe placement in CA1 pyramidal layer with Cre-GFP (which fluoresced green) and Dil (a lipophilic membrane stain, which fluoresced red). N=4 mice. Scale: 200 μm. FIG. 16B is a line graph showing normalized power spectra of network oscillations in Scg2-WT or KO mice during stationary periods. Average (mean±SEM) across Scg2-WT (black, N=4) or Scg2-KO (grey, N=5) mice, one session per mouse. FIG. 16C is a series of bar graphs showing the mean of the normalized power spectra within theta, slow gamma, and fast gamma bands during stationary periods, as shown in FIG. 16B, using a two-sided t-test, * p=0.037 and showing mean±SEM. FIG. 16D is a cumulative histogram of the mean firing rate for all Scg2-WT and Scg2-KO units. Mean firing rate was not significantly different (two-sided t-test, p=0.2138). Scg2-WT (n=67 units) and Scg2-KO (n=103 units). FIG. 16E shows an example local field potential (LFP), single-unit activity, and running speed in a Scg2-WT mouse. From top to bottom in FIG. 16E: denoised and downsampled LFP; 4-12 Hz bandpass filtered LFP; population spiking activity raster plot; and smoothed running speed. FIG. 16F shows an expanded snippet of the data from the example in FIG. 16E. From top to bottom in FIG. 16F: denoised and downsampled LFP; 4-12 Hz bandpass filtered LFP; and population spiking activity raster plot. FIG. 16G was as in FIG. 16F, but with example data from a Scg2-KO mouse. For the schematic image in FIG. 16A, see e.g., Franklin & Paxinos (2007), supra.

FIG. 17A-17B is a series of plots showing the gating strategy for flow cytometry analysis of data (see e.g., data shown in FIG. 3F-3G). Singlet DRAQS-positive nuclei were gated based on linearly proportional area and height signal for DRAQS. FIG. 17A shows DRAQS-stained nuclei from wild-type mice (no Sun1-GFP label), which were used to establish the GFP-positive gate for Sun1-GFP-positive nuclei. FIG. 17B shows that the gate from FIG. 17A was used to isolate Sun1-GFP-positive nuclei from the singlet DRAQS-positive population from CaMK2aCre; LSL-Sun1-sfGFP-Myc mice.

FIG. 18A-18B shows full scans of blots (see e.g., selected areas of blots in FIG. 13C and FIG. 15G). FIG. 18A shows Western blot confirmation of the efficacy of the Flp-OFF shRNA strategy, where Bdnf shRNA-containing plasmid was transfected in 293T cells along with BDNF-MYC, and excision of the shRNA expression cassette by introduction of Flp recombinase was confirmed. Loading controls (GAPDH) were run on a separate blot. Insets are cropped images shown in FIG. 13C. FIG. 18B shows Western blot confirmation of stable expression of SCG2 and the non-cleavable SCG2 9AA mutant (Mut) constructs containing an HA-tag in 293T cells. Samples in all other lanes not discussed herein. Expression levels were measured by immunoblot analysis with HA antibody. Loading controls (GAPDH) were run on a separate blot. Insets are cropped images shown in FIG. 15G.

### DETAILED DESCRIPTION

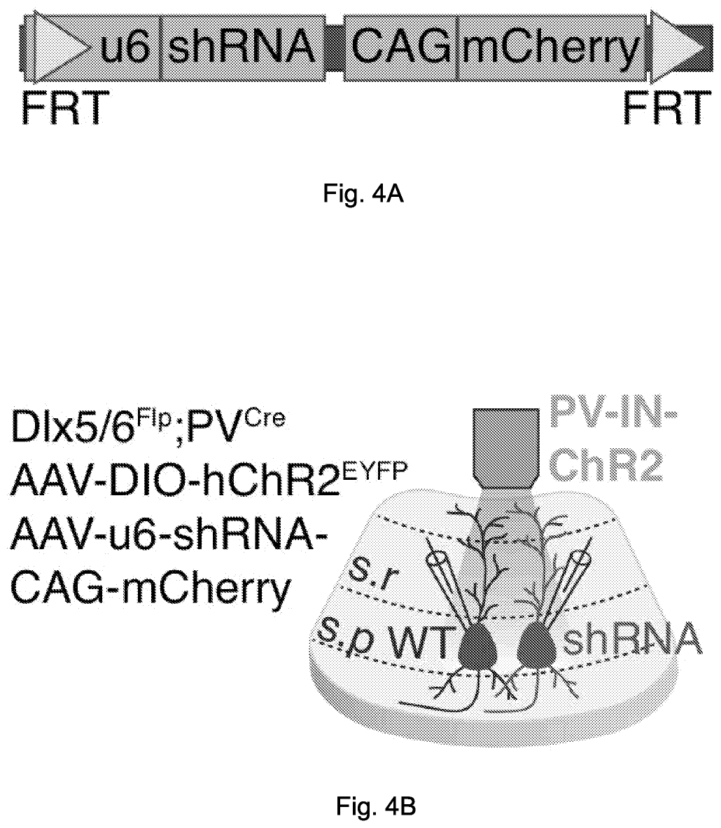

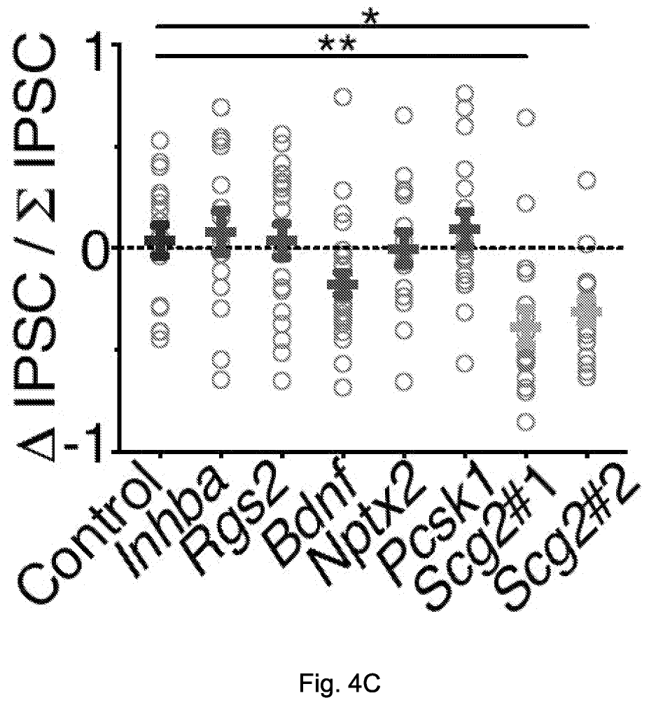

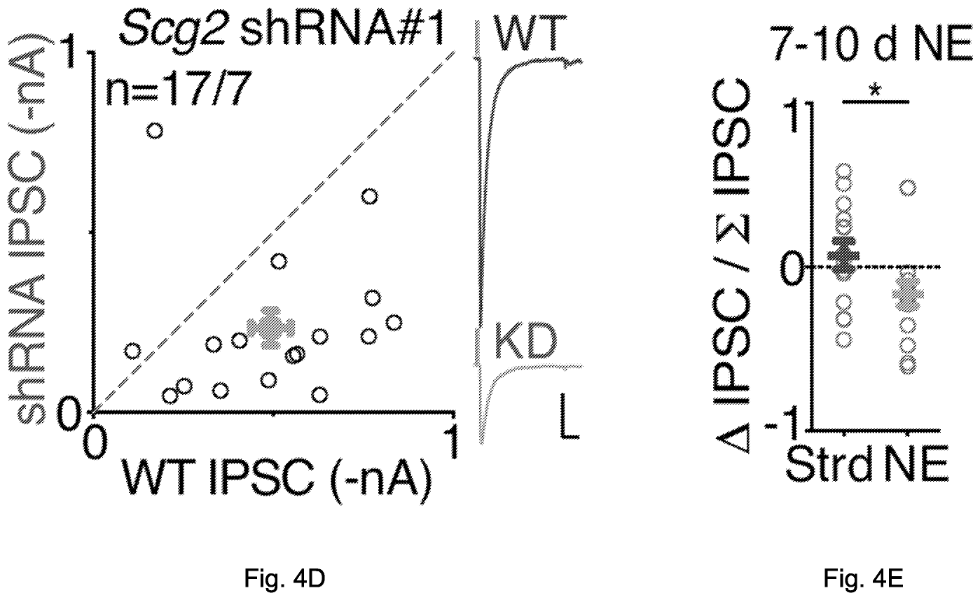

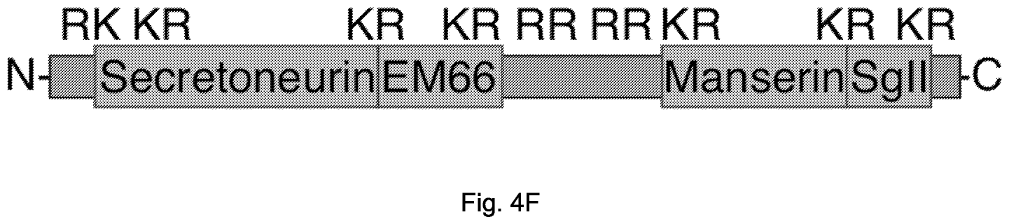

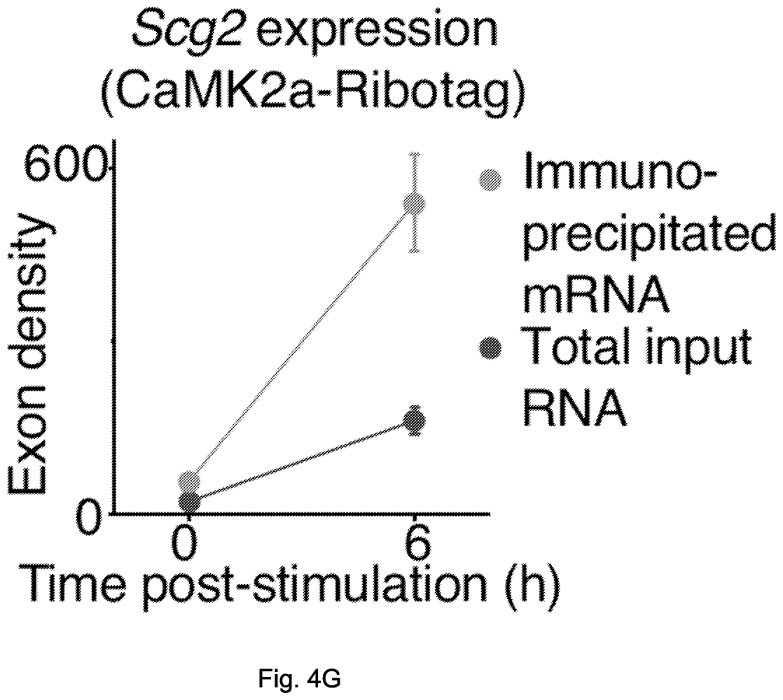

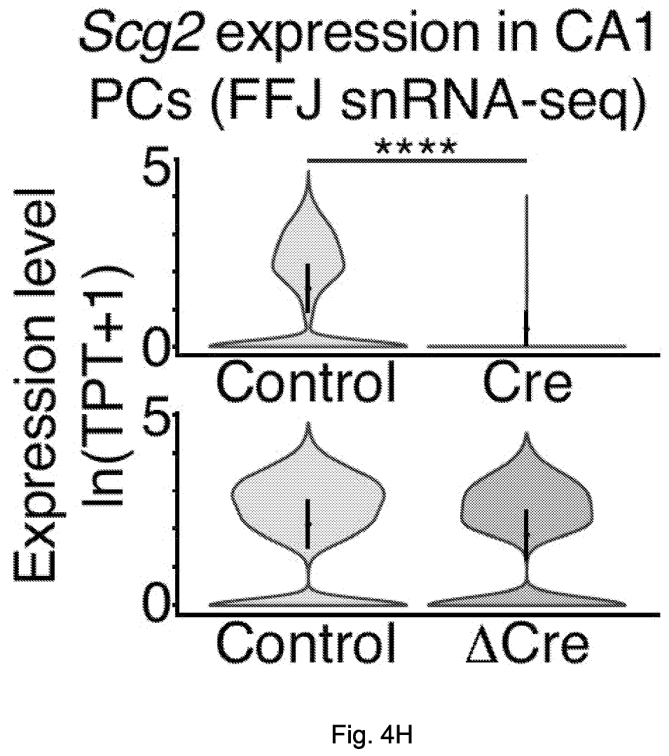

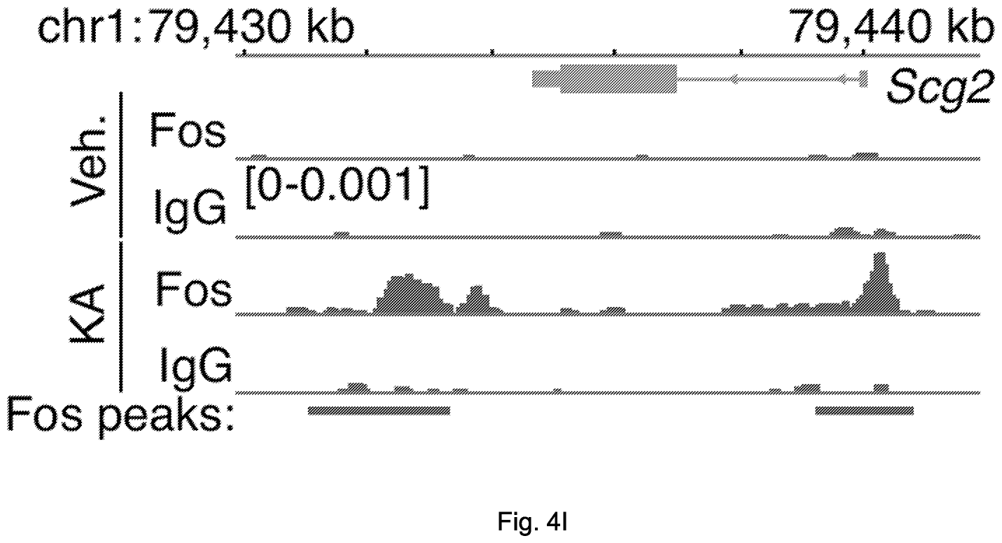

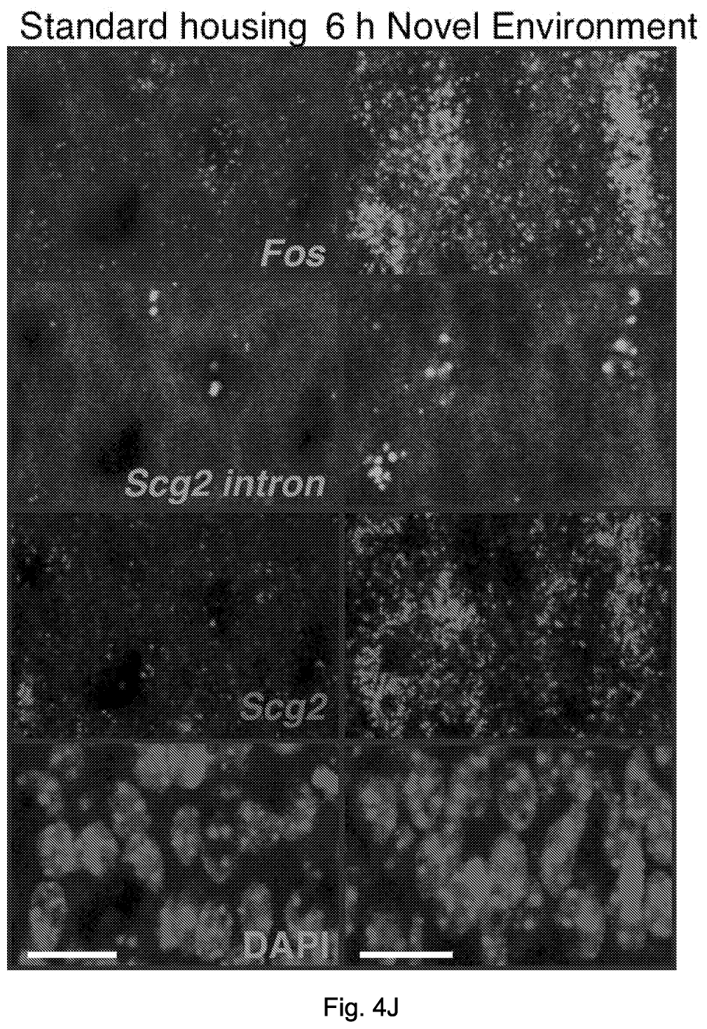

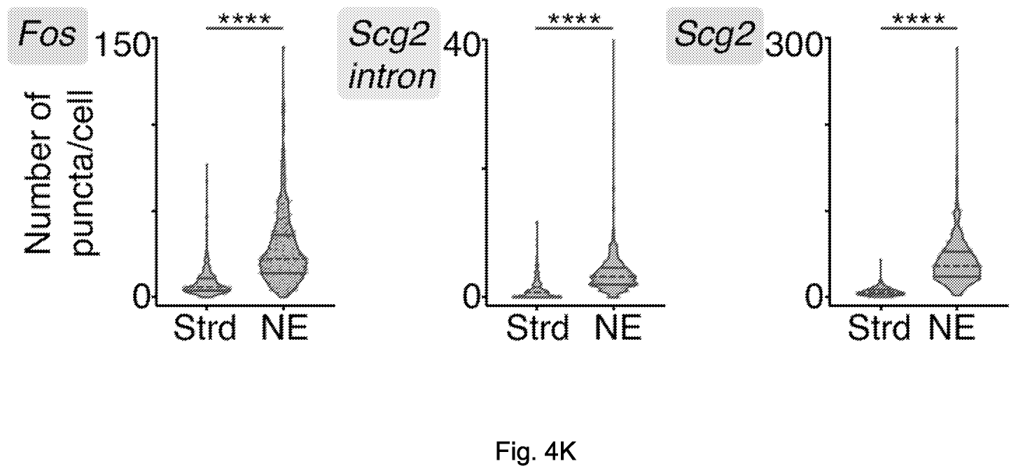

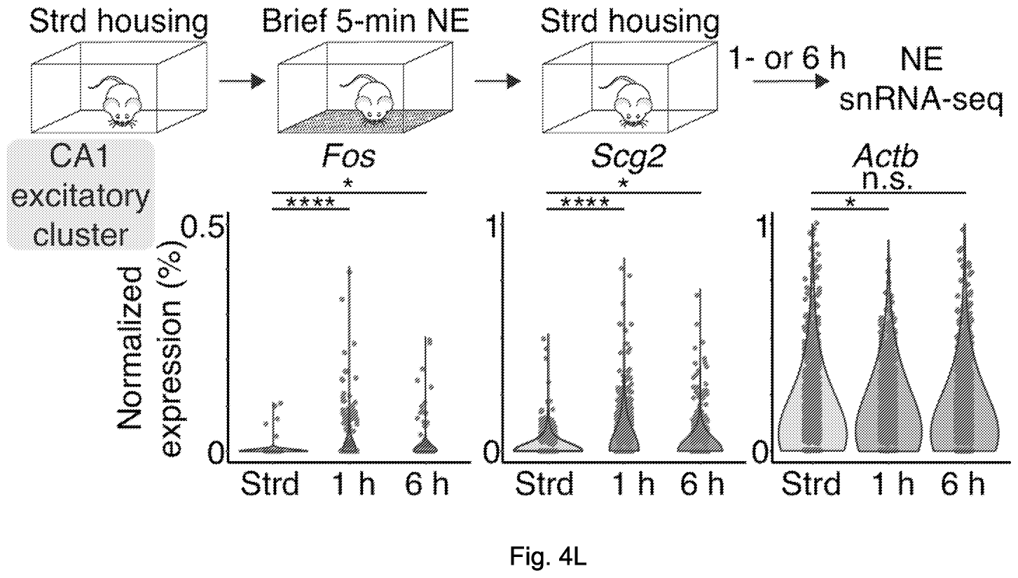

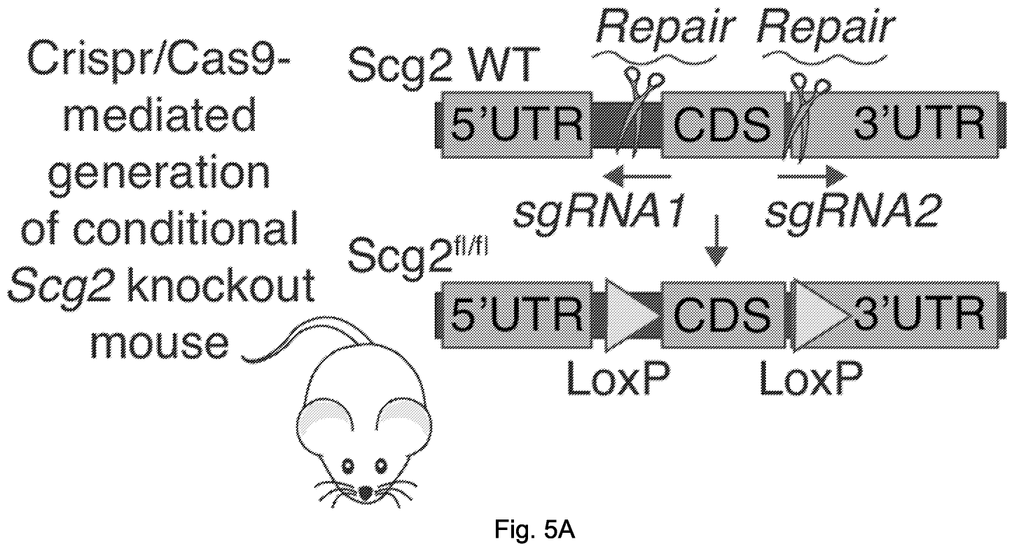

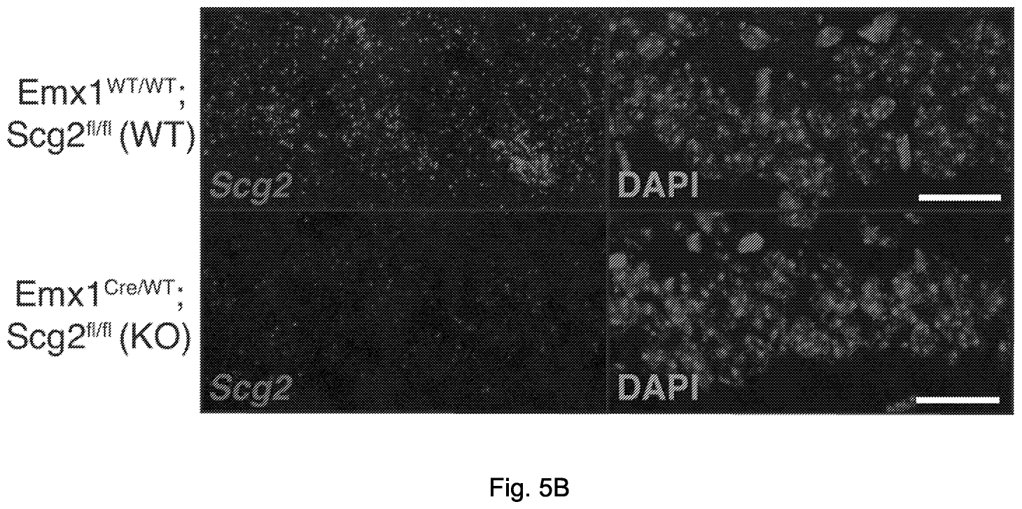

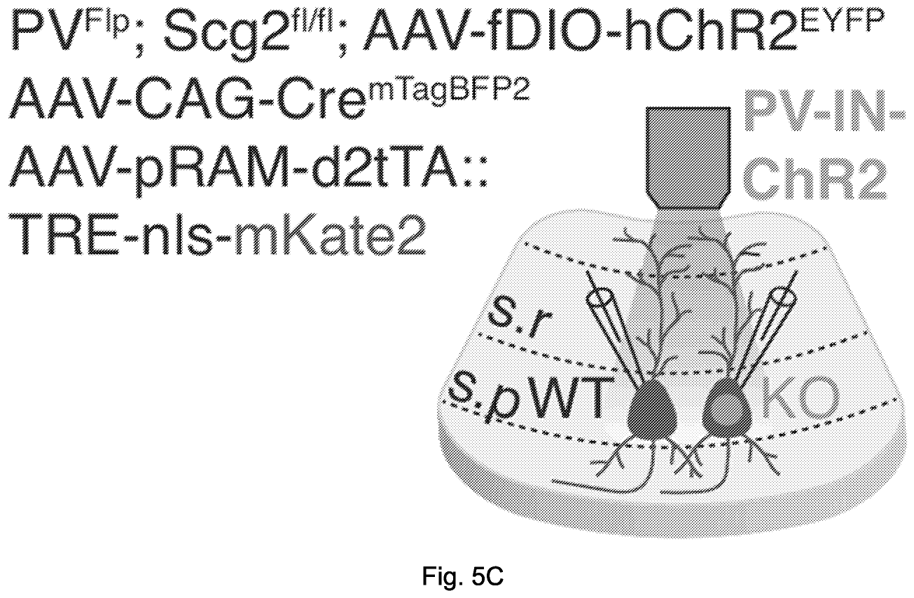

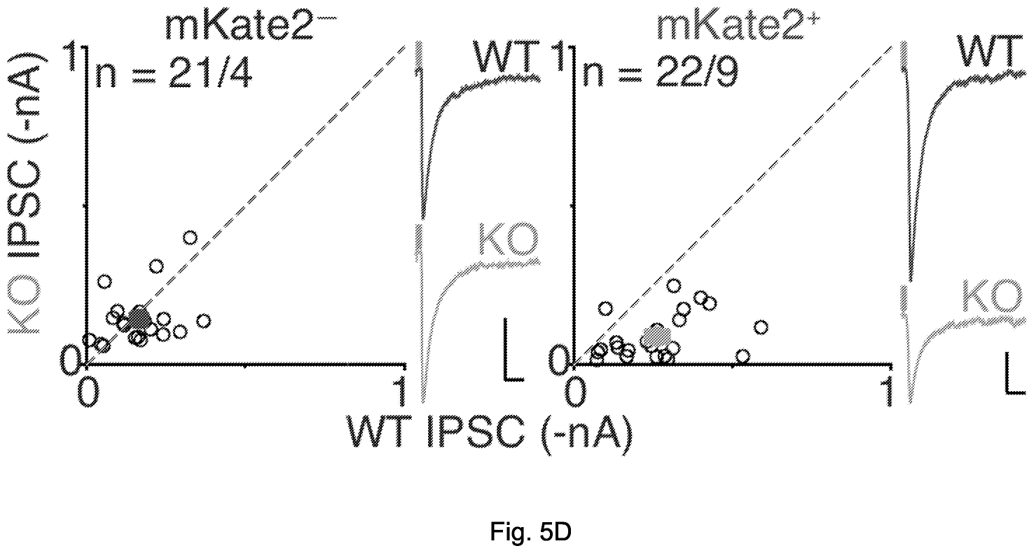

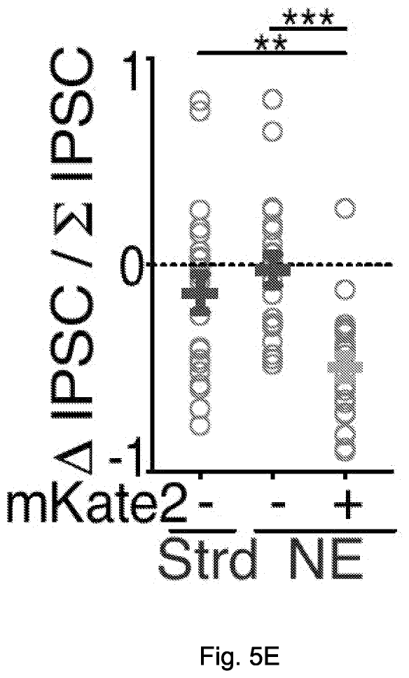

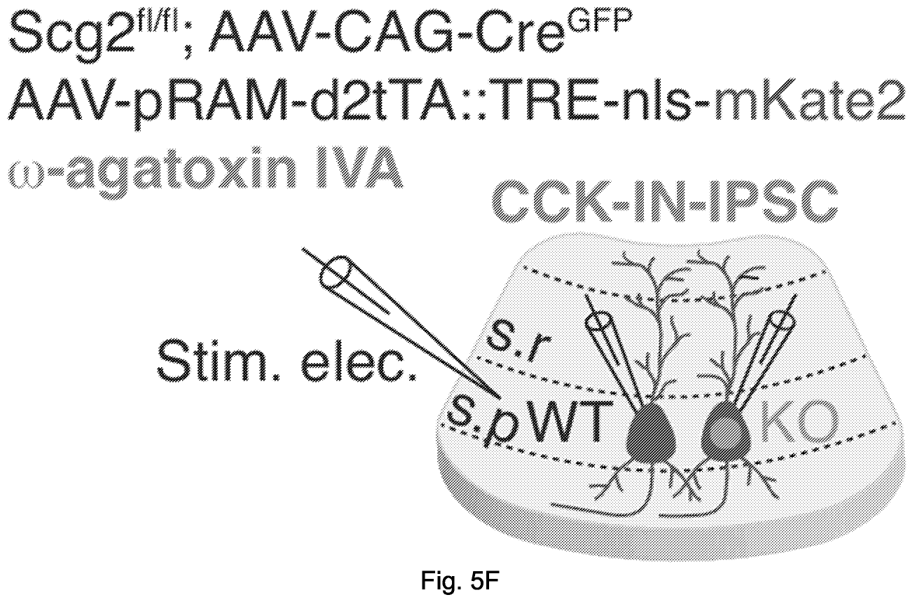

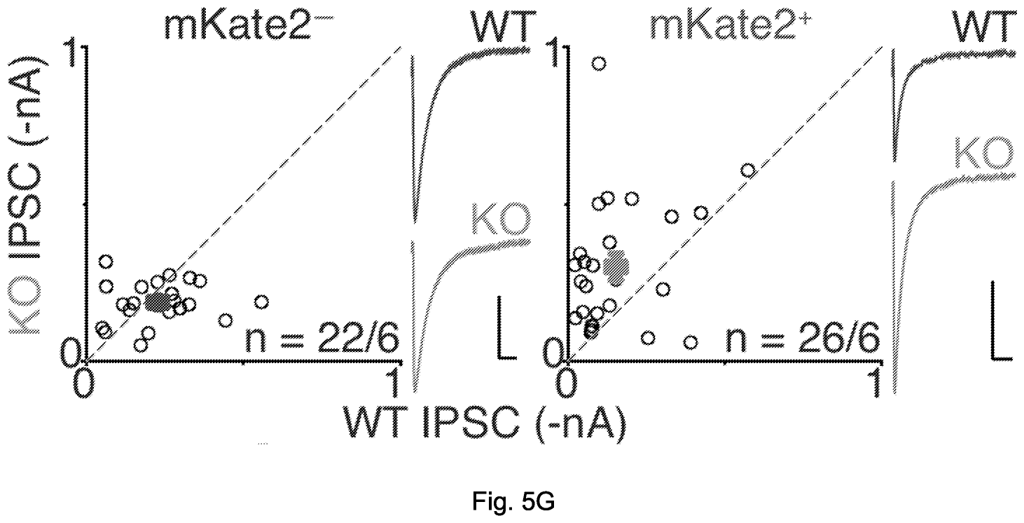

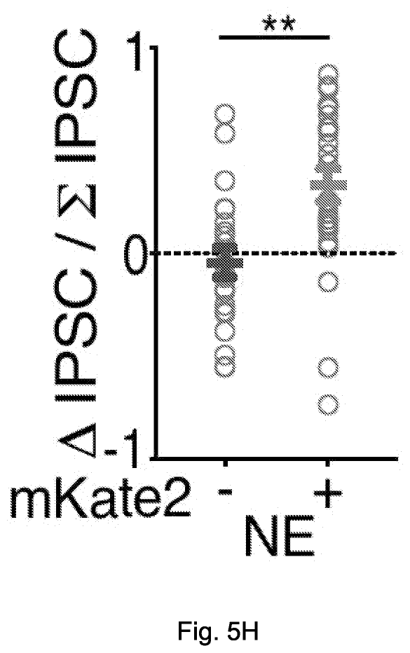

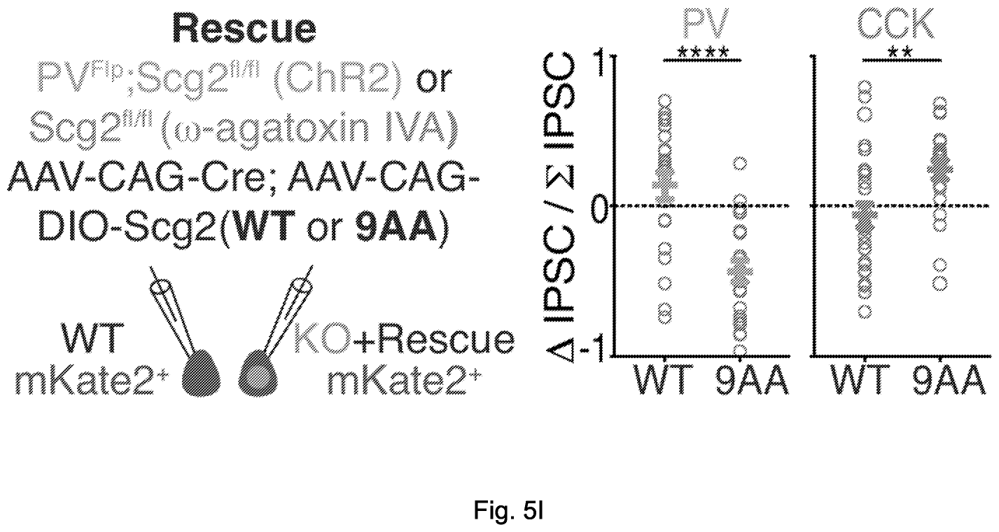

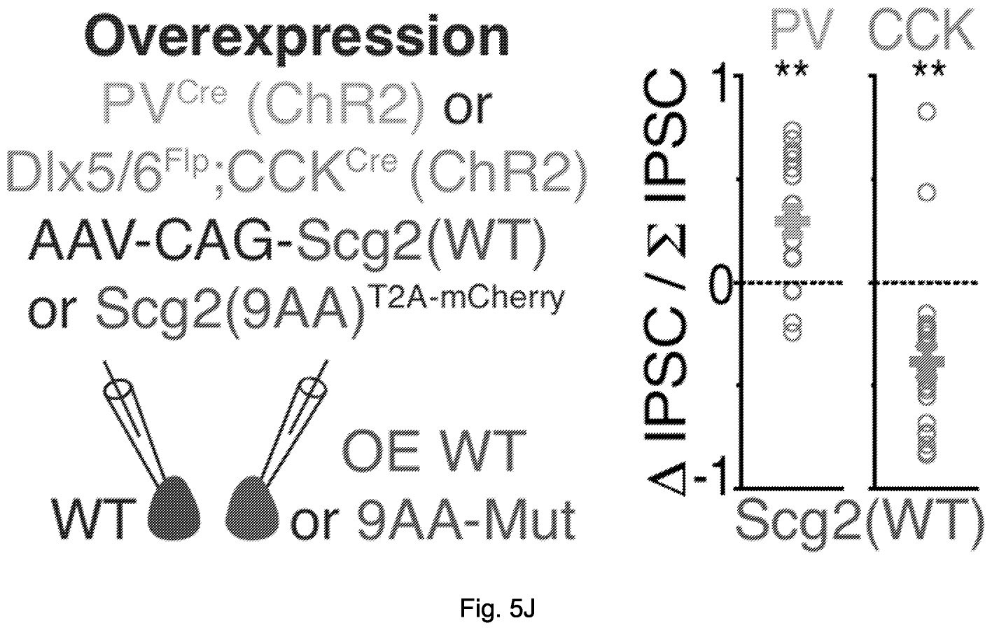

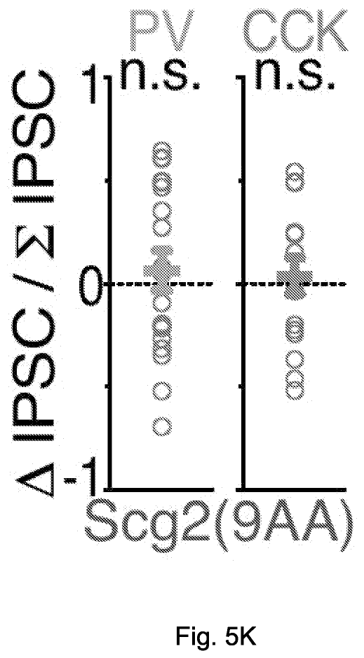

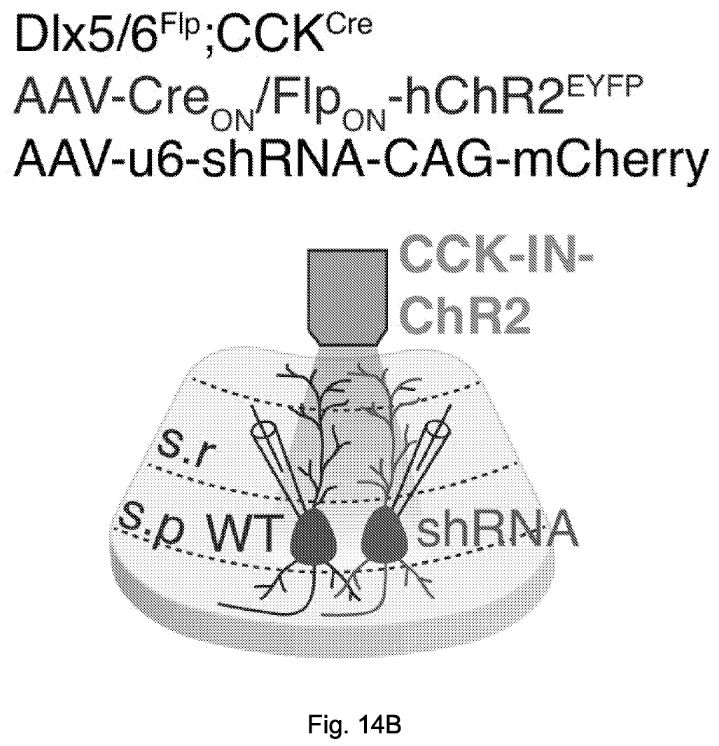

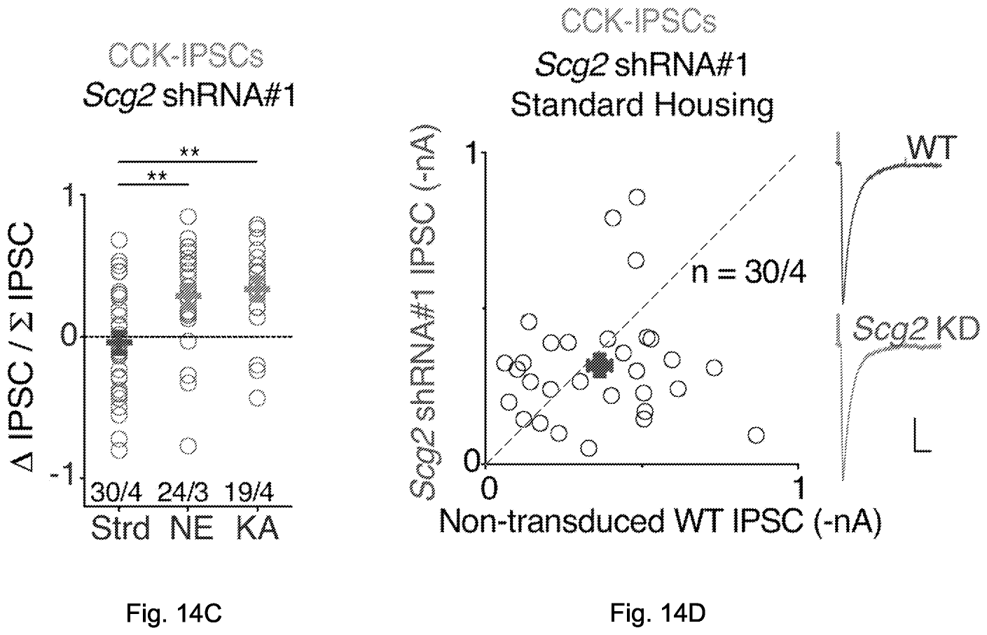

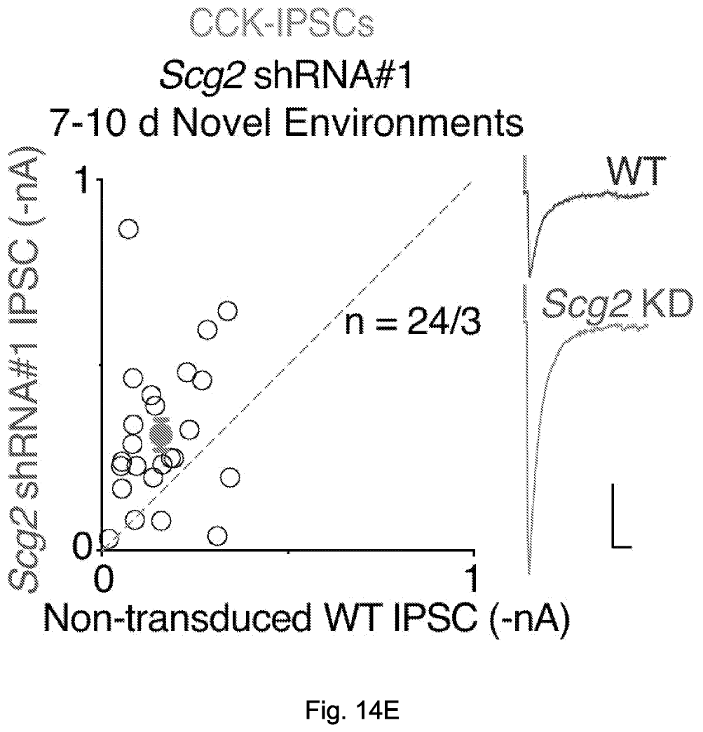

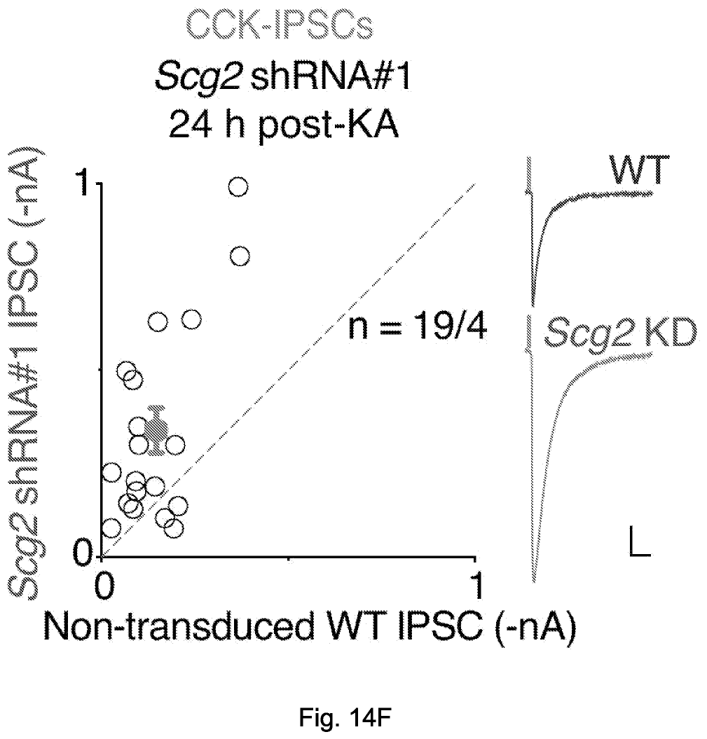

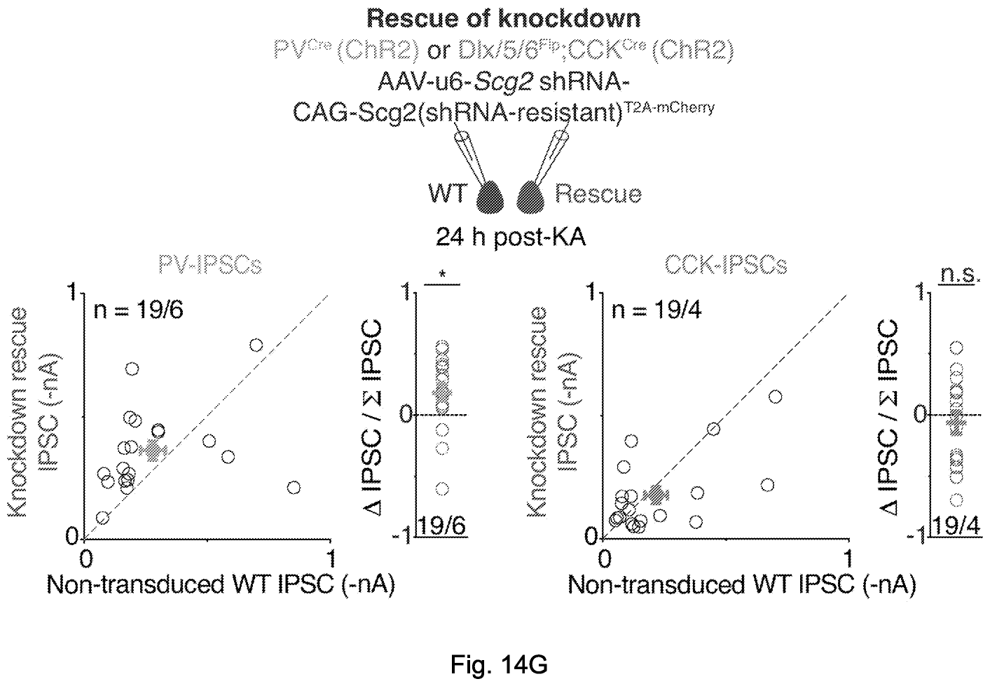

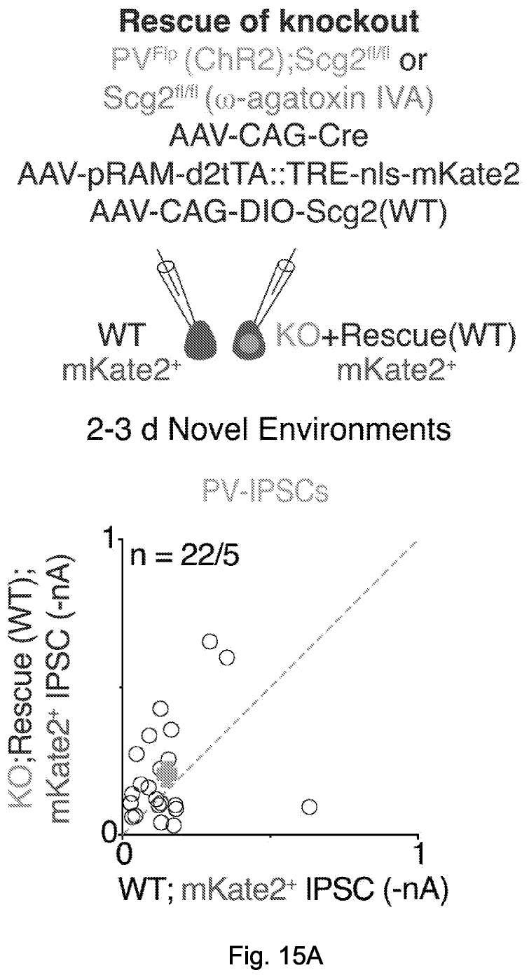

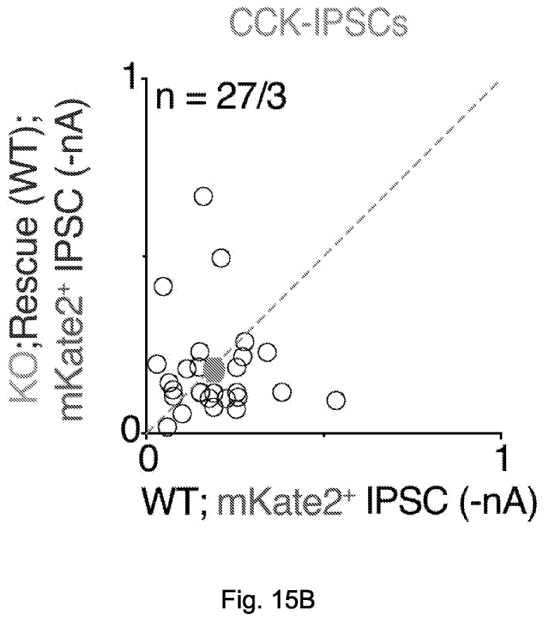

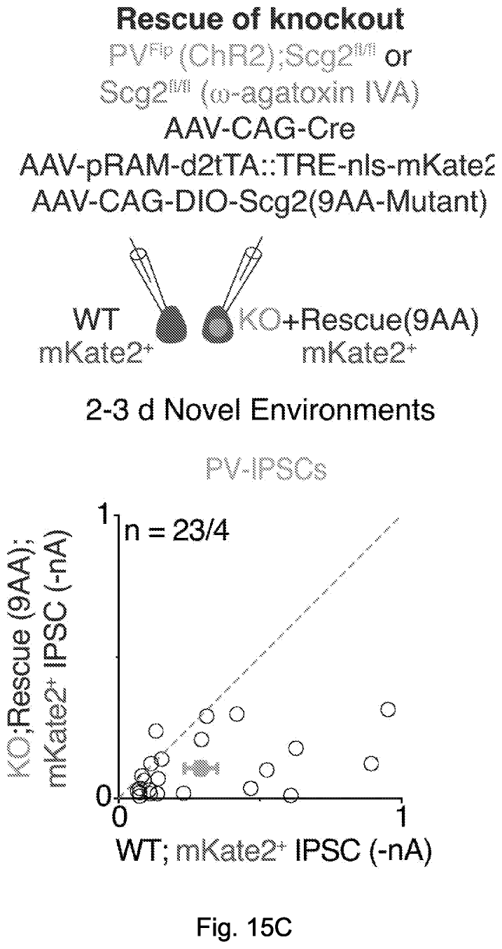

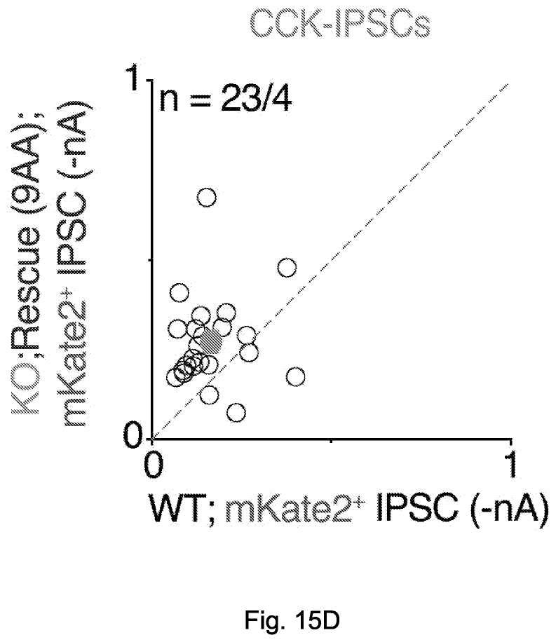

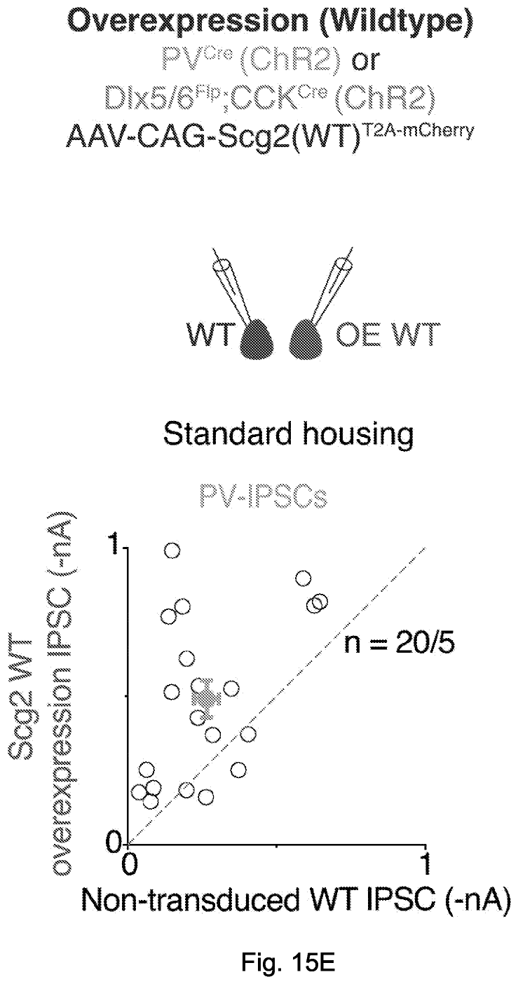

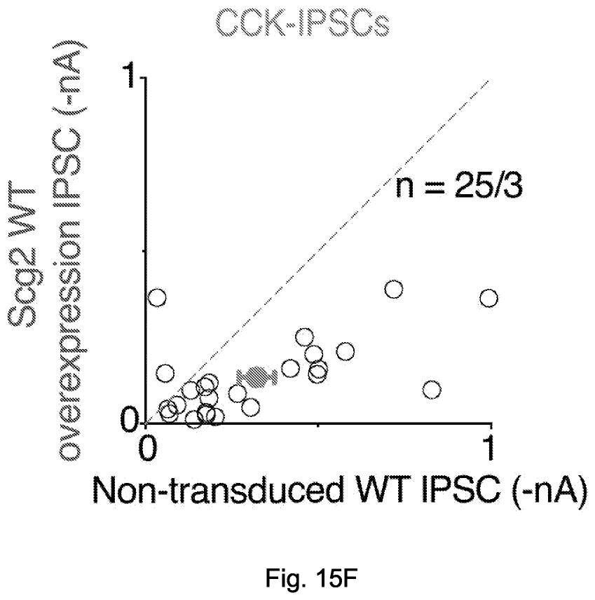

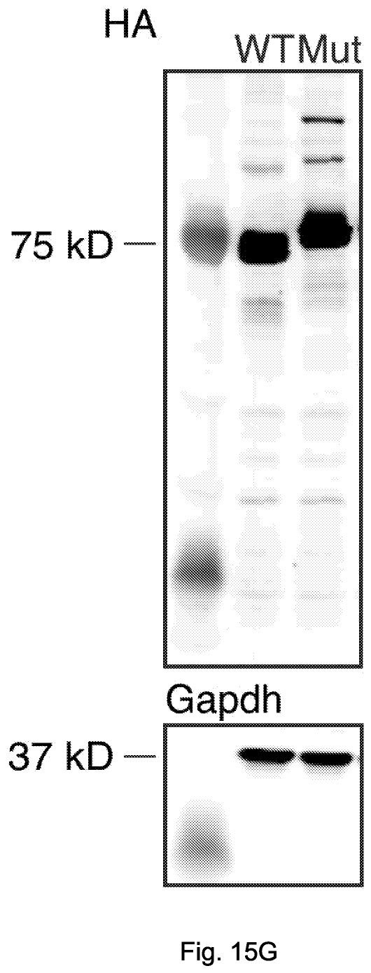

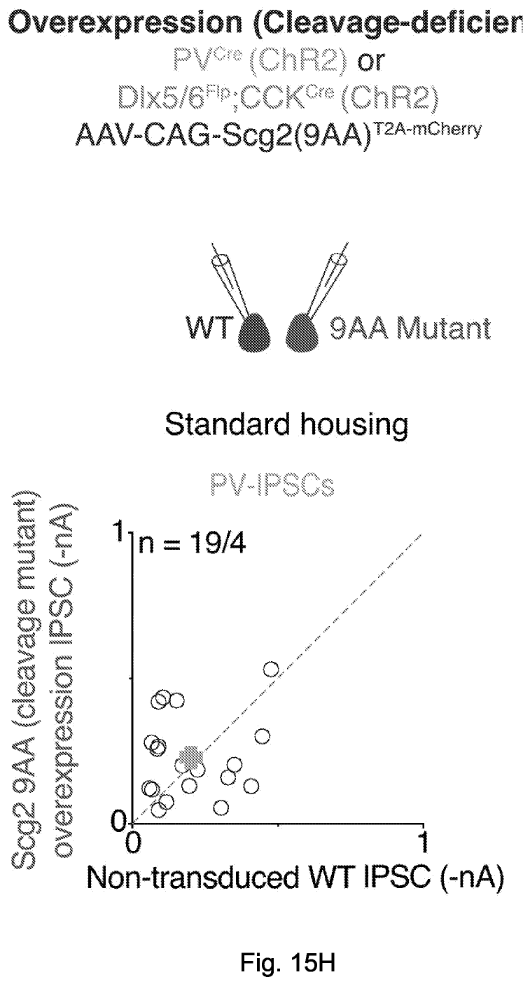

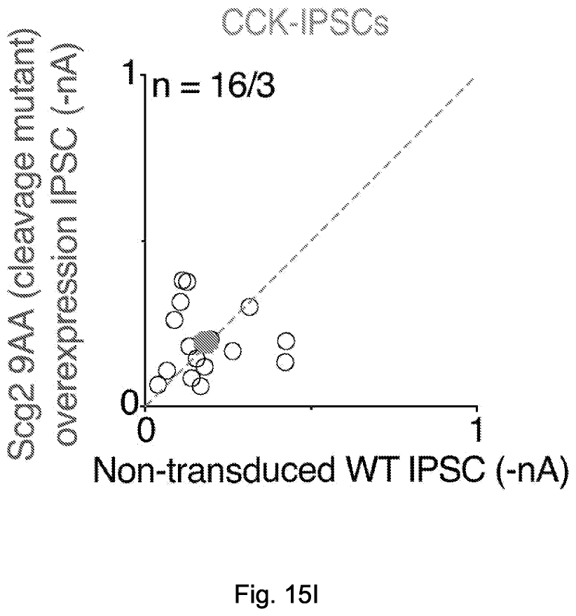

The technology described herein is associated with the following pathway elucidated herein: a novel environment was shown to lead to Fos (TF) activation in CA1 pyramidal cells, which led to Scg2 expression and subsequent cleavage and expression of four Scg2 neuropeptides, which are 33-66 amino acids long. Such Scg2 neuropeptides lead to modulation and re-wiring of interneurons, including increased parvalbumin (PV)-expressing interneuron inhibition of the pyramidal cells (PCs) and decreased cholecystokinin-expressing (CCK) interneuron inhibition of the PC. Such interneuron modulation and re-wiring subsequently led to modulation of hippocampal gamma rhythms as well as pyramidal cell coupling to theta phase, which were associated with consolidation and/or retention of memories. As such, Scg2 neuropeptides (see e.g., FIG. 4F) are associated with neural pathways that lead to unexpected beneficial neurological effects, such as memory consolidation, memory retention, and learning. For example, knockout of Scg2 led to the loss of the beneficial neurological effects as described herein, whereas rescue or overexpression restored such effects (see e.g., FIG. 4-6, FIG. 14-16). Furthermore, a cleavage-deficient Scg2, which cannot be cleaved into the Scg2 neuropeptides, did not have the same effects as WT Scg2, further demonstrating the beneficial effects of the Scg2 neuropeptides (see e.g., FIG. 5I-5K, FIG. 15A-15I).

Accordingly, the technology described herein is directed to pharmaceutical compositions comprising at least one Scg2 neuropeptide, as well as cell culture media or kits comprising such Scg2 neuropeptides. Also described herein are nucleic acids, vectors, or viral vectors encoding at least one Scg2 neuropeptide. In further aspects, described herein are methods of treating a memory-associated disorder, a learning disability, a neurodegenerative disease or disorder, or epilepsy with a pharmaceutical composition, nucleic acid, vector, or viral vector as described herein. In further aspects, described herein are detection methods of memory-associated analytes, such as Scg2 neuropeptides.

Described herein are pharmaceutical compositions comprising at least one secretogranin II (scg2) neuropeptide and a pharmaceutically acceptable carrier. Neuropeptides are chemical messengers made up of small chains of amino acids that are synthesized and released by neurons. Neuropeptides typically bind to G protein-coupled receptors (GPCRs) to modulate neural activity and other tissues like the gut, muscles, and heart. Neuropeptides are synthesized from large precursor proteins which are cleaved and post-translationally processed then packaged into dense core vesicles. Neuropeptides are often co-released with other neuropeptides and neurotransmitters in a single neuron, yielding a multitude of effects. Once released, neuropeptides can diffuse widely to affect a broad range of targets.

Scg2 (also referred to as secretogranin II or chromogranin C (CHGC)), is a protein which in humans is encoded by the SCG2 gene (see e.g., SEQ ID NO: 1; NCBI Gene ID: 7857). The Scg2 protein is a member of the chromogranin/secretogranin family of neuroendocrine secretory proteins. Scg2 protein is predominantly expressed in adrenal tissue, the brain, the appendix, the duodenum, the small intestine overall, and the stomach; see e.g., Fagerberg et al., Mol Cell Proteomics, 2014, 13(2):397-406, the content of which is incorporated herein by reference in its entirety. Studies in rodents indicate that the full-length Scg2 protein is involved in the packaging or sorting of peptide hormones and neuropeptides into secretory vesicles. The full-length Scg2 protein is cleaved to produce the active neuropeptide secretoneurin, which has been shown to exert chemotaxic effects on specific cell types, as well as the neuropeptides EM66, manserin, and SgII, whose functions were previously unknown.

In some embodiments of any of the aspects, the Scg2 polypeptide is a human Scg2 polypeptide. In some embodiments of any of the aspects, the Scg2 polypeptide is encoded by a nucleic acid sequence comprising one of SEQ ID NOs: 1-3 or a nucleic acid sequence that is at least 80%, at least 85%, at least 90%, at least 91%, at least 92%, at least 93%, at least 94%, least 95%, at least 96%, at least 97%, at least 98%, at least 99%, at least 99.5% or more identical to one of SEQ ID NOs: 1-3 that maintains the same function as a polypeptide (e.g., cleavage into Scg2 neuropeptides). In some embodiments of any of the aspects, the Scg2 polypeptide is encoded by a nucleic acid sequence comprising one of SEQ ID NOs: 1-3 or a nucleic acid sequence that is at least 95% identical to one of SEQ ID NOs: 1-3 that maintains the same function as a polypeptide (e.g., cleavage into Scg2 neuropeptides).

In some embodiments of any of the aspects, the Scg2 polypeptide comprises SEQ ID NO: 4 or an amino acid sequence that is at least 80%, at least 85%, at least 90%, at least 91%, at least 92%, at least 93%, at least 94%, least 95%, at least 96%, at least 97%, at least 98%, at least 99%, at least 99.5% or more identical to SEQ ID NO: 4 that maintains the same function (e.g., cleavage into Scg2 neuropeptides). In some embodiments of any of the aspects, the Scg2 polypeptide comprises SEQ ID NO: 4 or an amino acid sequence that is at least 95% identical to SEQ ID NO: 4 that maintains the same function (e.g., cleavage into Scg2 neuropeptides).

SEQ ID NO: 1, Homo sapiens secretogranin II (SCG2), REFSEQ gene on chromosome 2
NCBI Reference Sequence: NG_027998.1, region: 5139-10560 (see e.g., reverse complement of
223,596,940 to 223,602,361 of Homo sapiens chromosome 2, GRCh38.p13 Primary Assembly NCBI
Reference Sequence: NC_000002.12), 5422 base pairs (bp)
gaaacggcccgagaagctcgcccggagaacggggaggaatatgctgtggagctcctctgccatataaacaaaaagaggtaaggctgcttttctct
ttgatttattggaaagttccaaattgcatgctcttcttttaattcctagctgagaagaaaaagactgtttggcaagcttatgtcgataggataactgaatag
caacctgctgctcatcagccaatgtaaggacactacatatgtgtatatacatgatcgggtctatgctttatactacttgatgttggatatttactaaccaga
atgttacttagataatcatataaaaaagagtgaaaaagttaaggattttagtgtcaatacacagattaatgtaaagttaaccaagcccaatatcttgtttctt
tccaaaatttattatttctgccactaagagccagatacgaagccaggggtataatttatatgtttaatattcttaacagtaggtttccaatgatacaaaggct
gtcaagcatgttagtcataaaggcaacccattttggaactaggttttttttctttgatatttatcttacaagagctctgaattaactaaccacataaaattc
ttttctgaacttgtatatttgaaagagcttacattattatctcctaattttagttctaagatgttaacttaggattttcctgaacactggattcttcctttt
tccagggcaaaatattatcttgggttgtaatttggctgtgaccatgaattacagagaaatgatcattttgaatacatgatgaaaatactcataactttgtaa
attgttattgttattgctataatttgatttaaagaggatgttcaatatgttgctaaagattttgtctgggacaagtatttcattttttctgtatcccaacta
gttgaattgcttaaattacatatagtcacctgcatgtgtcaatgctctagcctgaattcaacagaaaagacattttaacttgagagattttaatgatagcta
tcaggggaaaaataatgttctaattatttggatttcagaaaattcaattttaaatcaaaccccatatagaatagtagagctgttgaaacatctttactactt
gaaagagctgctacctgggaaaagaatcctttgggtctcactcaaaggcaataactatgtcattcaaactgaagctgaatagttaagcaaagggtagcactt
cttaggctgtggtttaaacaactgcaatgagtagtctgcttcaactttacagcaaagtgcagttccaagataatgattttaaagatgagcatataatccaca
tgttaagactatttcagtattgtggcttcaaaaaaaataagttcagtaaggtttttaagattattatttcaaatgccaatgttgaaagttgattaccaaagc
tacattaatcctgaaataatataaacaagagtgcataatgtatttttatttgaacctcattattttatgaataaaattacttatagtaaacatgtatttata
ataaatattttggaagtatcctctcaaatgcctttcaagatgtttctttcaataaattaagtactctggtaaacgtgaatcatttaaataggtatatacgta
tgctaaaatgttttcataactaagccattattgggctctaaaaaactggaaacaaaatcatcaatatgatcttgtgaacaatggtattttctacaattgatg
tctaagctaacagaaaattgtatacatataacttagcttatctatcacaaaaccatgatctggtaaagatatgaacaaacttgtaaactcctcttacattca
ttttgtttacaacaaaatagccaatattaaaatagttttttcaatgttagcaatattaaatgtaaaacacataaaaactaatcctcttcctaattcctatgt
aagattttaaggcatcggaggaaataatgaatggagatttttaagtaggaagaattcagcacatcatctgtgtttatttgaattgggatttacatagtggtt
attttaagatttttctaaacagtatgagtgatttaagatttcacaggtccataccaaaatagacacagacagaagacaaactgaatctgaacactcacaagt
tcagaaatagactagctaaaaaaacttatgttctctctgtagcatctgttattatttactatacactctgaagggctgatgaaaagtataaccacaattgca
ttttgcaagacagtcataatataaattattagaagaaatatgaagcaaagagattttaaaacactttattggtttagcacgttcacacaaaaagaaacttca
ttggtttagtctactggctggaatctgagaagacccgtgttctggaggttatttatggatcattggattcggtatcagattgagaacaagttatttctgaaa
aatatatctggataggactccccagaaggtaattcggtatgattgacatataaaagtattactttgtcatatatggtcatgtaactgcagctgtgtttcagc
aacaaaattagatggacatttcaggaaataaaccacatgttttttgacacaactttaaggacataatttgtgggcgcgtatgtgtaaatgcatgttttaaat
taagccaacacattattttgccaatagacttcaatatataaaataattaaaacatcttgggaagtatttgctgctttattatagagaaacaacatgtactaa
acatcaaactacaagttctgtaaatgaaaaaataaaatttaagaaatctcatatcagtgtttttgctgttgttttaactaagagaatgctatgcaagttttt
ggtgaatgagtaataattttgctaaagattcctgtggtttgcttgtgggaatcatgtggaaaatattcatgaatttttaaaaagagttatcctaagcttaat
gtgaaataatatctactatgttttttcctcacctcaatgatcaattatttcttttgctatagtgtcttctagtttgtttcatttgtgtaactcatatttttt
atgttttaaggaaatctttcaaacatggctgaagcaaagacccactggcttggagcagccctgtctcttatccctttaattttcctcatctctggggctgaa
gcagcttcatttcagagaaaccagctgcttcagaaagaaccagacctcaggttggaaaatgtccaaaagtttcccagtcctgaaatgatcagggctttggag
tacatagaaaacctccgacaacaagctcataaggaagaaagcagcccagattataatccctaccaaggtgtctctgtcccccttcagcaaaaagaaaatggc
gatgaaagccacttgcccgagagggattcactgagtgaagaagactggatgagaataatactcgaagctttgagacaggctgaaaatgagcctcagtctgca
ccaaaagaaaataagccctatgccttgaattcagaaaagaactttccaatggacatgagtgatgattatgagacacagcagtggccagaaagaaagcttaag
cacatgcaattccctcctatgtatgaagagaattccagggataacccctttaaacgcacaaatgaaatagtggaggaacaatatactcctcaaagccttgct
acattggaatctgtcttccaagagctggggaaactgacaggaccaaacaaccagaaacgtgagaggatggatgaggagcaaaaactttatacggatgatga
agatgatatctacaaggctaataacattgcctatgaagatgtggtcgggggagaagactggaacccagtagaggagaaaatagagagtcaaacccagg
aagaggtgagagacagcaaagagaatatagaaaaaaatgaacaaatcaacgatgagatgaaacgctcagggcagcttggcatccaggaagaag
atcttcggaaagagagtaaagaccaactctcagatgatgtctccaaagtaattgcctatttgaaaaggttagtaaatgctgcaggaagtgggaggtta
cagaatgggcaaaatggggaaagggccaccaggctttttgagaaacctcttgattctcagtctatttatcagctgattgaaatctcaaggaatttacag
atacccccagaagacttaattgagatgctcaaaactggggagaagccgaatggatcagtggaaccggagcgggagcttgaccttcctgttgaccta
gatgacatctcagaggctgacttagaccatccagacctgttccaaaataggatgctctccaagagtggctaccctaaaacacctggtcgtgctggga
ctgaggccctaccagacgggctcagtgttgaggatattttaaatcttttagggatggagagtgcagcaaatcagaaaacgtcgtattttcccaatccat
ataaccaggagaaagttctgccaaggctcccttatggtgctggaagatctagatcgaaccagcttcccaaagctgcctggattccacatgttgaaaa
cagacagatggcatatgaaaacctgaacgacaaggatcaagaattaggtgagtacttggccaggatgctagttaaataccctgagatcattaattca
aaccaagtgaagcgagttcctggtcaaggctcatctgaagatgacctgcaggaagaggaacaaattgagcaggccatcaaagagcatttgaatca
aggcagctctcaggagactgacaagctggccccggtgagcaaaaggttccctgtggggcccccgaagaatgatgataccccaaataggcagtac
tgggatgaagatctgttaatgaaagtgctggaatacctcaaccaagaaaaggcagaaaagggaagggagcatattgctaagagagcaatggaaa
atatgtaagctgctttcattaattaccctactttcattcctcccaccccaagcaaatcccaacatttctcttcagtgtgttgacttctatcctgttaacac
tgtaatatctttaaatgatgtacaggcagatgaaaccaggtcactggggagtctgcttcatttcctctgagctgttatcttgtgtatggatatgtgtaaat
gttatgactccttgataaaaaatttattatgtccattattcaagaaagatatctatgactgtgtttaatagtatatctaatggctgtggcattgttgatgc
tcacatatgataaaaaagtgtcctataattctattgaaagtttttaatatttattgaattattttgttactgtctgtagtgttttgtggagtactggacca
aaaaaataaagcattataaatatatagttttatttataaggccttttctattgtgtgttttactgttgattaataaatgttatttctggacaa
SEQ ID NO: 2, Homo sapiens secretogranin II (SCG2), mRNA, NCBI Reference
Sequence: NM_003469.5, 2434 bp
gaaacggcccgagaagctcgcccggagaacggggaggaatatgctgtggagctcctctgccatataaacaaaaagaggaaatctttcaaacatg
gctgaagcaaagacccactggcttggagcagccctgtctcttatccctttaattttcctcatctctggggctgaagcagcttcatttcagagaaaccag
ctgcttcagaaagaaccagacctcaggttggaaaatgtccaaaagtttcccagtcctgaaatgatcagggctttggagtacatagaaaacctccgac
aacaagctcataaggaagaaagcagcccagattataatccctaccaaggtgtctctgtcccccttcagcaaaaagaaaatggcgatgaaagccact
tgcccgagagggattcactgagtgaagaagactggatgagaataatactcgaagctttgagacaggctgaaaatgagcctcagtctgcaccaaaa
gaaaataagccctatgccttgaattcagaaaagaactttccaatggacatgagtgatgattatgagacacagcagtggccagaaagaaagcttaagc
acatgcaattccctcctatgtatgaagagaattccagggataacccctttaaacgcacaaatgaaatagtggaggaacaatatactcctcaaagcctt
gctacattggaatctgtcttccaagagctggggaaactgacaggaccaaacaaccagaaacgtgagaggatggatgaggagcaaaaactttatac
ggatgatgaagatgatatctacaaggctaataacattgcctatgaagatgtggtcgggggagaagactggaacccagtagaggagaaaatagaga
gtcaaacccaggaagaggtgagagacagcaaagagaatatagaaaaaaatgaacaaatcaacgatgagatgaaacgctcagggcagcttggca
tccaggaagaagatcttcggaaagagagtaaagaccaactctcagatgatgtctccaaagtaattgcctatttgaaaaggttagtaaatgctgcagga
agtgggaggttacagaatgggcaaaatggggaaagggccaccaggctttttgagaaacctcttgattctcagtctatttatcagctgattgaaatctca
aggaatttacagatacccccagaagacttaattgagatgctcaaaactggggagaagccgaatggatcagtggaaccggagcgggagcttgacct
tcctgttgacctagatgacatctcagaggctgacttagaccatccagacctgttccaaaataggatgctctccaagagtggctaccctaaaacacctg
gtcgtgctgggactgaggccctaccagacgggctcagtgttgaggatattttaaatcttttagggatggagagtgcagcaaatcagaaaacgtcgta
ttttcccaatccatataaccaggagaaagttctgccaaggctcccttatggtgctggaagatctagatcgaaccagcttcccaaagctgcctggattcc
acatgttgaaaacagacagatggcatatgaaaacctgaacgacaaggatcaagaattaggtgagtacttggccaggatgctagttaaataccctga
gatcattaattcaaaccaagtgaagcgagttcctggtcaaggctcatctgaagatgacctgcaggaagaggaacaaattgagcaggccatcaaaga
gcatttgaatcaaggcagctctcaggagactgacaagctggccccggtgagcaaaaggttccctgtggggcccccgaagaatgatgataccccaa
ataggcagtactgggatgaagatctgttaatgaaagtgctggaatacctcaaccaagaaaaggcagaaaagggaagggagcatattgctaagaga
gcaatggaaaatatgtaagctgctttcattaattaccctactttcattcctcccaccccaagcaaatcccaacatttctcttcagtgtgttgacttctatc
ctgttaacactgtaatatctttaaatgatgtacaggcagatgaaaccaggtcactggggagtctgcttcatttcctctgagctgttatcttgtgtatgga
tatgtgtaaatgttatgactccttgataaaaaatttattatgtccattattcaagaaagatatctatgactgtgtttaatagtatatctaatggctgtggc
attgttgatgctcacatatgataaaaaagtgtcctataattctattgaaagtttttaatatttattgaattattttgttactgtctgtagtgttttgtgg
agtactggaccaaaaaaataaagcattataaatatatagttttatttataaggccttttctattgtgtgttttactgttgattaataaatgttatttctg
gacaa
SEQ ID NO: 3, Homo sapiens secretogranin II (SCG2), mRNA coding sequence (CDS),
NCBI Reference Sequence: NM_003469.5, region 92-1945, 1854 nucleotides (nt)
atggctgaagcaaagacccactggcttggagcagccctgtctcttatccctttaattttcctcatctctggggctgaagcagcttcatttcagagaaacc
agctgcttcagaaagaaccagacctcaggttggaaaatgtccaaaagtttcccagtcctgaaatgatcagggctttggagtacatagaaaacctccg
acaacaagctcataaggaagaaagcagcccagattataatccctaccaaggtgtctctgtcccccttcagcaaaaagaaaatggcgatgaaagcca
cttgcccgagagggattcactgagtgaagaagactggatgagaataatactcgaagctttgagacaggctgaaaatgagcctcagtctgcaccaaa
agaaaataagccctatgccttgaattcagaaaagaactttccaatggacatgagtgatgattatgagacacagcagtggccagaaagaaagcttaag
cacatgcaattccctcctatgtatgaagagaattccagggataacccctttaaacgcacaaatgaaatagtggaggaacaatatactcctcaaagcctt
gctacattggaatctgtcttccaagagctggggaaactgacaggaccaaacaaccagaaacgtgagaggatggatgaggagcaaaaactttatac
ggatgatgaagatgatatctacaaggctaataacattgcctatgaagatgtggtcgggggagaagactggaacccagtagaggagaaaatagaga
gtcaaacccaggaagaggtgagagacagcaaagagaatatagaaaaaaatgaacaaatcaacgatgagatgaaacgctcagggcagcttggca
tccaggaagaagatcttcggaaagagagtaaagaccaactctcagatgatgtctccaaagtaattgcctatttgaaaaggttagtaaatgctgcagga
agtgggaggttacagaatgggcaaaatggggaaagggccaccaggctttttgagaaacctcttgattctcagtctatttatcagctgattgaaatctca
aggaatttacagatacccccagaagacttaattgagatgctcaaaactggggagaagccgaatggatcagtggaaccggagcgggagcttgacct
tcctgttgacctagatgacatctcagaggctgacttagaccatccagacctgttccaaaataggatgctctccaagagtggctaccctaaaacacctg
gtcgtgctgggactgaggccctaccagacgggctcagtgttgaggatattttaaatcttttagggatggagagtgcagcaaatcagaaaacgtcgta
ttttcccaatccatataaccaggagaaagttctgccaaggctcccttatggtgctggaagatctagatcgaaccagcttcccaaagctgcctggattcc
acatgttgaaaacagacagatggcatatgaaaacctgaacgacaaggatcaagaattaggtgagtacttggccaggatgctagttaaataccctga
gatcattaattcaaaccaagtgaagcgagttcctggtcaaggctcatctgaagatgacctgcaggaagaggaacaaattgagcaggccatcaaaga
gcatttgaatcaaggcagctctcaggagactgacaagctggccccggtgagcaaaaggttccctgtggggcccccgaagaatgatgataccccaa
ataggcagtactgggatgaagatctgttaatgaaagtgctggaatacctcaaccaagaaaaggcagaaaagggaagggagcatattgctaagaga
gcaatggaaaatatgtaa
SEQ ID NO: 4, secretogranin-2 precursor, Homo sapiens, NCBI Reference Sequence:
NP_003460.2, 617 amino acids (aa)
MAEAKTHWLGAALSLIPLIFLISGAEAASFQRNQLLQKEPDLRLENVQKFPSPEMIRALEYIEN
LRQQAHKEESSPDYNPYQGVSVPLQQKENGDESHLPERDSLSEEDWMRIILEALRQAENEPQ
SAPKENKPYALNSEKNFPMDMSDDYETQQWPERKLKHMQFPPMYEENSRDNPFKRTNEIVE
EQYTPQSLATLESVFQELGKLTGPNNQKRERMDEEQKLYTDDEDDIYKANNIAYEDVVGGE
DWNPVEEKIESQTQEEVRDSKENIEKNEQINDEMKRSGQLGIQEEDLRKESKDQLSDDVSKVI
AYLKRLVNAAGSGRLQNGQNGERATRLFEKPLDSQSIYQLIEISRNLQIPPEDLIEMLKTGEKP
NGSVEPERELDLPVDLDDISEADLDHPDLFQNRMLSKSGYPKTPGRAGTEALPDGLSVEDILN
LLGMESAANQKTSYFPNPYNQEKVLPRLPYGAGRSRSNQLPKAAWIPHVENRQMAYENLND
KDQELGEYLARMLVKYPEIINSNQVKRVPGQGSSEDDLQEEEQIEQAIKEHLNQGSSQETDKL
APVSKRFPVGPPKNDDTPNRQYWDEDLLMKVLEYLNQEKAEKGREHIAKRAMENM

In some embodiments of any of the aspects, the Scg2 polypeptide from a human, mouse, rat, or chimpanzee. In some embodiments of any of the aspects, the Scg2 polypeptide is a chimera of Scg2 sequences from a human, mouse, rat, or chimpanzee. In some embodiments of any of the aspects, the pharmaceutical composition comprises a first Scg2 neuropeptide from a first species (e.g., human, mouse, rat, or chimpanzee) and a second Scg2 neuropeptide from a second species that is different from the first species (e.g., human, mouse, rat, or chimpanzee). In some embodiments of any of the aspects, the Scg2 polypeptide is a mouse Scg2 polypeptide (see e.g., SEQ ID NOs: 33 or 36). In some embodiments of any of the aspects, the Scg2 polypeptide is a rat Scg2 polypeptide (see e.g., SEQ ID NOs: 34 or 37). In some embodiments of any of the aspects, the Scg2 polypeptide is a chimp Scg2 polypeptide (see e.g., SEQ ID NOs: 35 or 38).

In some embodiments of any of the aspects, the Scg2 polypeptide is encoded by a nucleic acid sequence comprising one of SEQ ID NOs: 36-38 or a nucleic acid sequence that is at least 80%, at least 85%, at least 90%, at least 91%, at least 92%, at least 93%, at least 94%, least 95%, at least 96%, at least 97%, at least 98%, at least 99%, at least 99.5% or more identical to one of SEQ ID NOs: 36-38 that maintains the same function as a polypeptide (e.g., cleavage into Scg2 neuropeptides). In some embodiments of any of the aspects, the Scg2 polypeptide is encoded by a nucleic acid sequence comprising one of SEQ ID NOs: 36-38 or a nucleic acid sequence that is at least 95% identical to one of SEQ ID NOs: 36-38 that maintains the same function as a polypeptide (e.g., cleavage into Scg2 neuropeptides).

In some embodiments of any of the aspects, the Scg2 polypeptide comprises one of SEQ ID NOs: 33-35 or an amino acid sequence that is at least 80%, at least 85%, at least 90%, at least 91%, at least 92%, at least 93%, at least 94%, least 95%, at least 96%, at least 97%, at least 98%, at least 99%, at least 99.5% or more identical to one of SEQ ID NOs: 33-35 that maintains the same function (e.g., cleavage into Scg2 neuropeptides). In some embodiments of any of the aspects, the Scg2 polypeptide comprises one of SEQ ID NOs: 33-35 or an amino acid sequence that is at least 95% identical to one of SEQ ID NOs: 33-35 that maintains the same function (e.g., cleavage into Scg2 neuropeptides).

SEQ ID NO: 33, secretogranin-2 isoform 2 precursor Mus musculus, NCBI Reference
Sequence: NP_001297609.1, 577 aa, bolded text indicates secretoneurin (e.g., aa 184-216 of SEQ ID
NO: 33); italicized text indicates EM66 (e.g., aa 219-284 of SEQ ID NO: 33); bolded italicized text
indicates manserin (e.g., aa 487-526 of SEQ ID NO: 33); and double-underlined text indicates SgII
(e.g., aa 529-570 of SEQ ID NO: 33):
MAGAKAYRLGAVLLLIHLIFLISGAEAASFQRNQLLQKEPDLRLENVQKFPSPEMIRALEYIEK
LRQQAHREESSPDYNPYQGVSVPLQLKENGEESHLAESSRDALSEDEWMRIILEALRQAENEP
PSAPKENKPYALNLEKNFPVDTPDDYETQQWPERKLKHMRFPLMYEENSRENPFKRTNEIVE
EQYTPQSLATLESVFQELGKLTGPSNQKRERVDEEQKLYTDDEDDVYKTNNIAYEDVVGGED
WSPIEEKIETQTQEEVRDSKENTEKNEQINEEMKRSGQLGLPDEENRRESKDQLSEDASKVITYL
RRNLQIPPEDLIEMLKAGEKPNGLVEPEQDLELAVDLDDIPEADLDRPDMFQSKMLSKGGYP
KAPGRGMVEALPDGLSVEDILNVLGMENVVNQKSPYFPNQYSQDKALMRLPYGPGKSRAN
QIPKVAWIPDVESRQAPYENLNDQELGEYLARMLVKYPELLNTNQLKR [symbol] [symbol] [symbol]
KRIPVGSLKNEDTPNRQYLDEDMLLKVLEYLNQEQ
AEQGREHLAKRAMENM
SEQ ID NO: 34, secretogranin-2 precursor Rattus norvegicus, NCBI Reference
Sequence: NP_073160.2, 619 aa; bolded text indicates secretoneurin (e.g., aa 184-216 of SEQ ID
NO: 34); italicized text indicates EM66 (e.g., aa 219-284 of SEQ ID NO: 34); bolded italicized text
indicates manserin (e.g., aa 529-568 of SEQ ID NO: 34); and double-underlined text indicates SgII
(e.g., aa 571-612 of SEQ ID NO: 34):
MAESKAYRFGAVLLLIHLIFLVPGTEAASFQRNQLLQKEPDLRLENVQKFPSPEMIRALEYIEK
LRQQAHREESSPDYNPYQGISVPLQLKENGEESHLAESSRDVLSEDEWMRIILEALRQAENEP
PSALKENKPYALNLEKNFPVDTPDDYETQQWPERKLKHMRFPLMYEENSRENPFKRTNEIV
EEQYTPQSLATLESVFQELGKLTGPSNQKRERVDEEQKLYTDDEDDVYKTNNIAYEDVVGGE
DWSPMEEKIETQTQEEVRDSKENTEKNEQINEEMKRSGHLGLPDEGNRKESKDQLSEDASKVIT
YLRRLVNAVGSGRSQSGQNGDRAARLLERPLDSQSIYQLIEISRNLQIPPEDLIEMLKAGEKPN
GLVEPEQDLELAVDLDDIPEADIDRPDMFQSKTLSKGGYPKAPGRGMMEALPDGLSVEDILN
VLGMENVANQKSPYFPNQYSRDKALLRLPYGPGKSRANQIPKVAWIPDVESRQAPYDNLND
KDQELGEYLARMLVKYPELMNTNQLKR [symbol] [symbol] [symbol] [symbol]
KRIPAGSLKNEDTPNRQYLDEDMLLKVLEYLNQEQAEQGREHLAKRAMENM
SEQ ID NO: 35, secretogranin-2 Pan troglodytes NCBI Reference Sequence:
XP_516120.2, 616 aa; bolded text indicates secretoneurin (e.g., aa 182-214 of SEQ ID NO: 35);
italicized text indicates EM66 (e.g., aa 217-282 of SEQ ID NO: 35); bolded italicized text indicates
manserin (e.g., aa 526-565 of SEQ ID NO: 35); and double-underlined text indicates SgII (e.g., aa
568-609 of SEQ ID NO: 35):
MAEAKTHWLGAALSLIPLIFLISGAEAASFQRNQLLQKEPDLRLENVQKFPSPEMIRALEYIEK
LRQQAHKEESSPDYNPYQGVSVPLQQKENGDESHLPERDSLSEEDWMRIILEALRQAENEPQ
SAPKENKPYALNSEKNFPMDMSDDYETQQWPERKLKHMQFPPMYEENSRDNPFKRTNEIVE
EQYTPQSLATLESVFQELGKLTGPNNQKRERMDEEQKLYTDDDDIYKANNIAYEDVVGGED
WNPVEEKIESQTQEEVRDSKENIEKNEQINDEMKRSGQLGIQEEDLRKESKDQLSDDVSKVIAY
LKRLVNAAGSGRLQNGQNGERATRLFEKPLDSQSIYQLIEISRNLQIPPEDLIEMLKTGEKPNG
SVEPERELDLPVDLDDISEADLDHPDLFQNKMLSKSGYPKTPGRAGTEALPDGLSVEDILNLL
GMESAANQKTSYFPNPYNQEKVLPRLPYGPGRSRSNQLPKAAWIPYVENRQMAYENLNDKD
QELGEYLARMLVKYPEIINSNQVKR [symbol] [symbol] [symbol] [symbol]
KRFPVGPPKNDDTPNRQYLDEDLLMKVLEYLNQEKAEKGREHIAKRAMENM
SEQ ID NO: 36, Scg2 secretogranin II Mus musculus (house mouse), Gene ID: 20254,
transcript variant 2, mRNA, NCBI Reference Sequence: NM_001310680.2 (CDS region nt 86-1819,
as indicated by bolded text), 2604 nt
aaacggcccgagccctcactcagcggcagagaggagcatgcttggagccttccacataatataagacagaggaaatctttaagacatggctgga
gctaaggcgtaccgacttggagcagttctgcttcttatccacttaattttcctcatctctggagccgaagcagcttccttccagcgaaaccag
ctgcttcagaaagaaccagacctcagattggagaatgtccaaaagtttcctagtccagaaatgatcagggctttggagtacatagaaaag
ctcaggcagcaagctcacagagaagaaagcagcccagactacaatccctaccaaggcgtctctgttcctcttcaactcaaagaaaacgg
agaagaaagccacttggcagagagctcaagggatgcactgagtgaagacgagtggatgcggataatactcgaggctctgaggcaggct
gaaaatgagccgccatctgcccccaaagagaacaagccctatgccttgaatctggagaagaacttcccagtggacacgcctgatgactat
gagactcaacagtggcctgagaggaaactcaagcacatgcggttccctctcatgtatgaagagaattccagagaaaaccccttcaaacgc
acaaatgaaatagtcgaggaacaatacacaccccaaagtcttgctaccctggagtctgtgttccaagagcttgggaaactgacagggcca
agcaaccagaagcgtgagagggttgacgaggaacaaaagctgtacacagatgatgaagacgacgtgtacaagaccaacaacattgcct
atgaagatgtcgtggggggagaagactggagccccatagaggagaaaatagagactcaaacccaggaagaggtgagagacagcaaa
gagaacacagaaaaaaatgaacaaatcaatgaagagatgaaacgttcagggcagttggggctcccagatgaagaaaaccggagaga
gagtaaagaccaactctcagaggatgcctccaaagttatcacctacctgagaaggaatttgcagataccccctgaagatttaattgagatg
ctcaaagctggagagaagccaaatgggttggtggagccagagcaggatctggagcttgctgttgacctagatgacatcccagaggctga
cctagaccgtccagacatgtttcaaagtaagatgctctccaaggggggtatcccaaggcacctggtcgtggtatggtagaggccttgcct
gatgggctgagtgtcgaggacattttaaatgttttagggatggagaatgtagtaaatcagaagtccccatattttcccaaccaatatagcca
agacaaggctctgatgaggctcccttatggtcctgggaaatctagagccaaccagattcccaaagtagcctggatccctgatgttgaaagc
agacaagcaccttatgaaaatctgaatgaccaagaattgggagagtacttagccaggatgctagttaagtaccctgagctcctgaatacc
aaccagctgaagagagtgcccagtccagtctcctcagaggatgacctccaagaagaagagcagctcgagcaggccatcaaggaacatc
tggggccaggaagctcccaggaaatggagagactggccaaggtgagcaaaaggatccccgtaggatccctgaagaatgaggacaccc
caaacagacagtacctggatgaagatatgctcctgaaagtgctggagtacctcaaccaagagcaggcagagcaggggagggagcatct
tgccaagcgggccatggaaaacatgtaaacagctttaatgcccaatttcccttctttcccccaagtaagccccctacatttctcttaagtgtgttgatc
tctatcctgttgacagtgtaatatctttaaagtgatgtataggcagatgactccaggtcattttgggggatctgcttcacttattctgagctgttacgttg
tgtgtggatgtgtgtaaatgttatgattcccagattgaaaaaaaatgttctttattcaagaaagatatctatgatagtgttggctaatgtatctaatggtc
atggaattgatgatgctcacatatgataaagagtatcctataattatcttggaagtttttaacatttattgaattattttgttactgtctgtagtgttttg
tggagttctggagcaaaaccaataaagcattataaatatatagttttacttataaggccttttctattgtgtgttttattgttgattaataaatgttattt
ctggatacctttggactttttattctggaaaccagagacaactggtatggatcaagcagcatggagccagaggagaaaattattactgtccacaggcaaccc
aggtaagagatgaatcttatatgtgatcatattttctgcctacaggatgttgtgaacattcccgaacagccttacatcttttcatgttttccatatacctca
ttaacaaaacgagactttgggtataattcttacacttcacattgattcatataagtaaaagatattaaactttccccactcatcacaatttgaaaatgaaa
gaaaa
SEQ ID NO: 37, Scg2 secretogranin II Rattus norvegicus (Norway rat), Gene ID: 24765,
NCBI Reference Sequence: NM_022669.2 (CDS region nt 31-1890, as indicated by bolded text),
mRNA 2291 nt
acaatataagacagaggaaaattttaagacatggctgaatcgaaggcttaccgatttggagcagttctgcttcttatccacttaattttccttgtcc
ctggaaccgaagcagcttccttccagcgaaaccagctgcttcagaaagaaccagacctcagattggagaatgtccagaagtttcctagtc
cagaaatgatcagggctttggagtacatagaaaagctcaggcagcaggcccacagagaagaaagcagcccagactacaatccctacca
aggcatctctgttccccttcaactcaaagaaaacggagaagaaagtcacttggcagagagctcaagggatgtcttgagtgaagacgagtg
gatgcggataatacttgaggctttgaggcaggctgaaaatgagccgccatctgccctcaaggagaacaagccctatgccttgaatctgga
gaagaacttccctgtggacacgcctgatgactatgagactcaacaatggcctgagaggaaactcaagcacatgcggttccctctcatgtat
gaagagaattccagggaaaaccccttcaaacgcacaaacgaaatagtagaagaacagtacacaccccaaagtcttgctaccctggagtc
tgtgttccaagagcttgggaaactgacagggccaagcaaccagaagcgtgagagggttgacgaggaacagaagctctacacggacgat
gaagatgacgtgtacaagaccaacaacattgcctatgaagatgtggtcgggggagaagactggagtcctatggaggagaaaatagaga
ctcaaacccaggaagaggtgagagacagcaaagagaacacagaaaaaaacgaacaaatcaatgaagagatgaaacggtcagggca
cttggggctcccagatgaaggtaaccggaaagagagcaaagaccagctctcagaggacgcctccaaggtcatcacctacttgagaaggt
tagtgaatgctgtgggcagtgggaggtcccagagtgggcaaaacggggacagggcagccaggcttcttgagaggccccttgattctcagt
ctatttatcagctgattgaaatctccaggaatttgcagataccccctgaagacttaattgagatgctcaaagctggggagaaaccaaatgg
gttggtggagcccgagcaggatctggagcttgctgttgacctagatgacatcccggaagctgacatagaccgcccagacatgtttcaaagt
aagacgctctccaagggtgggtatcccaaggcacctggtcgaggtatgatggaggccttgccagatggcctcagtgttgaagacattttaa
atgttttagggatggagaatgtagcaaatcagaagtccccatatttccccaaccaatacagccgagacaaggctctgctgaggcttccttat
ggtcctgggaaatctagagccaaccagattcccaaagtagcctggatcccagacgttgaaagcagacaagccccctatgacaatctgaat
gataaggaccaagaattgggagagtacttagccaggatgctagttaagtaccctgagctcatgaataccaaccagctgaagagagtgcc
cagcccaggctcctcagaagatgacctccaagaagaagagcagctcgagcaggccatcaaggagcatctgggtcaaggaagctcccag
gaaatggagaaactggccaaggtgagcaaaaggatccctgcaggatccctgaagaatgaggataccccaaatagacagtacctggatg
aagatatgctcctgaaagtgctagagtatctcaatcaagaacaggcagagcagggaagggaacatcttgccaaacgggccatggaaaa
catgtaaacagctttaatgcccaatttcccttcttttccccaagtgaatcccctccctttctcttaagtgtgttaatctctatcctgttaacactgtaat
atctttaagtgatgtacaagcagatgactccagatagttttggggatctgctttacttattctgagctgttatgttgtgtatggatgtgtataaatgttat
gactctcagatttaaaaaatatgtcctttattcaagaaagatatctatgatagtgttgactaatgtatccaatggtcatggtattgacaatgctcacata
tgatgaagagtatcctataattatcttggaagtttttaacatttattgaattattttgttactgtctgtagtgttttgtggagttctggagcaaaatcaa
taaagca
SEQ ID NO: 38, SCG2 secretogranin II Pan troglodytes (chimpanzee), Gene ID:
459977, NCBI Reference Sequence: XM_516120.3, (CDS region nt 230-2080, as indicated by bolded
text) mRNA, 2568 nt
ataaagtgattattttctcttggttctttgaaaaacctcgcttgtgctggggtttgtggctgaacccggtgacgtcagtgtggcagtgcggagtcaggcg
cagcggctccctataagcagaggagctgtccgtgtgctgaaacggcccgagaagctcgcccggagaacggggaggaatatgctgtggagctcc
tctgccatataaacaaaaagaggaaatctttcagacatggctgaagcaaagacccactggcttggagcagccctgtctcttatccctttaattttc
ctcatctctggggctgaagcagcttcatttcagagaaaccagctgcttcagaaagaaccagacctcaggttggaaaatgtccaaaagtttc
ccagtcctgaaatgatcagggctttggagtacatagaaaagctccgacaacaggctcataaggaagaaagcagcccagattataatccct
accaaggtgtctctgtcccccttcagcaaaaagaaaatggcgatgaaagtcacttgcccgagagggattcactgagtgaagaagactgg
atgagaataatactcgaagctttgagacaggctgaaaatgagcctcagtctgcaccaaaagaaaataagccctatgccttgaattcagaa
aagaactttccaatggacatgagtgatgattatgagacacagcagtggccagaaagaaagcttaagcacatgcaattccctcctatgtatg
aagagaattccagggataacccctttaaacgcacaaatgaaatagtggaggaacaatatactcctcaaagccttgctacattggaatctgt
cttccaagagctggggaaactgacaggaccaaacaaccagaaacgtgagaggatggatgaggagcaaaaactttatacggatgatgat
gatatctacaaggctaataacattgcctatgaagatgtggtggggggagaagattggaacccagtagaggagaaaatagagagtcaaa
cccaggaagaggtgagagacagcaaagagaatatagaaaaaaatgaacaaatcaatgatgagatgaaacgctcagggcagcttggca
tccaggaagaagatcttcggaaagagagtaaagaccaactctcagatgatgtctccaaagtaattgcctatttgaaaaggttagtaaatgc
tgcaggaagtgggaggttacagaatgggcaaaatggggaaagggccaccaggctttttgagaaacctcttgattctcagtctatttatcag
ctgattgaaatctcaaggaatttacagatacccccagaagacttaattgagatgctcaaaactggggagaagccgaatggatcagtggaa
ccggagcgggagcttgaccttcctgttgacctagatgacatctcagaggctgacttagaccatccagacctgttccaaaataagatgctctc
caagagtggctaccctaaaacacctggtcgtgctgggactgaggccctaccagacgggctcagtgttgaggatattttaaatcttttaggg
atggagagtgcagcaaatcagaaaacttcgtattttcccaatccatataaccaggagaaagttctgccaaggctcccttatggtcctggaa
gatctagatcgaaccagcttcccaaagctgcctggattccatatgttgaaaacagacagatggcatatgaaaacctgaacgacaaggatc
aagaattaggtgagtacttggccaggatgctagttaaataccctgagatcattaattcaaaccaagtgaagcgagttcctggtcaaggctc
atctgaagatgacctacaggaagaggaacaaattgagcaggccatcaaagagcatttgaatcaaggcagctctcaggagactgacaag
ctggccccggtgagcaaaaggttccctgtggggcccccgaagaatgatgataccccaaataggcagtacttggatgaagatctgttaatg
aaagtgctggaatacctcaaccaagaaaaggcagaaaagggaagggagcatattgctaagagagcaatggaaaatatgtaagctgcttt
cattaattaccctactttcattcctcccaccccaagcaaatcccaacatttctctttagtgtgttgacttctatcctgttaacactgtaatatcttta
aatgatgtacaggcagatgaaaccaggtcactggggagtctgcttcatttcctctgagctgttatcttgtgtatggatacgtgtaaatgttatgactcc
ttgataaaaaatttattatgtccattattcaagaaagatatctatgactgtgtttaatagtgtatctaatggctgtggcattgttgatgctcacatatga
taaaaagtgtcctataattctattgaaagtttttaatatttattgaattattttgttactgtctgtagtgttttgtggagtactggaccaaaaaaataaa
gcattataaatatatagttttatttataaggccttttctattgtgtgttttactgttgattaataaatgttatttctggacaa

In some embodiments of any of the aspects, the scg2 neuropeptide is a cleavage product of secretogranin II (scg2) polypeptide. In some embodiments of any of the aspects, the scg2 neuropeptide, when present in the Scg2 polypeptide, is flanked at its N-terminus and/or at its C-terminus by a dibasic cleavage residue. In some embodiments of any of the aspects, the dibasic cleavage residue is selected from the group consisting of: arginine-lysine (RK); lysine-arginine (KR); and arginine-arginine (RR). In some embodiments of any of the aspects, the dibasic cleavage residue is lysine-arginine (KR). In some embodiments of any of the aspects, the dibasic cleavage residue is arginine-lysine (RK). In some embodiments of any of the aspects, the dibasic cleavage residue is arginine-arginine (RR).

In some embodiments of any of the aspects, the scg2 neuropeptide is produced by a proprotein convertase, e.g., Pcsk1 protease and/or Pcsk2 protease, cleaving the scg2 polypeptide. In some embodiments of any of the aspects, the dibasic cleavage residue is a specific cleavage site for a proprotein convertase, e.g., Pcsk1 protease and/or Pcsk2 protease. Pcsk1 is naturally expressed only in neuroendocrine cells such as in the brain, pituitary, and adrenal tissues; Pcsk1 most often cleaves after a pair of basic residues within prohormones but can occasionally cleave after a single arginine. Pcsk1 and Pcsk2 are calcium (Ca 2⁺) activated serine endoproteases, meaning that a serine residue is part of the active site that hydrolyzes the peptide bond within the substrate.

In some embodiments of any of the aspects, the at least one scg2 neuropeptide is selected from the group consisting of: secretoneurin; EM66; manserin; and SgII; or any combination thereof. Non-limiting examples of scg2 neuropeptide combinations, which can be used in the pharmaceutical compositions and methods described herein, are provided in Table 2.

TABLE 2
Exemplary scg2 neuropeptide combinations

| secretoneurin | EM66 | manserin | SgII |
|---|---|---|---|
| X | | | |
| | X | | |
| X | X | | |
| | | X | |
| X | | X | |
| | X | X | |
| X | X | X | |
| | | | X |
| X | | | X |
| | X | | X |
| X | X | | X |
| | | X | X |
| X | | X | X |
| | X | X | X |
| X | X | X | X |

In some embodiments of any of the aspects, the scg2 neuropeptide comprises one of SEQ ID NOs: 5-8 or an amino acid sequence that is at least 80%, at least 85%, at least 90%, at least 91%, at least 92%, at least 93%, at least 94%, least 95%, at least 96%, at least 97%, at least 98%, at least 99%, at least 99.5% or more identical to one of SEQ ID NOs: 5-8 that maintains the same function (e.g., modulation of interneurons; e.g., increasing memory consolidation and/or memory retention; e.g., treating a memory-associated disorder, learning disability, neurodegenerative disease or disorder and/or epilepsy).

In some embodiments of any of the aspects, the scg2 neuropeptide comprises one of: aa 184-216 of SEQ ID NO: 33; aa 219-284 of SEQ ID NO: 33; aa 487-526 of SEQ ID NO: 33; aa 529-570 of SEQ ID NO: 33; aa 184-216 of SEQ ID NO: 34; aa 219-284 of SEQ ID NO: 34; aa 529-568 of SEQ ID NO: 34; aa 571-612 of SEQ ID NO: 34; aa 182-214 of SEQ ID NO: 35; aa 217-282 of SEQ ID NO: 35; aa 526-565 of SEQ ID NO: 35; or aa 568-609 of SEQ ID NO: 35, or an amino acid sequence that is at least 80%, at least 85%, at least 90%, at least 91%, at least 92%, at least 93%, at least 94%, least 95%, at least 96%, at least 97%, at least 98%, at least 99%, at least 99.5% or more identical to one of aa 184-216 of SEQ ID NO: 33; aa 219-284 of SEQ ID NO: 33; aa 487-526 of SEQ ID NO: 33; aa 529-570 of SEQ ID NO: 33; aa 184-216 of SEQ ID NO: 34; aa 219-284 of SEQ ID NO: 34; aa 529-568 of SEQ ID NO: 34; aa 571-612 of SEQ ID NO: 34; aa 182-214 of SEQ ID NO: 35; aa 217-282 of SEQ ID NO: 35; aa 526-565 of SEQ ID NO: 35; or aa 568-609 of SEQ ID NO: 35, that maintains the same function (e.g., modulation of interneurons; e.g., increasing memory consolidation and/or memory retention; e.g., treating a memory-associated disorder, learning disability, neurodegenerative disease or disorder and/or epilepsy).

In some embodiments of any of the aspects, the scg2 neuropeptide is secretoneurin. In some embodiments of any of the aspects, secretoneurin comprises TNEIVEEQYTPQSLATLESVFQELGKLTGPNNQ (SEQ ID NO: 5; 33 aa; see e.g., residues 182-214 of SEQ ID NO: 4). In some embodiments of any of the aspects, secretoneurin comprises aa 184-216 of SEQ ID NO: 33; aa 184-216 of SEQ ID NO: 34; or aa 182-214 of SEQ ID NO: 35.

In some embodiments of any of the aspects, the scg2 neuropeptide is EM66. In some embodiments of any of the aspects, EM66 comprises ERMDEEQKLYTDDEDDIYKANNIAYEDVVGGEDWNPVEEKIESQTQEEVRDSKENIEKNEQI NDEM (SEQ ID NO: 6; 66 aa; see e.g., residues 217-282 of SEQ ID NO: 4). In some embodiments of any of the aspects, EM66 comprises aa 219-284 of SEQ ID NO: 33; aa 219-284 of SEQ ID NO: 34; or aa 217-282 of SEQ ID NO: 35.

In some embodiments of any of the aspects, the scg2 neuropeptide is manserin. In some embodiments of any of the aspects, manserin comprises VPGQGSSEDDLQEEEQIEQAIKEHLNQGSSQETDKLAPVS (SEQ ID NO: 7; 40 aa; see e.g., residues 527-566 of SEQ ID NO: 4). In some embodiments of any of the aspects, manserin comprises aa 487-526 of SEQ ID NO: 33; aa 529-568 of SEQ ID NO: 34; or aa 526-565 of SEQ ID NO: 35.

In some embodiments of any of the aspects, the scg2 neuropeptide is SgII. In some embodiments of any of the aspects, SgII comprises FPVGPPKNDDTPNRQYWDEDLLMKVLEYLNQEKAEKGREHIA (SEQ ID NO: 8; 42 aa; see e.g., residues 569-610 of SEQ ID NO: 4). In some embodiments of any of the aspects, SgII comprises aa 529-570 of SEQ ID NO: 33; aa 571-612 of SEQ ID NO: 34; or aa 568-609 of SEQ ID NO: 35.

In some embodiments of any of the aspects, the scg2 neuropeptide comprises a functional fragment of one of SEQ ID NOs: 5-8 that retains at least 50%, at least 60%, at least 70%, at least 80%, at least 85%, at least 90%, at least 91%, at least 92%, at least 93%, at least 94%, least 95%, at least 96%, at least 97%, at least 98%, at least 99%, at least 99.5% or more of the wild-type scg2 neuropeptide's activity (e.g., modulation of interneurons; e.g., increasing memory consolidation and/or memory retention; e.g., treating a memory-associated disorder, learning disability, neurodegenerative disease or disorder and/or epilepsy) according to the assays described below herein. A functional fragment of the scg2 neuropeptide can comprise conservative substitutions of the sequences disclosed herein (e.g., SEQ ID NOs: 5-8).

In some embodiments of any of the aspects, the scg2 neuropeptide comprises a functional fragment of one of: aa 184-216 of SEQ ID NO: 33; aa 219-284 of SEQ ID NO: 33; aa 487-526 of SEQ ID NO: 33; aa 529-570 of SEQ ID NO: 33; aa 184-216 of SEQ ID NO: 34; aa 219-284 of SEQ ID NO: 34; aa 529-568 of SEQ ID NO: 34; aa 571-612 of SEQ ID NO: 34; aa 182-214 of SEQ ID NO: 35; aa 217-282 of SEQ ID NO: 35; aa 526-565 of SEQ ID NO: 35; or aa 568-609 of SEQ ID NO: 35; that retains at least 50%, at least 60%, at least 70%, at least 80%, at least 85%, at least 90%, at least 91%, at least 92%, at least 93%, at least 94%, least 95%, at least 96%, at least 97%, at least 98%, at least 99%, at least 99.5% or more of the wild-type scg2 neuropeptide's activity.

In some embodiments of any of the aspects, the scg2 neuropeptide comprises at least 10 to at most 66 amino acid residues. In some embodiments of any of the aspects, the scg2 neuropeptide comprises at least 33 to at most 66 amino acid residues. In some embodiments of any of the aspects, the scg2 neuropeptide comprises at least 40 to at most 66 amino acid residues. In some embodiments of any of the aspects, the scg2 neuropeptide comprises at least 42 to at most 66 amino acid residues. In some embodiments of any of the aspects, the scg2 neuropeptide comprises at least 33 to at most 42 amino acid residues. In some embodiments of any of the aspects, the scg2 neuropeptide comprises at least 33 to at most 40 amino acid residues.

In some embodiments of any of the aspects, the scg2 neuropeptide comprises at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 44, at least 45, at least 46, at least 47, at least 48, at least 49, at least 50, at least 51, at least 52, at least 53, at least 54, at least 55, at least 56, at least 57, at least 58, at least 59, at least 60, at least 61, at least 62, at least 63, at least 64, at least 65, at least 66 amino acid residues.

In some embodiments of any of the aspects, the scg2 neuropeptide comprises at most 10, at most 11, at most 12, at most 13, at most 14, at most 15, at most 16, at most 17, at most 18, at most 19, at most 20, at most 21, at most 22, at most 23, at most 24, at most 25, at most 26, at most 27, at most 28, at most 29, at most 30, at most 31, at most 32, at most 33, at most 34, at most 35, at most 36, at most 37, at most 38, at most 39, at most 40, at most 41, at most 42, at most 43, at most 44, at most at most 46, at most 47, at most 48, at most 49, at most 50, at most 51, at most 52, at most 53, at most 54, at most 55, at most 56, at most 57, at most 58, at most 59, at most 60, at most 61, at most 62, at most 63, at most 64, at most 65, at most 66 amino acid residues.

In multiple aspects, described herein are pharmaceutical compositions comprising a nucleic acid, vector, or viral vector that encodes for at least one of the scg2 neuropeptides described herein, and a pharmaceutically acceptable carrier. In one aspect, described herein is a pharmaceutical composition comprising a cell that expresses at least one of the scg2 neuropeptides described herein, and a pharmaceutically acceptable carrier. Such nucleic acids, vectors, viral vectors, and cells are described further herein.

In some embodiments, the technology described herein relates to a pharmaceutical composition comprising at least one of the scg2 neuropeptides as described herein or a nucleic acid, vector, or viral vector encoding at least one of the scg2 neuropeptides as described herein, and optionally a pharmaceutically acceptable carrier. In some embodiments, the active ingredients of the pharmaceutical composition comprise at least one of the scg2 neuropeptides as described herein or a nucleic acid, vector, or viral vector encoding at least one of the scg2 neuropeptides as described herein. In some embodiments, the active ingredients of the pharmaceutical composition consist essentially of at least one of the scg2 neuropeptides as described herein or a nucleic acid, vector, or viral vector encoding at least one of the scg2 neuropeptides as described herein. In some embodiments, the active ingredients of the pharmaceutical composition consist of at least one of the scg2 neuropeptides as described herein or a nucleic acid, vector, or viral vector encoding at least one of the scg2 neuropeptides as described herein.

Pharmaceutically acceptable carriers and diluents include saline, aqueous buffer solutions, solvents and/or dispersion media. The use of such carriers and diluents is well known in the art. Some non-limiting examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, methylcellulose, ethyl cellulose, microcrystalline cellulose and cellulose acetate; (4) powdered tragacanth; (5) malt; (6) gelatin; (7) lubricating agents, such as magnesium stearate, sodium lauryl sulfate and talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol (PEG); (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; (22) bulking agents, such as polypeptides and amino acids; (23) serum component, such as serum albumin, HDL and LDL; (24) C₂-C₁₂ alcohols, such as ethanol; and (25) other non-toxic compatible substances employed in pharmaceutical formulations. Wetting agents, coloring agents, release agents, coating agents, sweetening agents, flavoring agents, perfuming agents, preservative and antioxidants can also be present in the formulation. The terms such as “excipient”, “carrier”, “pharmaceutically acceptable carrier” or the like are used interchangeably herein. In some embodiments, the carrier inhibits the degradation of the active agent, e.g. at least one of the scg2 neuropeptides as described herein or a nucleic acid, vector, or viral vector encoding at least one of the scg2 neuropeptides as described herein.

In some embodiments of any of the aspects, the pharmaceutical composition is formulated for delivery to the central nervous system (CNS). In some embodiments of any of the aspects, the pharmaceutical composition is formulated for delivery across the blood-brain barrier. In some embodiments of any of the aspects, the pharmaceutical composition is formulated for delivery to the brain. As used herein, the term “formulated for” refers to formulations that permit delivery of the pharmaceutical compositions described herein to the specific locations, organs, tissues, or cells indicated, e.g., across the tightly controlled barrier of the blood-brain barrier and into the CNS. The central nervous system (CNS) functions in a tightly controlled and stable environment. This is maintained by highly specialized blood vessels that physically seal the CNS and control substance influx/efflux, known as the “blood brain barrier” (BBB). Specialized tight junctions between endothelial cells comprising a single layer that lines the CNS capillaries are the physical seal between blood and brain. BBB selectivity is facilitated by an array of endothelial transporters responsible for the supply of nutrients and for the clearance of waste or toxins. In concert with pericytes and astrocytes, the BBB protects the brain from various toxins and pathogens and provides the proper chemical composition for synaptic transmissions. Accordingly, provided herein are exemplary formulations for delivery across the blood-brain barrier and/or delivery to the brain. Non-limiting examples of formulations which permit delivery of pharmaceutical compositions across the BBB and into the brain include: direct injection or infusion into the CNS; formulation as a solution, e.g., comprising a carrier protein; formulation as a nanoparticle; formulation as a liposome; formulation as a nucleic acid; formulation as a CNS-tropic viral vector; formulation with or linkage to an agent that is endogenously transported across the BBB; formulation with or linkage to a cell penetrating peptide (CPP); formulation with or linkage to a BBB-shuttle; formulation with or linkage to an agent that increases permeability of the BBB. In embodiments wherein at least one Scg2 neuropeptide is linked to another agent (e.g., cationic substrate; an agent that is endogenously transported across the BBB; a cell penetrating peptide (CPP); a BBB-shuttle; or an agent that increases permeability of the BBB), the N-terminus and/or the C-terminus of the Scg2 neuropeptide can be linked to the other agent; as non-limiting examples, such a linkage can be a flexible amino acid linker (e.g., a Gly-Ser motif), or a cleavage linker as known in the art.

In some embodiments of any of the aspects, the pharmaceutical composition, nucleic acid, vector, or viral vector is administered to the central nervous system. In some embodiments of any of the aspects, the pharmaceutical composition, nucleic acid, vector, or viral vector is administered intracranially, epidurally, intrathecally, intraparenchymally, intraventricularly, or subarachnoidly. In some embodiments of any of the aspects, the pharmaceutical composition, nucleic acid, vector, or viral vector is administered intranasally. In some embodiments of any of the aspects, the pharmaceutical composition, nucleic acid, vector, or viral vector is administered in a formulation that crosses the blood-brain barrier, as described further herein. In some embodiments of any of the aspects, the pharmaceutical composition, nucleic acid, vector, or viral vector is administered via direct injection into the CNS or brain, e.g., a specific region of the brain such as in or near the hippocampus. In some embodiments of any of the aspects, the pharmaceutical composition, nucleic acid, vector, or viral vector is administered via infusion into the CNS or brain, e.g., via a shunt. In some embodiments of any of the aspects, the pharmaceutical composition, nucleic acid, vector, or viral vector is administered into the brain using an invasive method, such as the use of polymers or microchip systems, stereotactically guided drug insertion through a catheter, or transient disruption of the BBB.

In some embodiments of any of the aspects, the pharmaceutical composition is formulated as a solution comprising at least one of the Scg2 neuropeptides, wherein the solution is a liquid pharmaceutically acceptable carrier, as described herein or known in the art. In some embodiments of any of the aspects, the solution is saline (e.g., PBS). In some embodiments of any of the aspects, the solution further comprises a carrier protein, such as BSA. In some embodiments of any of the aspects, the solution further comprises a carrier protein that increases delivery across the BBB, such as the carrier protein CRM197, which is the non-toxic mutant of diphtheria toxin that uses the membrane-bound precursor of heparin-binding epidermal growth factor (HBEGF) as its transport receptor, which is constitutively expressed on the blood-brain barrier. In some embodiments of any of the aspects, the least one Scg2 neuropeptide is at a concentration of at least 0.1 nM/mL, at least 1 nM/mL, at least 10 nM/mL, at least 100 nM/mL, at least 1 uM/mL, at least 10 uM/mL, at least 100 uM/mL, at least 1 mM/mL, at least 10 nM/mL, at least 100 mM/mL or more.

In some embodiments of any of the aspects, the pharmaceutical composition is formulated as a nanoparticle, e.g., that can cross the BBB. Non-limiting examples of such nanoparticle formulations include liposomes, polymeric nanoparticles, carbon nanotubes, nanofibers, dendrimers, micelles, inorganic nanoparticles made of iron oxide, or gold nanoparticles. In some embodiments of any of the aspects, the pharmaceutical composition is formulated as a liposome, polyarginine, protamine, or cyclodextrin-based nanoparticle. In some embodiments of any of the aspects, the pharmaceutical composition is formulated as liposomes. Liposomes are roughly nano- or microsize vesicles consisting of one or more lipid bilayers surrounding an aqueous compartment. In some embodiments of any of the aspects, the liposomes comprise DMPC, dimyristoylphosphatidylcholine; DMPG, dimyristoylphosphatidylglycerol; DOPC, dioleoylphosphatidylcholine; DPPG, dipalmitoylphosphatidylglycerol; DSPC, distearoylphosphatidylcholine; DSPE, distearoylphosphatidylethanolamine; DSPG, distearoylphosphatidylglycerol; EPC, egg phosphatidylcholine; HSPC, hydrogenated soy phosphatidylcholine; PEG, polyethylene glycol; DSPE-PEG2,000; cholesterol; and/or triolein. In some embodiments of any of the aspects, the liposome is cationized. In some embodiments of any of the aspects, at least one Scg2 neuropeptide is linked to a poly-cationic polymer such as poly-ethyleneimine, or otherwise incorporated into a liposomal delivery system. In some embodiments of any of the aspects, the liposome comprising a targeting ligand (e.g., brain-targeted aptamers or antibodies, such as the cell-penetrating peptides or BBB-shuttles, as described further herein, or known in the art). In some embodiments of any of the aspects, the liposome can be triggered to release the at least one Scg2 neuropeptide, e.g., using external stimuli, such as variations in magnetic field, temperature, ultrasound intensity, light or electric pulses, and others. See e.g., Vieira and Gamarra, Int J Nanomedicine. 2016; 11: 5381-5414, the content of which is incorporated herein by reference in its entirety.

In some embodiments of any of the aspects, the at least one Scg2 neuropeptide is formulated as a nucleic acid, e.g., mRNA, non-limiting examples of which are provided herein. In some embodiments of any of the aspects, the at least one Scg2 neuropeptide nucleic acid is formulated as liposome, e.g., a cationic liposome formulation comprising mRNA that encodes for at least one Scg2 neuropeptide. In some embodiments of any of the aspects, the pharmaceutical composition is formulated as a CNS-tropic viral vector. Viral tropism is the ability of a given virus to productively infect a particular cell (cellular tropism), tissue (tissue tropism) or host species (host tropism). As a non-limiting example, the pharmaceutical composition is formulated as an AAV (e.g., AAV2/1, AAVDJ8, or AAV9); a herpes simplex virus (e.g., HSV-1); or a lentivirus (e.g., pseudotyped with a glycoprotein that targets neurons or glial cells; e.g., glycoprotein from a neurotropic virus such as vesicular stomatitis virus G (VSV-G), lymphocytic choriomeningitis virus (LCMV), rabies, or Mokola lyssavirus); see e.g., Gray et al., Ther Deliv. 2010, 1(4): 517-534, the content of which is incorporated herein by reference in its entirety.

In some embodiments of any of the aspects, the at least one Scg2 neuropeptide is linked to an agent that is endogenously transported across the BBB, e.g., insulin, transferrin, insulin like growth factor (IGF), leptin, low density lipoprotein (LDL) and fragments or peptidomimetics or derivatives thereof, which can undergo receptor-mediated transport (RMT) across the BBB in vivo. In some embodiments of any of the aspects, the at least one Scg2 neuropeptide is linked to a peptidomimetic monoclonal antibody (MAb) of an agent that is endogenously transported across the BBB, e.g., mAbs for the insulin receptor, the transferrin receptor, the IGF receptor, the leptin receptor, or the LDL receptor. In some embodiments of any of the aspects, the at least one Scg2 neuropeptide is linked to a cationic substance that can cross the BBB by adsorption-mediated transcytosis or endocytosis.

In some embodiments of any of the aspects, the at least one Scg2 neuropeptide is linked to a cell penetrating peptide (CPP). CPPs are short peptides that facilitate cellular intake and uptake of molecules through endocytosis. CPPs typically have an amino acid composition that either contains a high relative abundance of positively charged amino acids such as lysine or arginine or has sequences that contain an alternating pattern of polar, charged amino acids and non-polar, hydrophobic amino acids. These two types of structures are referred to as polycationic or amphipathic, respectively. A third class of CPPs are the hydrophobic peptides, containing only apolar residues with low net charge or hydrophobic amino acid groups that are crucial for cellular uptake. In some embodiments of any of the aspects, the CPP is selected from pVEC (LLIILRRRIRKQAHAHSK, SEQ ID NO: 39), SynB3 (RRLSYSRRRF, SEQ ID NO: 40), Tat 47-57 (YGRKKRRQRRR, SEQ ID NO: 41), transportan 10 (TP10; AGYLLGKINLKALAALAKKIL, SEQ ID NO: 42). In some embodiments of any of the aspects, the CPP is Rabies Virus Glycoprotein, which is a 29-amino-acid cell penetrating peptide derived from a rabies virus glycoprotein that can cross the blood-brain barrier (BBB) and enter brain cells (YTIWMPENPRPGTPCDIFTNSRGKRASNG, SEQ ID NO: 43). RVG peptide is successfully used to carry a variety of cargos into brain cells such as plasmids, siRNAs, proteins, and nanoparticles; see e.g., US Patent Publication 2018-0028677A1, the content of which is incorporated herein by reference in its entirety.

In some embodiments of any of the aspects, the at least one Scg2 neuropeptide is linked to a BBB-shuttle. BBB-shuttles are peptides designed to target BBB receptors in order to gain access to the brain by transcytosis. In some embodiments of any of the aspects, the BBB-shuttle is selected from one of SEQ ID NOs: 44-68 or an amino acid sequence that is at least 80%, at least 85%, at least 90%, at least 91%, at least 92%, at least 93%, at least 94%, least 95%, at least 96%, at least 97%, at least 98%, at least 99%, at least 99.5% or more identical to one of SEQ ID NOs: 44-68 that maintains the same function (e.g., BBB transcytosis) (see e.g., Table 3). In some embodiments of any of the aspects, nanoparticles, comprising at least one Scg2 neuropeptide, and the nanoparticles are linked to at least one BBB-shuttle. See e.g., McCully et al., Curr Pharm Des. 2018 April, 24(13): 1366-1376, the content of which is incorporated herein by reference in its entirety.

TABLE 3
Exemplary BBB-shuttles

| BBB- | | | SEQ ID |
|---|---|---|---|
| Shuttle | Target | Sequence | NO |
| Ang-2 | LDR 1 | TFFYGGSRGKRNNFKTEEY | 44 |
| | | Yeetkfnnrkgrsggyfft | 45 |
| ApoE | LDLR | LRKLRKRLLR | 46 |
| (141-150) | | | |
| B6 | hTrR | CGHKAKGPRK | 47 |
| Cyclic- | Integrin | RGDfK (cyclized peptide) | 48 |
| RGD | R | | |
| CDX | nAchR | FKESWREARGTRIERG | 49 |
| ^{D}CDX | nAchR | GreirtGraerwsekf | 50 |
| Enk | Opioid | YGGFLGGYTGFLS-O-beta-glucoside | 51 |
| Gly-copep | receptor | | |
| g7 | | GFtGFLS-(monosaccharide) | 52 |
| gH625 | | HGLASTLTRWAHYNALIRAFGGG | 53 |
| Gluthatione | Mrp/ | GSH | 54 |
| | Abcc | | |
| LPFFD | RAGE | LPFFD | 55 |
| MiniAp-4 | | Dap-KAPETALD (cyclized peptide); Dap | 56 |
| | | stands for diaminopropionic acid | |
| Penetratin | CPP | RQIKIWFQNRRMKWKK | 57 |
| RDP | nAchR | KSVRTWNEIIPSKGCLRVGGRCHPHVNGGGRRRRRRRRR | 58 |
| peptide | | | |
| RVG29 | nAchR | YTWMPENPRPGTPCDIFTNSRGKRASNGGGGGGC | 59 |
| CTX | MMP-2, | MCMPCFTTDHQMARKCDDCCGGKGRGKCYGPQCLCR | 60 |
| | Annexin | | |
| | A, Cl- | | |
| | channels | | |
| T7-HAI | TfR | HAIYPRH | 61 |
| TAT | AME | GRKKRRQRRRPPQGWC | 62 |
| | | YGRKKRRQRRR | 63 |
| TATre | AME | rrrqrrkkrGy | 64 |
| TGN | | TGNYKALHPHNG | 65 |
| THR | TfR | THRPPMWSPVWP | 66 |
| THRre | TfR | pwvpswmpprht | 67 |
| Peptide-22 | LDLR | C-MPRLRGC (cyclized peptide) | 68 |

In some embodiments of any of the aspects, the pharmaceutical composition further comprises at least one agent that increases the permeability of the blood-brain barrier, e.g., so as to allow the Scg2 neuropeptides(s) described herein to cross the BBB and enter the CNS. In some embodiments of any of the aspects, the pharmaceutical composition is co-administered with at least one agent that increases the permeability of the blood-brain barrier. Non-limiting examples of agents that increase the permeability of the blood-brain barrier include: claudin-5 and/or occludin inhibitors; peptides derived from zonula occludens toxin; synthetic peptides targeting the extracellular loops of tight junctions; adenosine 2A receptors (A2AR) agonists; an inhibitor of a gene or gene expression product selected from the group consisting of: Mfsd2A; Slco1C1; Slc38A5; LRP8; Slc3A2; Slc7A5; Slc7A1; Slc6A6; IGFBP7; Glutl; Slc40A1; and Slc30A1; See e.g., US20160120893A1, the content of which is incorporated herein by reference in its entirety.

In some embodiments of any of the aspects, the pharmaceutical composition is formulated for delivery to a specific cell. In some embodiments of any of the aspects, the pharmaceutical composition is formulated for delivery to the hippocampus (e.g., using direct injection or infusion into the hippocampus; using a nucleic acid, vector, or viral vector comprising a promoter that is specifically expressed in the hippocampus such as the Scg2 native promoter or the CaMK2a promoter). In some embodiments of any of the aspects, the pharmaceutical composition is formulated for delivery to pyramidal cells (e.g., using a nucleic acid, vector, or viral vector comprising a promoter that is specifically expressed in pyramidal cells, such as the Scg2 native promoter or the CaMK2a promoter).

In some embodiments, the pharmaceutical composition comprising at least one of the scg2 neuropeptides as described herein or a nucleic acid, vector, or viral vector encoding at least one of the scg2 neuropeptides as described herein can be a parenteral dose form. Since administration of parenteral dosage forms typically bypasses the patient's natural defenses against contaminants, parenteral dosage forms are preferably sterile or capable of being sterilized prior to administration to a patient. Examples of parenteral dosage forms include, but are not limited to, solutions ready for injection, dry products ready to be dissolved or suspended in a pharmaceutically acceptable vehicle for injection, suspensions ready for injection, and emulsions. In addition, controlled-release parenteral dosage forms can be prepared for administration of a patient, including, but not limited to, DUROS®-type dosage forms and dose-dumping.

Suitable vehicles that can be used to provide parenteral dosage forms of at least one of the scg2 neuropeptides as described herein are well known to those skilled in the art. Examples include, without limitation: sterile water; water for injection USP; saline solution; glucose solution; aqueous vehicles such as but not limited to, sodium chloride injection, Ringer's injection, dextrose injection, dextrose and sodium chloride injection, and lactated Ringer's injection; water-miscible vehicles such as, but not limited to, ethyl alcohol, polyethylene glycol, and propylene glycol; and non-aqueous vehicles such as, but not limited to, corn oil, cottonseed oil, peanut oil, sesame oil, ethyl oleate, isopropyl myristate, and benzyl benzoate.

Pharmaceutical compositions comprising at least one of the scg2 neuropeptides as described herein or a nucleic acid, vector, or viral vector encoding at least one of the scg2 neuropeptides as described herein can also be formulated to be suitable for oral administration, for example as discrete dosage forms, such as, but not limited to, tablets (including without limitation scored or coated tablets), pills, caplets, capsules, chewable tablets, powder packets, cachets, troches, wafers, aerosol sprays, or liquids, such as but not limited to, syrups, elixirs, solutions or suspensions in an aqueous liquid, a non-aqueous liquid, an oil-in-water emulsion, or a water-in-oil emulsion. Such compositions contain a predetermined amount of the pharmaceutically acceptable salt of the disclosed compounds, and may be prepared by methods of pharmacy well known to those skilled in the art. See generally, Remington: The Science and Practice of Pharmacy, 21st Ed., Lippincott, Williams, and Wilkins, Philadelphia PA. (2005).

Conventional dosage forms generally provide rapid or immediate drug release from the formulation. Depending on the pharmacology and pharmacokinetics of the drug, use of conventional dosage forms can lead to wide fluctuations in the concentrations of the drug in a patient's blood and other tissues. These fluctuations can impact a number of parameters, such as dose frequency, onset of action, duration of efficacy, maintenance of therapeutic blood levels, toxicity, side effects, and the like. Advantageously, controlled-release formulations can be used to control a drug's onset of action, duration of action, plasma levels within the therapeutic window, and peak blood levels. In particular, controlled- or extended-release dosage forms or formulations can be used to ensure that the maximum effectiveness of a drug is achieved while minimizing potential adverse effects and safety concerns, which can occur both from under-dosing a drug (i.e., going below the minimum therapeutic levels) as well as exceeding the toxicity level for the drug. In some embodiments, a pharmaceutical composition comprising at least one of the scg2 neuropeptides described herein or a nucleic acid, vector, or viral vector encoding at least one of the scg2 neuropeptides as described herein can be administered in a sustained release formulation.

Controlled-release pharmaceutical products have a common goal of improving drug therapy over that achieved by their non-controlled release counterparts. Ideally, the use of an optimally designed controlled-release preparation in medical treatment is characterized by a minimum of drug substance being employed to cure or control the condition in a minimum amount of time. Advantages of controlled-release formulations include: 1) extended activity of the drug; 2) reduced dosage frequency; 3) increased patient compliance; 4) usage of less total drug; 5) reduction in local or systemic side effects; 6) minimization of drug accumulation; 7) reduction in blood level fluctuations; 8) improvement in efficacy of treatment; 9) reduction of potentiation or loss of drug activity; and 10) improvement in speed of control of diseases or conditions. Kim, Cherng-ju, Controlled Release Dosage Form Design, 2 (Technomic Publishing, Lancaster, Pa.: 2000).

Most controlled-release formulations are designed to initially release an amount of drug (active ingredient) that promptly produces the desired therapeutic effect, and gradually and continually release other amounts of drug to maintain this level of therapeutic or prophylactic effect over an extended period of time. In order to maintain this constant level of drug in the body, the drug must be released from the dosage form at a rate that will replace the amount of drug being metabolized and excreted from the body. Controlled-release of an active ingredient can be stimulated by various conditions including, but not limited to, pH, ionic strength, osmotic pressure, temperature, enzymes, water, and other physiological conditions or compounds.

A variety of known controlled- or extended-release dosage forms, formulations, and devices can be adapted for use with the salts and compositions of the disclosure. Examples include, but are not limited to, those described in U.S. Pat. Nos. 3,845,770; 3,916,899; 3,536,809; 3,598,123; 4,008,719; 5,674,533; 5,059,595; 5,591,767; 5,120,548; 5,073,543; 5,639,476; 5,354,556; 5,733,566; and 6,365,185 B1; each of which is incorporated herein by reference. These dosage forms can be used to provide slow or controlled-release of one or more active ingredients using, for example, hydroxypropyl methylcellulose, other polymer matrices, gels, permeable membranes, osmotic systems (such as OROS® (Alza Corporation, Mountain View, Calif. USA)), or a combination thereof to provide the desired release profile in varying proportions.

In some embodiments of any of the aspects, the Scg2 neuropeptide(s) comprises an Scg2 peptidomimetic. In some embodiments of any of the aspects, the entire Scg2 neuropeptide is a peptidomimetic. In some embodiments of any of the aspects, a portion of the Scg2 neuropeptide comprises a peptidomimetic. A peptidomimetic is a small protein-like chain designed to mimic a peptide. A peptidomimetic can comprise a peptoid or β-peptide. There are at least two different approaches to design peptidomimetics: a medicinal chemistry approach, where parts of the peptide are successively replaced by non-peptide moieties until getting a non-peptide molecule and a biophysical approach, where a bioactive form of the peptide is sketched and peptidomimetics are designed based on hanging the appropriate chemical moieties on diverse scaffolds. See e.g., Perez, Curr Top Med Chem. 2018, 18(7):566-590; Ripka and Rich, Current Opinion in Chemical Biology Volume 2, Issue 4, 1998, Pages 441-452; D'Annessa et al., Front. Mol. Biosci., 5 May 2020; the contents of each of which are incorporated herein by reference in their entireties.

In some embodiments, a polypeptide, e.g., an Scg2 neuropeptide, as described herein can comprise at least one peptide bond replacement. An Scg2 neuropeptide as described herein can comprise one type of peptide bond replacement or multiple types of peptide bond replacements, e.g. 2 types, 3 types, 4 types, 5 types, or more types of peptide bond replacements. Non-limiting examples of peptide bond replacements include urea, thiourea, carbamate, sulfonyl urea, trifluoroethylamine, ortho-(aminoalkyl)-phenylacetic acid, para-(aminoalkyl)-phenylacetic acid, meta-(aminoalkyl)-phenylacetic acid, thioamide, tetrazole, boronic ester, olefinic group, and derivatives thereof.

In some embodiments, a polypeptide, e.g., an Scg2 neuropeptide, as described herein can comprise naturally occurring amino acids commonly found in polypeptides and/or proteins produced by living organisms, e.g. Ala (A), Val (V), Leu (L), Ile (I), Pro (P), Phe (F), Trp (IV), Met (M), Gly (G), Ser (S), Thr (T), Cys (C), Tyr (Y), Asn (N), Gln (Q), Asp (D), Glu (E), Lys (K), Arg (R), and His (H). In some embodiments, an Scg2 neuropeptide as described herein can comprise alternative amino acids. Non-limiting examples of alternative amino acids include, D-amino acids; beta-amino acids; homocysteine, phosphoserine, phosphothreonine, phosphotyrosine, hydroxyproline, gamma-carboxyglutamate; hippuric acid, octahydroindole-2-carboxylic acid, statine, 1,2,3,4,-tetrahydroisoquinoline-3-carboxylic acid, penicillamine (3-mercapto-D-valine), ornithine, citruline, alpha-methyl-alanine, para-benzoylphenylalanine, para-amino phenylalanine, p-fluorophenylalanine, phenylglycine, propargylglycine, sarcosine, and tert-butylglycine), diaminobutyric acid, 7-hydroxy-tetrahydroisoquinoline carboxylic acid, naphthylalanine, biphenylalanine, cyclohexylalanine, amino-isobutyric acid, norvaline, norleucine, tert-leucine, tetrahydroisoquinoline carboxylic acid, pipecolic acid, phenylglycine, homophenylalanine, cyclohexylglycine, dehydroleucine, 2,2-diethylglycine, 1-amino-1-cyclopentanecarboxylic acid, 1-amino-1-cyclohexanecarboxylic acid, amino-benzoic acid, amino-naphthoic acid, gamma-aminobutyric acid, difluorophenylalanine, nipecotic acid, alpha-amino butyric acid, thienyl-alanine, t-butylglycine, trifluorovaline; hexafluoroleucine; fluorinated analogs; azide-modified amino acids; alkyne-modified amino acids; cyano-modified amino acids; and derivatives thereof.

In some embodiments, a polypeptide, e.g. an Scg2 neuropeptide, can be modified, e.g. by addition of a moiety to one or more of the amino acids that together comprise the peptide. In some embodiments, a polypeptide as described herein can comprise one or more moiety molecules, e.g. 1 or more moiety molecules per polypeptide, 2 or more moiety molecules per polypeptide, 5 or more moiety molecules per polypeptide, 10 or more moiety molecules per polypeptide or more moiety molecules per polypeptide. In some embodiments, a polypeptide as described herein can comprise one more types of modifications and/or moieties, e.g. 1 type of modification, 2 types of modifications, 3 types of modifications or more types of modifications. Non-limiting examples of modifications and/or moieties include PEGylation; glycosylation; HESylation; ELPylation; lipidation; acetylation; amidation; end-capping modifications; cyano groups; phosphorylation; albumin, and cyclization. In some embodiments, an end-capping modification can comprise acetylation at the N-terminus, N-terminal acylation, and N-terminal formylation. In some embodiments, an end-capping modification can comprise amidation at the C-terminus, introduction of C-terminal alcohol, aldehyde, ester, and thioester moieties. The half-life of a polypeptide can be increased by the addition of moieties, e.g. PEG, albumin, or other fusion partners (e.g. Fc fragment of an immunoglobulin).

In some embodiments of any of the aspects, the Scg2 neuropeptide(s) present in a composition, or combination, of the disclosure exhibit an increased utility that is not exhibited when said Scg2 neuropeptide(s) occur alone or when said Scg2 neuropeptide(s) are present at a naturally occurring concentration. In some embodiments of any of the aspects, compositions of the disclosure, comprising Scg2 neuropeptide(s) as taught herein, exhibit a synergistic effect on imparting at least one improved trait in a cell contacted therewith or a subject treated therewith. In some embodiments of any of the aspects, the compositions of the disclosure-comprising Scg2 neuropeptide(s) as taught herein-exhibit markedly different characteristics/properties compared to their closest naturally occurring counterpart. That is, the compositions of the disclosure exhibit markedly different functional and/or structural characteristics/properties, as compared to their closest naturally occurring counterpart. For instance, the Scg2 neuropeptide(s) of the disclosure are structurally different from an Scg2 neuropeptide as it naturally exists in a cell (e.g., a neuron) or extracellular fluid surrounding said cell, for at least the following reasons: said Scg2 neuropeptide(s) can be isolated and purified, such that it is not found in the milieu of the cell (e.g., a neuron) or extracellular fluid surrounding said cell, said Scg2 neuropeptide(s) can be present at concentrations that do not occur in the cell (e.g., a neuron) or extracellular fluid surrounding said cell, said Scg2 neuropeptide(s) can be associated with acceptable carriers that do not occur in the cell (e.g., a neuron) or extracellular fluid surrounding said cell, said Scg2 neuropeptide(s) can be formulated to be shelf-stable and exist outside the environment of the cell (e.g., a neuron) or extracellular fluid surrounding said cell, and said Scg2 neuropeptide(s) can be combined with other Scg2 neuropeptide(s), e.g., at concentrations that do not exist in the cell (e.g., a neuron) or extracellular fluid surrounding said cell. Further, the Scg2 neuropeptide(s) of the disclosure are functionally different from an Scg2 neuropeptide as it naturally exists in a cell (e.g., a neuron) or extracellular fluid surrounding said cell, for at least the following reasons: said Scg2 neuropeptide(s) when applied in an isolated and purified form can lead to increased nervous system effects (e.g., modulation of interneurons; e.g., increasing memory consolidation and/or memory retention; e.g., treating a memory-associated disorder, learning disability, neurodegenerative disease or disorder and/or epilepsy), said Scg2 neuropeptide(s) can be formulated to be shelf-stable and able to exist outside the environment of the cell (e.g., a neuron) or extracellular fluid surrounding said cell, such that the Scg2 neuropeptide(s) now has a new utility as a supplement capable of administration to a subject in need thereof or to a cell culture, wherein the Scg2 neuropeptide(s) could not have such a utility in its natural state in the cell (e.g., a neuron) or extracellular fluid surrounding said cell, as the Scg2 neuropeptide(s) would be unable to survive outside the cell (e.g., a neuron) or extracellular fluid surrounding said cell without the intervention of the hand of man to formulate the Scg2 neuropeptide(s) into a shelf-stable state and impart this new utility that has the aforementioned functional characteristics not possessed by the Scg2 neuropeptide(s) in its natural state of existence in the cell (e.g., a neuron) or extracellular fluid surrounding said cell.

In one aspect, described herein is a cell culture medium comprising at least one Scg2 neuropeptide. As used herein the term “cell culture medium” or “culture medium” refers to a solid, liquid or semi-solid designed to support the growth of cells. In some embodiments of any of the aspects, the culture medium is a liquid. In some embodiments of any of the aspects, the culture medium comprises both the liquid medium and the cells within it (e.g., neuronal cells).

In some embodiments of any of the aspects, the scg2 neuropeptide in the cell culture medium is selected from the group consisting of: secretoneurin; EM66; manserin; and SgII; or any combination thereof. Such scg2 neuropeptide combinations can be selected from the examples provided in Table 2. In some embodiments of any of the aspects, the concentration of the at least one scg2 neuropeptide in the cell culture medium is at a level sufficient to promote activation of a cultured neuron, e.g., at least 0.1 nM/mL, at least 1 nM/mL, at least 10 nM/mL, at least 100 nM/mL, at least 1 uM/mL, at least 10 uM/mL, at least 100 uM/mL, at least 1 mM/mL, at least 10 nM/mL, at least 100 mM/mL or more.

In some embodiments, the cell culture medium comprises NEUROBASAL media (GIBCO). In some embodiments, the cell culture medium comprises BrainPhys™ Neuronal Culture Medium. In some embodiments, the cell culture medium further comprises B27 supplement (e.g., at least 2%), penicillin (e.g., at least 50 U/ml), streptomycin (e.g., at least 50 U ml/L), and/or GLUTA-MAX (e.g., at least 1 mM). In some embodiments, the cell culture medium further comprises brain-derived neurotrophic factor (BDNF).

In one aspect, described herein is a method for culturing a cell, comprising contacting the cell with the cell culture medium that comprises at least one Scg2 neuropeptide, as described herein. In one aspect, described herein is a method for culturing a neuron, comprising contacting the neuron with the cell culture medium that comprises at least one Scg2 neuropeptide, as described herein. In some embodiments, such methods of culturing a cell or neuron can: increase the growth rate of the cell or neuron and/or modulate the activity of the cell or neuron (e.g., as compared to a cell or neuron contacted with a cell culture medium that does not comprise at least one Scg2 neuropeptide).

In some embodiments of any of the aspects, the cell is a neuronal cell. In some embodiments of any of the aspects, the cell is from a neuronal cell line (e.g., SH-SYSY neuroblastoma cells; NT2 cells, such as NTERA-2 CL.D1 (NT2/D1) ATCC® CRL-1973™; PC-12 cells (e.g., ATCC® CRL-1721™)), In some embodiments of any of the aspects, the cell is from a primary neuronal culture (e.g., murine, rat, non-human primate, or human primary neuronal cultures). In some embodiments of any of the aspects, the cell is a hippocampal cell. In some embodiments of any of the aspects, the cell is a pyramidal cell. In some embodiments of any of the aspects, the cell is a CA1 pyramidal cell. In some embodiments of any of the aspects, the cell is an interneuron (e.g., a PV-IN or CCK-IN). In some embodiments of any of the aspects, the cell is a CNS neuron. In some embodiments of any of the aspects, the cell is a peripheral nervous system (PNS) neuron. In some embodiments of any of the aspects, the cell is a motor neuron. In some embodiments of any of the aspects, the cell is a sensory neuron. In some embodiments of any of the aspects, the cell is a dorsal root ganglion neuron. In some embodiments of any of the aspects, the cell is a neuron from the enteric nervous system. In some embodiments of any of the aspects, the cell is a neuron from the Vagal nerve. In some embodiments of any of the aspects, the cell is a neuroendocrine cell. In some embodiments of any of the aspects, the cell is a pancreatic islet cell (e.g., that is polarizable). In some embodiments of any of the aspects, the cell is a central nervous system (CNS) glial cell selected from microglia, astrocytes, oligodendrocytes, radial glial cells, and ependymal cells. In some embodiments of any of the aspects, the cell is a PNS glial cell selected from Schwann cells, enteric glial cells, and satellite glial cells.

In multiple aspects, described herein are nucleic acid sequences encoding a secretogranin II (scg2) neuropeptide. In some embodiments, the Scg2 nucleic acids are mRNA. In some embodiments, the Scg2 mRNA is formulated as a liposome, e.g., for delivery across the BBB and into the brain. In some embodiments of any of the aspects, the at least one Scg2 neuropeptide nucleic acid is formulated as a cationic liposome. In some embodiments of any of the aspects, the scg2 neuropeptide is selected from the group consisting of: secretoneurin; EM66; manserin; and SgII; or any combination thereof. Such scg2 neuropeptide-encoding nucleic acid combinations can be selected from the examples provided in Table 2.

In some embodiments of any of the aspects, the nucleic acid sequence encoding a scg2 neuropeptide comprises one of SEQ ID NOs: 9-12 or a nucleic acid sequence that is at least 80%, at least 85%, at least 90%, at least 91%, at least 92%, at least 93%, at least 94%, least 95%, at least 96%, at least 97%, at least 98%, at least 99%, at least 99.5% or more identical to one of SEQ ID NOs: 9-12 that as a polypeptide maintains the same function (e.g., modulation of interneurons; e.g., increasing memory consolidation and/or memory retention; e.g., treating a memory-associated disorder, learning disability, neurodegenerative disease or disorder and/or epilepsy).

In some embodiments of any of the aspects, the nucleic acid sequence encoding a scg2 neuropeptide comprises one of SEQ ID NOs: 36-38 (or a portion thereof that encodes for the neuropeptides indicated in one of SEQ ID NOs: 33-35) or a nucleic acid sequence that is at least 80%, at least 85%, at least 90%, at least 91%, at least 92%, at least 93%, at least 94%, least 95%, at least 96%, at least 97%, at least 98%, at least 99%, at least 99.5% or more identical to one of SEQ ID NOs: 36-38 (or a portion thereof that encodes for the neuropeptides indicated in one of SEQ ID NOs: 33-35) that as a polypeptide maintains the same function (e.g., modulation of interneurons; e.g., increasing memory consolidation and/or memory retention; e.g., treating a memory-associated disorder, learning disability, neurodegenerative disease or disorder and/or epilepsy).

In some embodiments of any of the aspects, the nucleic acid sequence encodes secretoneurin. In some embodiments of any of the aspects, the nucleic acid sequence encoding secretoneurin comprises

ACAAATGAAATAGTGGAGGAACAATATACTCCTCAAAGCCTTGCTACATT
GGAATCTGTCTTCCAAGAGCTGGGGAAACTGACAGGACCAAACAACCAG
(SEQ ID NO: 9; 99 nucleotides (nt); see e.g.,
nt 544-642 of SEQ ID NO: 3).

In some embodiments of any of the aspects, the nucleic acid sequence encodes EM66. In some embodiments of any of the aspects, the nucleic acid sequence encoding EM66 comprises

GAGAGGATGGATGAGGAGCAAAAACTTTATACGGATGATGAAGATGATAT
CTACAAGGCTAATAACATTGCCTATGAAGATGTGGTCGGGGGAGAAGACT
GGAACCCAGTAGAGGAGAAAATAGAGAGTCAAACCCAGGAAGAGGTGAGA
GACAGCAAAGAGAATATAGAAAAAAA
TGAACAAATCAACGATGAGATG (SEQ ID NO: 10; 198 nt;
see e.g., nt 649-846 of SEQ ID NO: 3).

In some embodiments of any of the aspects, the nucleic acid sequence encodes manserin. In some embodiments of any of the aspects, the nucleic acid sequence encoding manserin comprises

GTTCCTGGTCAAGGCTCATCTGAAGATGACCTGCAGGAAGAGGAACAAAT
TGAGCAGGCCATCAAAGAGCATTTGAATCAAGGCAGCTCTCAGGAGACTG
ACAAGCTGGCCCCGGTGAGC (SEQ ID NO: 11; 120 nt;
see e.g., nt 1579-1698 of SEQ ID NO: 3).

In some embodiments of any of the aspects, the nucleic acid sequence encodes SgII. In some embodiments of any of the aspects, the nucleic acid sequence encoding SgII comprises

TTCCCTGTGGGGCCCCCGAAGAATGATGATACCCCAAATAGGCAGTACTG
GGATGAAGATCTGTTAATGAAAGTGCTGGAATACCTCAACCAAGAAAAGG
CAGAAAAGGGAAGGGAGCATATTGCT (SEQ ID NO: 12; 126 nt;
see e.g., nt 1705-1830 of SEQ ID NO: 3).

In some embodiments of any of the aspects, the nucleic acid sequence encodes a functional fragment of a scg2 neuropeptide. In some embodiments of any of the aspects, the nucleic acid sequence comprises a functional fragment of one of SEQ ID NOs: 9-12 that as a polypeptide retains at least 50%, at least 60%, at least 70%, at least 80%, at least 85%, at least 90%, at least 91%, at least 92%, at least 93%, at least 94%, least 95%, at least 96%, at least 97%, at least 98%, at least 99%, at least 99.5% or more of the wild-type scg2 neuropeptide's activity (e.g., modulation of interneurons; e.g., increasing memory consolidation and/or memory retention; e.g., treating a memory-associated disorder, learning disability, neurodegenerative disease or disorder and/or epilepsy) according to the assays described below herein. A nucleic acid encoding a functional fragment of the scg2 neuropeptide can comprise conservative substitutions of the sequences disclosed herein (e.g., SEQ ID NOs: 9-12).

In some embodiments of any of the aspects, the nucleic acid sequence encodes a functional fragment of a scg2 neuropeptide. In some embodiments of any of the aspects, the nucleic acid sequence comprises a functional fragment of one of SEQ ID NOs: 36-38 (or a portion thereof that encodes for the neuropeptides indicated in one of SEQ ID NOs: 33-35) that as a polypeptide retains at least 50%, at least 60%, at least 70%, at least 80%, at least 85%, at least 90%, at least 91%, at least 92%, at least 93%, at least 94%, least 95%, at least 96%, at least 97%, at least 98%, at least 99%, at least 99.5% or more of the wild-type scg2 neuropeptide's activity.

In some embodiments of any of the aspects, the nucleic acid sequence encoding the scg2 neuropeptide comprises at least 30 to at most 198 nucleic acid residues. In some embodiments of any of the aspects, the nucleic acid sequence encoding the scg2 neuropeptide comprises at least 99 to at most 198 nucleic acid residues. In some embodiments of any of the aspects, the nucleic acid sequence encoding the scg2 neuropeptide comprises at least 120 to at most 198 nucleic acid residues. In some embodiments of any of the aspects, the nucleic acid sequence encoding the scg2 neuropeptide comprises at least 126 to at most 198 nucleic acid residues. In some embodiments of any of the aspects, the nucleic acid sequence encoding the scg2 neuropeptide comprises at least 99 to at most 126 nucleic acid residues. In some embodiments of any of the aspects, the nucleic acid sequence encoding the scg2 neuropeptide comprises at least 99 to at most 120 nucleic acid residues.

In some embodiments of any of the aspects, the nucleic acid sequence encoding the scg2 neuropeptide comprises at least 30, at least 33, at least 36, at least 39, at least 42, at least 45, at least 48, at least 51, at least 54, at least 57, at least 60, at least 63, at least 66, at least 69, at least 72, at least at least 78, at least 81, at least 84, at least 87, at least 90, at least 93, at least 96, at least 99, at least 102, at least 105, at least 108, at least 111, at least 114, at least 117, at least 120, at least 123, at least 126, at least 129, at least 132, at least 135, at least 138, at least 141, at least 144, at least 147, at least 150, at least 153, at least 156, at least 159, at least 162, at least 165, at least 168, at least 171, at least 174, at least 177, at least 180, at least 183, at least 186, at least 189, at least 192, at least 195, at least 198 nucleic acid residues.

In some embodiments of any of the aspects, the nucleic acid sequence encoding the scg2 neuropeptide comprises at most 30, at most 33, at most 36, at most 39, at most 42, at most 45, at most 48, at most 51, at most 54, at most 57, at most 60, at most 63, at most 66, at most 69, at most 72, at most 75, at most 78, at most 81, at most 84, at most 87, at most 90, at most 93, at most 96, at most 99, at most 102, at most 105, at most 108, at most 111, at most 114, at most 117, at most 120, at most 123, at most 126, at most 129, at most 132, at most 135, at most 138, at most 141, at most 144, at most 147, at most 150, at most 153, at most 156, at most 159, at most 162, at most 165, at most 168, at most 171, at most 174, at most 177, at most 180, at most 183, at most 186, at most 189, at most 192, at most 195, at most 198 nucleic acid residues.

In some embodiments of any of the aspects, a nucleic acid (e.g., mRNA encoding at least one Scg2 neuropeptide) is chemically modified to enhance stability or other beneficial characteristics. The nucleic acids described herein may be synthesized and/or modified by methods well established in the art, such as those described in “Current protocols in nucleic acid chemistry,” Beaucage, S. L. et al. (Edrs.), John Wiley & Sons, Inc., New York, NY, USA, which is hereby incorporated herein by reference. Modifications include, for example, (a) end modifications, e.g., 5′ end modifications (phosphorylation, conjugation, inverted linkages, etc.) 3′ end modifications (conjugation, DNA nucleotides, inverted linkages, etc.), (b) base modifications, e.g., replacement with stabilizing bases, destabilizing bases, or bases that base pair with an expanded repertoire of partners, removal of bases (abasic nucleotides), or conjugated bases, (c) sugar modifications (e.g., at the 2′ position or 4′ position) or replacement of the sugar, as well as (d) backbone modifications, including modification or replacement of the phosphodiester linkages. Specific examples of nucleic acid compounds useful in the embodiments described herein include, but are not limited to nucleic acids containing modified backbones or no natural internucleoside linkages. nucleic acids having modified backbones include, among others, those that do not have a phosphorus atom in the backbone. For the purposes of this specification, and as sometimes referenced in the art, modified nucleic acids that do not have a phosphorus atom in their internucleoside backbone can also be considered to be oligonucleosides. In some embodiments of any of the aspects, the modified nucleic acid will have a phosphorus atom in its internucleoside backbone.

Modified nucleic acid backbones can include, for example, phosphorothioates, chiral phosphorothioates, phosphorodithioates, phosphotriesters, aminoalkylphosphotriesters, methyl and other alkyl phosphonates including 3′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those) having inverted polarity wherein the adjacent pairs of nucleoside units are linked 3′-5′ to 5′-3′ or 2′-5′ to 5′-2′. Various salts, mixed salts and free acid forms are also included. Modified nucleic acid backbones that do not include a phosphorus atom therein have backbones that are formed by short chain alkyl or cycloalkyl internucleoside linkages, mixed heteroatoms and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; others having mixed N, O, S and CH2 component parts, and oligonucleosides with heteroatom backbones, and in particular —CH2-NH—CH2-, —CH2-N(CH3)-O—CH2- [known as a methylene (methylimino) or MMI backbone], —CH2-O—N(CH3)-CH2-, —CH2-N(CH3)-N(CH3)-CH2- and —N(CH3)-CH2-CH2- [wherein the native phosphodiester backbone is represented as —O—P—O—CH2-].

In other nucleic acid mimetics, both the sugar and the internucleoside linkage, i.e., the backbone, of the nucleotide units are replaced with novel groups. The base units are maintained for hybridization with an appropriate nucleic acid target compound. One such oligomeric compound, an RNA mimetic that has been shown to have excellent hybridization properties, is referred to as a peptide nucleic acid (PNA). In PNA compounds, the sugar backbone of an RNA is replaced with an amide containing backbone, in particular an aminoethylglycine backbone. The nucleobases are retained and are bound directly or indirectly to aza nitrogen atoms of the amide portion of the backbone.

The nucleic acid can also be modified to include one or more locked nucleic acids (LNA). A locked nucleic acid is a nucleotide having a modified ribose moiety in which the ribose moiety comprises an extra bridge connecting the 2′ and 4′ carbons. This structure effectively “locks” the ribose in the 3′-endo structural conformation. The addition of locked nucleic acids to siRNAs has been shown to increase siRNA stability in serum, and to reduce off-target effects (Elmen, J. et al., (2005) Nucleic Acids Research 33(1):439-447; Mook, O R. et al., (2007) Mol. Canc. Ther. 6(3):833-843; Grunweller, A. et al., (2003) Nucleic Acids Research 31(12):3185-3193).

Modified nucleic acids can also contain one or more substituted sugar moieties. The nucleic acids described herein can include one of the following at the 2′ position: OH; F; O-, S-, or N-alkyl; O-, S-, or N-alkenyl; O-, S- or N-alkynyl; or O-alkyl-O-alkyl, wherein the alkyl, alkenyl and alkynyl may be substituted or unsubstituted C1 to C10 alkyl or C2 to C10 alkenyl and alkynyl. Exemplary suitable modifications include O[(CH2)nO]mCH3, O(CH2)nOCH3, O(CH2)nNH2, O(CH2)nCH3, O(CH2)nONH2, and O(CH2)nO[(CH2)nCH3)]2, where n and m are from 1 to about 10. In some embodiments of any of the aspects, nucleic acids include one of the following at the 2′ position: C1 to C10 lower alkyl, substituted lower alkyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of a nucleic acid, or a group for improving the pharmacodynamic properties of a nucleic acid, and other substituents having similar properties. In some embodiments of any of the aspects, the modification includes a 2′ methoxyethoxy (2′-O—CH₂CH₂OCH₃, also known as 2′-O-(2-methoxyethyl) or 2′-MOE) (Martin et al., Helv. Chim. Acta, 1995, 78:486-504) i.e., an alkoxy-alkoxy group. Another exemplary modification is 2′-dimethylaminooxyethoxy, i.e., a O(CH2)2ON(CH3)2 group, also known as 2′-DMAOE, as described in examples herein below, and 2′-dimethylaminoethoxyethoxy (also known in the art as 2′-O-dimethylaminoethoxyethyl or 2′-DMAEOE), i.e., 2′-O—CH2-O—CH2-N(CH2)2, also described in examples herein below.

Other modifications include 2′-methoxy (2′-OCH3), 2′-aminopropoxy (2′-OCH2CH2CH2NH2) and 2′-fluoro (2′-F). Similar modifications can also be made at other positions on the nucleic acid, particularly the 3′ position of the sugar on the 3′ terminal nucleotide or in 2′-5′ linked dsRNAs and the 5′ position of 5′ terminal nucleotide. Nucleic acids may also have sugar mimetics such as cyclobutyl moieties in place of the pentofuranosyl sugar.

A nucleic acid can also include nucleobase (often referred to in the art simply as “base”) modifications or substitutions. As used herein, “unmodified” or “natural” or “canonical” nucleobases include the purine bases adenine (A) and guanine (G), and the pyrimidine bases thymine (T), cytosine (C) and uracil (U). Modified or “non-canonical” nucleobases can include other synthetic and natural nucleobases including but not limited to as inosine, isocytosine, isoguanine, 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl anal other 8-substituted adenines and guanines, 5-halo, particularly 5-bromo, 5-trifluoromethyl and other 5-substituted uracils and cytosines, 7-methylguanine and 7-methyladenine, 8-azaguanine and 8-azaadenine, 7-deazaguanine and 7-deazaadenine and 3-deazaguanine and 3-deazaadenine. Certain of these nucleobases are particularly useful for increasing the binding affinity of the inhibitory nucleic acids featured in the invention. These include 5-substituted pyrimidines, 6-azapyrimidines and N-2, N-6 and 0-6 substituted purines, including 2-aminopropyladenine, 5-propynyluracil and 5-propynylcytosine. 5-methylcytosine substitutions have been shown to increase nucleic acid duplex stability by 0.6-1.2° C. (Sanghvi, Y. S., Crooke, S. T. and Lebleu, B., Eds., dsRNA Research and Applications, CRC Press, Boca Raton, 1993, pp. 276-278) and are exemplary base substitutions, even more particularly when combined with 2′-O-methoxyethyl sugar modifications. In some embodiments of any of the aspects, modified nucleobases can include d5SICS and dNAM, which are a non-limiting example of unnatural nucleobases that can be used separately or together as base pairs (see e.g., Leconte et. al. J. Am. Chem. Soc. 2008, 130, 7, 2336-2343; Malyshev et. al. PNAS. 2012. 109 (30) 12005-12010). In some embodiments of any of the aspects, the nucleic acid comprises any modified nucleobases known in the art, i.e., any nucleobase that is modified from an unmodified and/or natural nucleobase.

The preparation of the modified nucleic acids, backbones, and nucleobases described above are well known in the art.

Another modification of a nucleic acid featured in the invention involves chemically linking to the nucleic acid to one or more ligands, moieties or conjugates that enhance the activity, cellular distribution, pharmacokinetic properties, or cellular uptake of the nucleic acid. Such moieties include but are not limited to lipid moieties such as a cholesterol moiety (Letsinger et al., Proc. Natl. Acid. Sci. USA, 1989, 86: 6553-6556), cholic acid (Manoharan et al., Biorg. Med. Chem. Let., 1994, 4:1053-1060), a thioether, e.g., beryl-S-tritylthiol (Manoharan et al., Ann. N.Y. Acad. Sci., 1992, 660:306-309; Manoharan et al., Biorg. Med. Chem. Let., 1993, 3:2765-2770), a thiocholesterol (Oberhauser et al., Nucl. Acids Res., 1992, 20:533-538), an aliphatic chain, e.g., dodecandiol or undecyl residues (Saison-Behmoaras et al., EMBO J, 1991, 10:1111-1118; Kabanov et al., FEBS Lett., 1990, 259:327-330; Svinarchuk et al., Biochimie, 1993, 75:49-54), a phospholipid, e.g., di-hexadecyl-rac-glycerol or triethyl-ammonium 1,2-di-O-hexadecyl-rac-glycero-3-phosphonate (Manoharan et al., Tetrahedron Lett., 1995, 36:3651-3654; Shea et al., Nucl. Acids Res., 1990, 18:3777-3783), a polyamine or a polyethylene glycol chain (Manoharan et al., Nucleosides & Nucleotides, 1995, 14:969-973), or adamantane acetic acid (Manoharan et al., Tetrahedron Lett., 1995, 36:3651-3654), a palmityl moiety (Mishra et al., Biochim. Biophys. Acta, 1995, 1264:229-237), or an octadecylamine or hexylamino-carbonyloxycholesterol moiety (Crooke et al., J. Pharmacol. Exp. Ther., 1996, 277:923-937).

In multiple aspects, described herein are vectors or viral vectors comprising at least one nucleic acid sequence encoding a secretogranin II (scg2) neuropeptide. In some embodiments of any of the aspects, the scg2 neuropeptide is selected from the group consisting of: secretoneurin; EM66; manserin; and SgII; or any combination thereof. Such scg2 neuropeptide-encoding nucleic acid combinations can be selected from the examples provided in Table 2.

In some embodiments, one or more of the genes (e.g., scg2 neuropeptide(s)) described herein is expressed in a recombinant expression vector or plasmid. As used herein, the term “vector” refers to a polynucleotide sequence suitable for transferring transgenes into a host cell. The term “vector” includes plasmids, mini-chromosomes, phage, naked DNA and the like. See, for example, U.S. Pat. Nos. 4,980,285; 5,631,150; 5,707,828; 5,759,828; 5,888,783 and, 5,919,670, and, Sambrook et al, Molecular Cloning: A Laboratory Manual, 2nd Ed., Cold Spring Harbor Press (1989). One type of vector is a “plasmid,” which refers to a circular double stranded DNA loop into which additional DNA segments are ligated. Another type of vector is a viral vector, wherein additional DNA segments are ligated into the viral genome. Certain vectors are capable of autonomous replication in a host cell into which they are introduced (e.g., bacterial vectors having a bacterial origin of replication and episomal mammalian vectors). Moreover, certain vectors are capable of directing the expression of genes to which they are operatively linked. Such vectors are referred to herein as “expression vectors”. In general, expression vectors of utility in recombinant DNA techniques are often in the form of plasmids. In the present specification, “plasmid” and “vector” is used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

A cloning vector is one which is able to replicate autonomously or integrated in the genome in a host cell, and which is further characterized by one or more endonuclease restriction sites at which the vector may be cut in a determinable fashion and into which a desired DNA sequence can be ligated such that the new recombinant vector retains its ability to replicate in the host cell. In the case of plasmids, replication of the desired sequence can occur many times as the plasmid increases in copy number within the host cell such as a host bacterium or just a single time per host before the host reproduces by mitosis. In the case of phage, replication can occur actively during a lytic phase or passively during a lysogenic phase.

An expression vector is one into which a desired DNA sequence can be inserted by restriction and ligation such that it is operably joined to regulatory sequences and can be expressed as an RNA transcript. Vectors can further contain one or more marker sequences suitable for use in the identification of cells which have or have not been transformed or transformed or transfected with the vector. Markers include, for example, genes encoding proteins which increase or decrease either resistance or sensitivity to antibiotics or other compounds, genes which encode enzymes whose activities are detectable by standard assays known in the art (e.g., β-galactosidase, luciferase or alkaline phosphatase), and genes which visibly affect the phenotype of transformed or transfected cells, hosts, colonies or plaques (e.g., green fluorescent protein). In certain embodiments, the vectors used herein are capable of autonomous replication and expression of the structural gene products present in the DNA segments to which they are operably joined.

In some embodiments of any of the aspects, the vector further comprises a promoter that is operatively linked to the nucleic acid sequence encoding the at least one scg2 neuropeptide. As used herein, a coding sequence and regulatory sequences are said to be “operably” joined when they are covalently linked in such a way as to place the expression or transcription of the coding sequence under the influence or control of the regulatory sequences. If it is desired that the coding sequences be translated into a functional protein, two DNA sequences are said to be operably joined if induction of a promoter in the 5′ regulatory sequences results in the transcription of the coding sequence and if the nature of the linkage between the two DNA sequences does not (1) result in the introduction of a frame-shift mutation, (2) interfere with the ability of the promoter region to direct the transcription of the coding sequences, or (3) interfere with the ability of the corresponding RNA transcript to be translated into a protein. Thus, a promoter region would be operably joined to a coding sequence if the promoter region were capable of effecting transcription of that DNA sequence such that the resulting transcript can be translated into the desired protein or polypeptide.

When the nucleic acid molecule that encodes any of the e.g., scg2 neuropeptide(s) described herein is expressed in a cell, a variety of transcription control sequences (e.g., promoter/enhancer sequences) can be used to direct its expression. The promoter can be a native promoter, i.e., the promoter of the Scg2 gene in its endogenous context, which provides normal regulation of expression of the Scg2 gene. As a non-limiting example, the native Scg2 promoter is upstream (e.g., 100-1000 bp) on the reverse complement strand of approximately position 223,600,000 (e.g., 223,602,361) on Homo sapiens Chromosome 2 (see e.g., NCBI Gene ID: 7857), or upstream (e.g., 100-1000 bp) on the reverse complement strand of approximately position 79,440,000 on Mus musculus Chromosome 1 (see e.g., FIG. 4I; NCBI Gene ID: 20254).

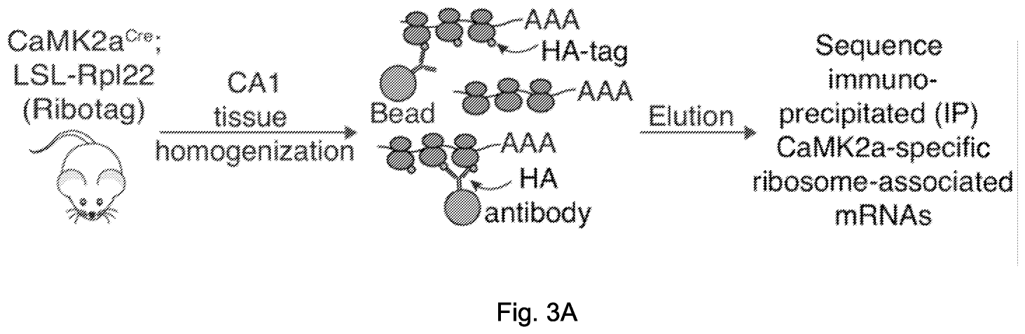

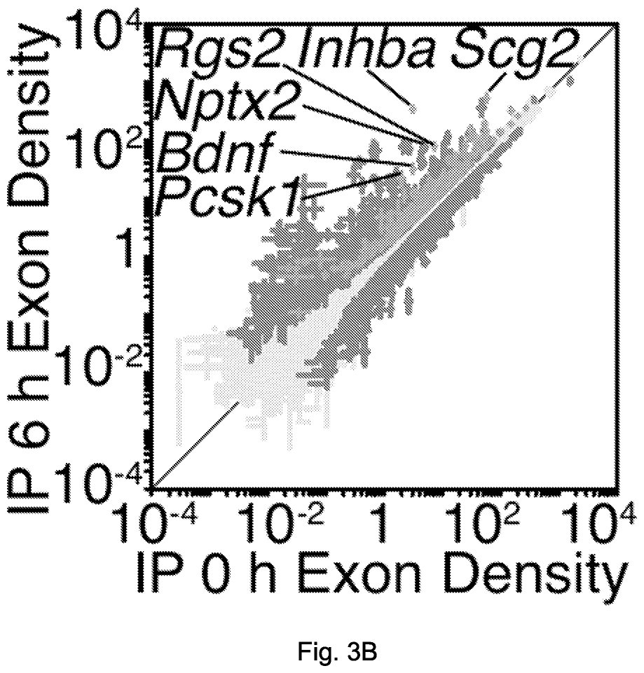

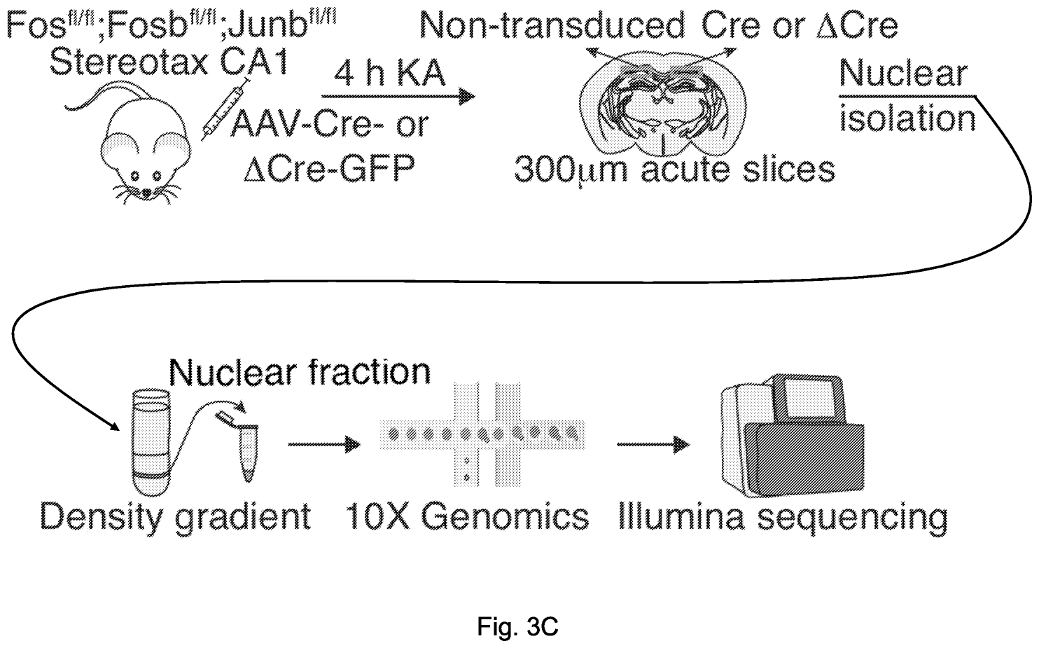

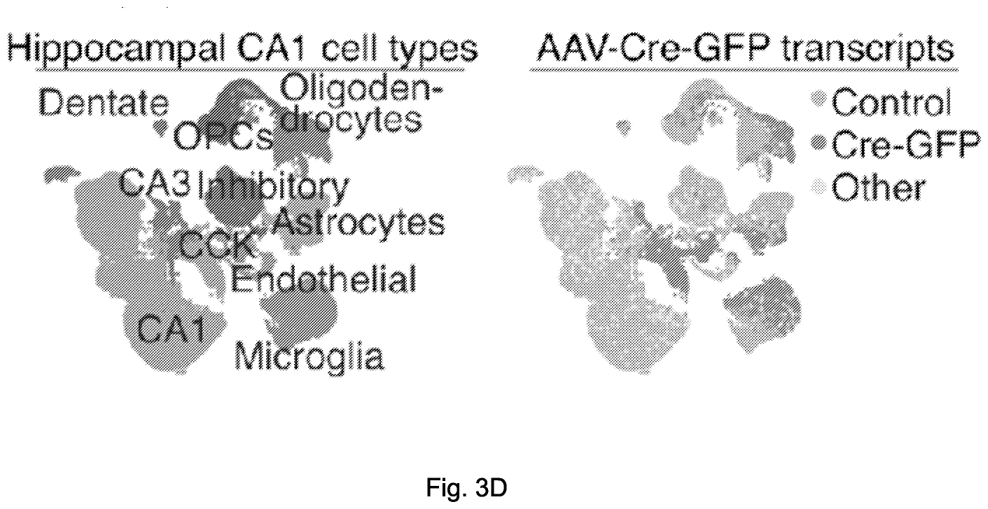

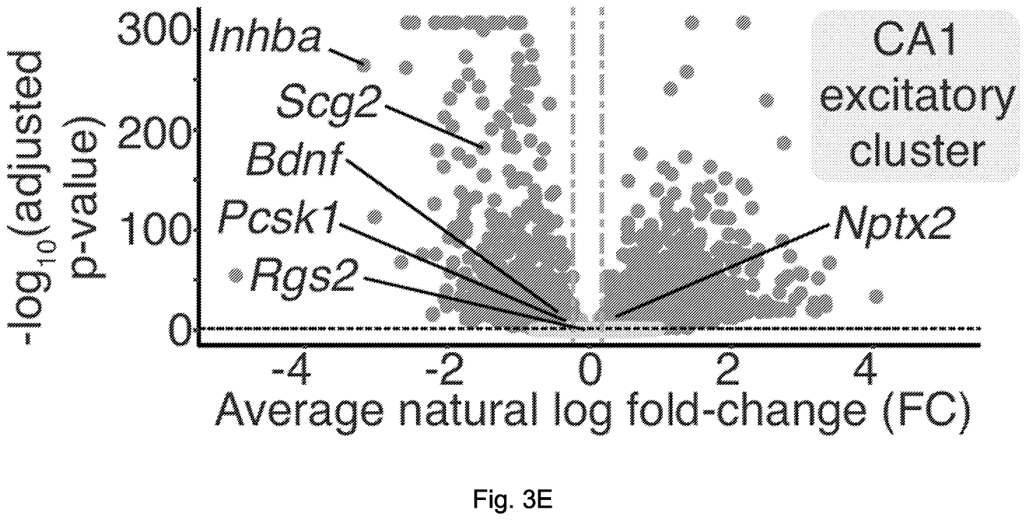

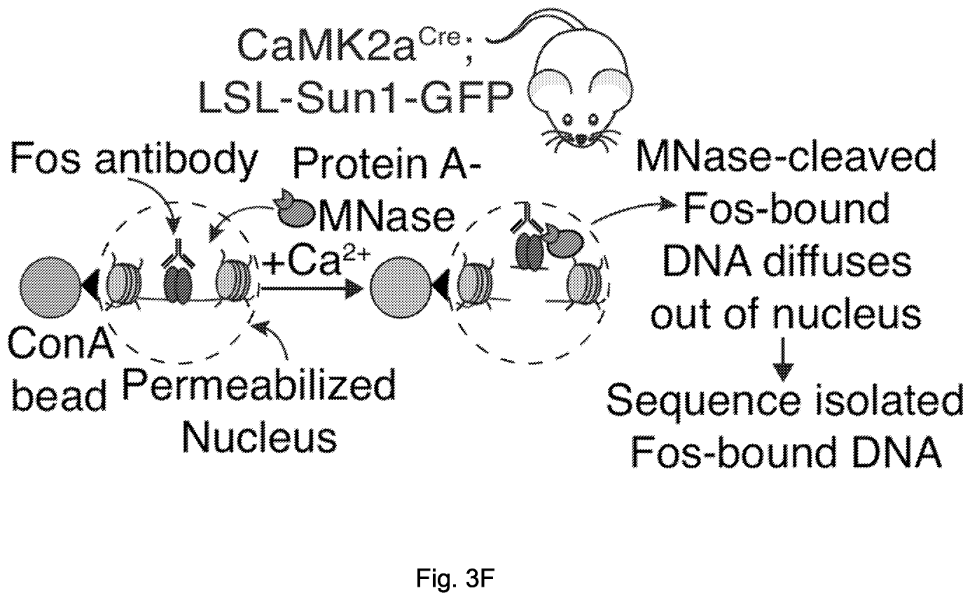

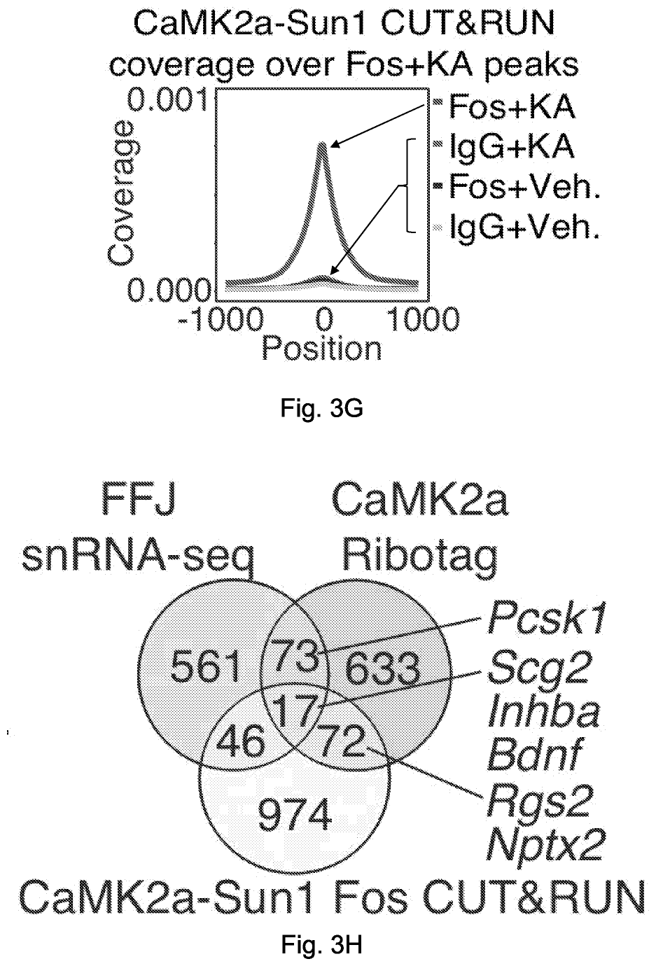

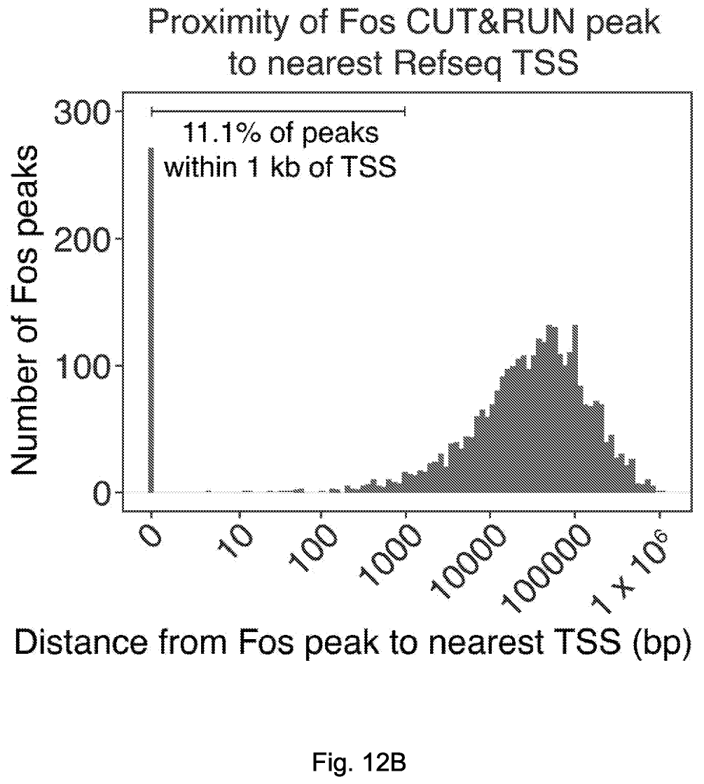

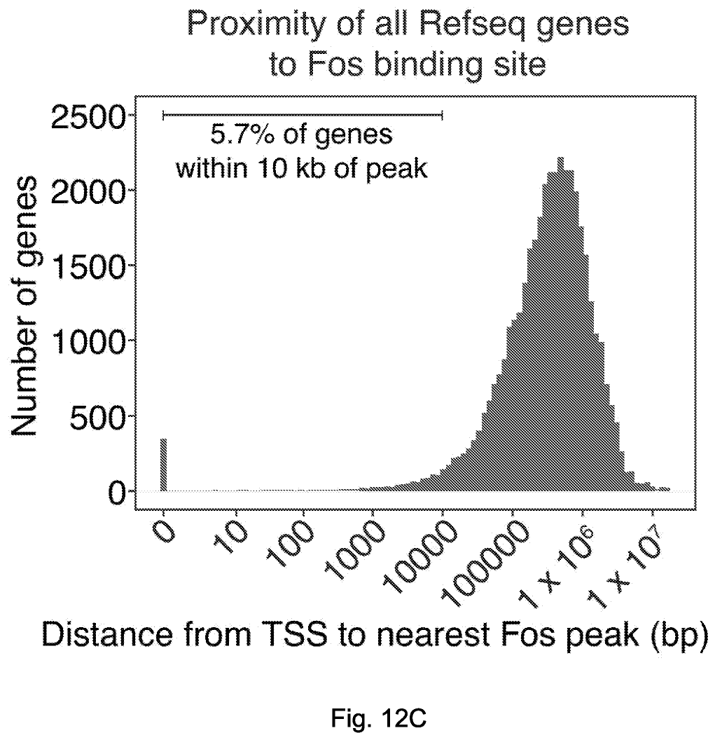

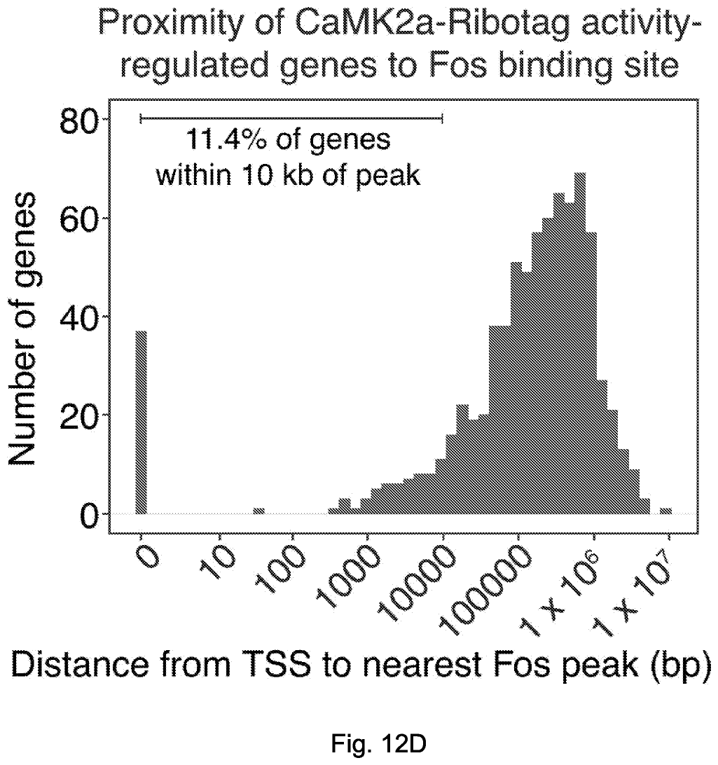

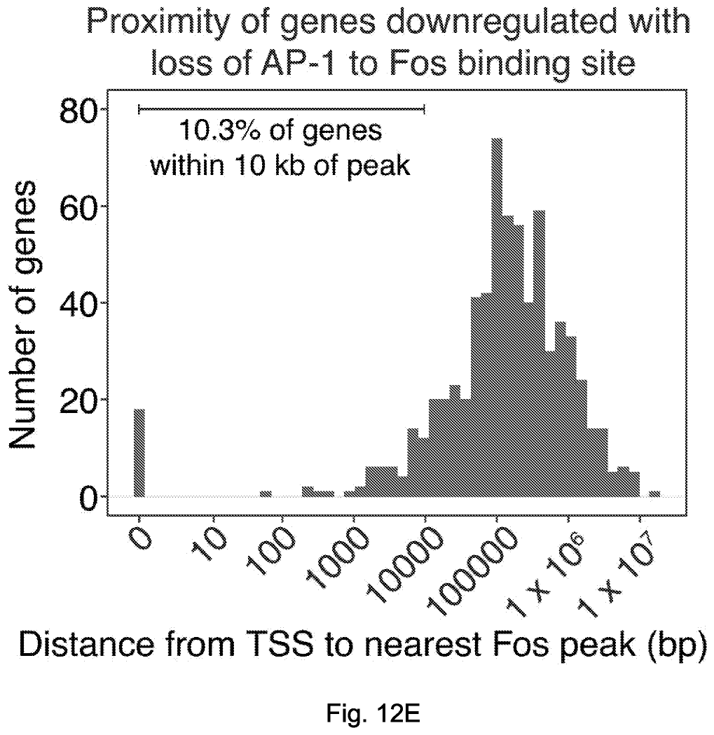

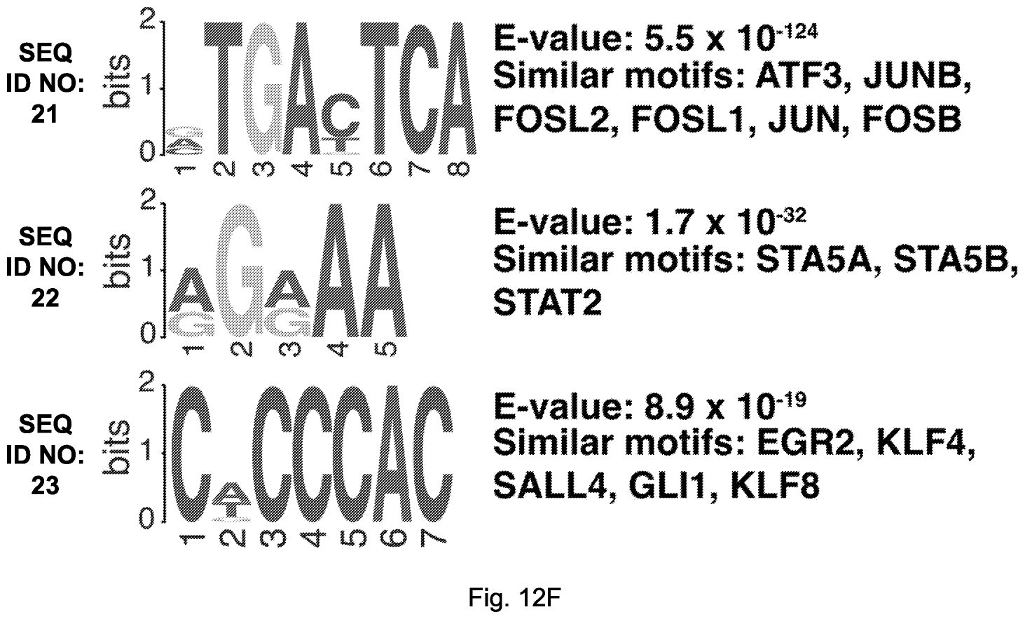

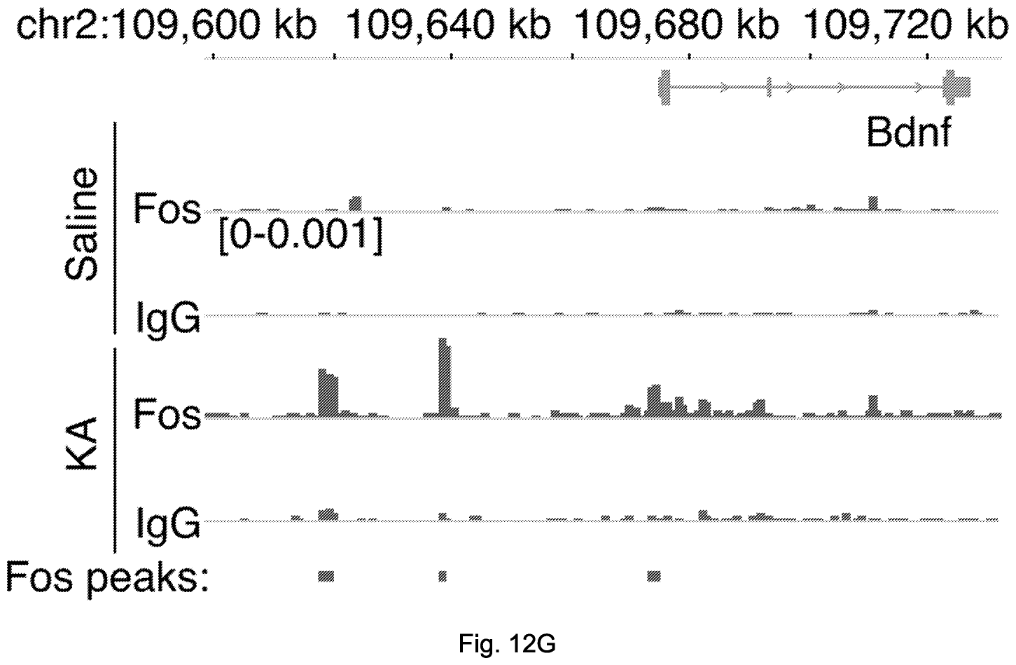

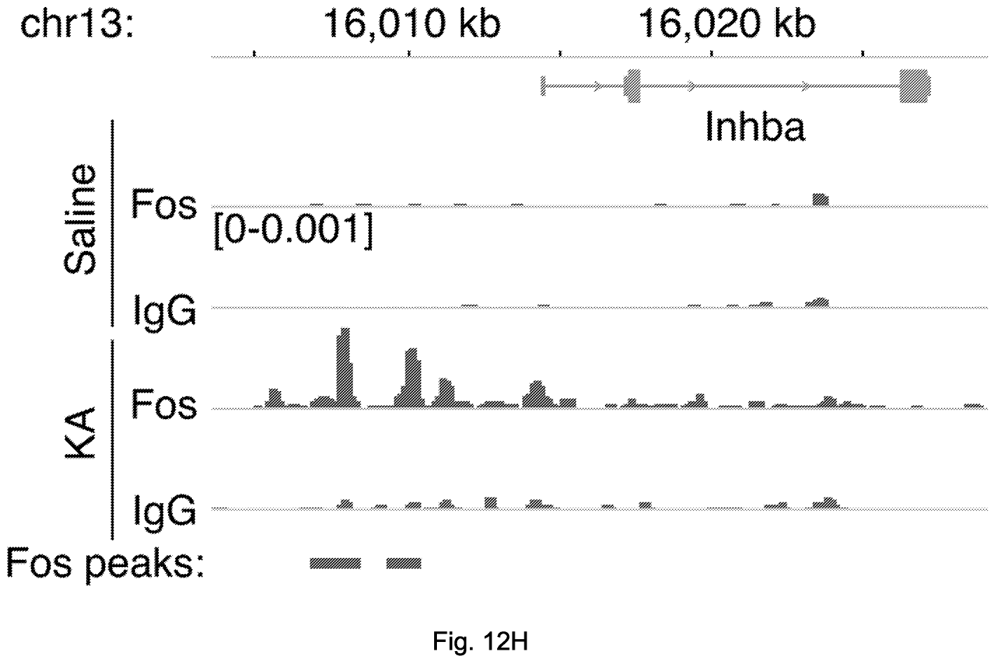

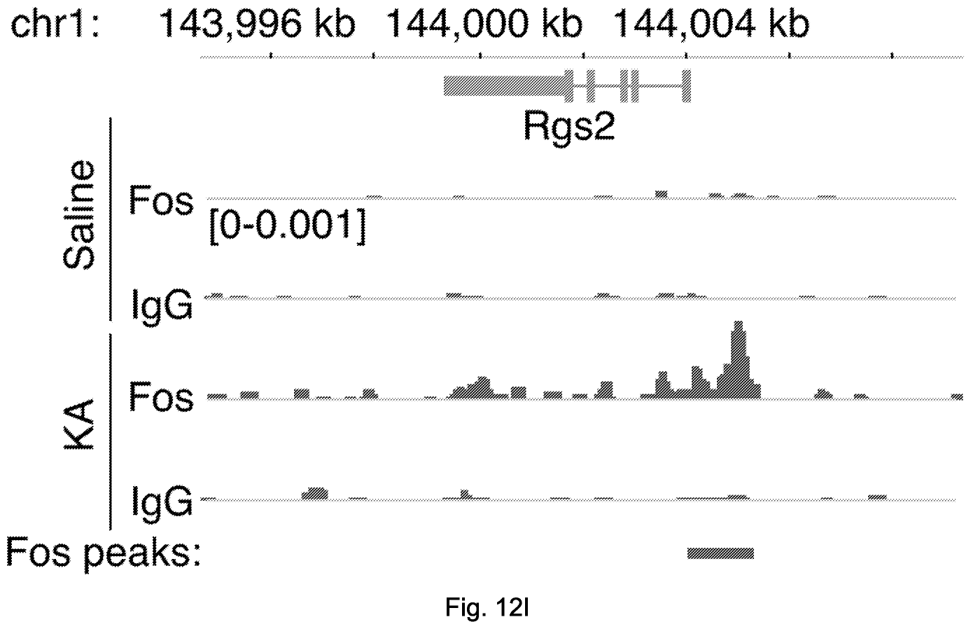

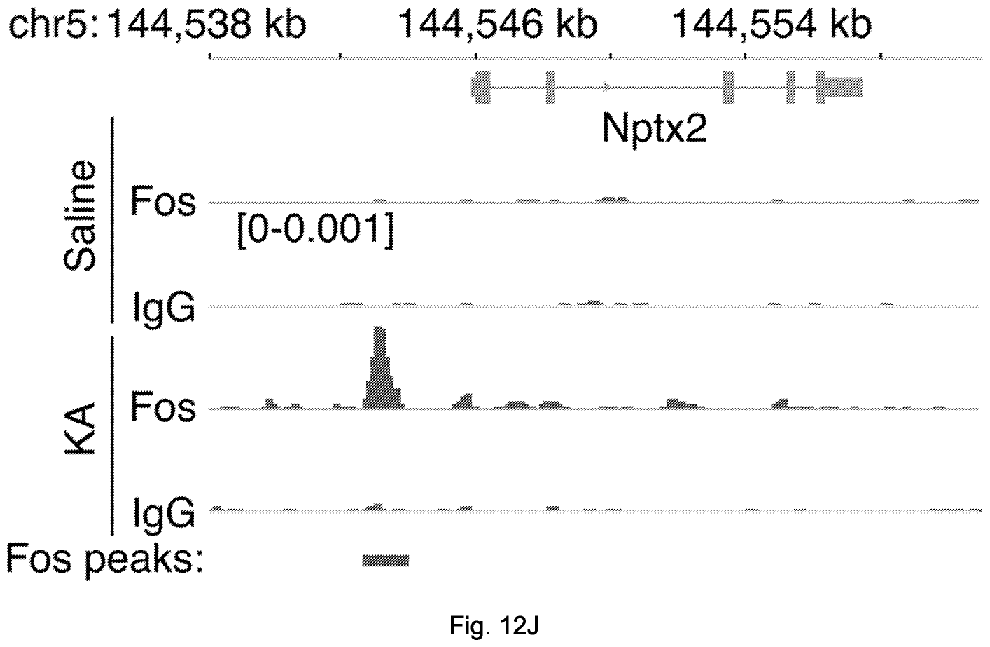

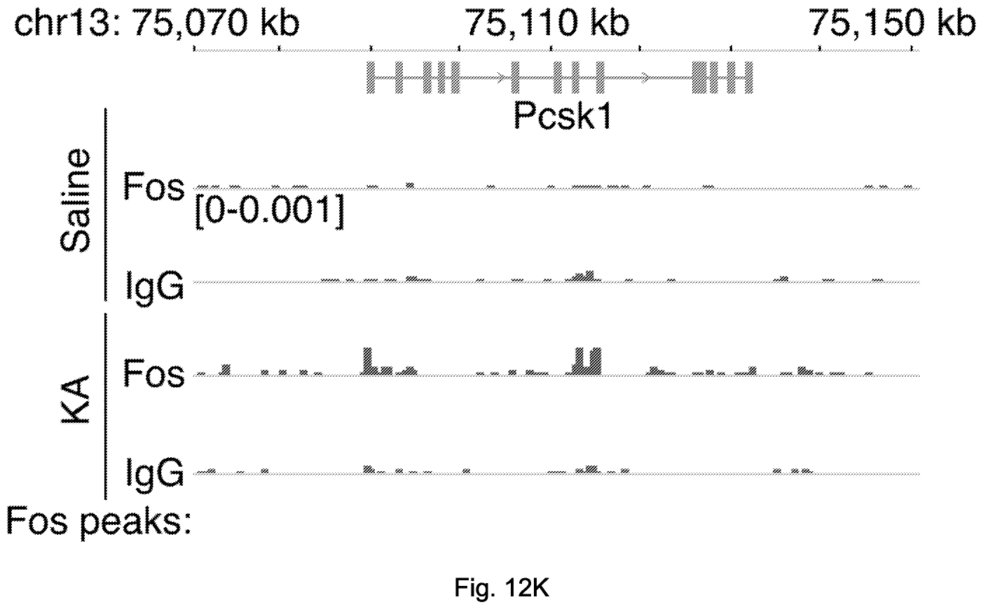

In some embodiments of any of the aspects, the promoter comprises an AP-1 TF family driven promoter (e.g., driven by binding of Fos, Fosb, and/or Junb). In some embodiments of any of the aspects, the promoter comprises a Fos-specific, Fosb-specific, or Junb-specific promoter. In some embodiments of any of the aspects, the nucleic acid or vector comprises a Fos-specific, Fosb-specific, or Junb-specific motif. In some embodiments of any of the aspects, the Fos-specific, Fosb-specific, or Junb-specific motif comprises SEQ ID NO: 21 (nTGAnTCA; see e.g., FIG. 12F) or a nucleic acid sequence that is at least 80%, at least 85%, at least 90%, at least 91%, at least 92%, at least 93%, at least 94%, least 95%, at least 96%, at least 97%, at least 98%, at least 99%, at least 99.5% or more identical to SEQ ID NO: 21 that maintains the same function (e.g., Fos-specific, Fosb-specific, or Junb-specific transcription). In some embodiments of any of the aspects, the Fos-specific, Fosb-specific, or Junb-specific motif within 10 kb (e.g., upstream or downstream) of the transcription start site (TSS) of the Scg2 gene (see e.g., FIG. 3G, FIG. 4I).

In some embodiments the promoter can be constitutive, i.e., the promoter is unregulated allowing for continual transcription of its associated gene. Non-limiting examples of constitutive promoters include: cytomegalovirus (CMV) promoter, the strong synthetic CAG promoter, human elongation factor-1 alpha (EF-1alpha), silencing-prone spleen focus forming virus (SFFV), beta actin/ACTB promoter and the like.

In some embodiments, the nucleic acid encoding the scg2 neuropeptide is operatively linked to an inducible promoter, which is active in the presence of the promoter activator or the absence of the promoter repressor, and inactive in the absence of the promoter inducer or the presence of the promoter repressor. Non-limiting examples of inducible promoters include: a doxycycline-inducible promoter, the lac promoter, the lacUV5 promoter, the tac promoter, the trc promoter, the T5 promoter, the T7 promoter, the T7-lac promoter, the araBAD promoter, the rha promoter, the tet promoter, an isopropyl β-D-1-thiogalactopyranoside (IPTG)-dependent promoter, an AlcA promoter, a LexA promoter, a temperature inducible promoter (e.g., Hsp70 or Hsp90-derived promoters), or a light inducible promoter (e.g., pDawn/YFI/FixK2 promoter/CI/pR promoter system).

In some embodiments of any of the aspects, the promoter comprises a tissue-specific promoter, e.g., specific to the brain, the central nervous system, the hippocampus, neurons, and the like. In some embodiments of any of the aspects, the promoter comprises a nervous tissue-specific promoter. In some embodiments of any of the aspects, the nervous tissue-specific promoter is a neuron-specific promoter. In some embodiments of any of the aspects, the neuron-specific promoter is the synapsin promoter (e.g., Human synapsin 1 promoter) or the caMK2a promoter (e.g., human Calcium/Calmodulin Dependent Protein Kinase II Alpha promoter). The synapsin I promoter has been used to achieve highly neuron-specific long-term transgene expression in vivo. The CaMK2a promoter is a neuron-specific promoter with expression restricted to excitatory neurons in the neocortex and hippocampus, including pyramidal neurons.

The precise nature of the regulatory sequences needed for gene expression can vary between species or cell types, but in general can include, as necessary, 5′ non-transcribed and 5′ non-translated sequences involved with the initiation of transcription and translation respectively, such as a TATA box, capping sequence, CAAT sequence, and the like. In particular, such 5′ non-transcribed regulatory sequences will include a promoter region which includes a promoter sequence for transcriptional control of the operably joined gene. Regulatory sequences can also include enhancer sequences or upstream activator sequences as desired. The vectors of the invention may optionally include 5′ leader or signal sequences. The choice and design of an appropriate vector is within the ability and discretion of one of ordinary skill in the art.

Expression vectors containing all the necessary elements for expression are commercially available and known to those skilled in the art. See, e.g., Sambrook et al., Molecular Cloning: A Laboratory Manual, Second Edition, Cold Spring Harbor Laboratory Press, 1989. Cells are genetically engineered by the introduction into the cells of heterologous DNA (RNA). That heterologous DNA (RNA) is placed under operable control of transcriptional elements to permit the expression of the heterologous DNA in the host cell.

In some embodiments of any of the aspects, the vector is a viral vector. Accordingly, in one aspect, described herein is a viral vector comprising a vector or nucleic acid as described herein, e.g., encoding at least one Scg2 neuropeptide. In some embodiments of any of the aspects, the viral vector is an adenovirus-associated virus (AAV). AAV vectors are non-enveloped 25 nm particles with a foreign DNA packaging capacity of 4.6 kb. They have been clinically demonstrated to be safe in the CNS, and certain serotypes display strong neural tropism. In some embodiments of any of the aspects, the AAV is serotype AAV2/1, which is a hybrid of serotypes 2 and 1 and exhibits neuronal tropism and expression. In some embodiments of any of the aspects, the AAV is AAV1, which is an efficient viral vector in various brain regions and leads to extensive anterograde and retrograde expression. In some embodiments of any of the aspects, the AAV is AAV2, which in the brain, is strongly neuron-specific. In some embodiments of any of the aspects, the AAV is serotype AAV2/1, AAVDJ8, AAV9, AAV8, AAVDJ9, or AAV1, which have tropism for primary murine astrocyte and neuronal cell cultures. In some embodiments of any of the aspects, the AAV is serotype AAV2/1, AAVDJ8, or AAV9, which have tropism for the olfactory bulb, striatum, cortex, hippocampus, substantia nigra (SN) and cerebellum. In some embodiments of any of the aspects, the AAV is AAV serotype 6 (AAV6), which is retrogradely transported from terminals to neuronal cell bodies in the rat brain. In some embodiments of any of the aspects, the AAV is AAV7. In some embodiments of any of the aspects, the AAV is AAV9. Infusion (e.g., through the cisterna magna) (CM) of either AAV7 or AAV9 is associated with a high level of cell transduction distributed throughout brain cortex and along the spinal cord, including dorsal root ganglia, corticospinal tracts, astrocytes, and neurons. In some embodiments of any of the aspects, the AAV is a rhesus monkey AAV, designated as “AAVrh,” which exhibits CNS-tropism. In some embodiments of any of the aspects, the AAV is AAVrh.10, AAVrh.39, rAAVrh.43, which are capable of crossing the blood-brain barrier (BBB). In some embodiments of any of the aspects, the AAV has tropism for the brain and/or neurons, thus allowing delivery of the nucleic acid across the BBB and into the brain, e.g., where the Scg2 neuropeptide(s) can be expressed under the control of the operatively linked promoter. See e.g., Hammond et al., PLoS One. 2017; 12(12): e0188830, the content of which is incorporated herein by reference in its entirety.

In some embodiments of any of the aspects, the viral vector is a herpes simplex virus (e.g., HSV-1). Herpes simplex virus type 1 (HSV-1) vectors are enveloped 100 nm particles with a foreign DNA packaging capacity of more than 100 kb. The greatest advantages are the high packaging capacity and natural neurotropism via retrograde axonal transport. In some embodiments of any of the aspects, the viral vector is a lentivirus (e.g., human immunodeficiency virus (HIV) or a self-inactivating (SIN) lentiviral vector). Lentiviral vectors are enveloped 100 nm particles with a foreign DNA packaging capacity of 9 kb. When pseudotyped, they have high neuronal tropism. In some embodiments of any of the aspects, the lentivirus is pseudotyped with a glycoprotein that targets neurons or glial cells. Non-limiting examples of such glycoproteins include the glycoproteins from neurotropic virus such as vesicular stomatitis virus G (VSV-G), lymphocytic choriomeningitis virus (LCMV), rabies, or Mokola lyssavirus. See e.g., Gray et al., Ther Deliv. 2010, 1(4): 517-534, the content of which is incorporated herein by reference in its entirety.

Without limitations, the nucleic acids encoding Scg2 neuropeptides genes described herein can be included in one vector or separate vectors. For example, the nucleic acids encoding secretoneurin, EM66, manserin, and SgII can be included in the same vector. As another example, nucleic acids encoding at least 2, at least 3, or 4 of secretoneurin, EM66, manserin, and SgII can be included in the same vector. Such scg2 neuropeptide-encoding nucleic acid combinations can be selected from the examples provided in Table 2.

In some embodiments, the nucleic acid encoding secretoneurin can be included in a first vector, the nucleic acid encoding EM66 can be included in a second vector, the nucleic acid encoding manserin can be included in a third vector, and/or the nucleic acid encoding SgII can be included in a fourth vector.

In some embodiments, one or more of the recombinantly expressed nucleic acids encoding at least one Scg2 neuropeptides can be integrated into the genome of the cell. A nucleic acid molecule that encodes at least one Scg2 neuropeptide as described herein can be introduced into a cell or cells using methods and techniques that are standard in the art. For example, nucleic acid molecules can be introduced by standard protocols such as transformation including chemical transformation and electroporation, transduction, particle bombardment, etc. Expressing the nucleic acid molecule encoding at least one Scg2 neuropeptide can may be accomplished by integrating the nucleic acid molecule into the genome or through stable episomes. For example, AAV is a virus that can be maintained in an extrachromosomal form (i.e., episome) in the nucleic of transduced cells. Vector integration of AAV has also been observed in various experimental settings, either at non-homologous sites where DNA damage may have occurred or by homologous recombination

Accordingly, in one aspect described herein is a cell comprising a pharmaceutical composition as described herein, a nucleic acid as described herein, a vector as described herein, or a viral vector as described herein, any of which comprise, encode, or express at least one Scg2 neuropeptide as described herein. In some embodiments of any of the aspects, the cell is a neuronal cell. In some embodiments of any of the aspects, the cell is from a neuronal cell line (e.g., SH-SY5Y neuroblastoma cells; NT2 cells, such as NTERA-2 CL.D1 (NT2/D1) ATCC® CRL-1973™; PC-12 cells (e.g., ATCC® CRL-1721™)), In some embodiments of any of the aspects, the cell is from a primary neuronal culture (e.g., murine, rat, non-human primate, or human primary neuronal cultures). In some embodiments of any of the aspects, the cell is a hippocampal cell. In some embodiments of any of the aspects, the cell is a pyramidal cell. In some embodiments of any of the aspects, the cell is a CA1 pyramidal cell. In some embodiments of any of the aspects, the cell is an interneuron (e.g., a PV-IN or CCK-IN). In some embodiments of any of the aspects, the cell is a CNS neuron. In some embodiments of any of the aspects, the cell is a peripheral nervous system (PNS) neuron. In some embodiments of any of the aspects, the cell is a motor neuron. In some embodiments of any of the aspects, the cell is a sensory neuron. In some embodiments of any of the aspects, the cell is a dorsal root ganglion neuron. In some embodiments of any of the aspects, the cell is a neuron from the enteric nervous system. In some embodiments of any of the aspects, the cell is a neuron from the Vagal nerve. In some embodiments of any of the aspects, the cell is a neuroendocrine cell. In some embodiments of any of the aspects, the cell is a pancreatic islet cell (e.g., that is polarizable). In some embodiments of any of the aspects, the cell is a central nervous system (CNS) glial cell selected from microglia, astrocytes, oligodendrocytes, radial glial cells, and ependymal cells. In some embodiments of any of the aspects, the cell is a PNS glial cell selected from Schwann cells, enteric glial cells, and satellite glial cells.

In some embodiments, the methods described herein relate to administering a pharmaceutical composition comprising at least one Scg2 neuropeptide, or a nucleic acid, vector, or a viral vector encoding at least one Scg2 neuropeptide, as described herein. In some embodiments, the methods described herein relate to treating a subject having or diagnosed as having a memory-associated disorder, a learning disability, a neurodegenerative disease or disorder, or epilepsy with a pharmaceutical composition, nucleic acid, vector, or viral vector as described herein.

In one aspect, described herein is a method of increasing memory consolidation and/or memory retention, comprising administering an effective amount of a pharmaceutical composition, nucleic acid, vector, or viral vector as described herein to a subject in need thereof. In some embodiments of any of the aspects, the treatment increases memory consolidation and/or memory retention in the subject. In some embodiments of any of the aspects, the treatment increases memory consolidation. As used herein, “memory consolidation” refers to a time-dependent process by which recent learned experiences are transformed into long-term memory, by structural and chemical changes in the nervous system (e.g., the strengthening of synaptic connections between neurons). Specifically, consolidation is the process by which the hippocampus guides the reorganization of the information stored in the neocortex such that it eventually becomes independent of the hippocampus. In some embodiments of any of the aspects, the treatment increases memory retention in the subject. As used herein, “memory retention” refers to the ability to remember or recall information over a period of time; strong memory retention indicates that a subject can easily put knowledge to use without occupying or overloading working memory, since background knowledge is readily available. In some embodiments of any of the aspects, memory consolidation and/or memory retention is increased by at least 10%, at least 20%, at least 30%, at least 40%, at least 50%, at least 60%, at least 70%, at least 80%, at least 90%, at least 95%, at least 100% or more compared to a subject that is not administered a pharmaceutical composition, nucleic acid, vector, or viral vector as described herein.

In some embodiments of any of the aspects, the administration increases spatial learning of the subject by at least 10%, at least 20%, at least 30%, at least 40%, at least 50%, at least 60%, at least 70%, at least 80%, at least 90%, at least 95%, at least 100% or more compared to a subject that is not administered the pharmaceutical composition, nucleic acid, vector, or viral vector. As used herein, “spatial learning” refers to the process by which an organism acquires a mental representation of its environment, i.e., acquires a spatial memory. Spatial memory is a form of memory responsible for the recording and recovery of information needed to plan a course to a location and to recall the location of an object or the occurrence of an event. Spatial memory is necessary for orientation in space. Spatial learning and spatial memory can be tested in mammals, e.g., by using the Morris water maze experiment as described herein (see e.g., Methods of Example 1).

In some embodiments, a subject in need of increasing memory consolidation and/or memory retention is any subject that has the desire or need to increase their memory consolidation and/or memory retention. In some embodiments, a subject in need of increasing memory consolidation and/or memory retention is a subject with a learning disability, a neurodegenerative disease or disorder, or another memory-associated disorder (e.g., amnesia, dementia, Alzheimer's disease, mild cognitive impairment, vascular cognitive impairment, or hydrocephalus). Accordingly, in one aspect, described herein is a method of treating a memory-associated disorder, comprising administering an effective amount of a pharmaceutical composition, nucleic acid, vector, or viral vector as described herein to a subject in need thereof.

Subjects having a memory-associated disorder can be identified by a physician using current methods of diagnosing memory-associated disorders. Symptoms and/or complications of a memory-associated disorder which characterize these conditions and aid in diagnosis are well known in the art and include but are not limited to, memory loss, confusion, restlessness, personality and behavior changes, problems with judgment, problems communicating with others, inability to follow directions, or lack of emotion. Tests that may aid in a diagnosis of a memory-associated disorder include, but are not limited to, the Mini-Mental State Exam (MMSE) and the Mini-Cog test. A family history of a memory-associated disorder, or exposure to risk factors for a memory-associated disorder (e.g., nutritional deficiency, lower education level, older age, history of head trauma, illness, medications (including alcohol or illicit drugs), vision or hearing impairment, uncontrolled chronic medical conditions, stroke, or psychological factors such as depression and stress) can also aid in determining if a subject is likely to have a memory-associated disorder or in making a diagnosis of a memory-associated disorder.

The compositions and methods described herein can be administered to a subject having or diagnosed as having a memory-associated disorder. In some embodiments, the methods described herein comprise administering an effective amount of compositions described herein, e.g., comprising or encoding at least one Scg2 neuropeptide, to a subject in order to alleviate a symptom of a memory-associated disorder. As used herein, “alleviating a symptom of a memory-associated disorder” is ameliorating any condition or symptom associated with the memory-associated disorder. As compared with an equivalent untreated control, such reduction is by at least 5%, 10%, 20%, 40%, 50%, 60%, 80%, 90%, 95%, 99% or more as measured by any standard technique.

In one aspect, described herein is a method of treating a learning disability, comprising administering an effective amount of a pharmaceutical composition, nucleic acid, vector, or viral vector as described herein to a subject in need thereof. Non-limiting examples of learning disabilities include dyscalculia, dysgraphia, dyslexia, a non-verbal leaning disability, an oral and/or written language disorder and specific reading comprehension deficit, attention deficit hyperactivity disorder (ADHD), attention deficit disorder (ADD), dyspraxia, an executive mal-functioning, an auditory processing disorder, a language processing disorder, a visual perceptual/visual motor deficit, and the like.

Subjects having a learning disability can be identified by a physician using current methods of diagnosing learning disabilities. Symptoms and/or complications of learning disabilities which characterize these conditions and aid in diagnosis are well known in the art and include but are not limited to, problems reading and/or writing, problems with math, poor memory, problems paying attention, trouble following directions, clumsiness, trouble telling time, problems staying organized, and hyperactivity. Tests that may aid in a diagnosis of learning disabilities include, but are not limited to, Woodcock-Johnson Tests of Achievement (WJ), the Wechsler Individual Achievement Test (WIAT), the Wide Range Achievement Test (WRAT), and the Kaufman Test of Educational Achievement (KTEA). A family history of learning disabilities, or exposure to risk factors for learning disabilities (e.g. poor fetal growth in the uterus (e.g., severe intrauterine growth restriction), exposure to alcohol or drugs before being born, premature birth, very low birthweight, psychological trauma, physical trauma (e.g., head injuries or nervous system infections), environmental exposure to high levels of toxins, such as lead) can also aid in determining if a subject is likely to have a learning disability or in making a diagnosis of a learning disability.

The compositions and methods described herein can be administered to a subject having or diagnosed as having a learning disability. In some embodiments, the methods described herein comprise administering an effective amount of compositions described herein, e.g. comprising or encoding at least one Scg2 neuropeptide, to a subject in order to alleviate a symptom of a learning disability. As used herein, “alleviating a symptom of a learning disability” is ameliorating any condition or symptom associated with the learning disability. As compared with an equivalent untreated control, such reduction is by at least 5%, 10%, 20%, 40%, 50%, 60%, 80%, 90%, 95%, 99% or more as measured by any standard technique.

In one aspect, described herein is a method of treating a neurodegenerative disease or disorder, comprising administering an effective amount of a pharmaceutical composition, nucleic acid, vector, or viral vector as described herein to a subject in need thereof. Non-limiting examples of neurodegenerative diseases or disorders include Alzheimer's disease, Parkinson's disease, Huntington's disease, amyotrophic lateral sclerosis (ALS), frontotemporal dementia, chronic traumatic encephalopathy (CTE), multiple sclerosis, and neuroinflammation, among others.

Subjects having a neurodegenerative disease or disorder can be identified by a physician using current methods of diagnosing neurodegenerative diseases or disorders. Symptoms and/or complications of neurodegenerative diseases or disorders, which characterize these conditions and aid in diagnosis are well known in the art and include but are not limited to memory loss, forgetfulness, apathy, anxiety, agitation, a loss of inhibition, or mood changes. Tests that may aid in a diagnosis of a neurodegenerative disease or disorder include, but are not limited to, imaging (e.g., of the brain by a CT scan, PET scan, MRI, or the like), genetic testing for associated disease markers, cognitive testing (e.g., the clock-drawing test for neurodegenerative diseases or disorders), behavioral testing, physical stamina testing, etc. A family history of a neurodegenerative disease or disorder, or exposure to risk factors for a neurodegenerative disease or disorder can also aid in determining if a subject is likely to have a neurodegenerative disease or disorder or in making a diagnosis of a neurodegenerative disease or disorder.

The compositions and methods described herein can be administered to a subject having or diagnosed as having a neurodegenerative disease or disorder. In some embodiments, the methods described herein comprise administering an effective amount of compositions described herein, e.g., comprising or encoding at least one Scg2 neuropeptide, to a subject in order to alleviate a symptom of a neurodegenerative disease or disorder. As used herein, “alleviating a symptom of a neurodegenerative disease or disorder” is ameliorating any condition or symptom associated with the neurodegenerative disease or disorder. As compared with an equivalent untreated control, such reduction is by at least 5%, 10%, 20%, 40%, 50%, 60%, 80%, 90%, 95%, 99% or more as measured by any standard technique.

In one aspect, described herein is a method of treating epilepsy, comprising administering an effective amount of a pharmaceutical composition, nucleic acid, vector, or viral vector as described herein to a subject in need thereof. Non-limiting examples of epilepsy include: focal seizures without loss of consciousness (simple partial seizures); focal seizures with impaired awareness (complex partial seizures); absence seizures (petit mal seizures); tonic seizures; atonic seizures; clonic seizures; myoclonic seizures; or tonic-clonic seizures, among others.

Subjects having epilepsy can be identified by a physician using current methods of diagnosing epilepsy. Symptoms and/or complications of epilepsy, which characterize these conditions and aid in diagnosis are well known in the art and include but are not limited to temporary confusion, staring spells, stiff muscles, uncontrollable jerking movements of the arms and legs, loss of consciousness or awareness, and/or psychological symptoms such as fear, anxiety, or deja vu. Tests that may aid in a diagnosis of epilepsy include, but are not limited to, neurological exams, blood tests, electroencephalogram (EEG), high-density EEG, brain imaging (e.g., computerized tomography (CT) scan, magnetic resonance imaging (MRI), functional MRI (fMRI), positron emission tomography (PET), single-photon emission computerized tomography (SPECT)), neuropsychological tests (e.g., testing thinking, memory, and/or speech skills), statistical parametric mapping (SPM), electrical source imaging (ESI), or magnetoencephalography (MEG), etc. A family history of epilepsy, or exposure to risk factors for epilepsy (e.g., head injury; brain abnormalities (e.g., brain tumors or vascular malformations such as arteriovenous malformations (AVMs) and cavernous malformations; stroke) infections (e.g., Meningitis, HIV, viral encephalitis and some parasitic infections (e.g., Taenia solium, the pork tapeworm)); pre-natal brain injury (e.g., caused by infection in the mother, poor nutrition, or oxygen deficiencies); developmental disorders such as autism; high fevers in children; etc.) can also aid in determining if a subject is likely to have epilepsy or in making a diagnosis of epilepsy.

The compositions and methods described herein can be administered to a subject having or diagnosed as having epilepsy. In some embodiments, the methods described herein comprise administering an effective amount of compositions described herein, e.g., comprising or encoding at least one Scg2 neuropeptide, to a subject in order to alleviate a symptom of epilepsy. As used herein, “alleviating a symptom of epilepsy” is ameliorating any condition or symptom associated with epilepsy. As compared with an equivalent untreated control, such reduction is by at least 5%, 10%, 20%, 40%, 50%, 60%, 80%, 90%, 95%, 99% or more as measured by any standard technique.

A variety of means for administering the compositions described herein to subjects are known to those of skill in the art. Such methods can include, but are not limited to oral, parenteral, intravenous, intramuscular, subcutaneous, transdermal, airway (aerosol), pulmonary, cutaneous, topical, injection, or intratumoral administration. Administration can be local or systemic. In some embodiments of any of the aspects, the pharmaceutical composition, nucleic acid, vector, or viral vector is administered to the central nervous system. In some embodiments of any of the aspects, the pharmaceutical composition, nucleic acid, vector, or viral vector is administered intracranially, epidurally, intrathecally, intraparenchymally, intraventricularly, or subarachnoidly. In some embodiments of any of the aspects, the pharmaceutical composition, nucleic acid, vector, or viral vector is administered intranasally. In some embodiments of any of the aspects, the pharmaceutical composition, nucleic acid, vector, or viral vector is administered in a formulation that crosses the blood-brain barrier, as described further herein. In some embodiments of any of the aspects, the pharmaceutical composition, nucleic acid, vector, or viral vector is administered via direct injection into the CNS or brain, e.g., a specific region of the brain such as in or near the hippocampus. In some embodiments of any of the aspects, the pharmaceutical composition, nucleic acid, vector, or viral vector is administered via infusion into the CNS or brain, e.g., via a shunt. In some embodiments of any of the aspects, the pharmaceutical composition, nucleic acid, vector, or viral vector is administered into the brain using an invasive method, such as the use of polymers or microchip systems, stereotactically guided drug insertion through a catheter, or transient disruption of the BBB.

In some embodiments of any of the aspects, the scg2 neuropeptide binds to a G-protein coupled receptor (GPCR). G protein-coupled receptors (GPCRs), also known as seven-(pass)-transmembrane domain receptors, 7TM receptors, heptahelical receptors, serpentine receptors, and G protein-linked receptors (GPLR), form a large group of evolutionarily-related proteins that are cell surface receptors that detect molecules outside the cell and activate cellular responses. Coupling with G proteins, GPCRs are called seven-transmembrane receptors as they pass through the cell membrane seven times. Ligands can bind either to extracellular N-terminus and loops (e.g., glutamate receptors) or to the binding site within transmembrane helices (e.g., Rhodopsin-like family).

There are two principal signal transduction pathways involving the G protein-coupled receptors: the cAMP signal pathway and the phosphatidylinositol signal pathway. When a ligand binds to the GPCR it causes a conformational change in the GPCR, which allows it to act as a guanine nucleotide exchange factor (GEF). The GPCR can then activate an associated G protein by exchanging the GDP bound to the G protein for a GTP. The G protein's a subunit, together with the bound GTP, can then dissociate from the β and γ subunits to further affect intracellular signaling proteins or target functional proteins directly depending on the a subunit type (G_{αs}, G_{αi/o}, G_{αq/11}, G_{α12/13}).

In some embodiments of any of the aspects, the administration modulates activity of interneurons in the central nervous system of the subject. Interneurons (also called internuncial neurons, relay neurons, association neurons, connector neurons, intermediate neurons, or local circuit neurons) are neurons that connect two brain regions or neurons. Interneurons are the central nodes of neural circuits, permitting communication between sensory or motor neurons and the central nervous system (CNS). [2] Interneurons play vital roles in reflexes, neuronal oscillations, and neurogenesis in the adult mammalian brain. Local interneurons have short axons and form circuits with nearby neurons to analyze small pieces of information. The interaction between interneurons allow the brain to perform complex functions such as learning, and decision-making. Interneurons in the CNS are primarily inhibitory, and use the neurotransmitter γ-aminobutyric acid (GABA) or glycine. In some embodiments of any of the aspects, the interneurons are γ-aminobutyric acid-releasing (GABAergic) interneurons. In some embodiments of any of the aspects, the interneurons are inhibitory interneurons.

In some embodiments of any of the aspects, the administration modulates activity of interneurons in the hippocampus of the subject. As used herein, the term “modulates activity of interneurons” refers to increasing or decreasing the excitatory or inhibitory activity of at least one interneuron, e.g., by increasing or decreasing the number of synapses between the interneuron and another cell (e.g., a pyramidal cell), or increasing or decreasing the strength of the synapses. The hippocampus is a complex brain structure embedded deep into temporal lobe, and it has a major role in learning and memory. The hippocampus proper, which refers to the actual structure of the hippocampus, is made up of four regions or subfields: CA1, CA2, CA3, and CA4. CA1 is the first region in the hippocampal circuit, from which a major output pathway goes to layer V of the entorhinal cortex. Another significant output of CA1 is to the subiculum. In some embodiments of any of the aspects, the administration modulates activity of γ-aminobutyric acid-releasing (GABAergic) interneurons in the CA1 region of the hippocampus of the subject.

In some embodiments of any of the aspects, the interneurons are parvalbumin-expressing interneurons (PV-IN) or cholecystokinin-expressing interneurons (CCK-IN). In some embodiments of any of the aspects, the interneurons (e.g., PV-INs or CCK-INs) are associated with at least one pyramidal neuron. As used herein, “pyramidal neuron” or “pyramidal cell” refers to a type of multipolar neuron found in areas of the brain including the cerebral cortex, the hippocampus, and the amygdala; pyramidal neurons are the primary excitation units of the mammalian prefrontal cortex and the corticospinal tract. In some embodiments of any of the aspects, the interneurons are parvalbumin-expressing interneurons (PV-IN). In some embodiments of any of the aspects, the administration increases PV-IN perisomatic inhibitory activity, e.g., on an associated pyramidal cell. In some embodiments of any of the aspects, the interneurons are cholecystokinin-expressing interneurons (CCK-IN). In some embodiments of any of the aspects, the administration decreases CCK-IN perisomatic inhibitory activity, e.g., on an associated pyramidal cell. In some embodiments of any of the aspects, the boutons of interneurons (e.g., PV-INs or CCK-INs) are associated through a perisomatic interaction with at least one neuron. As used herein, “bouton” refers to an enlarged part of a nerve fiber or cell, especially an axon, where it forms a synapse with another nerve. As used herein, “perisomatic” refers to the domain of plasma membrane surrounding the soma (cell body) of a neuron. Pyramidal cells receive almost exclusively GABAergic synapses through perisomatic interactions with axons of other neurons (e.g., interneurons).

In some embodiments of any of the aspects, the administration increases the power of fast gamma waves (60 Hz-90 Hz), e.g., in the CA1 region of the hippocampus. As used herein, “gamma wave” or “gamma rhythm” refers to a pattern of neural oscillation in humans, which are correlated with large scale brain network activity and cognitive phenomena such as working memory, attention, and perceptual grouping, and can be increased in amplitude via meditation or neurostimulation. Fast gamma activity can allow the transfer of neocortical information to the hippocampus by boosting connectivity between the entorhinal cortex (MEC) and CAL In some embodiments of any of the aspects, the administration increases the power of gamma waves that are at least 60 Hz, at least 65 Hz, at least 70 Hz, at least 75 Hz, at least 80 Hz, at least 85 Hz, or 90 Hz. In some embodiments of any of the aspects, the administration increases the power of gamma waves that are 60 Hz, at most 65 Hz, at most 70 Hz, at most 75 Hz, at most 80 Hz, at most 85 Hz, or at most 90 Hz. In some embodiments of any of the aspects, the administration increases the power of gamma waves that are 60-65 Hz, 65-70 Hz, 70-75 Hz, 75-80 Hz, 80-85 Hz, or 85-90 Hz. In some embodiments of any of the aspects, the administration increases the power of fast gamma waves (60 Hz-90 Hz) in the CA1 region of the hippocampus by at least 10%, at least 20%, at least 30%, at least 40%, at least 50%, at least 60%, at least 70%, at least 80%, at least 90%, at least 95%, at least 100% or more.

In some embodiments of any of the aspects, the administration increases firing of pyramidal cells in the CA1 region of the hippocampus during the descending phase of the theta_{pyr} cycle. As used herein, “theta_{pyr} cycle” refers to the theta waves or theta cycles in pyramidal cells, e.g., in the CA1 region of the hippocampus. Theta waves generate the theta rhythm, a neural oscillation in the brain that underlies various aspects of cognition and behavior, including learning, memory, and spatial navigation in many animals. The hippocampal theta rhythm is a strong oscillation that can be observed in the hippocampus and other brain structures in numerous species of mammals including rodents, rabbits, dogs, cats, bats, and marsupials. Hippocampal theta waves, with a frequency range of 4-12 Hz, appear when a mammal is engaged in active motor behavior such as walking or exploratory sniffing, and also during rapid eye movement (REM) sleep. In humans, hippocampal theta rhythm has been observed and linked to memory formation and navigation.

In some embodiments of any of the aspects, the theta_{pyr} cycle is at least 4 Hz, at least 5 Hz, at least 6 Hz, at least 7 Hz, at least 8 Hz, at least 9 Hz, at least 10 Hz, at least 11 Hz, or 12 Hz. In some embodiments of any of the aspects, the theta_{pyr} cycle is 4 Hz, at most 5 Hz, at most 6 Hz, at most 7 Hz, at most 8 Hz, at most 9 Hz, at most 10 Hz, at most 11 Hz, or at most 12 Hz. In some embodiments of any of the aspects, the theta pyr cycle is 4-5 Hz, 5-6 Hz, 6-7 Hz, 7-8 Hz, 8-9 Hz, 9-10 Hz, 10-11 Hz, or 11-12 Hz. In some embodiments of any of the aspects, the administration increases firing of pyramidal cells in the CA1 region of the hippocampus during the descending phase of the theta_{pyr} cycle by at least 10%, at least 20%, at least 30%, at least 40%, at least 50%, at least 60%, at least 70%, at least 80%, at least 90%, at least 95%, at least 100% or more.

As described herein, levels of a memory-associated analyte can be low in subjects with and/or in need of treatment for memory-associated disorders, learning disabilities, neurodegenerative diseases or disorders, and/or epilepsy. In some embodiments of any of the aspects, the memory-associated analyte is Scg2 mRNA, polypeptide, or neuropeptide. In some embodiments of any of the aspects, the memory-associated analyte is secretoneurin, EM66, manserin, and/or SgII. In some embodiments of any of the aspects, the memory-associated analyte is Fos, Fosb, or Junb mRNA or polypeptide. In some embodiments of any of the aspects, the memory-associated analyte is Fos+, Fosb+, or Junb+ cells, e.g., Fos+, Fosb+, or Junb+ neurons.

Accordingly, in one aspect of any of the embodiments, described herein is a method of treating a memory-associated disorder, a learning disability, a neurodegenerative disease or disorder, and/or epilepsy in a subject in need thereof, the method comprising administering an Scg2 neuropeptide pharmaceutical composition, nucleic acid, vector, or viral vector as described herein to a subject determined to have a level of a memory-associated analyte that is decreased relative to a reference. In one aspect of any of the embodiments, described herein is a method of treating a memory-associated disorder, a learning disability, a neurodegenerative disease or disorder, and/or epilepsy in a subject in need thereof, the method comprising: a) determining the level of a memory-associated analyte in a sample obtained from a subject; and b) administering an Scg2 neuropeptide pharmaceutical composition, nucleic acid, vector, or viral vector as described herein to the subject if the level of a memory-associated analyte is decreased relative to a reference.

In some embodiments of any of the aspects, the method comprises administering an Scg2 neuropeptide pharmaceutical composition, nucleic acid, vector, or viral vector as described herein to a subject previously determined to have a level of a memory-associated analyte that is decreased relative to a reference. In some embodiments of any of the aspects, described herein is a method of treating a memory-associated disorder, a learning disability, a neurodegenerative disease or disorder, and/or epilepsy in a subject in need thereof, the method comprising: a) first determining the level of a memory-associated analyte in a sample obtained from a subject; and b) then administering an Scg2 neuropeptide pharmaceutical composition, nucleic acid, vector, or viral vector as described herein to the subject if the level of a memory-associated analyte is decreased relative to a reference.

In one aspect of any of the embodiments, described herein is a method of treating a memory-associated disorder, a learning disability, a neurodegenerative disease or disorder, and/or epilepsy in a subject in need thereof, the method comprising: a) determining if the subject has a decreased level of a memory-associated analyte; and b) administering an Scg2 neuropeptide pharmaceutical composition, nucleic acid, vector, or viral vector as described herein to the subject if the level of a memory-associated analyte is decreased relative to a reference. In some embodiments of any of the aspects, the step of determining if the subject has a decreased level of a memory-associated analyte can comprise i) obtaining or having obtained a sample from the subject and ii) performing or having performed an assay on the sample obtained from the subject to determine/measure the level of a memory-associated analyte in the subject. In some embodiments of any of the aspects, the step of determining if the subject has a decreased level of a memory-associated analyte can comprise performing or having performed an assay on a sample obtained from the subject to determine/measure the level of a memory-associated analyte in the subject. In some embodiments of any of the aspects, the step of determining if the subject has a decreased level of a memory-associated analyte can comprise ordering or requesting an assay on a sample obtained from the subject to determine/measure the level of a memory-associated analyte in the subject. In some embodiments of any of the aspects, the step of determining if the subject has a decreased level of a memory-associated analyte can comprise receiving the results of an assay on a sample obtained from the subject to determine/measure the level of a memory-associated analyte in the subject. In some embodiments of any of the aspects, the step of determining if the subject has a decreased level of a memory-associated analyte can comprise receiving a report, results, or other means of identifying the subject as a subject with a decreased level of a memory-associated analyte.

In one aspect of any of the embodiments, described herein is a method of treating a memory-associated disorder, a learning disability, a neurodegenerative disease or disorder, and/or epilepsy in a subject in need thereof, the method comprising: a) determining if the subject has a decreased level of a memory-associated analyte; and b) instructing or directing that the subject be administered an Scg2 neuropeptide pharmaceutical composition, nucleic acid, vector, or viral vector as described herein if the level of a memory-associated analyte is decreased relative to a reference. In some embodiments of any of the aspects, the step of determining if the subject has a decreased level of a memory-associated analyte can comprise i) obtaining or having obtained a sample from the subject and ii) performing or having performed an assay on the sample obtained from the subject to determine/measure the level of a memory-associated analyte in the subject. In some embodiments of any of the aspects, the step of determining if the subject has a decreased level of a memory-associated analyte can comprise performing or having performed an assay on a sample obtained from the subject to determine/measure the level of a memory-associated analyte in the subject. In some embodiments of any of the aspects, the step of determining if the subject has a decreased level of a memory-associated analyte can comprise ordering or requesting an assay on a sample obtained from the subject to determine/measure the level of a memory-associated analyte in the subject. In some embodiments of any of the aspects, the step of instructing or directing that the subject be administered a particular treatment can comprise providing a report of the assay results. In some embodiments of any of the aspects, the step of instructing or directing that the subject be administered a particular treatment can comprise providing a report of the assay results and/or treatment recommendations in view of the assay results.

In one aspect, described herein is a method of increasing memory consolidation and/or memory retention in a subject in need thereof, comprising: (a) obtaining results detecting the level of a memory-associated analyte in a sample from the subject; and (b) administering to the subject: (i) a pharmaceutical composition, nucleic acid, vector, or viral vector as described herein, if the analyte level is below a pre-determined level; or (ii) an alternative treatment, if the analyte level is at or above a pre-determined level.

In one aspect, described herein is a method of treating a memory-associated disorder in a subject in need thereof, comprising: (a) obtaining results detecting the level of a memory-associated analyte in a sample from the subject; and (b) administering to the subject: (i) a pharmaceutical composition, nucleic acid, vector, or viral vector as described herein, if the analyte level is below a pre-determined level; or (ii) an alternative treatment, if the analyte level is at or above a pre-determined level.

In one aspect, described herein is a method of treating a learning disability in a subject in need thereof, comprising: (a) obtaining results detecting the level of a memory-associated analyte in a sample from the subject; and (b) administering to the subject: (i) a pharmaceutical composition, nucleic acid, vector, or viral vector as described herein, if the analyte level is below a pre-determined level; or (ii) an alternative treatment, if the analyte level is at or above a pre-determined level.

In one aspect, described herein is a method of treating a neurodegenerative disease or disorder in a subject in need thereof, comprising: (a) obtaining results detecting the level of a memory-associated analyte in a sample from the subject; and (b) administering to the subject: (i) a pharmaceutical composition, nucleic acid, vector, or viral vector as described herein, if the analyte level is below a pre-determined level; or (ii) an alternative treatment, if the analyte level is at or above a pre-determined level.

In one aspect, described herein is a method of treating epilepsy in a subject in need thereof, comprising: (a) obtaining results detecting the level of a memory-associated analyte in a sample from the subject; and (b) administering to the subject: (i) a pharmaceutical composition, nucleic acid, vector, or viral vector as described herein, if the analyte level is below a pre-determined level; or (ii) an alternative treatment, if the analyte level is at or above a pre-determined level.

In some embodiments, the subject has previously been determined to have a decreased level of a memory-associated analyte described herein relative to a reference. In some embodiments, the reference level can be the level in a sample of similar cell type, sample type, sample processing, and/or obtained from a subject of similar age, sex and other demographic parameters as the sample/subject. In some embodiments, the test sample and control reference sample are of the same type, that is, obtained from the same biological source, and comprising the same composition, e.g. the same number and type of cells.

The term “sample” or “test sample” as used herein denotes a sample taken or isolated from a biological organism, e.g., a blood or plasma sample from a subject. In some embodiments of any of the aspects, the sample is a cerebrospinal fluid sample or a CNS sample (e.g., a brain biopsy). In some embodiments of any of the aspects, the technology described herein encompasses several examples of a biological sample. In some embodiments of any of the aspects, the biological sample is cells, or tissue, or peripheral blood, or bodily fluid. Exemplary biological samples include, but are not limited to, a biopsy, a tumor sample, biofluid sample; blood; serum; plasma; urine; semen; mucus; tissue biopsy; organ biopsy; synovial fluid; bile fluid; cerebrospinal fluid; mucosal secretion; effusion; sweat; saliva; and/or tissue sample etc. The term also includes a mixture of the above-mentioned samples. The term “test sample” also includes untreated or pretreated (or pre-processed) biological samples. In some embodiments of any of the aspects, a test sample can comprise cells from a subject.

As described herein an alternative treatment can be administered to a subject, e.g., if the level of a memory-associated analyte is below a pre-determined level. As used herein, “alternative treatment” refers to a treatment other than the Scg2 neuropeptide pharmaceutical compositions, nucleic acids, vectors, or viral vectors described herein that can be administered to a subject to alleviate the symptoms of a specific memory-associated disorder, learning disability, or neurodegenerative disease or disorder. In embodiments, the alternative treatment is administered in addition to the Scg2 neuropeptide pharmaceutical compositions, nucleic acids, vectors, or viral vectors described herein. Alternative treatments can be selected according to the specific disease or disorder of the subject.

Non-limiting examples of alternative or additional treatments for memory-associated disorders, including but not limited to Alzheimer's disease or dementia, include: medications to treat symptoms related to memory and thinking (e.g., cholinesterase inhibitors such as Donepezil (Aricept®), Rivastigmine (Exelon®), or Galantamine (Razadyne®); glutamate regulators such as Memantine (Namenda®); Donepezil and memantine (Namzaric®); orexin receptor antagonist for insomnia such as Suvorexant (Belsomra®); an amyloid beta-directed monoclonal antibody such as Aducanumab (Aduhelm™)); therapies such as rehabilitation (e.g., retraining the brain's pathways) or occupational therapy; and the like.

Non-limiting examples of alternative or additional treatments for learning disabilities, including but not limited to ADHD, include: occupational therapy; individualized education program (IEP); or medication (e.g., ADDERALL (amphetamine); CONCERTA (methylphenidate, e.g., long acting); DEXEDRINE (dextroamphetamine); DEXTROSTAT (dextroamphetamine); FOCALIN (desmethylphenidate); METADATE ER (methylphenidate, e.g., extended release); METADATE CD (methylphenidate, e.g., extended release); RITALIN (methylphenidate); RITALIN SR (methylphenidate, e.g., extended release); RITALIN LA (methylphenidate, e.g., long acting); STRATTERA (atomoxetine); or VYVANSE (lisdexamfetamine dimesylate)).

Non-limiting examples of alternative or additional treatments for neurodegenerative diseases or disorders, including but not limited to Parkinson's disease, include: medications (e.g., LEVODOPA; CARBIDOPA-LEVODOPA; carbidopa (LODOSYN); dopamine agonists including pramipexole (MIRAPEX), ropinirole (REQUIP), rotigotine (NEUPRO, given as a patch), or apomorphine (APOKYN, a short-acting injectable dopamine); MAO B inhibitors including selegiline (ZELAPAR), rasagiline (AZILECT), or safinamide (XADAGO); catechol O-methyltransferase (COMT) inhibitors such as Entacapone (COMTAN), or opicapone (ONGENTYS); anticholinergics such as benztropine (COGENTIN) or trihexyphenidyl; amantadine); deep brain stimulation; or autologous stem cell therapy.

Non-limiting examples of alternative or additional treatments for neurodegenerative diseases or disorders, including but not limited to Huntington's disease, include: medications to control movement including tetrabenazine (XENAZINE) or deutetrabenazine (AUSTEDO); antipsychotic medications, such as haloperidol (HALDOL), fluphenazine, isperidone (RISPERDAL), olanzapine (ZYPREXA), or quetiapine (SEROQUEL); medications that can help suppress chorea, including amantadine (GOCOVRI ER, OSMOLEX ER), levetiracetam (KEPPRA, ELEPSIA XR, SPRITAM), or clonazepam (Klonopin); psychotherapy; speech therapy; physical therapy; or occupational therapy.

Non-limiting examples of alternative or additional treatments for neurodegenerative diseases or disorders, including but not limited to amyotrophic lateral sclerosis, include: medications such as Riluzole (RILUTEK) or Edaravone (RADICAVA); breathing therapy; speech therapy; physical therapy; occupational therapy; nutritional support; or psychological and social support.

Non-limiting examples of alternative or additional treatments for neurodegenerative diseases or disorders, including but not limited to multiple sclerosis, include: corticosteroids; plasma exchange (plasmapheresis); medications (e.g., ocrelizumab (OCREVUS); Interferon beta medications; Glatiramer acetate (COPAXONE, GLATOPA); Fingolimod (GILENYA); Dimethyl fumarate (TECFIDERA); Diroximel fumarate (VUMERITY); Teriflunomide (AUBAGIO); Siponimod (MAYZENT); Cladribine (MAVENCLAD); Natalizumab (TYSABRI); Alemtuzumab (CAMPATH, LEMTRADA)); physical therapy; muscle relaxants; medications to reduce fatigue such as Amantadine (GOCOVRI, OSMOLEX), modafinil (PROVIGIL), or methylphenidate (RITALIN); medication to increase walking speed such as Dalfampridine (AMPYRA); or medications for depression, pain, sexual dysfunction, insomnia, or bladder or bowel control problems.

Non-limiting examples of alternative or additional treatments for epilepsy include: anti-seizure medication (e.g., Carbamazepine (CARBATROL, TEGRETOL, others), Phenytoin (DILANTIN, PHENYTEK), Valproic acid (DEPAKENE), Oxcarbazepine (OXTELLAR, TRILEPTAL), Lamotrigine (LAMICTAL), Gabapentin (GRALISE, NEURONTIN), Topiramate (TOPAMAX), Phenobarbital, Zonisamide (ZONEGRAN)); brain surgery; Vagus nerve stimulation; ketogenic diet; deep brain stimulation; responsive neurosimulation; continuous stimulation of the seizure onset zone (subthreshold stimulation); MRI-guided focused ultrasound; transcranial magnetic stimulation (TMS); epilepsy pacemaker; or external trigeminal nerve stimulation.

In some embodiments of any of the aspects, the pharmaceutical composition, nucleic acid, vector, or viral vector described herein is administered as a monotherapy, e.g., another treatment for the learning disability, neurodegenerative disease or disorder, and/or epilepsy is not administered to the subject.

In some embodiments of any of the aspects, the methods described herein can further comprise administering a second agent and/or treatment to the subject, e.g. as part of a combinatorial therapy. In some embodiments of any of the aspects, the methods described herein can further comprise administering an alternative treatment, as described herein above, to the subject, e.g. as part of a combinatorial therapy, in addition to the Scg2 neuropeptide compositions described herein. By way of non-limiting example, if a subject is to be treated for pain or inflammation according to the methods described herein, the subject can also be administered a second agent and/or treatment known to be beneficial for subjects suffering from pain or inflammation. Examples of such agents and/or treatments include, but are not limited to, non-steroidal anti-inflammatory drugs (NSAIDs—such as aspirin, ibuprofen, or naproxen); corticosteroids, including glucocorticoids (e.g. cortisol, prednisone, prednisolone, methylprednisolone, dexamethasone, betamethasone, triamcinolone, and beclometasone); methotrexate; sulfasalazine; leflunomide; anti-TNF medications; cyclophosphamide; pro-resolving drugs; mycophenolate; or opiates (e.g. endorphins, enkephalins, and dynorphin), steroids, analgesics, barbiturates, oxycodone, morphine, lidocaine, and the like.

The term “effective amount” as used herein refers to the amount of an Scg2 neuropeptide pharmaceutical composition, nucleic acid, vector, or viral vector as described herein needed to alleviate at least one or more symptom of the disease or disorder, and relates to a sufficient amount of composition to provide the desired effect. The term “therapeutically effective amount” therefore refers to an amount of an Scg2 neuropeptide pharmaceutical composition, nucleic acid, vector, or viral vector as described herein that is sufficient to provide a particular anti-memory-associated disorder, anti-learning disability, and/or anti-neurodegenerative disease or disorder effect when administered to a typical subject. An effective amount as used herein, in various contexts, would also include an amount sufficient to delay the development of a symptom of the disease, alter the course of a symptom disease (for example but not limited to, slowing the progression of a symptom of the disease), or reverse a symptom of the disease. Thus, it is not generally practicable to specify an exact “effective amount”. However, for any given case, an appropriate “effective amount” can be determined by one of ordinary skill in the art using only routine experimentation.

Effective amounts, toxicity, and therapeutic efficacy can be determined by standard pharmaceutical procedures in cell cultures or experimental animals, e.g., for determining the LD50 (the dose lethal to 50% of the population) and the ED50 (the dose therapeutically effective in 50% of the population). The dosage can vary depending upon the dosage form employed and the route of administration utilized. The dose ratio between toxic and therapeutic effects is the therapeutic index and can be expressed as the ratio LD50/ED50. Compositions and methods that exhibit large therapeutic indices are preferred. A therapeutically effective dose can be estimated initially from cell culture assays. Also, a dose can be formulated in animal models to achieve a circulating plasma concentration range that includes the IC50 (i.e., the concentration of Scg2 neuropeptide, which achieves a half-maximal inhibition of symptoms) as determined in cell culture, or in an appropriate animal model. Levels in plasma can be measured, for example, by high performance liquid chromatography. The effects of any particular dosage can be monitored by a suitable bioassay, e.g., assays or tests for PV-IN or CCK-IN activity, power of fast gamma waves, CA1 pyramidal cell firing during the descending phase of the theta_{pyr} cycle, spatial learning, memory consolidation, and/or memory retention among others. The dosage can be determined by a physician and adjusted, as necessary, to suit observed effects of the treatment.

In embodiments wherein the administration is in the form of a nucleic acid, vector, or viral vector encoding at least one Scg2 neuropeptide, effective amounts, toxicity, and therapeutic efficacy can be determined by standard pharmaceutical procedures in cell cultures or experimental animals, e.g., for determining the minimal effective dose and/or maximal tolerated dose. The dosage can vary depending upon the dosage form employed and the route of administration utilized. A therapeutically effective dose can be estimated initially from cell culture assays. Also, a dose can be formulated in animal models to achieve a dosage range between the minimal effective dose and the maximal tolerated dose. The effects of any particular dosage can be monitored by a suitable bioassay, e.g., assay for tumor growth and/or size among others. The dosage can be determined by a physician and adjusted, as necessary, to suit observed effects of the treatment.

In certain embodiments, an effective dose of a composition comprising at least one Scg2 neuropeptide as described herein can be administered to a patient once. In certain embodiments, an effective dose of a composition comprising at least one Scg2 neuropeptide can be administered to a patient repeatedly. For systemic administration, subjects can be administered a therapeutic amount of a composition comprising at least one Scg2 neuropeptide, such as, e.g. 0.1 mg/kg, 0.5 mg/kg, 1.0 mg/kg, 2.0 mg/kg, 2.5 mg/kg, 5 mg/kg, 10 mg/kg, 15 mg/kg, 20 mg/kg, 25 mg/kg, 30 mg/kg, 40 mg/kg, 50 mg/kg, or more.

In some embodiments, after an initial treatment regimen, the treatments can be administered on a less frequent basis. For example, after treatment biweekly for three months, treatment can be repeated once per month, for six months or a year or longer. Treatment according to the methods described herein can reduce levels of a marker or symptom of a learning disability or a neurodegenerative disease or disorder, e.g. by at least 10%, at least 15%, at least 20%, at least 25%, at least 30%, at least 40%, at least 50%, at least 60%, at least 70%, at least 80% or at least 90% or more.

The dosage of a composition as described herein can be determined by a physician and adjusted, as necessary, to suit observed effects of the treatment. With respect to duration and frequency of treatment, it is typical for skilled clinicians to monitor subjects in order to determine when the treatment is providing therapeutic benefit, and to determine whether to increase or decrease dosage, increase or decrease administration frequency, discontinue treatment, resume treatment, or make other alterations to the treatment regimen. The dosing schedule can vary from once a week to daily depending on a number of clinical factors, such as the subject's sensitivity to the Scg2 neuropeptides. The desired dose or amount can be administered at one time or divided into subdoses, e.g., 2-4 subdoses and administered over a period of time, e.g., at appropriate intervals through the day or other appropriate schedule. In some embodiments, administration can be chronic, e.g., one or more doses and/or treatments daily over a period of weeks or months. Examples of dosing and/or treatment schedules are administration daily, twice daily, three times daily or four or more times daily over a period of 1 week, 2 weeks, 3 weeks, 4 weeks, 1 month, 2 months, 3 months, 4 months, 5 months, or 6 months, or more. A composition comprising Scg2 neuropeptides or nucleic acids, vectors, or viral vectors encodings at least one Scg2 neuropeptide can be administered over a period of time, such as over a 5 minute, 10 minute, 15 minute, 20 minute, or 25 minute period.

The dosage ranges for the administration of Scg2 neuropeptide pharmaceutical compositions, or nucleic acids, vectors, or viral vectors encoding at least one Scg2 neuropeptide, according to the methods described herein depend upon, for example, the form of the Scg2 neuropeptide (e.g., polypeptide or nucleic acid; specific pharmaceutically acceptable carrier) its potency, and the extent to which symptoms, markers, or indicators of a condition described herein are desired to be reduced, for example the percentage reduction desired for symptoms of a memory-associated, a learning disability, a neurodegenerative disease or disorder, and/or epilepsy, or the extent to which, for example, memory consolidation and/or memory retention are desired to be induced. The dosage should not be so large as to cause adverse side effects, such as overstimulation of the brain. Generally, the dosage will vary with the age, condition, and sex of the patient and can be determined by one of skill in the art. The dosage can also be adjusted by the individual physician in the event of any complication.

The efficacy of Scg2 neuropeptide pharmaceutical compositions or nucleic acids, vectors, or viral vectors encodings at least one Scg2 neuropeptide in, e.g. the treatment of a condition described herein, or to induce a response as described herein (e.g. memory consolidation and/or memory retention) can be determined by the skilled clinician. However, a treatment is considered “effective treatment,” as the term is used herein, if one or more of the signs or symptoms of a condition described herein are altered in a beneficial manner, other clinically accepted symptoms are improved, or even ameliorated, or a desired response is induced e.g., by at least 10% following treatment according to the methods described herein. Efficacy can be assessed, for example, by measuring a marker, indicator, symptom, and/or the incidence of a condition treated according to the methods described herein or any other measurable parameter appropriate, e.g. learning or memory acuity. Efficacy can also be measured by a failure of an individual to worsen as assessed by hospitalization, or need for medical interventions (i.e., progression of the disease is halted). Methods of measuring these indicators are known to those of skill in the art and/or are described herein. Treatment includes any treatment of a disease in an individual or an animal (some non-limiting examples include a human or an animal) and includes: (1) inhibiting the disease, e.g., preventing a worsening of symptoms as described herein; or (2) relieving the severity of the disease, e.g., causing regression of symptoms. An effective amount for the treatment of a disease means that amount which, when administered to a subject in need thereof, is sufficient to result in effective treatment as that term is defined herein, for that disease. Efficacy of an agent can be determined by assessing physical indicators of a condition or desired response, (e.g. memory consolidation and/or memory retention). It is well within the ability of one skilled in the art to monitor efficacy of administration and/or treatment by measuring any one of such parameters, or any combination of parameters. Efficacy can be assessed in animal models of a condition described herein, for example treatment of a memory-associated disorder, a learning disability, a neurodegenerative disease or disorder, and/or epilepsy. When using an experimental animal model, efficacy of treatment is evidenced when a statistically significant change in a marker is observed, e.g. Scg2 neuropeptide levels in the CNS or cerebrospinal fluid (CSF); PV-IN or CCK-IN activity, power of fast gamma waves, CA1 pyramidal cell firing during the descending phase of the theta_{pyr} cycle, spatial learning, memory consolidation, and/or memory retention among others.

In vitro and animal model assays are provided herein which allow the assessment of a given dose of a Scg2 neuropeptide pharmaceutical composition or a nucleic acid, vector, or viral vector encodings at least one Scg2 neuropeptide. A non-limiting example of an in vitro assay that can be performed to test efficacy or dosage includes: (1) exposure of a neuronal cell line (e.g., SH-SY5Y neuroblastoma cells; NT2 cells, such as NTERA-2 CL.D1 (NT2/D1) ATCC® CRL-1973™; PC-12 cells (e.g., ATCC® CRL-1721™)) or a primary neuronal culture (e.g., murine, rat, non-human primate, or human primary neuronal cultures) to an Scg2 neuropeptide pharmaceutical composition or a nucleic acid, vector, or viral vector encoding at least one Scg2 neuropeptide; and (2) assaying for neuronal activity using electrophysiology techniques such as patch-clamping. In some embodiments, the primary neuronal cultures can be isolated from the animal models described herein, e.g., for Alzheimer's disease, ADHD, Parkinson's disease, Huntington's disease, amyotrophic lateral sclerosis, multiple sclerosis, or epilepsy. In some embodiments, the primary neuronal cultures comprise interneurons (e.g., PV-INs, CCK-INs) and/or pyramidal cells (e.g., isolated from the CA1 region). In some embodiments, the neuronal cultures can be derived from human stem cells (e.g., induced pluripotent stem cells (iPSCs), e.g., from a human patient with a condition described herein). The neuronal cells can be monitored for signs that indicate efficacy of the treatment, including modulated interneuron activity such as increased PV-IN activity or decreased CCK-IN activity, increased power of fast gamma waves, and/or increased CA1 pyramidal cell firing during the descending phase of the theta_{pyr} cycle. The cells can also be monitored for viability, e.g., using live-dead staining; an optimal dose would exhibit a minimal or no decrease in viability, coupled with signs of efficacy in the neurons.

The efficacy of a given dosage combination can also be assessed in an animal model, e.g. for a memory-associated disorder, a learning disability, a neurodegenerative disease or disorder, and/or epilepsy. A non-limiting example of an in vivo assay that can be performed to test efficacy or dosage includes: (1) administration of an Scg2 neuropeptide pharmaceutical composition or a nucleic acid, vector, or viral vector encoding at least one Scg2 neuropeptide to the animal; and (2) assaying for neuronal activity, learning, memory consolidation, or memory retention in the animal. In some embodiments, the animal can be a mouse, rat, or non-human primate. In some embodiments, the animal can be a human in a clinical trial, e.g., using dosages determined in non-human animal trials. The animals can also be monitored for morbidity and mortality; an optimal dose would exhibit no mortality and minimal morbidity, coupled with signs of efficacy in the animals. See e.g., Drummond ad Wisniewski, Acta Neuropathol. 2017, 133(2): 155-175; Loscher, Neurochem Res. 2017, 42(7):1873-1888; Russell et al., Behav Brain Funct. 2005; 1: 9; Ramaswamy, ILAR J. 2007, 48(4):356-73; Morris et al., Neural Regen Res. 2018, 13(12): 2050-2054; Procaccini et al., Eur J Pharmacol. 2015, 759:182-91; Konnova and Swanberg. Chapter 5, Animal Models of Parkinson's Disease, Parkinson's Disease: Pathogenesis and Clinical Aspects, Codon Publications, 2018; the contents of each of which are incorporated herein by reference in their entireties.

Non-limiting examples of animal models for Alzheimer's disease include: transgenic mice that overexpress human genes associated with familial AD (FAD) (e.g., by expression of human APP alone or in combination with human PSEN1) that result in the formation of amyloid plaques; e.g., PDAPP, Tg2576, APP23, J20, APP/PS1, APPswe/PSldE9, TgSwDI, APP E693A-Tg, APP knock-in^{NL-G-F}, 3×Tg, 5×FAD, McGill-R-Thy1-APP, TgF344-AD, PSAPP.

Non-limiting examples of animal models for learning disabilities, including but not limited to ADHD, include: spontaneously hypertensive rats (SHR), Coloboma mutant mouse, 6-OHDA-Lesioned Rat, DAT-Knockout Mouse, poor 5-CSRT task performer ratanoxia in neonatal rat, Naples high-excitability rat (NHE), WKHA rat, acallosal mouse, hyposexual rat, PCB-exposed rat, lead-exposed mouse, or a rat reared in social isolation.

Non-limiting examples of animal models for neurodegenerative diseases or disorders, including but not limited to Parkinson's disease, include: neurotoxin-based approaches include exposure of rodents or non-human primates to 6-OHDA, MPTP, and agrochemicals such as the pesticide rotenone, the herbicide paraquat, and the fungicide maneb; genetic-based approaches to model Parkinson's disease include transgenic models and viral vector-mediated models based on genes linked to monogenic Parkinson's disease, including SNCA, LRRK2, UCH-L1, PRKN, PINK1, and DJ-1, as well as manipulation of dopaminergic transcription factors.

Non-limiting examples of animal models for neurodegenerative diseases or disorders, including but not limited to Huntington's disease, include: transgenic and knock-in rodents with the huntingtin (HTT) mutation (e.g., a mutation from a specific Huntington's disease human patient); toxin-induced models (e.g., 3-nitropropionic acid and/or quinolinic acid) to study mitochondrial impairment and excitotoxicity-induced cell death; or a viral vector to encode the HTT gene mutation in specific areas of the brain, e.g., in nonhuman primates.

Non-limiting examples of animal models for neurodegenerative diseases or disorders, including but not limited to amyotrophic lateral sclerosis, include: genetic ALS models, including C9orf72, mutant Cu/Zn superoxide dismutase 1 and TAR DNA-binding protein 43 mouse and zebrafish models; mouse or zebrafish models of environmentally-induced motor neuron degeneration (e.g., Bisphenol A (BPA) exposure; β-Sitosterol-β-d-glucoside (BSSG) exposure).

Non-limiting examples of animal models for neurodegenerative diseases or disorders, including but not limited to multiple sclerosis, include: (1) the experimental autoimmune/allergic encephalomyelitis (EAE) model; (2) the virally-induced chronic demyelinating disease, known as Theiler's murine encephalomyelitis virus (TMEV) infection; and (3) the toxin-induced demyelination

Non-limiting examples of animal models for epilepsy include: phenytoin- or lamotrigine-resistant kindled rat; the 6-Hz mouse model of partial seizures; the intrahippocampal kainate (e.g., kainic acid) model in mice; or rats in which spontaneous recurrent seizures develops after inducing status epilepticus by chemical or electrical stimulation.

In one aspect of any of the embodiments, described herein is a method of determining if a subject has a memory-associated disorder, learning disability, neurodegenerative disease or disorder, or epilepsy in a subject, or is in need of treatment for a memory-associated disorder, learning disability, neurodegenerative disease or disorder, or epilepsy in a subject, the method comprising: determining the level of a memory-associated analyte in a sample obtained from the subject, wherein a level of the memory-associated analyte which is decreased relative to a reference indicates the subject has a memory-associated disorder, learning disability, neurodegenerative disease or disorder, or epilepsy or is in need of treatment for a memory-associated disorder, learning disability, neurodegenerative disease or disorder, or epilepsy.

In one aspect, described herein is a method of diagnosing a memory-associated disorder, learning disability, neurodegenerative disease or disorder, or epilepsy in a subject; comprising: (a) obtaining a sample from the subject; (b) detecting the level of a memory-associated analyte in the sample; and (c) determining that the subject: (i) has or is at risk of developing a memory-associated disorder, a learning disability, a neurodegenerative disease or disorder, or epilepsy if the analyte level is below a pre-determined level; or (ii) does not have or is not at risk of developing a memory-associated disorder, a learning disability, a neurodegenerative disease or disorder, or epilepsy if the analyte level is at or above a pre-determined level.

In one aspect, described herein is a method of diagnosing a memory-associated disorder in a subject; comprising: (a) obtaining a sample from the subject; (b) detecting the level of a memory-associated analyte in the sample; and (c) determining that the subject: (i) has or is at risk of developing a memory-associated disorder if the analyte level is below a pre-determined level; or (ii) does not have or is not at risk of developing a memory-associated disorder if the analyte level is at or above a pre-determined level.

In one aspect, described herein is a method of diagnosing a learning disability in a subject; comprising: (a) obtaining a sample from the subject; (b) detecting the level of a memory-associated analyte in the sample; and (c) determining that the subject: (i) has or is at risk of developing a learning disability if the analyte level is below a pre-determined level; or (ii) does not have or is not at risk of developing a learning disability if the analyte level is at or above a pre-determined level.

In one aspect, described herein is a method of diagnosing a neurodegenerative disease or disorder in a subject; comprising: (a) obtaining a sample from the subject; (b) detecting the level of a memory-associated analyte in the sample; and (c) determining that the subject: (i) has or is at risk of developing a neurodegenerative disease or disorder if the analyte level is below a pre-determined level; or (ii) does not have or is not at risk of developing a neurodegenerative disease or disorder if the analyte level is at or above a pre-determined level.

In one aspect, described herein is a method of diagnosing epilepsy in a subject; comprising: (a) obtaining a sample from the subject; (b) detecting the level of a memory-associated analyte in the sample; and (c) determining that the subject: (i) has or is at risk of developing epilepsy if the analyte level is below a pre-determined level; or (ii) does not have or is not at risk of developing epilepsy if the analyte level is at or above a pre-determined level.

In one aspect, described herein is a method for detecting a memory-associated analyte in a sample from a subject comprising: (a) obtaining a sample from the subject; and (b) detecting the level of the memory-associated analyte in the sample. In some embodiments of any of the aspects, the sample is a cerebrospinal fluid sample or a CNS sample (e.g., a brain biopsy).

As described herein, levels of a memory-associated analyte can be low in subjects diagnosed with memory-associated disorders, learning disabilities, neurodegenerative diseases or disorders, and/or epilepsy. In some embodiments of any of the aspects, the memory-associated analyte is Scg2 mRNA, polypeptide, or neuropeptide. In some embodiments of any of the aspects, the memory-associated analyte is secretoneurin, EM66, manserin, and/or SgII. In some embodiments of any of the aspects, the memory-associated analyte is Fos, Fosb, or Junb mRNA or polypeptide. In some embodiments of any of the aspects, the memory-associated analyte is Fos+, Fosb+, or Junb+ cells, e.g., Fos+, Fosb+, or Junb+ neurons.

In some embodiments of any of the aspects, the diagnosis and/or detection method further comprises administering to the subject an Scg2 neuropeptide pharmaceutical composition, nucleic acid, vector, or viral vector as described herein, e.g., if the subject is determined to have or be at risk for developing a learning disability or a neurodegenerative disease or disorder. In some embodiments, the subject is also administered an additional treatment for the memory-associated disorder, learning disability, neurodegenerative disease or disorder, or epilepsy, as described herein or known in the art, e.g., if the subject is determined to have or be at risk for developing a learning disability or a neurodegenerative disease or disorder. In some embodiments, the subject is administered an alternative treatment for the memory-associated disorder, learning disability, neurodegenerative disease or disorder, or epilepsy, as described herein or known in the art, e.g., if the subject is determined to not have or not be at risk for developing a learning disability or a neurodegenerative disease or disorder.

In some embodiments of any of the aspects, the step of detecting the level of the memory-associated analyte comprises mRNA detection or polypeptide detection. In some embodiments of any of the aspects, the mRNA detection comprises reverse transcription polymerase chain reaction (RT-PCR); quantitative RT-PCR; Northern blot analysis; differential gene expression; RNase protection assay; microarray based analysis; next-generation sequencing; or hybridization methods. In some embodiments of any of the aspects, the polypeptide detection comprises immunoassays, Western blot; immunoprecipitation; enzyme-linked immunosorbent assay (ELISA); radioimmunological assay (RIA); sandwich assay; immunohistological staining; radioimmunometric assay; immunofluorescence assay; mass spectroscopy; or immunoelectrophoresis assay.

In some embodiments of any of the aspects, measurement of the level of a target and/or detection of the level or presence of a target, e.g. of an expression product (nucleic acid or polypeptide of one of the genes described herein) or a mutation can comprise a transformation. As used herein, the term “transforming” or “transformation” refers to changing an object or a substance, e.g., biological sample, nucleic acid or protein, into another substance. The transformation can be physical, biological or chemical. Exemplary physical transformation includes, but is not limited to, pre-treatment of a biological sample, e.g., from whole blood to blood serum by differential centrifugation. A biological/chemical transformation can involve the action of at least one enzyme and/or a chemical reagent in a reaction. For example, a DNA sample can be digested into fragments by one or more restriction enzymes, or an exogenous molecule can be attached to a fragmented DNA sample with a ligase. In some embodiments of any of the aspects, a DNA sample can undergo enzymatic replication, e.g., by polymerase chain reaction (PCR).

Transformation, measurement, and/or detection of a target molecule, (e.g. a Scg2 mRNA or polypeptide; secretoneurin, EM66, manserin, and/or SgII mRNA or polypeptide; Fos, Fosb, or Junb mRNA or polypeptide) can comprise contacting a sample obtained from a subject with a reagent (e.g. a detection reagent) which is specific for the target, e.g., a target-specific reagent. In some embodiments of any of the aspects, the target-specific reagent is detectably labeled. In some embodiments of any of the aspects, the target-specific reagent is capable of generating a detectable signal. In some embodiments of any of the aspects, the target-specific reagent generates a detectable signal when the target molecule is present.

Methods to measure gene expression products are known to a skilled artisan. Such methods to measure gene expression products, e.g., protein level, include ELISA (enzyme linked immunosorbent assay), western blot, immunoprecipitation, and immunofluorescence using detection reagents such as an antibody or protein binding agents. Alternatively, a peptide can be detected in a subject by introducing into a subject a labeled anti-peptide antibody and other types of detection agent. For example, the antibody can be labeled with a detectable marker whose presence and location in the subject is detected by standard imaging techniques.

For example, antibodies for the various targets described herein are commercially available and can be used for the purposes of the invention to measure protein expression levels, e.g. anti-Scg2 (THERMO FISHER SCIENTIFIC TA811982; THERMO FISHER SCIENTIFIC TA811869); anti-Fos (SYNAPTIC SYSTEMS 226003; ABCAM ab208942; LSBIO LS-B6420-50); anti-Fosb (CELL SIGNALING TECHNOLOGY 2251S; ABCAM ab184938); anti-Junb (CELL SIGNALING TECHNOLOGY 3753S; MYBIOSOURCE.COM MBS120282). Alternatively, since the amino acid sequences for the targets described herein are known and publically available at the NCBI website, one of skill in the art can raise their own antibodies against these polypeptides of interest for the purpose of the methods described herein.

The amino acid sequences of the polypeptides described herein have been assigned NCBI accession numbers for different species such as human, mouse and rat. In particular, the NCBI accession numbers for the amino acid sequence of human Scg2 is included herein, see e.g. SEQ ID NO: 4. Furthermore, the NCBI accession numbers for the amino acid sequence of human Fos, Fosb, or Junb is included herein, see e.g. SEQ ID NOs: 24-26.

SEQ ID NO: 24, proto-oncogene c-Fos Homo sapiens,
NCBI Reference Sequence:
NP_005243.1, 380 aa
mmfsgfnadyeasssressaspagdslsyyhspadsfssmgspvnaqdf
ctdlavssanfiptvtaistspdlqwlvqpalvssvapsqtraphpfgv
papsagaysragvvktmtggraqsigrrgkveqlspeeeekrrirrern
kmaaakcrnrrreltdtlqaetdqledeksalqteianllkekeklefi
laahrpackipddlgfpeemsvasldltgglpevatpeseeaftlplln
dpepkpsvepvksissmelktepfddflfpassrpsgsetarsvpdmdl
sgsfyaadweplhsgslgmgpmateleplctpvvtctpsctaytssfvf
typeadsfpscaaahrkgsssnepssdslssptllal
SEQ ID NO: 25, protein fosB isoform 2
Homo sapiens, NCBI Reference Sequence:
NP_001107643.1, 302 aa
mfqafpgdydsgsrcssspsaesqylssvdsfgspptaaasqecaglge
mpgsfvptvtaittsqdlqwlvqptlissmaqsqgqplasqppvvdpyd
mpgtsystpgmsgyssggasgsggpstsgttsgpgparpararprrpre
etetdqleeekaeleseiaelqkekerlefvlvahkpgckipyeegpgp
gplaevrdlpgsapakedgfswllppppppplpfqtsqdappnltaslf
thsevqvlgdpfpvvnpsytssfvltcpevsafagaqrtsgsdqpsdpl
nspsllal
SEQ ID NO: 26, transcription factor jun-B
Homo sapiens, NCBI Reference Sequence:
NP_002220.1, 347 aa
mctkmeqpfyhddsytatgygrapgglslhdykllkpslavnladpyrs
lkapgargpgpegggggsyfsgqgsdtgaslklasselerlivpnsngv
itttptppgqyfyprgggsgggaggagggvteeqegfadgfvkalddlh
kmnhvtppnvslgatggppagpggvyagpepppvytnlssyspasassg
gagaavgtgssyptttisylphappfagghpaqlglgrgastfkeepqt
vpearsrdatppvspinmedqerikverkrlrnrlaatkcrkrkleria
rledkvktlkaenaglsstagllreqvaqlkqkvmthvsngcqlllgvk
ghaf

In some embodiments of any of the aspects, immunohistochemistry (“IHC”) and immunocytochemistry (“ICC”) techniques can be used. IHC is the application of immunochemistry to tissue sections, whereas ICC is the application of immunochemistry to cells or tissue imprints after they have undergone specific cytological preparations such as, for example, liquid-based preparations. Immunochemistry is a family of techniques based on the use of an antibody, wherein the antibodies are used to specifically target molecules inside or on the surface of cells. The antibody typically contains a marker that will undergo a biochemical reaction, and thereby experience a change of color, upon encountering the targeted molecules. In some instances, signal amplification can be integrated into the particular protocol, wherein a secondary antibody, that includes the marker stain or marker signal, follows the application of a primary specific antibody.

In some embodiments of any of the aspects, the assay can be a Western blot analysis. Alternatively, proteins can be separated by two-dimensional gel electrophoresis systems. Two-dimensional gel electrophoresis is well known in the art and typically involves iso-electric focusing along a first dimension followed by SDS-PAGE electrophoresis along a second dimension. These methods also require a considerable amount of cellular material. The analysis of 2D SDS-PAGE gels can be performed by determining the intensity of protein spots on the gel, or can be performed using immune detection. In other embodiments, protein samples are analyzed by mass spectroscopy.

Immunological tests can be used with the methods and assays described herein and include, for example, competitive and non-competitive assay systems using techniques such as Western blots, radioimmunoassay (RIA), ELISA (enzyme linked immunosorbent assay), “sandwich” immunoassays, immunoprecipitation assays, immunodiffusion assays, agglutination assays, e.g. latex agglutination, complement-fixation assays, immunoradiometric assays, fluorescent immunoassays, e.g. FIA (fluorescence-linked immunoassay), chemiluminescence immunoassays (CLIA), electrochemiluminescence immunoassay (ECLIA, counting immunoassay (CIA), lateral flow tests or immunoassay (LFIA), magnetic immunoassay (MIA), and protein A immunoassays. Methods for performing such assays are known in the art, provided an appropriate antibody reagent is available. In some embodiments of any of the aspects, the immunoassay can be a quantitative or a semi-quantitative immunoassay.

An immunoassay is a biochemical test that measures the concentration of a substance in a biological sample, typically a fluid sample such as blood or serum, using the interaction of an antibody or antibodies to its antigen. The assay takes advantage of the highly specific binding of an antibody with its antigen. For the methods and assays described herein, specific binding of the target polypeptides with respective proteins or protein fragments, or an isolated peptide, or a fusion protein described herein occurs in the immunoassay to form a target protein/peptide complex. The complex is then detected by a variety of methods known in the art. An immunoassay also often involves the use of a detection antibody.

Enzyme-linked immunosorbent assay, also called ELISA, enzyme immunoassay or EIA, is a biochemical technique used mainly in immunology to detect the presence of an antibody or an antigen in a sample. The ELISA has been used as a diagnostic tool in medicine and plant pathology, as well as a quality control check in various industries.

In one embodiment, an ELISA involving at least one antibody with specificity for the particular desired antigen (e.g., any of the targets as described herein) can also be performed. A known amount of sample and/or antigen is immobilized on a solid support (usually a polystyrene micro titer plate). Immobilization can be either non-specific (e.g., by adsorption to the surface) or specific (e.g. where another antibody immobilized on the surface is used to capture antigen or a primary antibody). After the antigen is immobilized, the detection antibody is added, forming a complex with the antigen. The detection antibody can be covalently linked to an enzyme, or can itself be detected by a secondary antibody which is linked to an enzyme through bio-conjugation. Between each step the plate is typically washed with a mild detergent solution to remove any proteins or antibodies that are not specifically bound. After the final wash step, the plate is developed by adding an enzymatic substrate to produce a visible signal, which indicates the quantity of antigen in the sample. Older ELISAs utilize chromogenic substrates, though newer assays employ fluorogenic substrates with much higher sensitivity.

In another embodiment, a competitive ELISA is used. Purified antibodies that are directed against a target polypeptide or fragment thereof are coated on the solid phase of multi-well plate, i.e., conjugated to a solid surface. A second batch of purified antibodies that are not conjugated on any solid support is also needed. These non-conjugated purified antibodies are labeled for detection purposes, for example, labeled with horseradish peroxidase to produce a detectable signal. A sample (e.g., a blood sample) from a subject is mixed with a known amount of desired antigen (e.g., a known volume or concentration of a sample comprising a target polypeptide) together with the horseradish peroxidase labeled antibodies and the mixture is then added to coated wells to form competitive combination. After incubation, if the polypeptide level is high in the sample, a complex of labeled antibody reagent-antigen will form. This complex is free in solution and can be washed away. Washing the wells will remove the complex. Then the wells are incubated with TMB (3, 3′, 5, 5′-tetramethylbenzidene) color development substrate for localization of horseradish peroxidase-conjugated antibodies in the wells. There will be no color change or little color change if the target polypeptide level is high in the sample. If there is little or no target polypeptide present in the sample, a different complex in formed, the complex of solid support bound antibody reagents-target polypeptide. This complex is immobilized on the plate and is not washed away in the wash step. Subsequent incubation with TMB will produce significant color change. Such a competitive ELSA test is specific, sensitive, reproducible and easy to operate.

There are other different forms of ELISA, which are well known to those skilled in the art. The standard techniques known in the art for ELISA are described in “Methods in Immunodiagnosis”, 2nd Edition, Rose and Bigazzi, eds. John Wiley & Sons, 1980; and Oellerich, M. 1984, J. Clin. Chem. Clin. Biochem. 22:895-904. These references are hereby incorporated by reference in their entirety.

In one embodiment, the levels of a polypeptide in a sample can be detected by a lateral flow immunoassay test (LFIA), also known as the immunochromatographic assay, or strip test. LFIAs are a simple device intended to detect the presence (or absence) of antigen, e.g. a polypeptide, in a fluid sample. There are currently many LFIA tests used for medical diagnostics, either for home testing, point of care testing, or laboratory use. LFIA tests are a form of immunoassay in which the test sample flows along a solid substrate via capillary action. After the sample is applied to the test strip it encounters a colored reagent (generally comprising antibody specific for the test target antigen) bound to microparticles which mixes with the sample and transits the substrate encountering lines or zones which have been pretreated with another antibody or antigen. Depending upon the level of target polypeptides present in the sample the colored reagent can be captured and become bound at the test line or zone. LFIAs are essentially immunoassays adapted to operate along a single axis to suit the test strip format or a dipstick format. Strip tests are extremely versatile and can be easily modified by one skilled in the art for detecting an enormous range of antigens from fluid samples such as urine, blood, water, and/or homogenized tissue samples etc. Strip tests are also known as dip stick tests, the name bearing from the literal action of “dipping” the test strip into a fluid sample to be tested. LFIA strip tests are easy to use, require minimum training and can easily be included as components of point-of-care test (POCT) diagnostics to be use on site in the field. LFIA tests can be operated as either competitive or sandwich assays. Sandwich LFIAs are similar to sandwich ELISA. The sample first encounters colored particles which are labeled with antibodies raised to the target antigen. The test line will also contain antibodies to the same target, although it may bind to a different epitope on the antigen. The test line will show as a colored band in positive samples. In some embodiments of any of the aspects, the lateral flow immunoassay can be a double antibody sandwich assay, a competitive assay, a quantitative assay or variations thereof. Competitive LFIAs are similar to competitive ELISA. The sample first encounters colored particles which are labeled with the target antigen or an analogue. The test line contains antibodies to the target/its analogue. Unlabeled antigen in the sample will block the binding sites on the antibodies preventing uptake of the colored particles. The test line will show as a colored band in negative samples. There are a number of variations on lateral flow technology. It is also possible to apply multiple capture zones to create a multiplex test.

The use of “dip sticks” or LFIA test strips and other solid supports have been described in the art in the context of an immunoassay for a number of antigen biomarkers. U.S. Pat. Nos. 4,943,522; 6,485,982; 6,187,598; 5,770,460; 5,622,871; 6,565,808, U.S. patent application Ser. No. 10/278,676; U.S. Ser. No. 09/579,673 and U.S. Ser. No. 10/717,082, which are incorporated herein by reference in their entirety, are non-limiting examples of such lateral flow test devices. Examples of patents that describe the use of “dip stick” technology to detect soluble antigens via immunochemical assays include, but are not limited to U.S. Pat. Nos. 4,444,880; 4,305,924; and 4,135,884; which are incorporated by reference herein in their entireties. The apparatuses and methods of these three patents broadly describe a first component fixed to a solid surface on a “dip stick” which is exposed to a solution containing a soluble antigen that binds to the component fixed upon the “dip stick,” prior to detection of the component-antigen complex upon the stick. It is within the skill of one in the art to modify the teachings of this “dip stick” technology for the detection of polypeptides using antibody reagents as described herein.

Other techniques can be used to detect the level of a polypeptide in a sample. One such technique is the dot blot, an adaptation of Western blotting (Towbin et at., Proc. Nat. Acad. Sci. 76:4350 (1979)). In a Western blot, the polypeptide or fragment thereof can be dissociated with detergents and heat, and separated on an SDS-PAGE gel before being transferred to a solid support, such as a nitrocellulose or PVDF membrane. The membrane is incubated with an antibody reagent specific for the target polypeptide or a fragment thereof. The membrane is then washed to remove unbound proteins and proteins with non-specific binding. Detectably labeled enzyme-linked secondary or detection antibodies can then be used to detect and assess the amount of polypeptide in the sample tested. A dot blot immobilizes a protein sample on a defined region of a support, which is then probed with antibody and labelled secondary antibody as in Western blotting. The intensity of the signal from the detectable label in either format corresponds to the amount of enzyme present, and therefore the amount of polypeptide. Levels can be quantified, for example by densitometry.

In some embodiments of any of the aspects, the level of a target can be measured, by way of non-limiting example, by Western blot; immunoprecipitation; enzyme-linked immunosorbent assay (ELISA); radioimmunological assay (RIA); sandwich assay; fluorescence in situ hybridization (FISH); immunohistological staining; radioimmunometric assay; immunofluorescence assay; mass spectroscopy and/or immunoelectrophoresis assay.

In certain embodiments, the gene expression products as described herein can be instead determined by determining the level of messenger RNA (mRNA) expression of the genes described herein. Such molecules can be isolated, derived, or amplified from a biological sample, such as a blood sample. Techniques for the detection of mRNA expression are known by persons skilled in the art, and can include but not limited to, PCR procedures, RT-PCR, quantitative RT-PCR, Northern blot analysis, differential gene expression, RNase protection assay, microarray based analysis, next-generation sequencing; hybridization methods, etc.

In general, the PCR procedure describes a method of gene amplification which is comprised of (i) sequence-specific hybridization of primers to specific genes or sequences within a nucleic acid sample or library, (ii) subsequent amplification involving multiple rounds of annealing, elongation, and denaturation using a thermostable DNA polymerase, and (iii) screening the PCR products for a band of the correct size. The primers used are oligonucleotides of sufficient length and appropriate sequence to provide initiation of polymerization, i.e. each primer is specifically designed to be complementary to a strand of the genomic locus to be amplified. In an alternative embodiment, mRNA level of gene expression products described herein can be determined by reverse-transcription (RT) PCR or quantitative RT-PCR (QRT-PCR) or real-time PCR methods. Methods of RT-PCR and QRT-PCR are well known in the art.

In some embodiments of any of the aspects, the level of an mRNA can be measured by a quantitative sequencing technology, e.g. a quantitative next-generation sequencing technology. Methods of sequencing a nucleic acid sequence are well known in the art. Briefly, a sample obtained from a subject can be contacted with one or more primers which specifically hybridize to a single-strand nucleic acid sequence flanking the target gene sequence and a complementary strand is synthesized. In some next-generation technologies, an adaptor (double or single-stranded) is ligated to nucleic acid molecules in the sample and synthesis proceeds from the adaptor or adaptor compatible primers. In some third-generation technologies, the sequence can be determined, e.g. by determining the location and pattern of the hybridization of probes, or measuring one or more characteristics of a single molecule as it passes through a sensor (e.g. the modulation of an electrical field as a nucleic acid molecule passes through a nanopore). Exemplary methods of sequencing include, but are not limited to, Sanger sequencing (i.e., dideoxy chain termination), high-throughput sequencing, next generation sequencing, 454 sequencing, SOLiD sequencing, polony sequencing, Illumina sequencing, Ion Torrent sequencing, sequencing by hybridization, nanopore sequencing, Helioscope sequencing, single molecule real time sequencing, RNAP sequencing, and the like. Methods and protocols for performing these sequencing methods are known in the art, see, e.g. “Next Generation Genome Sequencing” Ed. Michal Janitz, Wiley-VCH; “High-Throughput Next Generation Sequencing” Eds. Kwon and Ricke, Humanna Press, 2011; and Sambrook et al., Molecular Cloning: A Laboratory Manual (4 ed.), Cold Spring Harbor Laboratory Press, Cold Spring Harbor, N.Y., USA (2012); which are incorporated by reference herein in their entireties.

The nucleic acid sequences of the genes described herein have been assigned NCBI accession numbers for different species such as human, mouse and rat. For example, the human Scg2 mRNA (e.g. SEQ ID NOs: 2-3), human Fos mRNA (e.g. SEQ ID NOs: 27-28), human Fosb mRNA (e.g. SEQ ID NOs: 29-30), human Junb mRNA (e.g. SEQ ID NOs: 31-32) are known. Accordingly, a skilled artisan can design appropriate primer(s) and/or probe(s) based on the known sequence for determining the mRNA level of the respective gene.

SEQ ID NO: 27, Fos proto-oncogene, AP-1 transcription factor subunit Homo sapiens
(human), Gene ID: 2353, mRNA, NCBI Reference Sequence: NM_005252.4 (CDS region nt
156-1298), 2104 nt:
aaccgcatctgcagcgagcatctgagaagccaagactgagccggcggccgcggcgcagcgaacgagcagtgaccgtgctcctacccagctct
gctccacagcgcccacctgtctccgcccctcggcccctcgcccggctttgcctaaccgccacgatgatgttctcgggcttcaacgcagacta
cgaggcgtcatcctcccgctgcagcagcgcgtccccggccggggatagcctctcttactaccactcacccgcagactccttctccagcatgg
gctcgcctgtcaacgcgcaggacttctgcacggacctggccgtctccagtgccaacttcattcccacggtcactgccatctcgaccagtccg
gacctgcagtggctggtgcagcccgccctcgtctcctccgtggccccatcgcagaccagagcccctcaccctttcggagtccccgccccctc
cgctggggcttactccagggctggcgttgtgaagaccatgacaggaggccgagcgcagagcattggcaggaggggcaaggtggaacagttat
ctccagaagaagaagagaaaaggagaatccgaagggaaaggaataagatggctgcagccaaatgccgcaaccggaggagggagctgactgat
acactccaagcggagacagaccaactagaagatgagaagtctgctttgcagaccgagattgccaacctgctgaaggagaaggaaaaactaga
gttcatcctggcagctcaccgacctgcctgcaagatccctgatgacctgggcttcccagaagagatgtctgtggcttcccttgatctgactg
ggggcctgccagaggttgccaccccggagtctgaggaggccttcaccctgcctctcctcaatgaccctgagcccaagccctcagtggaacct
gtcaagagcatcagcagcatggagctgaagaccgagccctttgatgacttcctgttcccagcatcatccaggcccagtggctctgagacagc
ccgctccgtgccagacatggacctatctgggtccttctatgcagcagactgggagcctctgcacagtggctccctggggatggggcccatgg
ccacagagctggagcccctgtgcactccggtggtcacctgtactcccagctgcactgcttacacgtcttccttcgtcttcacctaccccgag
gctgactccttccccagctgtgcagctgcccaccgcaagggcagcagcagcaatgagccttcctctgactcgctcagctcacccacgctgct
ggccctgtgagggggcagggaaggggaggcagccggcacccacaagtgccactgcccgagctggtgcattacagagaggagaaacacatctt
ccctagagggttcctgtagacctagggaggaccttatctgtgcgtgaaacacaccaggctgtgggcctcaaggacttgaaagcatccatgtg
tggactcaagtccttacctcttccggagatgtagcaaaacgcatggagtgtgtattgttcccagtgacacttcagagagctggtagttagta
gcatgttgagccaggcctgggtctgtgtctcttttctctttctccttagtcttctcatagcattaactaatctattgggttcattattggaa
ttaacctggtgctggatattttcaaattgtatctagtgcagctgattttaacaataactactgtgttcctggcaatagtgtgttctgattag
aaatgaccaatattatactaagaaaagatacgactttattttctggtagatagaaataaatagctatatccatgtactgtagtttttcttca
acatcaatgttcattgtaatgttactgatcatgcattgttgaggtggtctgaatgttctgacattaacagttttccatgaaaacgttttatt
gtgtttttaatttatttattaagatggattctcagatatttatatttttattttatttttttctaccttgaggtcttttgacatgtggaaag
tgaatttgaatgaaaaatttaagcattgtttgcttattgttccaagacattgtcaataaaagcatttaagttgaatgcga
SEQ ID NO: 28, Fos proto-oncogene, AP-1 transcription factor subunit Homo sapiens
(human), Gene ID: 2353, mRNA CDS, NCBI Reference Sequence: NM_005252.4 (CDS region nt
156-1298), 1143 nt:
atgatgttctcgggcttcaacgcagactacgaggcgtcatcctcccgctgcagcagcgcgtccccggccggggatagcctctcttactacca
ctcacccgcagactccttctccagcatgggctcgcctgtcaacgcgcaggacttctgcacggacctggccgtctccagtgccaacttcattc
ccacggtcactgccatctcgaccagtccggacctgcagtggctggtgcagcccgccctcgtctcctccgtggccccatcgcagaccagagcc
cctcaccctttcggagtccccgccccctccgctggggcttactccagggctggcgttgtgaagaccatgacaggaggccgagcgcagagcat
tggcaggaggggcaaggtggaacagttatctccagaagaagaagagaaaaggagaatccgaagggaaaggaataagatggctgcagccaaat
gccgcaaccggaggagggagctgactgatacactccaagcggagacagaccaactagaagatgagaagtctgctttgcagaccgagattgcc
aacctgctgaaggagaaggaaaaactagagttcatcctggcagctcaccgacctgcctgcaagatccctgatgacctgggcttcccagaaga
gatgtctgtggcttcccttgatctgactgggggcctgccagaggttgccaccccggagtctgaggaggccttcaccctgcctctcctcaatg
accctgagcccaagccctcagtggaacctgtcaagagcatcagcagcatggagctgaagaccgagccctttgatgacttcctgttcccagca
tcatccaggcccagtggctctgagacagcccgctccgtgccagacatggacctatctgggtccttctatgcagcagactgggagcctctgca
cagtggctccctggggatggggcccatggccacagagctggagcccctgtgcactccggtggtcacctgtactcccagctgcactgcttaca
cgtcttccttcgtcttcacctaccccgaggctgactccttccccagctgtgcagctgcccaccgcaagggcagcagcagcaatgagccttcc
tctgactcgctcagctcacccacgctgctggccctgtga
SEQ ID NO: 29, FosB proto-oncogene, AP-1 transcription factor subunit Homo sapiens
(human), transcript variant 2, Gene ID: 2354, mRNA, NCBI Reference Sequence: NM_001114171.2
(CDS region nt 592-1500), 3667 nt:
attcataagactcagagctacggccacggcagggacacgcggaaccaagacttggaaacttgattgttgtggttcttcttgggggttatgaa
atttcattaatctttttttttccggggagaaagtttttggaaagattcttccagatatttcttcattttcttttggaggaccgacttacttt
ttttggtcttctttattactcccctccccccgtgggacccgccggacgcgtggaggagaccgtagctgaagctgattctgtacagcgggaca
gcgctttctgcccctgggggagcaacccctccctcgcccctgggtcctacggagcctgcactttcaagaggtacagcggcatcctgtggggg
cctgggcaccgcaggaagactgcacagaaactttgccattgttggaacgggacgttgctccttccccgagcttccccggacagcgtactttg
aggactcgctcagctcaccggggactcccacggctcaccccggacttgcaccttacttccccaacccggccatagccttggcttcccggcga
cctcagcgtggtcacaggggcccccctgtgcccagggaaatgtttcaggctttccccggagactacgactccggctcccggtgcagctcctc
accctctgccgagtctcaatatctgtcttcggtggactccttcggcagtccacccaccgccgccgcctcccaggagtgcgccggtctcgggg
aaatgcccggttccttcgtgcccacggtcaccgcgatcacaaccagccaggacctccagtggcttgtgcaacccaccctcatctcttccatg
gcccagtcccaggggcagccactggcctcccagcccccggtcgtcgacccctacgacatgccgggaaccagctactccacaccaggcatgag
tggctacagcagtggcggagcgagtggcagtggtgggccttccaccagcggaactaccagtgggcctgggcctgcccgcccagcccgagccc
ggcctaggagaccccgagaggagacggagacagatcagttggaggaagaaaaagcagagctggagtcggagatcgccgagctccaaaaggag
aaggaacgtctggagtttgtgctggtggcccacaaaccgggctgcaagatcccctacgaagaggggcccgggccgggcccgctggcggaggt
gagagatttgccgggctcagcaccggctaaggaagatggcttcagctggctgctgccgcccccgccaccaccgcccctgcccttccagacca
gccaagacgcaccccccaacctgacggcttctctctttacacacagtgaagttcaagtcctcggcgaccccttccccgttgttaacccttcg
tacacttcttcgtttgtcctcacctgcccggaggtctccgcgttcgccggcgcccaacgcaccagcggcagtgaccagccttccgatcccct
gaactcgccctccctcctcgctctgtgaactctttagacacacaaaacaaacaaacacatgggggagagagacttggaagaggaggaggagg
aggagaaggaggagagagaggggaagagacaaagtgggtgtgtggcctccctggctcctccgtctgaccctctgcggccactgcgccactgc
catcggacaggaggattccttgtgttttgtcctgcctcttgtttctgtgccccggcgaggccggagagctggtgactttggggacagggggg
ggaaggggatggacacccccagctgactgttggctctctgacgtcaacccaagctctggggatgggtggggaggggggcgggtgacgcccac
cttcgggcagtcctgtgtgaggattaagggacggggggggaggtaggctgtgggggggctggagtcctctccagagaggctcaacaaggaaa
aatgccactccctacccaatgtctcccacacccaccctttttttggggtgcctaggttggtttcccctgcactcccgaccttagcttattga
tcccacatttccatggtgtgagatcctctttactctgggcagaagtgagccccccccttaaagggaattcgatgcccccctagaataatctc
atccccccacccgacttcttttgaaatgtgaacgtccttccttgactgtctagccactccctcccagaaaaactggctctgattggaatttc
tggcctcctaaggctccccaccccgaaatcagcccccagccttgtttctgatgacagtgttatcccaagaccctgccccctgccagccgacc
ctcctggccttcctcgttgggccgctctgatttcaggcagcaggggctgctgtgatgccgtcctgctggagtgatttatactgtgaaatgag
ttggccagattgtggggtgcagctgggtggggcagcacacctctggggggataatgtccccactcccgaaagcctttcctcggtctcccttc
cgtccatcccccttcttcctcccctcaacagtgagttagactcaagggggtgacagaaccgagaagggggtgacagtcctccatccacgtgg
cctctctctctctcctcaggaccctcagccctggcctttttctttaaggtcccccgaccaatccccagcctaggacgccaacttctcccacc
ccttggcccctcacatcctctccaggaagggagtgaggggctgtgacatttttccggagaagatttcagagctgaggctttggtacccccaa
acccccaatatttttggactggcagactcaaggggctggaatctcatgattccatgcccgagtccgcccatccctgaccatggttttggctc
tcccaccccgccgttccctgcgcttcatctcatgaggatttctttatgaggcaaatttatattttttaatatcggggggggaccacgccgcc
ctccatccgtgctgcatgaaaaacattccacgtgccccttgtcgcgcgtctcccatcctgatcccagacccattccttagctatttatccct
ttcctggtttccgaaaggcaattatatctattatgtataagtaaatatattatatatggatgtgtgtgtgtgcgtgcgcgtgagtgtgtgag
cgcttctgcagcctcggcctaggtcacgttggccctcaaagcgagccgttgaattggaaactgcttctagaaactctggctcagcctgtctc
gggctgacccttttctgatcgtctcggcccctctgattgttcccgatggtctctctccctctgtcttttctcctccgcctgtgtccatctga
ccgttttcacttgtctcctttctgactgtccctgccaatgctccagctgtcgtctgactctgggttcgttggggacatgagattttattttt
tgtgagtgagactgagggatcgtagatttttacaatctgtatctttgacaattctgggtgcgagtgtgagagtgtgagcagggcttgctcct
gccaaccacaattcaatgaatccccgacccccctaccccatgctgtacttgtggttctctttttgtattttgcatctgaccccggggggctg
ggacagattggcaatgggccgtcccctctccccttggttctgcactgttgccaataaaaagctcttaaaaacgca
SEQ ID NO: 30, FosB proto-oncogene, AP-1 transcription factor subunit Homo sapiens
(human), transcript variant 2, Gene ID: 2354, mRNA CDS, NCBI Reference Sequence:
NM_001114171.2 (CDS region nt 592-1500), 909 nt:
atgtttcaggctttccccggagactacgactccggctcccggtgcagctcctcaccctctgccgagtctcaatatctgtcttcggtggactc
cttcggcagtccacccaccgccgccgcctcccaggagtgcgccggtctcggggaaatgcccggttccttcgtgcccacggtcaccgcgatca
caaccagccaggacctccagtggcttgtgcaacccaccctcatctcttccatggcccagtcccaggggcagccactggcctcccagcccccg
gtcgtcgacccctacgacatgccgggaaccagctactccacaccaggcatgagtggctacagcagtggcggagcgagtggcagtggtgggcc
ttccaccagcggaactaccagtgggcctgggcctgcccgcccagcccgagcccggcctaggagaccccgagaggagacggagacagatcagt
tggaggaagaaaaagcagagctggagtcggagatcgccgagctccaaaaggagaaggaacgtctggagtttgtgctggtggcccacaaaccg
ggctgcaagatcccctacgaagaggggcccgggccgggcccgctggcggaggtgagagatttgccgggctcagcaccggctaaggaagatgg
cttcagctggctgctgccgcccccgccaccaccgcccctgcccttccagaccagccaagacgcaccccccaacctgacggcttctctcttta
cacacagtgaagttcaagtcctcggcgaccccttccccgttgttaacccttcgtacacttcttcgtttgtcctcacctgcccggaggtctcc
gcgttcgccggcgcccaacgcaccagcggcagtgaccagccttccgatcccctgaactcgccctccctcctcgctctgtga
SEQ ID NO: 31, JunB proto-oncogene, AP-1 transcription factor subunit Homo sapiens
(human), Gene ID: 3726, mRNA, NCBI Reference Sequence: NM_002229.3 (CDS region nt
287-1330), 1830 nt:
gggaccttgagagcggccaggccagcctcggagccagcagggagctgggagctgggggaaacgacgccaggaaagctatcgcgccagagagg
gcgacgggggctcgggaagcctgacagggcttttgcgcacagctgccggctggctgctacccgcccgcgccagcccccgagaacgcgcgacc
aggcacccagtccggtcaccgcagcggagagctcgccgctcgctgcagcgaggcccggagcggccccgcagggaccctccccagaccgcctg
ggccgcccggatgtgcactaaaatggaacagcccttctaccacgacgactcatacacagctacgggatacggccgggcccctggtggcctct
ctctacacgactacaaactcctgaaaccgagcctggggtcaacctggccgacccctaccggagtctcaaagcgcctggggctcgcggacccg
gcccagagggcggcggtggcggcagctacttttctggtcagggctcggacaccggcgcgtctctcaagctcgcctcttcggagctggaacgc
ctgattgtccccaacagcaacggcgtgatcacgacgacgcctacacccccgggacagtacttttacccccgcgggggtggcagcggtggagg
tgcagggggcgcagggggcggcgtcaccgaggagcaggagggcttcgccgacggctttgtcaaagccctggacgatctgcacaagatgaacc
acgtgacaccccccaacgtgtccctgggcgctaccggggggcccccggctgggcccgggggcgtctacgccggcccggagccacctcccgtt
tacaccaacctcagcagctactccccagcctctgcgtcctcgggaggcgccggggctgccgtcgggaccgggagctcgtacccgacgaccac
catcagctacctcccacacgcgccgcccttcgccggtggccacccggcgcagctgggcttgggccgcggcgcctccaccttcaaggaggaac
cgcagaccgtgccggaggcgcgcagccgggacgccacgccgccggtgtcccccatcaacatggaagaccaagagcgcatcaaagtggagcgc
aagcggctgcggaaccggctggcggccaccaagtgccggaagcggaagctggagcgcatcgcgcgcctggaggacaaggtgaagacgctcaa
ggccgagaacgcggggctgtcgagtaccgccggcctcctccgggagcaggtggcccagctcaaacagaaggtcatgacccacgtcagcaacg
gctgtcagctgctgcttggggtcaagggacacgccttctgaacgtcccctgcccctttacggacaccccctcgcttggacggctgggcacac
gcctcccactggggtccagggagcaggcggtgggcacccaccctgggacctaggggcgccgcaaaccacactggactccggccctcctaccc
tgcgcccagtccttccacctcgacgtttacaagcccccccttccacttttttttgtatgttttttttctgctggaaacagactcgattcata
ttgaatataatatatttgtgtatttaacagggaggggaagagggggcgatcgcggcggagctggccccgccgcctggtactcaagcccgcgg
ggacattgggaaggggacccccgccccctgccctcccctctctgcaccgtactgtggaaaagaaacacgcacttagtctctaaagagtttat
tttaagacgtgtttgtgtttgtgtgtgtttgttctttttattgaatctatttaagtaaaaaaaaaattggttctttattaa
SEQ ID NO: 32, JunB proto-oncogene, AP-1 transcription factor subunit Homo sapiens
(human), Gene ID: 3726, mRNA CDS, NCBI Reference Sequence: NM_002229.3 (CDS region nt
287-1330), 1044 nt:
atgtgcactaaaatggaacagcccttctaccacgacgactcatacacagctacgggatacggccgggcccctggtggcctctctctacacga
ctacaaactcctgaaaccgagcctggcggtcaacctggccgacccctaccggagtctcaaagcgcctggggctcgcggacccggcccagagg
gcggcggtggcggcagctacttttctggtcagggctcggacaccggcgcgtctctcaagctcgcctcttcggagctggaacgcctgattgtc
cccaacagcaacggcgtgatcacgacgacgcctacacccccgggacagtacttttacccccgcgggggtggcagcggtggaggtgcaggggg
cgcagggggcggcgtcaccgaggagcaggagggcttcgccgacggctttgtcaaagccctggacgatctgcacaagatgaaccacgtgacac
cccccaacgtgtccctgggcgctaccggggggcccccggctgggcccgggggcgtctacgccggcccggagccacctcccgtttacaccaac
ctcagcagctactccccagcctctgcgtcctcgggaggcgccggggctgccgtcgggaccgggagctcgtacccgacgaccaccatcagcta
cctcccacacgcgccgcccttcgccggtggccacccggcgcagctgggcttgggccgcggcgcctccaccttcaaggaggaaccgcagaccg
tgccggaggcgcgcagccgggacgccacgccgccggtgtcccccatcaacatggaagaccaagagcgcatcaaagtggagcgcaagcggctg
cggaaccggctggcggccaccaagtgccggaagcggaagctggagcgcatcgcgcgcctggaggacaaggtgaagacgctcaaggccgagaa
cgcggggctgtcgagtaccgccggcctcctccgggagcaggtggcccagctcaaacagaaggtcatgacccacgtcagcaacggctgtcagc
tgctgcttggggtcaagggacacgccttctga

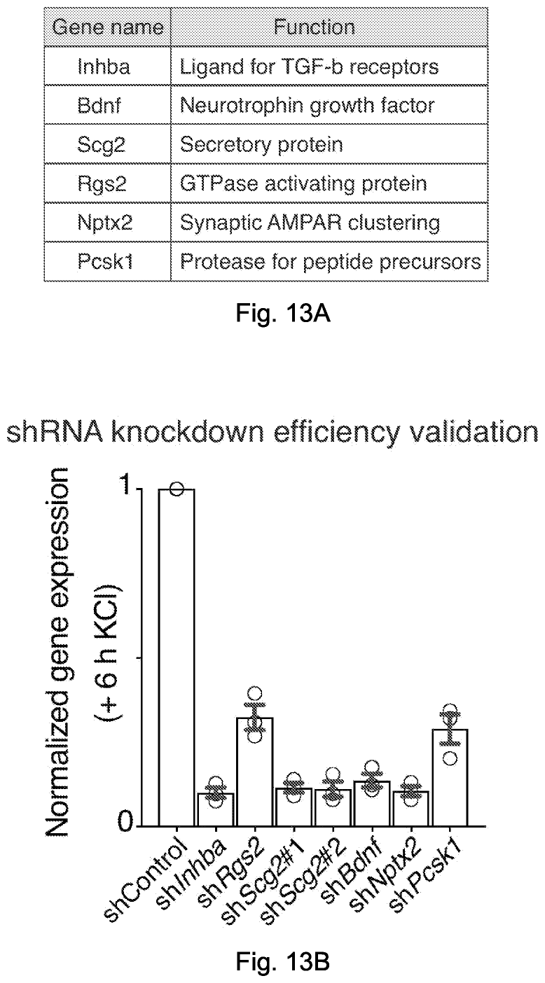

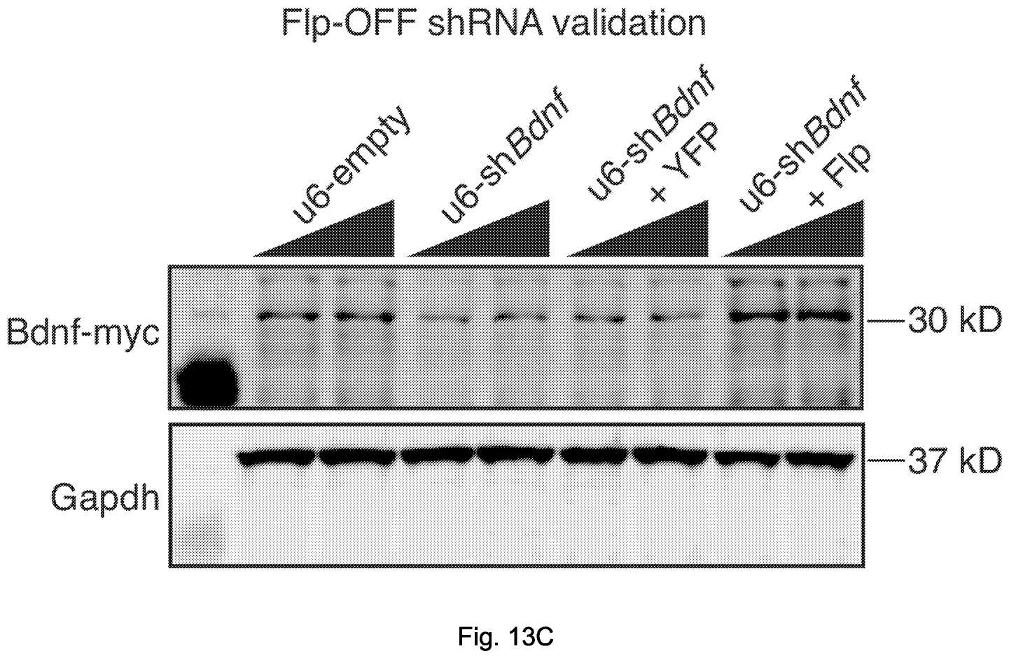

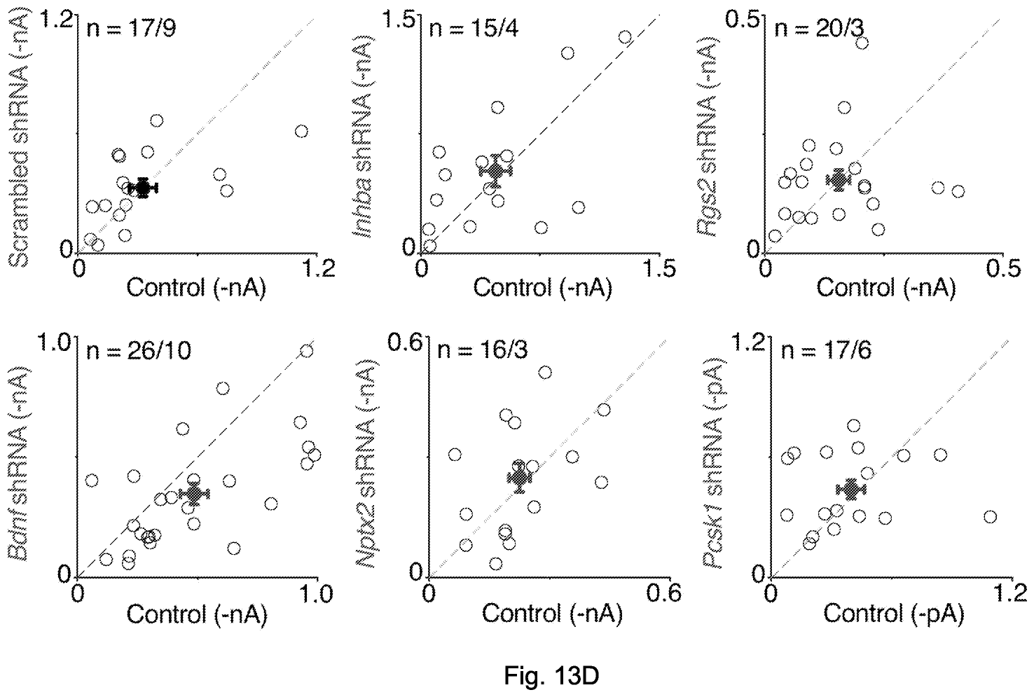

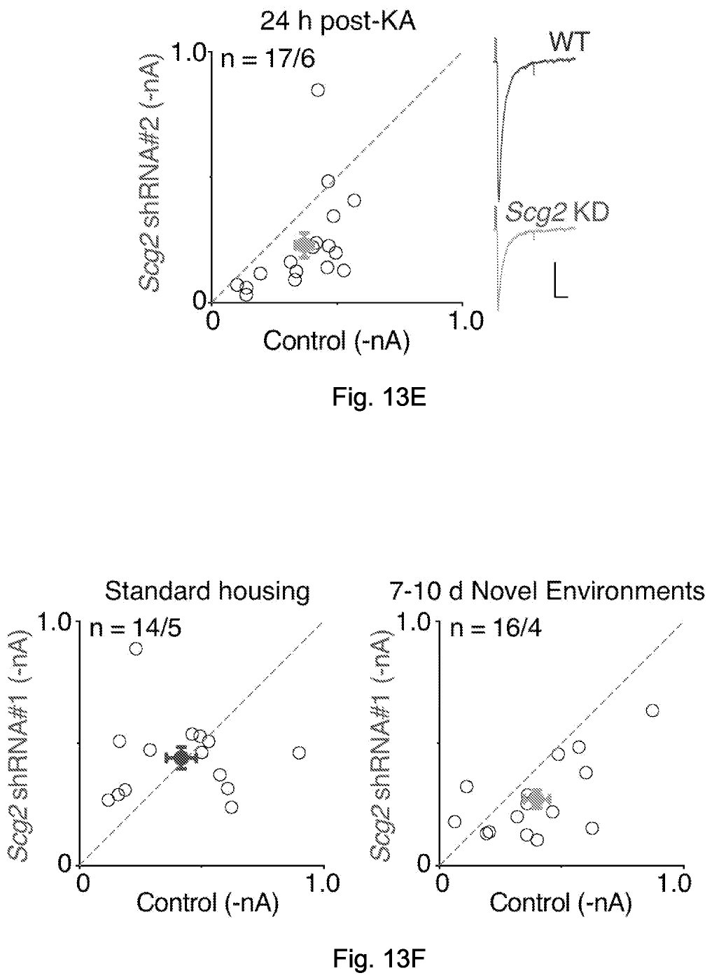

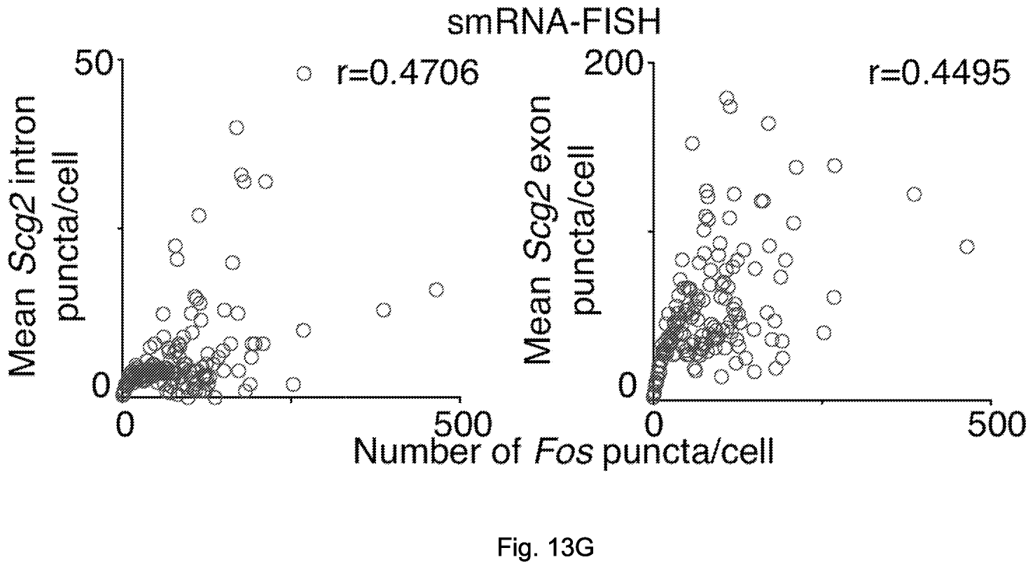

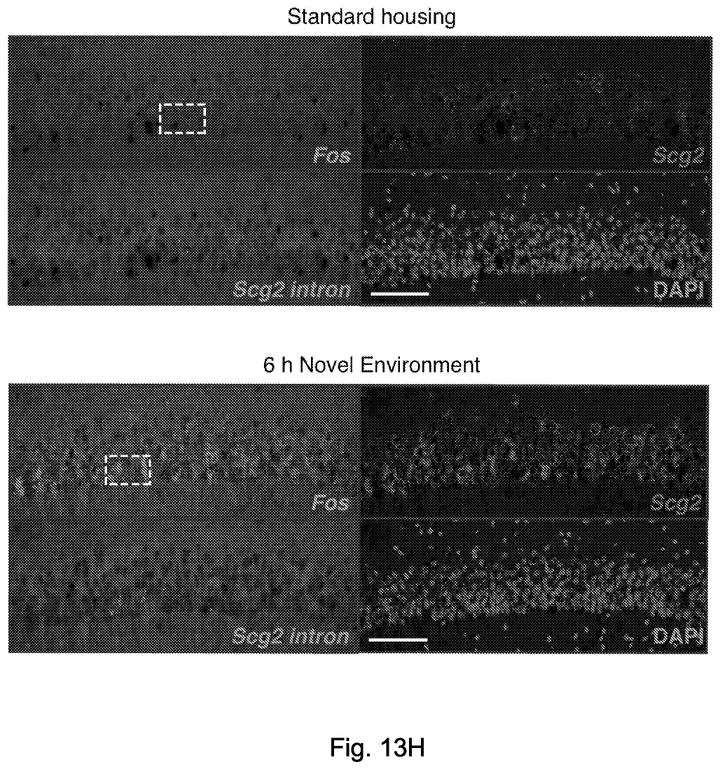

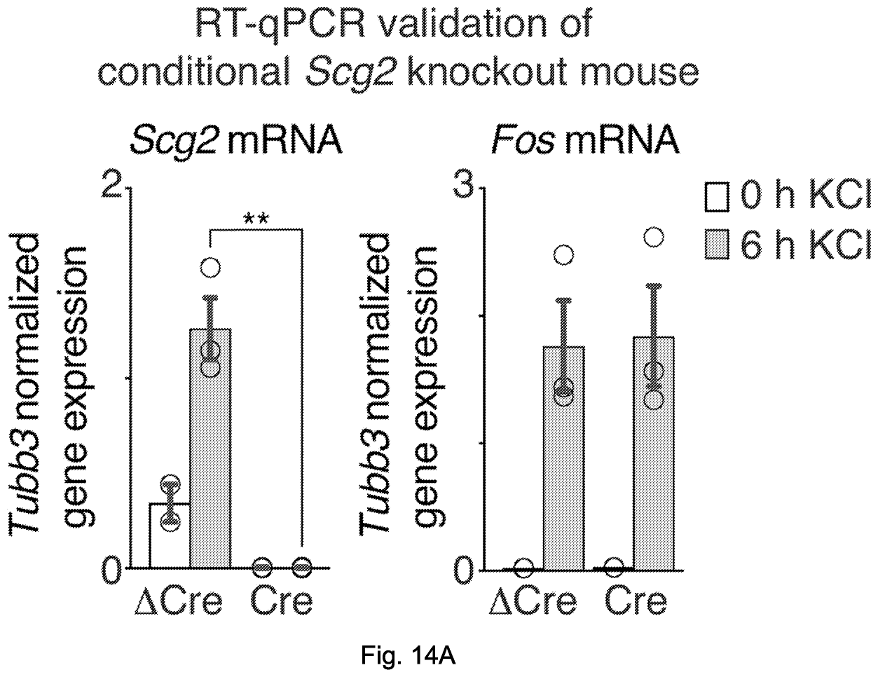

In some embodiments, mRNA molecules can be detected using single-molecule RNA fluorescence in situ hybridization (smRNA-FISH), as described herein (see e.g., FIG. 4J, FIG. 13H). For example, hybridization probes for the various targets described herein are commercially available and can be used for the purposes of the invention to measure protein expression levels, e.g., Mm-Fos (ADVANCED CELL DIAGNOSTICS Cat. #584741), Mm-Fosb (ADVANCED CELL DIAGNOSTICS Cat. #584751), Mm-Junb (ADVANCED CELL DIAGNOSTICS Cat. #584761), Mm-Scg2 (ADVANCED CELL DIAGNOSTICS Cat. #477691), or Mm-Scg2 intron (ADVANCED CELL DIAGNOSTICS Cat. #859141).

Nucleic acid, e.g., deoxyribonucleic acid (DNA) and ribonucleic acid (RNA), molecules can be isolated from a particular biological sample using any of a number of procedures, which are well-known in the art, the particular isolation procedure chosen being appropriate for the particular biological sample. For example, freeze-thaw and alkaline lysis procedures can be useful for obtaining nucleic acid molecules from solid materials; heat and alkaline lysis procedures can be useful for obtaining nucleic acid molecules from urine; and proteinase K extraction can be used to obtain nucleic acid from blood (Roiff, A et al. PCR: Clinical Diagnostics and Research, Springer (1994)).

In some embodiments of any of the aspects, one or more of the detection reagents (e.g. an antibody reagent and/or nucleic acid probe) can comprise a detectable label and/or comprise the ability to generate a detectable signal (e.g. by catalyzing a reaction converting a compound to a detectable product). Detectable labels can comprise, for example, a light-absorbing dye, a fluorescent dye, or a radioactive label. Detectable labels, methods of detecting them, and methods of incorporating them into reagents (e.g. antibodies and nucleic acid probes) are well known in the art.

In some embodiments of any of the aspects, detectable labels can include labels that can be detected by spectroscopic, photochemical, biochemical, immunochemical, electromagnetic, radiochemical, or chemical means, such as fluorescence, chemifluorescence, or chemiluminescence, or any other appropriate means. The detectable labels used in the methods described herein can be primary labels (where the label comprises a moiety that is directly detectable or that produces a directly detectable moiety) or secondary labels (where the detectable label binds to another moiety to produce a detectable signal, e.g., as is common in immunological labeling using secondary and tertiary antibodies). The detectable label can be linked by covalent or non-covalent means to the reagent. Alternatively, a detectable label can be linked such as by directly labeling a molecule that achieves binding to the reagent via a ligand-receptor binding pair arrangement or other such specific recognition molecules. Detectable labels can include, but are not limited to radioisotopes, bioluminescent compounds, chromophores, antibodies, chemiluminescent compounds, fluorescent compounds, metal chelates, and enzymes.

In other embodiments, the detection reagent is labeled with a fluorescent compound. When the fluorescently labeled reagent is exposed to light of the proper wavelength, its presence can then be detected due to fluorescence. In some embodiments of any of the aspects, a detectable label can be a fluorescent dye molecule, or fluorophore including, but not limited to fluorescein, phycoerythrin, phycocyanin, o-phthalaldehyde, fluorescamine, Cy3™, Cy5™, allophycocyanin, Texas Red, peridinin chlorophyll, cyanine, tandem conjugates such as phycoerythrin-Cy5™, green fluorescent protein (GFP), rhodamine, fluorescein isothiocyanate (FITC) and Oregon Green, rhodamine and derivatives (e.g., Texas red and tetramethylrhodamine isothiocyanate (TRITC)), biotin, phycoerythrin, AMCA, CyDyes™, 6-carboxyfluorescein (commonly known by the abbreviations FAM and F), 6-carboxy-2′,4′,7′,4,7-hexachlorofluorescein (HEX), 6-carboxy-4′,5′-dichloro-2′,7′-dimethoxyfiuorescein (JOE or J), N,N,N′,N′-tetramethyl-6carboxyrhodamine (TAMRA or T), 6-carboxy-X-rhodamine (ROX or R), 5-carboxyrhodamine-6G (R6G5 or G5), 6-carboxyrhodamine-6G (R6G6 or G6), and rhodamine 110; cyanine dyes, e.g. Cy3, Cy5 and Cy7 dyes; coumarins, e.g., umbelliferone; benzimide dyes, e.g. Hoechst 33258; phenanthridine dyes, e.g. Texas Red; ethidium dyes; acridine dyes; carbazole dyes; phenoxazine dyes; porphyrin dyes; polymethine dyes, e.g., cyanine dyes such as Cy3, Cy5, etc.; BODIPY dyes and quinoline dyes. In some embodiments of any of the aspects, a detectable label can be a radiolabel including, but not limited to ³H, ¹²⁵I, ³⁵S, ¹⁴C, ³²P, and ³³P. In some embodiments of any of the aspects, a detectable label can be an enzyme including, but not limited to horseradish peroxidase and alkaline phosphatase. An enzymatic label can produce, for example, a chemiluminescent signal, a color signal, or a fluorescent signal. Enzymes contemplated for use to detectably label an antibody reagent include, but are not limited to, malate dehydrogenase, staphylococcal nuclease, delta-V-steroid isomerase, yeast alcohol dehydrogenase, alpha-glycerophosphate dehydrogenase, triose phosphate isomerase, horseradish peroxidase, alkaline phosphatase, asparaginase, glucose oxidase, beta-galactosidase, ribonuclease, urease, catalase, glucose-VI-phosphate dehydrogenase, glucoamylase and acetylcholinesterase. In some embodiments of any of the aspects, a detectable label is a chemiluminescent label, including, but not limited to lucigenin, luminol, luciferin, isoluminol, theromatic acridinium ester, imidazole, acridinium salt and oxalate ester. In some embodiments of any of the aspects, a detectable label can be a spectral colorimetric label including, but not limited to colloidal gold or colored glass or plastic (e.g., polystyrene, polypropylene, and latex) beads.

In some embodiments of any of the aspects, detection reagents can also be labeled with a detectable tag, such as c-Myc, HA, VSV-G, HSV, FLAG, V5, HIS, or biotin. Other detection systems can also be used, for example, a biotin-streptavidin system. In this system, the antibodies immunoreactive (i. e. specific for) with the biomarker of interest is biotinylated. Quantity of biotinylated antibody bound to the biomarker is determined using a streptavidin-peroxidase conjugate and a chromogenic substrate. Such streptavidin peroxidase detection kits are commercially available, e.g., from DAKO; Carpinteria, CA. A reagent can also be detectably labeled using fluorescence emitting metals such as ¹⁵²Eu, or others of the lanthanide series. These metals can be attached to the reagent using such metal chelating groups as diethylenetriaminepentaacetic acid (DTPA) or ethylenediaminetetraacetic acid (EDTA).

A level which is less than a reference level can be a level which is less by at least about 10%, at least about 20%, at least about 50%, at least about 60%, at least about 80%, at least about 90%, or less relative to the reference level. In some embodiments of any of the aspects, a level which is less than a reference level can be a level which is statistically significantly less than the reference level.

A level which is more than a reference level can be a level which is greater by at least about 10%, at least about 20%, at least about 50%, at least about 60%, at least about 80%, at least about 90%, at least about 100%, at least about 200%, at least about 300%, at least about 500% or more than the reference level. In some embodiments of any of the aspects, a level which is more than a reference level can be a level which is statistically significantly greater than the reference level.

In some embodiments of any of the aspects, the reference can be a level of the target molecule in a population of subjects who do not have or are not diagnosed as having, and/or do not exhibit signs or symptoms of a memory-associated disorder, a learning disability, a neurodegenerative disease or disorder, or epilepsy. In some embodiments of any of the aspects, the reference can also be a level of expression of the target molecule in a control sample, a pooled sample of control individuals or a numeric value or range of values based on the same. In some embodiments of any of the aspects, the reference can be the level of a target molecule in a sample obtained from the same subject at an earlier point in time, e.g., the methods described herein can be used to determine if a subject's sensitivity or response to a given therapy is changing over time.

In some embodiments of any of the aspects, the level of expression products of no more than 200 other genes is determined. In some embodiments of any of the aspects, the level of expression products of no more than 100 other genes is determined. In some embodiments of any of the aspects, the level of expression products of no more than 20 other genes is determined. In some embodiments of any of the aspects, the level of expression products of no more than 10 other genes is determined.

In some embodiments of the foregoing aspects, the expression level of a given gene can be normalized relative to the expression level of one or more reference genes or reference proteins.

In some embodiments, the reference level can be the level in a sample of similar cell type, sample type, sample processing, and/or obtained from a subject of similar age, sex and other demographic parameters as the sample/subject for which the level of a memory-associated analyte (e.g., Scg2 mRNA, polypeptide, or neuropeptide or Fos, Fosb, or Junb mRNA or polypeptide) is to be determined. In some embodiments, the test sample and control reference sample are of the same type, that is, obtained from the same biological source, and comprising the same composition, e.g. the same number and type of cells.

The term “sample” or “test sample” as used herein denotes a sample taken or isolated from a biological organism, e.g., a blood or plasma sample from a subject. In some embodiments of any of the aspects, the sample is a cerebrospinal fluid sample or a CNS sample (e.g., a brain biopsy). In some embodiments of any of the aspects, the present invention encompasses several examples of a biological sample. In some embodiments of any of the aspects, the biological sample is cells, or tissue, or peripheral blood, or bodily fluid. Exemplary biological samples include, but are not limited to, a biopsy, a tumor sample, biofluid sample; blood; serum; plasma; urine; semen; mucus; tissue biopsy; organ biopsy; synovial fluid; bile fluid; cerebrospinal fluid; mucosal secretion; effusion; sweat; saliva; and/or tissue sample etc. The term also includes a mixture of the above-mentioned samples. The term “test sample” also includes untreated or pretreated (or pre-processed) biological samples. In some embodiments of any of the aspects, a test sample can comprise cells from a subject.

The test sample can be obtained by removing a sample from a subject, but can also be accomplished by using a previously isolated sample (e.g. isolated at a prior time point by the same or another person).

In some embodiments of any of the aspects, the test sample can be an untreated test sample. As used herein, the phrase “untreated test sample” refers to a test sample that has not had any prior sample pre-treatment except for dilution and/or suspension in a solution. Exemplary methods for treating a test sample include, but are not limited to, centrifugation, filtration, sonication, homogenization, heating, freezing and thawing, and combinations thereof. In some embodiments of any of the aspects, the test sample can be a frozen test sample, e.g., a frozen tissue. The frozen sample can be thawed before employing methods, assays and systems described herein. After thawing, a frozen sample can be centrifuged before being subjected to methods, assays and systems described herein. In some embodiments of any of the aspects, the test sample is a clarified test sample, for example, by centrifugation and collection of a supernatant comprising the clarified test sample. In some embodiments of any of the aspects, a test sample can be a pre-processed test sample, for example, supernatant or filtrate resulting from a treatment selected from the group consisting of centrifugation, homogenization, sonication, filtration, thawing, purification, and any combinations thereof. In some embodiments of any of the aspects, the test sample can be treated with a chemical and/or biological reagent. Chemical and/or biological reagents can be employed, for example, to protect and/or maintain the stability of the sample, including biomolecules (e.g., nucleic acid and protein) therein, during processing. One exemplary reagent is a protease inhibitor, which is generally used to protect or maintain the stability of protein during processing. The skilled artisan is well aware of methods and processes appropriate for pre-processing of biological samples required for determination of the level of an expression product as described herein.

In some embodiments of any of the aspects, the methods described herein can further comprise a step of obtaining or having obtained a test sample from a subject. In some embodiments of any of the aspects, the subject can be a human subject. In some embodiments of any of the aspects, the subject can be a subject in need of treatment for (e.g. having or diagnosed as having) a memory-associated disorder, a learning disability, a neurodegenerative disease or disorder, or epilepsy or a subject at risk of or at increased risk of developing a memory-associated disorder, a learning disability, a neurodegenerative disease or disorder, or epilepsy as described elsewhere herein.

Another aspect of the technology described herein relates to kits for detecting a memory associated analyte described herein, among others. Described herein are kit components that can be included in one or more of the kits described herein.

In some embodiments, the kit comprises an effective amount of an Scg2 neuropeptide pharmaceutical composition as described herein. In some embodiments, the kit comprises a nucleic acid, vector, or viral vector comprising a nucleic acid encoding an Scg2 neuropeptide, e.g., under the control of a promoter for expressing Scg2 neuropeptides in vitro or in vivo. In some embodiments, the kit comprises an effective amount of an Scg2 neuropeptide, e.g., for use in a cell culture media. In some embodiments, the kit comprises an effective amount of a detection reagent for memory associated analyte described herein (e.g. a Scg2 mRNA or polypeptide; secretoneurin, EM66, manserin, and/or SgII mRNA or polypeptide; Fos, Fosb, or Junb mRNA or polypeptide).

As will be appreciated by one of skill in the art, such reagents can be supplied in a lyophilized form or a concentrated form that can diluted or suspended in liquid prior to use, e.g., in treatment, detection, and/or exposure to cultured cells. Preferred formulations include those that are non-toxic to the subject, animals, or cells and/or does not affect growth rate or viability etc. The reagents described herein can be supplied in aliquots or in unit doses.

In some embodiments, the components described herein can be provided singularly or in any combination as a kit. Such a kit includes the components described herein, e.g., a pharmaceutical composition comprising an Scg2 neuropeptide; a composition(s) that includes a nucleic acid encoding an Scg2 neuropeptide as described herein; a composition(s) that includes a vector or a viral vector comprising a nucleic acid encoding an Scg2 neuropeptide as described herein; an Scg2 neuropeptide composition for use in a cell culture media; and/or one or more agents that permit the detection of a memory-associated analyte, or a set of memory-associated analytes, as described herein; or any combinations thereof. In addition, the kit optionally comprises informational material. The kit can also contain a substrate for coating culture dishes, such as laminin, fibronectin, Poly-L-Lysine, or methylcellulose.

In some embodiments, the compositions in the kit can be provided in a watertight or gas tight container which in some embodiments is substantially free of other components of the kit. For example, a composition described herein can be supplied in more than one container, e.g., it can be supplied in a container having sufficient reagent for a predetermined number of treatments, detections, assays, cell culture vessels, etc., e.g., 1, 2, 3 or greater. One or more components as described herein can be provided in any form, e.g., liquid, dried or lyophilized form. It is preferred that the components described herein are substantially pure and/or sterile. When the components described herein are provided in a liquid solution, the liquid solution preferably is an aqueous solution, with a sterile aqueous solution being preferred.

The informational material can be descriptive, instructional, marketing or other material that relates to the methods described herein. The informational material of the kits is not limited in its form. In one embodiment, the informational material can include information about production of the compositions described herein, concentration, date of expiration, batch or production site information, and so forth. In one embodiment, the informational material relates to methods for using or administering the components of the kit.

The kit can include a component for the detection of a memory-associated analyte. In addition, the kit can include one or more antibodies that bind a cell marker, or primers for an RT-PCR or PCR reaction, e.g., a semi-quantitative or quantitative RT-PCR or PCR reaction. If the detection reagent is an antibody, it can be supplied in dry preparation, e.g., lyophilized, or in a solution. The antibody or other detection reagent can be linked to a label, e.g., a radiological, fluorescent (e.g., GFP) or colorimetric label for use in detection. If the detection reagent is a primer, it can be supplied in dry preparation, e.g., lyophilized, or in a solution.

The kit will typically be provided with its various elements included in one package, e.g., a fiber-based, e.g., a cardboard, or polymeric, e.g., a Styrofoam box. The enclosure can be configured so as to maintain a temperature differential between the interior and the exterior, e.g., it can provide insulating properties to keep the reagents at a preselected temperature for a preselected time.

For convenience, the meaning of some terms and phrases used in the specification, examples, and appended claims, are provided below. Unless stated otherwise, or implicit from context, the following terms and phrases include the meanings provided below. The definitions are provided to aid in describing particular embodiments, and are not intended to limit the claimed invention, because the scope of the invention is limited only by the claims. Unless otherwise defined, all technical and scientific terms used herein have the same meaning as commonly understood by one of ordinary skill in the art to which this invention belongs. If there is an apparent discrepancy between the usage of a term in the art and its definition provided herein, the definition provided within the specification shall prevail.

For convenience, certain terms employed herein, in the specification, examples and appended claims are collected here.

The terms “decrease”, “reduced”, “reduction”, or “inhibit” are all used herein to mean a decrease by a statistically significant amount. In some embodiments, “reduce,” “reduction” or “decrease” or “inhibit” typically means a decrease by at least 10% as compared to a reference level (e.g. the absence of a given treatment or agent) and can include, for example, a decrease by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or more. As used herein, “reduction” or “inhibition” does not encompass a complete inhibition or reduction as compared to a reference level. “Complete inhibition” is a 100% inhibition as compared to a reference level. A decrease can be preferably down to a level accepted as within the range of normal, e.g., for an individual without a given disorder.

The terms “increased”, “increase”, “enhance”, or “activate” are all used herein to mean an increase by a statically significant amount. In some embodiments, the terms “increased”, “increase”, “enhance”, or “activate” can mean an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level. In the context of a marker or symptom, an “increase” is a statistically significant increase in such level.

As used herein, a “subject” means a human or animal. Usually the animal is a vertebrate such as a primate, rodent, domestic animal or game animal. Primates include chimpanzees, cynomolgus monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. In some embodiments, the subject is a mammal, e.g., a primate, e.g., a human. The terms, “individual,” “patient” and “subject” are used interchangeably herein.

Preferably, the subject is a mammal. The mammal can be a human, non-human primate, mouse, rat, dog, cat, horse, or cow, but is not limited to these examples. Mammals other than humans can be advantageously used as subjects that represent animal models of a memory-associated disorder, a learning disability, a neurodegenerative disease or disorder, or epilepsy, as described further herein. A subject can be male or female.

A subject can be one who has been previously diagnosed with or identified as suffering from or having a condition in need of treatment (e.g. a memory-associated disorder, a learning disability, a neurodegenerative disease or disorder, or epilepsy) or one or more complications related to such a condition, and optionally, have already undergone treatment for a memory-associated disorder, a learning disability, a neurodegenerative disease or disorder, or epilepsy or the one or more complications related to a memory-associated disorder, a learning disability, a neurodegenerative disease or disorder, or epilepsy. Alternatively, a subject can also be one who has not been previously diagnosed as having a memory-associated disorder, a learning disability, a neurodegenerative disease or disorder, or epilepsy, or has one or more complications related to a memory-associated disorder, a learning disability, a neurodegenerative disease or disorder, or epilepsy. For example, a subject can be one who exhibits one or more risk factors for a memory-associated disorder, a learning disability, a neurodegenerative disease or disorder, or epilepsy or one or more complications related to a memory-associated disorder, a learning disability, a neurodegenerative disease or disorder, or epilepsy or a subject who does not exhibit risk factors.

A “subject in need” of treatment for a particular condition can be a subject having that condition, diagnosed as having that condition, or at risk of developing that condition.

As used herein, the terms “protein” and “polypeptide” are used interchangeably to designate a series of amino acid residues, connected to each other by peptide bonds between the alpha-amino and carboxy groups of adjacent residues. The terms “protein”, and “polypeptide” refer to a polymer of amino acids, including modified amino acids (e.g., phosphorylated, glycated, glycosylated, etc.) and amino acid analogs, regardless of its size or function. “Protein” and “polypeptide” are often used in reference to relatively large polypeptides, whereas the term “peptide” is often used in reference to small polypeptides, but usage of these terms in the art overlaps. The terms “protein” and “polypeptide” are used interchangeably herein when referring to a gene product and fragments thereof. Thus, exemplary polypeptides or proteins include gene products, naturally occurring proteins, homologs, orthologs, paralogs, fragments and other equivalents, variants, fragments, and analogs of the foregoing.

In the various embodiments described herein, it is further contemplated that variants (naturally occurring or otherwise), alleles, homologs, conservatively modified variants, and/or conservative substitution variants of any of the particular polypeptides described are encompassed. As to amino acid sequences, one of skill will recognize that individual substitutions, deletions or additions to a nucleic acid, peptide, polypeptide, or protein sequence which alters a single amino acid or a small percentage of amino acids in the encoded sequence is a “conservatively modified variant” where the alteration results in the substitution of an amino acid with a chemically similar amino acid and retains the desired activity of the polypeptide. Such conservatively modified variants are in addition to and do not exclude polymorphic variants, interspecies homologs, and alleles consistent with the disclosure.

A given amino acid can be replaced by a residue having similar physiochemical characteristics, e.g., substituting one aliphatic residue for another (such as Ile, Val, Leu, or Ala for one another), or substitution of one polar residue for another (such as between Lys and Arg; Glu and Asp; or Gln and Asn). Other such conservative substitutions, e.g., substitutions of entire regions having similar hydrophobicity characteristics, are well known. Polypeptides comprising conservative amino acid substitutions can be tested in any one of the assays described herein to confirm that a desired activity (e.g., PV-IN or CCK-IN activity, power of fast gamma waves, CA1 pyramidal cell firing during the descending phase of the theta_{pyr} cycle, spatial learning, memory consolidation, and/or memory retention among others) and specificity of a native or reference polypeptide is retained.

Amino acids can be grouped according to similarities in the properties of their side chains (in A. L. Lehninger, in Biochemistry, second ed., pp. 73-75, Worth Publishers, New York (1975)): (1) non-polar: Ala (A), Val (V), Leu (L), Ile (I), Pro (P), Phe (F), Trp (W), Met (M); (2) uncharged polar: Gly (G), Ser (S), Thr (T), Cys (C), Tyr (Y), Asn (N), Gln (Q); (3) acidic: Asp (D), Glu (E); (4) basic: Lys (K), Arg (R), His (H). Alternatively, naturally occurring residues can be divided into groups based on common side-chain properties: (1) hydrophobic: Norleucine, Met, Ala, Val, Leu, Ile; (2) neutral hydrophilic: Cys, Ser, Thr, Asn, Gln; (3) acidic: Asp, Glu; (4) basic: His, Lys, Arg; (5) residues that influence chain orientation: Gly, Pro; (6) aromatic: Trp, Tyr, Phe. Non-conservative substitutions will entail exchanging a member of one of these classes for another class. Particular conservative substitutions include, for example; Ala into Gly or into Ser; Arg into Lys; Asn into Gln or into His; Asp into Glu; Cys into Ser; Gln into Asn; Glu into Asp; Gly into Ala or into Pro; His into Asn or into Gln; Ile into Leu or into Val; Leu into Ile or into Val; Lys into Arg, into Gln or into Glu; Met into Leu, into Tyr or into Ile; Phe into Met, into Leu or into Tyr; Ser into Thr; Thr into Ser; Trp into Tyr; Tyr into Trp; and/or Phe into Val, into Ile or into Leu.

In some embodiments, the polypeptide described herein (or a nucleic acid encoding such a polypeptide) can be a functional fragment of one of the amino acid sequences described herein. As used herein, a “functional fragment” is a fragment or segment of a polypeptide which retains at least 50% of the wild-type reference polypeptide's activity according to the assays described herein. A functional fragment can comprise conservative substitutions of the sequences disclosed herein.

In some embodiments, the polypeptide described herein can be a variant of a sequence described herein. In some embodiments, the variant is a conservatively modified variant. Conservative substitution variants can be obtained by mutations of native nucleotide sequences, for example. A “variant,” as referred to herein, is a polypeptide substantially homologous to a native or reference polypeptide, but which has an amino acid sequence different from that of the native or reference polypeptide because of one or a plurality of deletions, insertions or substitutions. Variant polypeptide-encoding DNA sequences encompass sequences that comprise one or more additions, deletions, or substitutions of nucleotides when compared to a native or reference DNA sequence, but that encode a variant protein or fragment thereof that retains activity. A wide variety of PCR-based site-specific mutagenesis approaches are known in the art and can be applied by the ordinarily skilled artisan to generate and test artificial variants.

A variant amino acid or DNA sequence can be at least 90%, at least 91%, at least 92%, at least 93%, at least 94%, at least 95%, at least 96%, at least 97%, at least 98%, at least 99%, or more, identical to a native or reference sequence. The degree of homology (percent identity) between a native and a mutant sequence can be determined, for example, by comparing the two sequences using freely available computer programs commonly employed for this purpose on the world wide web (e.g. BLASTp or BLASTn with default settings).

A variant amino acid sequence can be at least 50%, at least 60%, at least 70%, at least 80%, at least 90%, at least 91%, at least 92%, at least 93%, at least 94%, at least 95%, at least 96%, at least 97%, at least 98%, at least 99%, or more, similar to a native or reference sequence. As used herein, “similarity” refers to an identical amino acid or a conservatively substituted amino acid, as described herein. Accordingly, the percentage of “sequence similarity” is the percentage of amino acids which is either identical or conservatively changed; e.g., “sequence similarity”=(% sequence identity)+(% conservative changes). It should be understood that a sequence that has a specified percent similarity to a reference sequence necessarily encompasses a sequence with the same specified percent identity to that reference sequence. The skilled person will be aware of several different computer programs, using different mathematical algorithms, that are available to determine the identity or similarity between two sequences. For instance, use can be made of a computer program employing the Needleman and Wunsch algorithm (Needleman et al. (1970)); the GAP program in the Accelrys GCG software package (Accelerys Inc., San Diego U.S.A.); the algorithm of E. Meyers and W. Miller (Meyers et al. (1989)) which has been incorporated into the ALIGN program (version 2.0); or more preferably the BLAST (Basic Local Alignment Tool using default parameters); see e.g., U.S. Pat. No. 10,023,890, the content of which is incorporated by reference herein in its entirety.

Alterations of the native amino acid sequence can be accomplished by any of a number of techniques known to one of skill in the art. Mutations can be introduced, for example, at particular loci by synthesizing oligonucleotides containing a mutant sequence, flanked by restriction sites enabling ligation to fragments of the native sequence. Following ligation, the resulting reconstructed sequence encodes an analog having the desired amino acid insertion, substitution, or deletion. Alternatively, oligonucleotide-directed site-specific mutagenesis procedures can be employed to provide an altered nucleotide sequence having particular codons altered according to the substitution, deletion, or insertion required. Techniques for making such alterations are very well established and include, for example, those disclosed by Walder et al. (Gene 42:133, 1986); Bauer et al. (Gene 37:73, 1985); Craik (BioTechniques, January 1985, 12-19); Smith et al. (Genetic Engineering: Principles and Methods, Plenum Press, 1981); and U.S. Pat. Nos. 4,518,584 and 4,737,462, which are herein incorporated by reference in their entireties. Any cysteine residue not involved in maintaining the proper conformation of the polypeptide also can be substituted, generally with serine, to improve the oxidative stability of the molecule and prevent aberrant crosslinking. Conversely, cysteine bond(s) can be added to the polypeptide to improve its stability or facilitate oligomerization.

As used herein, the term “nucleic acid” or “nucleic acid sequence” refers to any molecule, preferably a polymeric molecule, incorporating units of ribonucleic acid, deoxyribonucleic acid or an analog thereof. The nucleic acid can be either single-stranded or double-stranded. A single-stranded nucleic acid can be one nucleic acid strand of a denatured double-stranded DNA. Alternatively, it can be a single-stranded nucleic acid not derived from any double-stranded DNA. In one aspect, the nucleic acid can be DNA. In another aspect, the nucleic acid can be RNA. Suitable DNA can include, e.g., genomic DNA or cDNA. Suitable RNA can include, e.g., mRNA.

The term “expression” refers to the cellular processes involved in producing RNA and proteins and as appropriate, secreting proteins, including where applicable, but not limited to, for example, transcription, transcript processing, translation and protein folding, modification and processing. Expression can refer to the transcription and stable accumulation of sense (e.g., mRNA) or antisense RNA derived from a nucleic acid fragment or fragments and/or to the translation of mRNA into a polypeptide.

In some embodiments, the expression of a biomarker(s), target(s), or gene/polypeptide described herein is/are tissue-specific. In some embodiments, the expression of a biomarker(s), target(s), or gene/polypeptide described herein is/are global. In some embodiments, the expression of a biomarker(s), target(s), or gene/polypeptide described herein is systemic.

“Expression products” include RNA transcribed from a gene, and polypeptides obtained by translation of mRNA transcribed from a gene. The term “gene” refers to the nucleic acid sequence which is transcribed (DNA) to RNA in vitro or in vivo when operably linked to appropriate regulatory sequences. The gene may or may not include regions preceding and following a coding region, e.g. 5′ untranslated (5′UTR) or “leader” sequences and 3′ UTR or “trailer” sequences, as well as intervening sequences (introns) between individual coding segments (exons).

“Marker” in the context of the present invention refers to an expression product, e.g., nucleic acid or polypeptide which is differentially present in a sample taken from subjects having a memory-associated disorder, a learning disability, a neurodegenerative disease or disorder, or epilepsy, as compared to a comparable sample taken from control subjects (e.g., a healthy subject). The term “biomarker” is used interchangeably with the term “marker.”

In some embodiments, the methods described herein relate to measuring, detecting, or determining the level of at least one marker. As used herein, the term “detecting” or “measuring” refers to observing a signal from, e.g. a probe, label, or target molecule to indicate the presence of an analyte in a sample. Any method known in the art for detecting a particular label moiety can be used for detection. Exemplary detection methods include, but are not limited to, spectroscopic, fluorescent, photochemical, biochemical, immunochemical, electrical, optical or chemical methods. In some embodiments of any of the aspects, measuring can be a quantitative observation.

In some embodiments of any of the aspects, a polypeptide, nucleic acid, or cell as described herein can be engineered. As used herein, “engineered” refers to the aspect of having been manipulated by the hand of man. For example, a polypeptide is considered to be “engineered” when at least one aspect of the polypeptide, e.g., its sequence, has been manipulated by the hand of man to differ from the aspect as it exists in nature. As is common practice and is understood by those in the art, progeny of an engineered cell are typically still referred to as “engineered” even though the actual manipulation was performed on a prior entity.

In some embodiments of any of the aspects, the pharmaceutical composition comprising an Scg2 neuropeptide or the nucleic acid, vector, or viral vector encoding an Scg2 neuropeptide described herein is exogenous. In some embodiments of any of the aspects, the pharmaceutical composition comprising an Scg2 neuropeptide or the nucleic acid, vector, or viral vector encoding an Scg2 neuropeptide described herein is ectopic. In some embodiments of any of the aspects, the pharmaceutical composition comprising an Scg2 neuropeptide or the nucleic acid, vector, or viral vector encoding an Scg2 neuropeptide described herein is not endogenous.

The term “exogenous” refers to a substance present in a cell other than its native source. The term “exogenous” when used herein can refer to a nucleic acid (e.g. a nucleic acid encoding a polypeptide) or a polypeptide that has been introduced by a process involving the hand of man into a biological system such as a cell or organism in which it is not normally found and one wishes to introduce the nucleic acid or polypeptide into such a cell or organism. Alternatively, “exogenous” can refer to a nucleic acid or a polypeptide that has been introduced by a process involving the hand of man into a biological system such as a cell or organism in which it is found in relatively low amounts and one wishes to increase the amount of the nucleic acid or polypeptide in the cell or organism, e.g., to create ectopic expression or levels. In contrast, the term “endogenous” refers to a substance that is native to the biological system or cell. As used herein, “ectopic” refers to a substance that is found in an unusual location and/or amount. An ectopic substance can be one that is normally found in a given cell, but at a much lower amount and/or at a different time. Ectopic also includes a substance, such as a polypeptide or nucleic acid that is not naturally found or expressed in a given cell in its natural environment.

In some embodiments, a nucleic acid encoding a polypeptide as described herein (e.g., an Scg2 neuropeptide) is comprised by a vector. In some of the aspects described herein, a nucleic acid sequence encoding a given polypeptide as described herein, or any module thereof, is operably linked to a vector. The term “vector”, as used herein, refers to a nucleic acid construct designed for delivery to a host cell or for transfer between different host cells. As used herein, a vector can be viral or non-viral. The term “vector” encompasses any genetic element that is capable of replication when associated with the proper control elements and that can transfer gene sequences to cells. A vector can include, but is not limited to, a cloning vector, an expression vector, a plasmid, phage, transposon, cosmid, chromosome, virus, virion, etc.

In some embodiments of any of the aspects, the vector is recombinant, e.g., it comprises sequences originating from at least two different sources. In some embodiments of any of the aspects, the vector comprises sequences originating from at least two different species. In some embodiments of any of the aspects, the vector comprises sequences originating from at least two different genes, e.g., it comprises a fusion protein or a nucleic acid encoding an expression product which is operably linked to at least one non-native (e.g., heterologous) genetic control element (e.g., a promoter, suppressor, activator, enhancer, response element, or the like).

In some embodiments of any of the aspects, the vector or nucleic acid described herein is codon-optimized, e.g., the native or wild-type sequence of the nucleic acid sequence has been altered or engineered to include alternative codons such that altered or engineered nucleic acid encodes the same polypeptide expression product as the native/wild-type sequence, but will be transcribed and/or translated at an improved efficiency in a desired expression system. In some embodiments of any of the aspects, the expression system is an organism other than the source of the native/wild-type sequence (or a cell obtained from such organism). In some embodiments of any of the aspects, the vector and/or nucleic acid sequence described herein is codon-optimized for expression in a mammal or mammalian cell, e.g., a mouse, a murine cell, or a human cell. In some embodiments of any of the aspects, the vector and/or nucleic acid sequence described herein is codon-optimized for expression in a human cell. In some embodiments of any of the aspects, the vector and/or nucleic acid sequence described herein is codon-optimized for expression in a yeast or yeast cell. In some embodiments of any of the aspects, the vector and/or nucleic acid sequence described herein is codon-optimized for expression in a bacterial cell. In some embodiments of any of the aspects, the vector and/or nucleic acid sequence described herein is codon-optimized for expression in an E. coli cell.

As used herein, the term “expression vector” refers to a vector that directs expression of an RNA or polypeptide from sequences linked to transcriptional regulatory sequences on the vector. The sequences expressed will often, but not necessarily, be heterologous to the cell. An expression vector may comprise additional elements, for example, the expression vector may have two replication systems, thus allowing it to be maintained in two organisms, for example in human cells for expression and in a prokaryotic host for cloning and amplification.

As used herein, the term “viral vector” refers to a nucleic acid vector construct that includes at least one element of viral origin and has the capacity to be packaged into a viral vector particle. The viral vector can contain the nucleic acid encoding a polypeptide as described herein in place of non-essential viral genes. The vector and/or particle may be utilized for the purpose of transferring any nucleic acids into cells either in vitro or in vivo. Numerous forms of viral vectors are known in the art. Non-limiting examples of a viral vector of this invention include an AAV vector, an adenovirus vector, a lentivirus vector, a retrovirus vector, a herpesvirus vector, an alphavirus vector, a poxvirus vector a baculovirus vector, and a chimeric virus vector.

It should be understood that the vectors described herein can, in some embodiments, be combined with other suitable compositions and therapies. In some embodiments, the vector is episomal. The use of a suitable episomal vector provides a means of maintaining the nucleotide of interest in the subject in high copy number extra chromosomal DNA thereby eliminating potential effects of chromosomal integration.

As used herein, the terms “treat,” “treatment,” “treating,” or “amelioration” refer to therapeutic treatments, wherein the object is to reverse, alleviate, ameliorate, inhibit, slow down or stop the progression or severity of a condition associated with a disease or disorder, e.g. a memory-associated disorder, a learning disability, a neurodegenerative disease or disorder, or epilepsy. The term “treating” includes reducing or alleviating at least one adverse effect or symptom of a condition, disease or disorder associated with a memory-associated disorder, a learning disability, a neurodegenerative disease or disorder, or epilepsy. Treatment is generally “effective” if one or more symptoms or clinical markers are reduced. Alternatively, treatment is “effective” if the progression of a disease is reduced or halted. That is, “treatment” includes not just the improvement of symptoms or markers, but also a cessation of, or at least slowing of, progress or worsening of symptoms compared to what would be expected in the absence of treatment. Beneficial or desired clinical results include, but are not limited to, alleviation of one or more symptom(s), diminishment of extent of disease, stabilized (i.e., not worsening) state of disease, delay or slowing of disease progression, amelioration or palliation of the disease state, remission (whether partial or total), and/or decreased mortality, whether detectable or undetectable. The term “treatment” of a disease also includes providing relief from the symptoms or side-effects of the disease (including palliative treatment).

As used herein, the term “pharmaceutical composition” refers to the active agent in combination with a pharmaceutically acceptable carrier e.g. a carrier commonly used in the pharmaceutical industry. The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio. In some embodiments of any of the aspects, a pharmaceutically acceptable carrier can be a carrier other than water. In some embodiments of any of the aspects, a pharmaceutically acceptable carrier can be a cream, emulsion, gel, liposome, nanoparticle, and/or ointment. In some embodiments of any of the aspects, a pharmaceutically acceptable carrier can be an artificial or engineered carrier, e.g., a carrier that the active ingredient would not be found to occur in or within nature.

As used herein, the term “administering,” refers to the placement of a compound as disclosed herein into a subject by a method or route which results in at least partial delivery of the agent at a desired site. Pharmaceutical compositions comprising the compounds disclosed herein can be administered by any appropriate route which results in an effective treatment in the subject. In some embodiments, administration comprises physical human activity, e.g., an injection, act of ingestion, an act of application, and/or manipulation of a delivery device or machine. Such activity can be performed, e.g., by a medical professional and/or the subject being treated.

As used herein, “contacting” refers to any suitable means for delivering, or exposing, an agent to at least one cell. Exemplary delivery methods include, but are not limited to, direct delivery to cell culture medium, transfection, transduction, perfusion, injection, or other delivery method known to one skilled in the art. In some embodiments, contacting comprises physical human activity, e.g., an injection; an act of dispensing, mixing, and/or decanting; and/or manipulation of a delivery device or machine.

In some embodiments of any of the aspects, cells can be maintained in culture. As used herein, “maintaining” refers to continuing the viability of a cell or population of cells. A maintained population of cells will have at least a subpopulation of metabolically active cells.

In some embodiments of any of the aspects, the disclosure described herein does not concern a process for cloning human beings, processes for modifying the germ line genetic identity of human beings, uses of human embryos for industrial or commercial purposes or processes for modifying the genetic identity of animals which are likely to cause them suffering without any substantial medical benefit to man or animal, and also animals resulting from such processes.

The term “statistically significant” or “significantly” refers to statistical significance and generally means a two standard deviation (2SD) or greater difference.

Other than in the operating examples, or where otherwise indicated, all numbers expressing quantities of ingredients or reaction conditions used herein should be understood as modified in all instances by the term “about.” The term “about” when used in connection with percentages can mean ±1%.

As used herein, the term “comprising” means that other elements can also be present in addition to the defined elements presented. The use of “comprising” indicates inclusion rather than limitation.

The term “consisting of” refers to compositions, methods, and respective components thereof as described herein, which are exclusive of any element not recited in that description of the embodiment.

As used herein the term “consisting essentially of” refers to those elements required for a given embodiment. The term permits the presence of additional elements that do not materially affect the basic and novel or functional characteristic(s) of that embodiment of the invention.

The singular terms “a,” “an,” and “the” include plural referents unless context clearly indicates otherwise. Similarly, the word “or” is intended to include “and” unless the context clearly indicates otherwise. Although methods and materials similar or equivalent to those described herein can be used in the practice or testing of this disclosure, suitable methods and materials are described below. The abbreviation, “e.g.” is derived from the Latin exempli gratia, and is used herein to indicate a non-limiting example. Thus, the abbreviation “e.g.” is synonymous with the term “for example.”

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

Unless otherwise defined herein, scientific and technical terms used in connection with the present application shall have the meanings that are commonly understood by those of ordinary skill in the art to which this disclosure belongs. It should be understood that this invention is not limited to the particular methodology, protocols, and reagents, etc., described herein and as such can vary. The terminology used herein is for the purpose of describing particular embodiments only, and is not intended to limit the scope of the present invention, which is defined solely by the claims. Definitions of common terms in cell biology, immunology, and molecular biology can be found in The Merck Manual of Diagnosis and Therapy, 20th Edition, published by Merck Sharp & Dohme Corp., 2018 (ISBN 0911910190, 978-0911910421); Robert S. Porter et al. (eds.), The Encyclopedia of Molecular Cell Biology and Molecular Medicine, published by Blackwell Science Ltd., 1999-2012 (ISBN 9783527600908); and Robert A. Meyers (ed.), Molecular Biology and Biotechnology: a Comprehensive Desk Reference, published by VCH Publishers, Inc., 1995 (ISBN 1-56081-569-8); Immunology by Werner Luttmann, published by Elsevier, 2006; Janeway's Immunobiology, Kenneth Murphy, Allan Mowat, Casey Weaver (eds.), W. W. Norton & Company, 2016 (ISBN 0815345054, 978-0815345053); Lewin's Genes XI, published by Jones & Bartlett Publishers, 2014 (ISBN-1449659055); Michael Richard Green and Joseph Sambrook, Molecular Cloning: A Laboratory Manual, 4th ed., Cold Spring Harbor Laboratory Press, Cold Spring Harbor, N.Y., USA (2012) (ISBN 1936113414); Davis et al., Basic Methods in Molecular Biology, Elsevier Science Publishing, Inc., New York, USA (2012) (ISBN 044460149X); Laboratory Methods in Enzymology: DNA, Jon Lorsch (ed.) Elsevier, 2013 (ISBN 0124199542); Current Protocols in Molecular Biology (CPMB), Frederick M. Ausubel (ed.), John Wiley and Sons, 2014 (ISBN 047150338X, 9780471503385), Current Protocols in Protein Science (CPPS), John E. Coligan (ed.), John Wiley and Sons, Inc., 2005; and Current Protocols in Immunology (CPI) (John E. Coligan, ADA M Kruisbeek, David H Margulies, Ethan M Shevach, Warren Strobe, (eds.) John Wiley and Sons, Inc., 2003 (ISBN 0471142735, 9780471142737), the contents of which are all incorporated by reference herein in their entireties.

Other terms are defined herein within the description of the various aspects of the invention.

All patents and other publications; including literature references, issued patents, published patent applications, and co-pending patent applications; cited throughout this application are expressly incorporated herein by reference for the purpose of describing and disclosing, for example, the methodologies described in such publications that might be used in connection with the technology described herein. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. For example, while method steps or functions are presented in a given order, alternative embodiments may perform functions in a different order, or functions may be performed substantially concurrently. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure. Moreover, due to biological functional equivalency considerations, some changes can be made in protein structure without affecting the biological or chemical action in kind or amount. These and other changes can be made to the disclosure in light of the detailed description. All such modifications are intended to be included within the scope of the appended claims.

Specific elements of any of the foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

It is to be understood that the foregoing description and the following examples are illustrative only and are not to be taken as limitations upon the scope of the invention. Various changes and modifications to the disclosed embodiments, which will be apparent to those of skill in the art, may be made without departing from the spirit and scope of the present invention.

Some embodiments of the technology described herein can be defined according to any of the following numbered paragraphs:
1. A pharmaceutical composition comprising at least one secretogranin II (scg2) neuropeptide and a pharmaceutically acceptable carrier.
2. The pharmaceutical composition of paragraph 1, wherein the pharmaceutical composition is formulated for delivery to the central nervous system (CNS).
3. The pharmaceutical composition of any one of paragraphs 1-2, wherein the pharmaceutical composition is formulated for delivery across the blood-brain barrier (BBB).
4. The pharmaceutical composition of any one of paragraphs 1-3, wherein the pharmaceutical composition is formulated for delivery to the brain.
5. The pharmaceutical composition of any one of paragraphs 1-4, wherein the pharmaceutical composition is formulated for delivery to the hippocampus.
6. The pharmaceutical composition of any one of paragraphs 1-5, wherein the pharmaceutical composition is formulated for delivery to pyramidal cells.
7. The pharmaceutical composition of any one of paragraphs 1-6, wherein the formulation of the pharmaceutical composition is selected from the group consisting of: direct injection or infusion into the CNS; formulation as a solution comprising a carrier protein; formulation as a nanoparticle; formulation as a liposome; formulation as a nucleic acid; formulation as a CNS-tropic viral vector; formulation with or linkage to an agent that is endogenously transported across the BBB; formulation with or linkage to a cell penetrating peptide (CPP); formulation with or linkage to a BBB-shuttle; formulation with or linkage to an agent that increases permeability of the BBB.
8. The pharmaceutical composition of any one of paragraphs 1-7, wherein the scg2 neuropeptide is a cleavage product of secretogranin II (scg2) polypeptide.
9. The pharmaceutical composition of any one of paragraphs 1-8, wherein the scg2 polypeptide comprises SEQ ID NO: 4.
10. The pharmaceutical composition of any one of paragraphs 1-9, wherein the scg2 neuropeptide, when present in the scg2 polypeptide, is flanked at its N-terminus and at its C-terminus by a dibasic cleavage residue.
11. The pharmaceutical composition of any one of paragraphs 1-10, wherein the dibasic cleavage residue is selected from the group consisting of:
   a) arginine-lysine (RK);
   b) lysine-arginine (KR); and
   c) arginine-arginine (RR).
12. The pharmaceutical composition of any one of paragraphs 1-11, wherein the dibasic cleavage residue is lysine-arginine (KR).
13. The pharmaceutical composition of any one of paragraphs 1-12, wherein the dibasic cleavage residue is a specific cleavage site for a Pcsk1/2 protease.
14. The pharmaceutical composition of any one of paragraphs 1-13, wherein the at least one scg2 neuropeptide is selected from the group consisting of:
   a) secretoneurin;
   b) EM66;
   c) manserin; and
   d) SgII.
15. The pharmaceutical composition of any one of paragraphs 1-14, wherein the scg2 neuropeptide is secretoneurin.
16. The pharmaceutical composition of any one of paragraphs 1-15, wherein the scg2 neuropeptide is EM66.
17. The pharmaceutical composition of any one of paragraphs 1-16, wherein the scg2 neuropeptide is manserin.
18. The pharmaceutical composition of any one of paragraphs 1-17, wherein the scg2 neuropeptide is SgII.
19. The pharmaceutical composition of any one of paragraphs 1-18, wherein secretoneurin comprises TNEIVEEQYTPQSLATLESVFQELGKLTGPNNQ (SEQ ID NO: 5).
20. The pharmaceutical composition of any one of paragraphs 1-19, wherein EM66 comprises

(SEQ ID NO: 6)
ERMDEEQKLYTDDEDDIYKANNIAYEDVVGGEDWNPVEEKIESQTQEEVR
DSKENIEKNEQINDEM.

21. The pharmaceutical composition of any one of paragraphs 1-20, wherein manserin comprises

(SEQ ID NO: 7)
VPGQGSSEDDLQEEEQIEQAIKEHLNQGSSQETDKLAPVS.

22. The pharmaceutical composition of any one of paragraphs 1-21, wherein SgII comprises

(SEQ ID NO: 8)
FPVGPPKNDDTPNRQYWDEDLLMKVLEYLNQEKAEKGREHIA.

23. The pharmaceutical composition of any one of paragraphs 1-22, wherein the scg2 neuropeptide comprises a human, mouse, rat, or chimpanzee scg2 neuropeptide or a chimera thereof.
24. The pharmaceutical composition of any one of paragraphs 1-23, wherein the scg2 neuropeptide comprises a peptidomimetic.
25. A nucleic acid comprising at least one nucleic acid sequence encoding a secretogranin II (scg2) neuropeptide.
26. The nucleic acid of paragraph 24, wherein the scg2 neuropeptide is selected from the group consisting of:
   a) secretoneurin;
   b) EM66;
   c) manserin; and
   d) SgII.
27. The nucleic acid of any one of paragraphs 25-26, wherein the nucleic acid sequence encodes secretoneurin.
28. The nucleic acid of any one of paragraphs 25-27, wherein the nucleic acid sequence encodes EM66.
29. The nucleic acid of any one of paragraphs 25-28, wherein the nucleic acid sequence encodes manserin.
30. The nucleic acid of any one of paragraphs 25-29, wherein the nucleic acid sequence encodes SgII.
31. The nucleic acid of any one of paragraphs 25-30, wherein the nucleic acid sequence encoding secretoneurin comprises

(SEQ ID NO: 9)
ACAAATGAAATAGTGGAGGAACAATATACTCCTCAAAGCCTTGCTACATT
GGAATCTGTCTTCCAAGAGCTGGGGAAACTGACAGGACCAAACAACCAG.

32. The nucleic acid of any one of paragraphs 25-31, wherein the nucleic acid sequence encoding EM66 comprises

(SEQ ID NO: 10)
GAGAGGATGGATGAGGAGCAAAAACTTTATACGGATGATGAAGATGATAT
CTACAAGGCTAATAACATTGCCTATGAAGATGTGGTCGGGGGAGAAGACT
GGAACCCAGTAGAGGAGAAAATAGAGAGTCAAACCCAGGAAGAGGTGAGA
GACAGCAAAGAGAATATAGAAAAAAATGAACAAATCAACGATGAGATG.

33. The nucleic acid of any one of paragraphs 25-32, wherein the nucleic acid sequence encoding manserin comprises

(SEQ ID NO: 11)
GTTCCTGGTCAAGGCTCATCTGAAGATGACCTGCAGGAAGAGGAACAAAT
TGAGCAGGCCATCAAAGAGCATTTGAATCAAGGCAGCTCTCAGGAGACTG
ACAAGCTGGCCCCGGTGAGC.

34. The nucleic acid of any one of paragraphs 25-33, wherein the nucleic acid sequence encoding SgII comprises

(SEQ ID NO: 12)
TTCCCTGTGGGGCCCCCGAAGAATGATGATACCCCAAATAGGCAGTACTG
GGATGAAGATCTGTTAATGAAAGTGCTGGAATACCTCAACCAAGAAAAGG
CAGAAAAGGGAAGGGAGCATATTGCT.

35. A vector comprising the nucleic acid of any one of paragraphs 25-34.
36. The vector of paragraph 35, wherein the vector further comprises a promoter that is operatively linked to the nucleic acid sequence encoding the scg2 neuropeptide.
37. The vector of paragraph 36, wherein the promoter comprises an Activator protein 1 (AP-1) family driven promoter.
38. The vector of paragraph 36, wherein the promoter comprises a constitutive promoter.
39. The vector of paragraph 36, wherein the promoter comprises a nervous tissue-specific promoter.
40. A viral vector comprising the nucleic acid of any one of paragraphs 25-34 or the vector of any one of paragraphs 35-39.
41. The viral vector of paragraph 40, wherein the viral vector is an adenovirus-associated virus (AAV).
42. The viral vector of paragraph 21, wherein the AAV is serotype AAV2/1.
43. A cell comprising the pharmaceutical composition of any one of paragraphs 1-24, the nucleic acid of any one of paragraphs 25-34, the vector of any one of paragraphs 35-39, or the viral vector of any one of paragraphs 40-42.
44. The cell of paragraph 43, wherein the cell is a neuronal cell.
45. The cell of paragraph 43 or 44, wherein the cell is a hippocampal cell.
46. The cell of any one of paragraphs 43-45, wherein the cell is a pyramidal cell.
47. The cell of any one of paragraphs 43-46, wherein the cell is a CA1 pyramidal cell.
48. A composition comprising the nucleic acid of any one of paragraphs 25-34, the vector of any one of paragraphs 35-39, the viral vector of any one of paragraphs 40-42, or the cell of any one of paragraphs 43-47, and a pharmaceutically acceptable carrier.
49. A method of increasing memory consolidation and/or memory retention, comprising administering an effective amount of the pharmaceutical composition of any one of paragraphs 1-24, the nucleic acid of any one of paragraphs 25-34, the vector of any one of paragraphs 35-39, or the viral vector of any one of paragraphs 40-42 to a subject in need thereof
50. A method of treating a memory-associated disorder, comprising administering an effective amount of the pharmaceutical composition of any one of paragraphs 1-24, the nucleic acid of any one of paragraphs 25-34, the vector of any one of paragraphs 35-39, or the viral vector of any one of paragraphs 40-42 to a subject in need thereof
51. A method of treating a learning disability, comprising administering an effective amount of the pharmaceutical composition of any one of paragraphs 1-24, the nucleic acid of any one of paragraphs 25-34, the vector of any one of paragraphs 35-39, or the viral vector of any one of paragraphs 40-42 to a subject in need thereof
52. A method of treating a neurodegenerative disease or disorder, comprising administering an effective amount of the pharmaceutical composition of any one of paragraphs 1-24, the nucleic acid of any one of paragraphs 25-34, the vector of any one of paragraphs 35-39, or the viral vector of any one of paragraphs 40-42 to a subject in need thereof.
53. A method of treating epilepsy, comprising administering an effective amount of the pharmaceutical composition of any one of paragraphs 1-24, the nucleic acid of any one of paragraphs 25-34, the vector of any one of paragraphs 35-39, or the viral vector of any one of paragraphs 40-42.
54. The method of any one of paragraphs 49-53, wherein the pharmaceutical composition, nucleic acid, vector, or viral vector is administered intracranially, epidurally, intrathecally, intraparenchymally, intraventricularly, or subarachnoidly.
55. The method of any one of paragraphs 49-54, wherein the pharmaceutical composition, nucleic acid, vector, or viral vector is administered in a formulation that crosses the blood-brain barrier.
56. The method of any one of paragraphs 49-55, wherein the scg2 neuropeptide binds to a G-protein coupled receptor (GPCR).
57. The method of any one of paragraphs 49-56, wherein the administration modulates activity of interneurons in the central nervous system of the subject.
58. The method of any one of paragraphs 49-57, wherein the administration modulates activity of interneurons in the hippocampus of the subject.
59. The method of any one of paragraphs 49-58, wherein the administration modulates activity of γ-aminobutyric acid-releasing (GABAergic) interneurons in the CA1 region of the hippocampus of the subject.
60. The method of any one of paragraphs 49-59, wherein the interneurons are parvalbumin-expressing interneurons (PV-IN) or cholecystokinin-expressing interneurons (CCK-IN).
61. The method of any one of paragraphs 49-60, wherein the administration increases PV-IN perisomatic inhibitory activity on an associated pyramidal cell.
62. The method of any one of paragraphs 49-61, wherein the administration decreases CCK-IN perisomatic inhibitory activity on an associated pyramidal cell.
63. The method of any one of paragraphs 49-62, wherein the administration increases the power of fast gamma waves (60 Hz-90 Hz) in the CA1 region of the hippocampus.
64. The method of any one of paragraphs 49-63, wherein the administration increases firing of pyramidal cells in the CA1 region of the hippocampus during the descending phase of the theta_{pyr} cycle.
65. The method of any one of paragraphs 49-64, wherein the administration increases spatial learning of the subject by at least 10% compared to a subject that is not administered the pharmaceutical composition, nucleic acid, vector, or viral vector.
66. The method of any one of paragraphs 49-65, wherein memory consolidation and/or memory retention is increased by at least 10% compared to a subject that is not administered the pharmaceutical composition, nucleic acid, vector, or viral vector.
67. The method of any one of paragraphs 49-66, wherein the memory-associated disorder is a learning disability or a neurodegenerative disease or disorder.
68. The method of any one of paragraphs 49-67, wherein the memory-associated disorder is selected from the group consisting of amnesia, dementia, Alzheimer's disease, mild cognitive impairment, vascular cognitive impairment, and hydrocephalus.
69. The method of any one of paragraphs 49-68, wherein the learning disability is selected from the group consisting of dyscalculia, dysgraphia, dyslexia, a non-verbal leaning disability, an oral and/or written language disorder and specific reading comprehension deficit, attention deficit hyperactivity disorder (ADHD), attention deficit disorder (ADD), dyspraxia, an executive mal-functioning, an auditory processing disorder, a language processing disorder, and a visual perceptual/visual motor deficit.
70. The method of any one of paragraphs 49-69, wherein the neurodegenerative disease or disorder is selected from the group consisting of Alzheimer's disease, Parkinson's disease, Huntington's disease, amyotrophic lateral sclerosis (ALS), frontotemporal dementia, chronic traumatic encephalopathy (CTE), multiple sclerosis, and neuroinflammation.
71. The method of any one of paragraphs 49-70, wherein the epilepsy is selected from the group consisting of focal seizures without loss of consciousness (simple partial seizures); focal seizures with impaired awareness (complex partial seizures); absence seizures (petit mal seizures); tonic seizures; atonic seizures; clonic seizures; myoclonic seizures; and tonic-clonic seizures.
72. A method of diagnosing a memory-associated disorder, learning disability, neurodegenerative disease or disorder, or epilepsy in a subject; comprising:
   a) obtaining a sample from the subject;
   b) detecting the level of a memory-associated analyte in the sample; and
   c) determining that the subject:
      i) has or is at risk of developing a memory-associated disorder, learning disability neurodegenerative disease or disorder, or epilepsy if the analyte level is below a pre-determined level; or
      ii) does not have or is not at risk of developing a memory-associated disorder, learning disability, neurodegenerative disease or disorder, or epilepsy if the analyte level is at or above a pre-determined level.
73. The method of paragraph 73, further comprising administering to the subject the pharmaceutical composition of any one of paragraphs 1-24, the nucleic acid of any one of paragraphs 25-34, the vector of any one of paragraphs 35-39, or the viral vector of any one of paragraphs 40-42, if the subject is determined to have or be at risk for developing a memory-associated disorder, learning disability, neurodegenerative disease or disorder, or epilepsy.
74. A method for detecting a memory-associated analyte in a sample from a subject comprising:
   a) obtaining a sample from the subject; and
   b) detecting the level of the memory-associated analyte in the sample.
75. The method of any one of paragraphs 74, wherein the step of detecting the level of the memory-associated analyte comprises mRNA detection or polypeptide detection.
76. The method of paragraph 75, wherein the mRNA detection comprises reverse transcription polymerase chain reaction (RT-PCR); quantitative RT-PCR; Northern blot analysis; differential gene expression; RNase protection assay; microarray based analysis; next-generation sequencing; or hybridization methods.
77. The method of paragraph 75, wherein the polypeptide detection comprises immunoassays, Western blot; immunoprecipitation; enzyme-linked immunosorbent assay (ELISA); radioimmunological assay (RIA); sandwich assay; immunohistological staining; radioimmunometric assay; immunofluorescence assay; mass spectroscopy; or immunoelectrophoresis assay.
78. A method of increasing memory consolidation and/or memory retention in a subject in need thereof, comprising:
   a) obtaining results detecting the level of a memory-associated analyte in a sample from the subject; and
   b) administering to the subject:
      i) the pharmaceutical composition of any one of paragraphs 1-24, the nucleic acid of any one of paragraphs 25-34, the vector of any one of paragraphs 35-39, or the viral vector of any one of paragraphs 40-42, if the analyte level is below a pre-determined level; or
      ii) an alternative treatment, if the analyte level is at or above a pre-determined level.
79. A method of treating a memory-associated disorder in a subject in need thereof, comprising:
   a) obtaining results detecting a memory-associated analyte in a sample from the subject; and
   b) administering to the subject:
      i) the pharmaceutical composition of any one of paragraphs 1-24, the nucleic acid of any one of paragraphs 25-34, the vector of any one of paragraphs 35-39, or the viral vector of any one of paragraphs 40-42, if the analyte level is below a pre-determined level; or
      ii) an alternative treatment, if the analyte level is at or above a pre-determined level.
80. A method of treating a learning disability in a subject in need thereof, comprising:
   a) obtaining results detecting a memory-associated analyte in a sample from the subject; and
   b) administering to the subject:
      i) the pharmaceutical composition of any one of paragraphs 1-24, the nucleic acid of any one of paragraphs 25-34, the vector of any one of paragraphs 35-39, or the viral vector of any one of paragraphs 40-42, if the analyte level is below a pre-determined level; or
      ii) an alternative treatment, if the analyte level is at or above a pre-determined level.
81. A method of treating a neurodegenerative disease or disorder in a subject in need thereof, comprising:
   a) obtaining results detecting the level of a memory-associated analyte in a sample from the subject; and
   b) administering to the subject:
      i) the pharmaceutical composition of any one of paragraphs 1-24, the nucleic acid of any one of paragraphs 25-34, the vector of any one of paragraphs 35-39, or the viral vector of any one of paragraphs 40-42, if the analyte level is below a pre-determined level; or
      ii) an alternative treatment, if the analyte level is at or above a pre-determined level.
82. A method of treating epilepsy in a subject in need thereof, comprising:
   a) obtaining results detecting the level of a memory-associated analyte in a sample from the subject; and
   b) administering to the subject:
      i) the pharmaceutical composition of any one of paragraphs 1-24, the nucleic acid of any one of paragraphs 25-34, the vector of any one of paragraphs 35-39, or the viral vector of any one of paragraphs 40-42, if the analyte level is below a pre-determined level; or
      ii) an alternative treatment, if the analyte level is at or above a pre-determined level.
83. The method of any one of paragraphs 72-82, wherein the sample is a cerebrospinal fluid sample or a CNS sample.
84. The method of any one of paragraphs 72-83, wherein the memory-associated analyte is Scg2 mRNA, polypeptide, or neuropeptide.
85. The method of any one of paragraphs 72-83, wherein the memory-associated analyte is Fos, Fosb, or Junb mRNA or polypeptide.
86. The method of any one of paragraphs 72-83, wherein the memory-associated analyte is Fos+, Fosb+, or Junb+ neurons.
87. A cell culture medium comprising at least one Scg2 neuropeptide.
88. A method for culturing a neuron, comprising contacting the neuron with the cell culture medium of paragraph 87.

### EXAMPLES

### Example 1: Bidirectional Perisomatic Inhibitory Plasticity of a Fos Neuronal Network

Behavioral experiences activate the Fos transcription factor (TF) in sparse populations of neurons that are critical for encoding and recalling specific events. However, there is limited understanding of the mechanisms by which experience drives circuit reorganization to establish a network of Fos-activated cells. It is also unknown if Fos is required in this process beyond serving as a marker of recent neural activity and, if so, which of its many gene targets underlie circuit reorganization. It is demonstrated herein that when mice engaged in spatial exploration of novel environments, perisomatic inhibition of Fos-activated hippocampal CA1 pyramidal neurons by parvalbumin (PV)-expressing interneurons (INs) was enhanced, while perisomatic inhibition by cholecystokinin (CCK)-expressing INs was weakened. This bidirectional modulation of inhibition was abolished when the function of the Fos TF complex was disrupted. Single-cell RNA-sequencing, ribosome-associated mRNA profiling, and chromatin analyses, combined with electrophysiology, revealed that Fos activates the transcription of Scg2 (secretogranin II), a gene that encodes multiple distinct neuropeptides, to coordinate these changes in inhibition. As PV-expressing INs and CCK-expressing INs mediate distinct features of pyramidal cell activity, the Scg2-dependent reorganization of inhibitory synaptic input can affect network function in vivo. Hippocampal gamma rhythms and pyramidal cell coupling to theta phase were significantly altered in the absence of Scg2. These findings reveal an instructive role for Fos and Scg2 in establishing a network of Fos-activated neurons via the rewiring of local inhibition to form a selectively modulated state. The opposing plasticity mechanisms acting on distinct inhibitory pathways can support the consolidation of memories over time.

### Bidirectional Modulation of IN Inputs

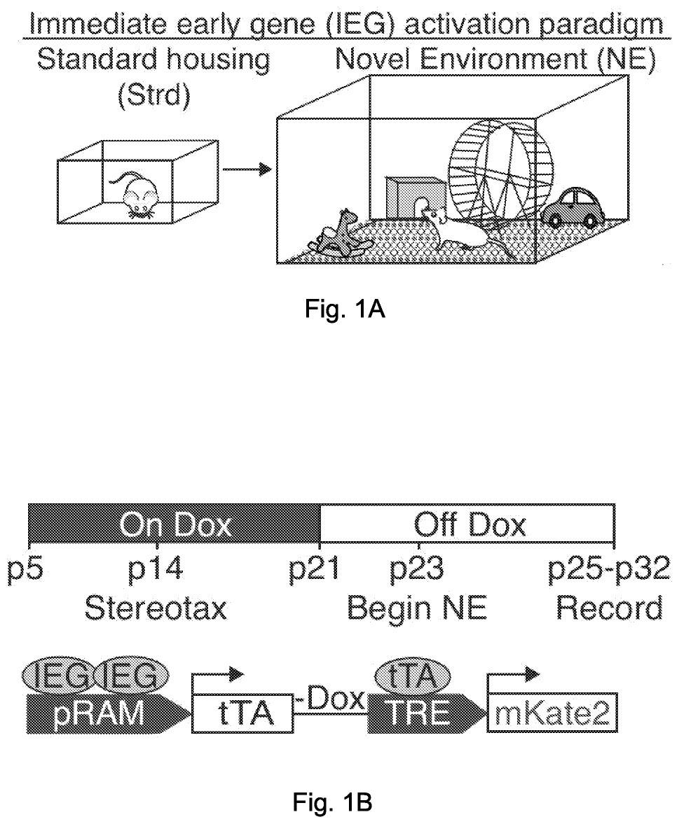

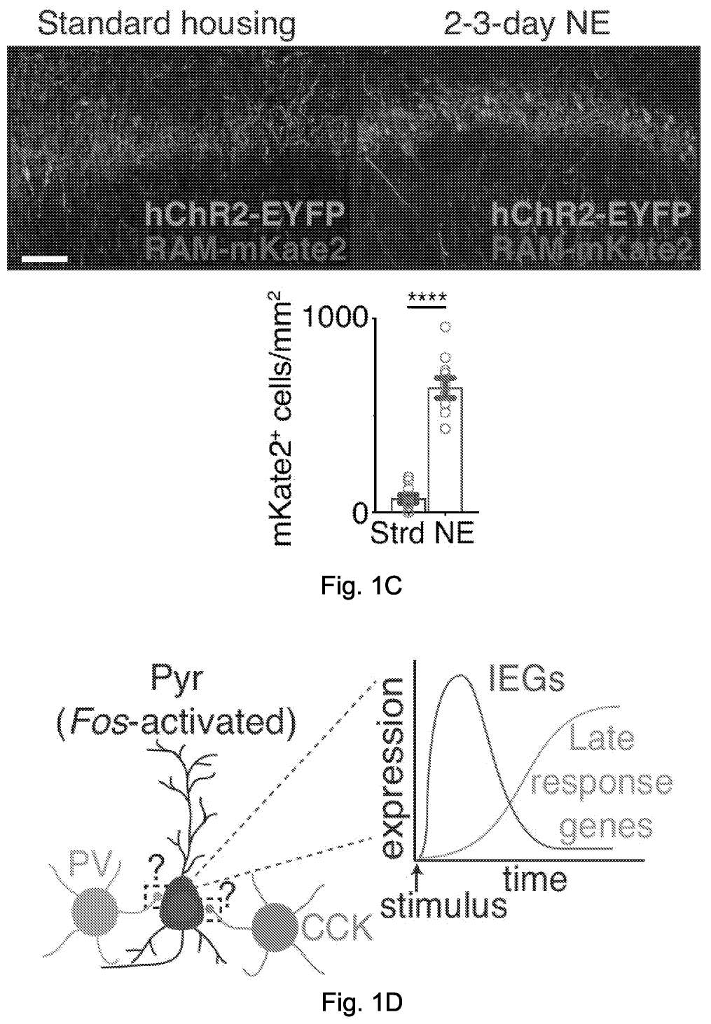

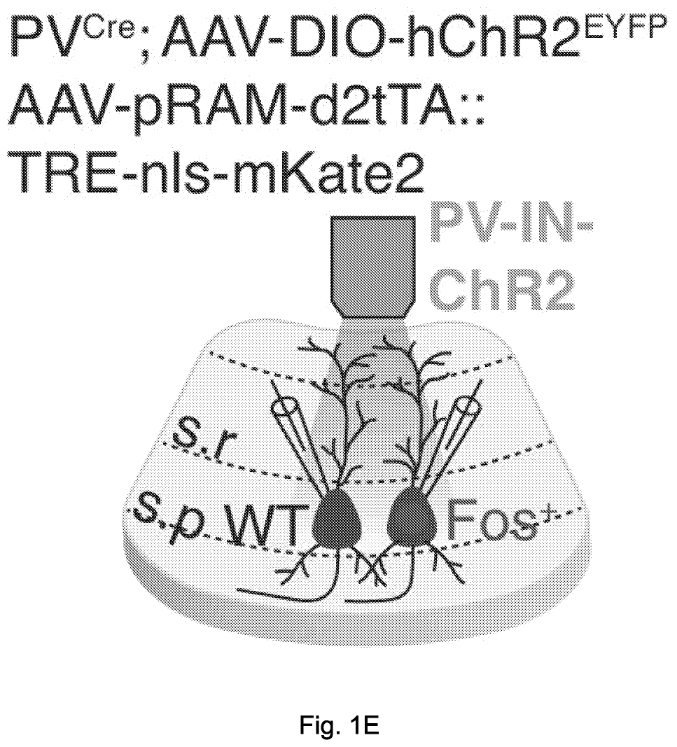

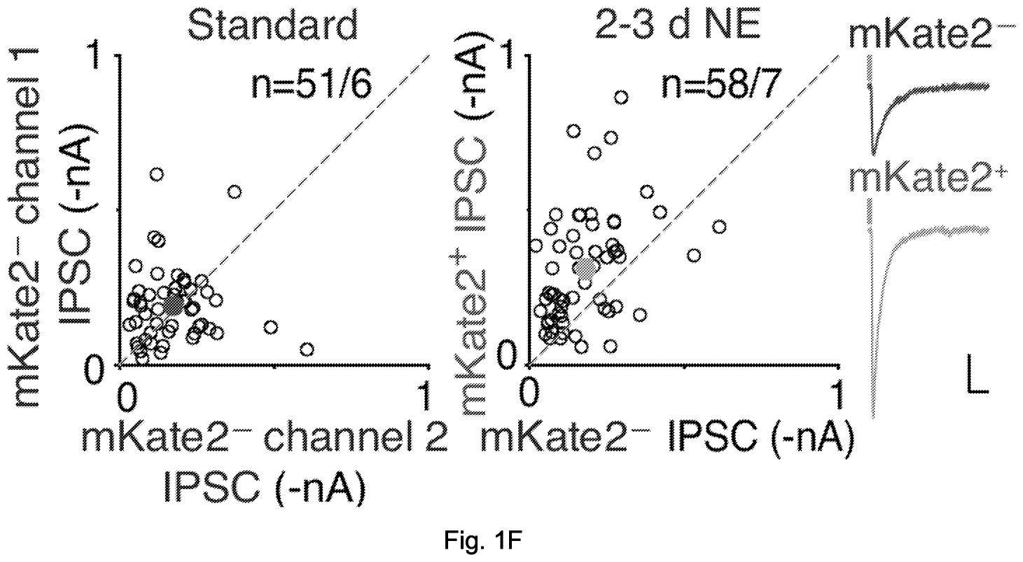

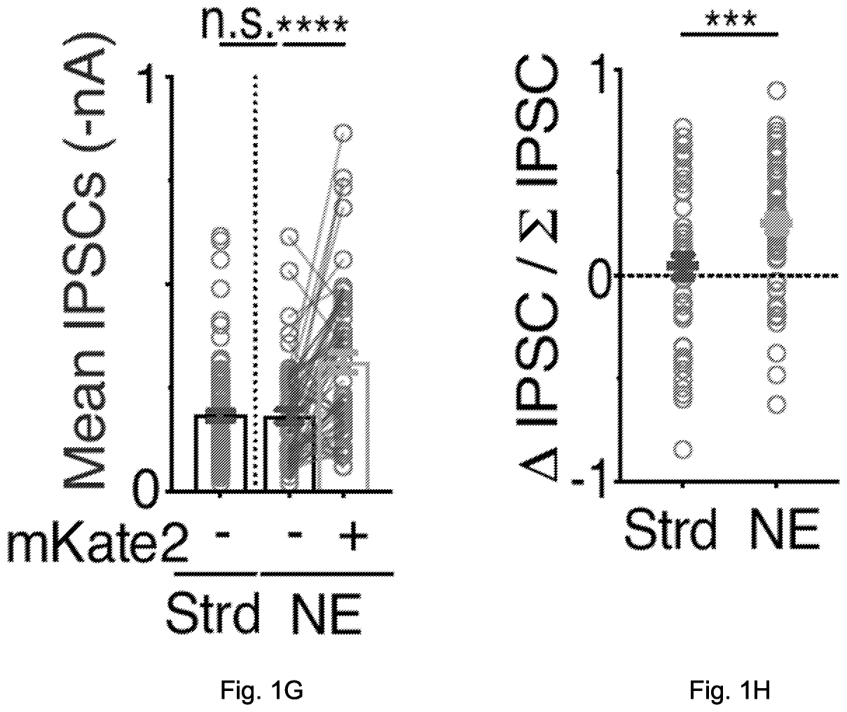

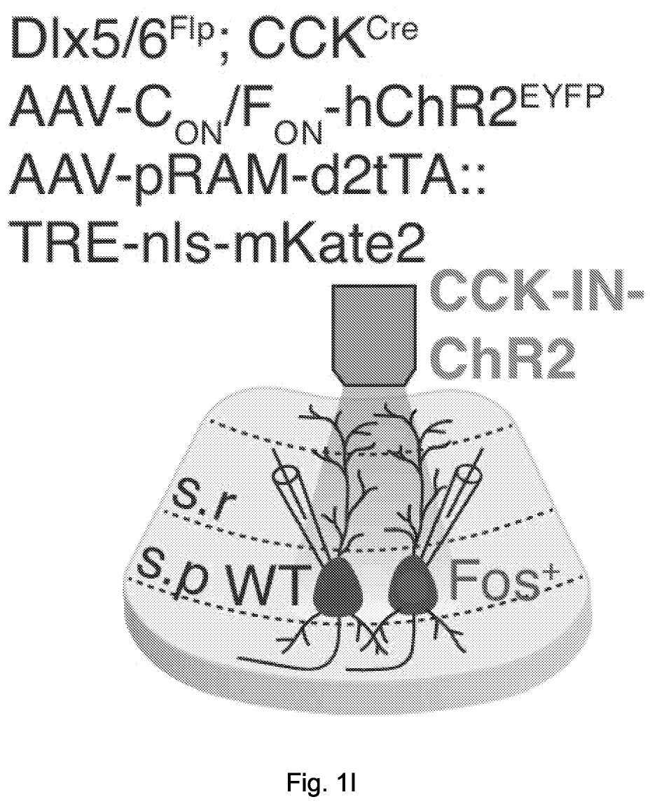

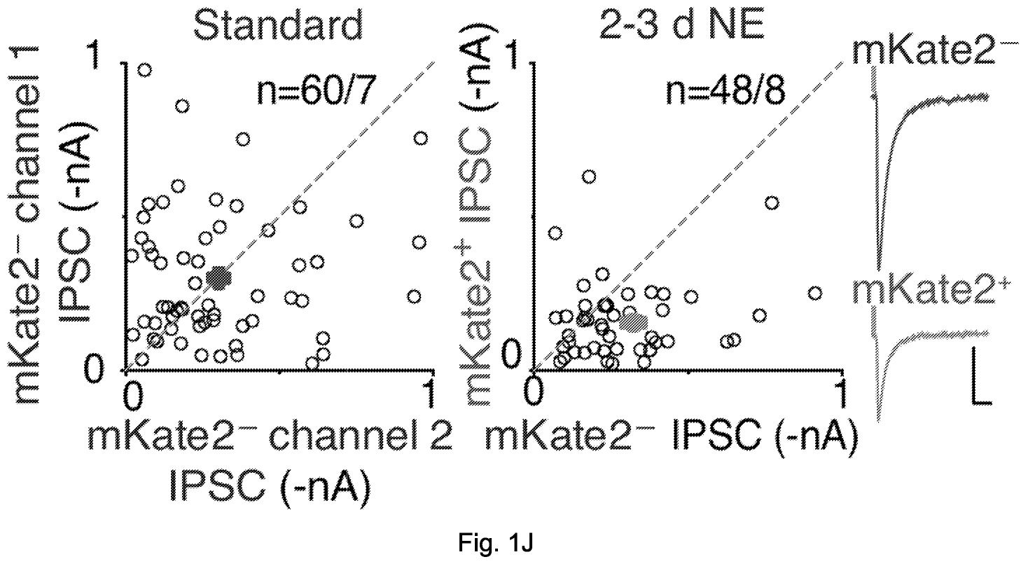

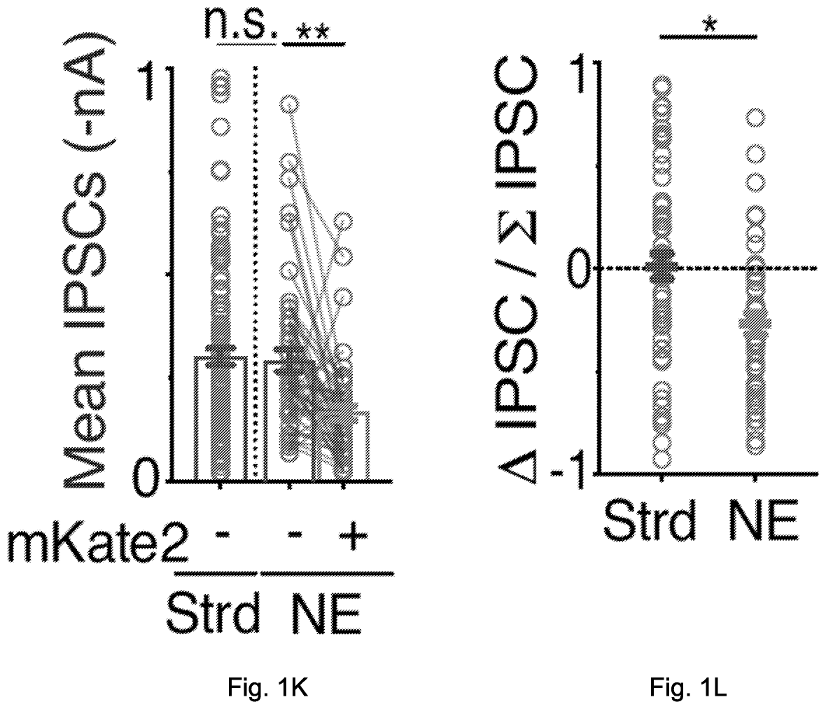

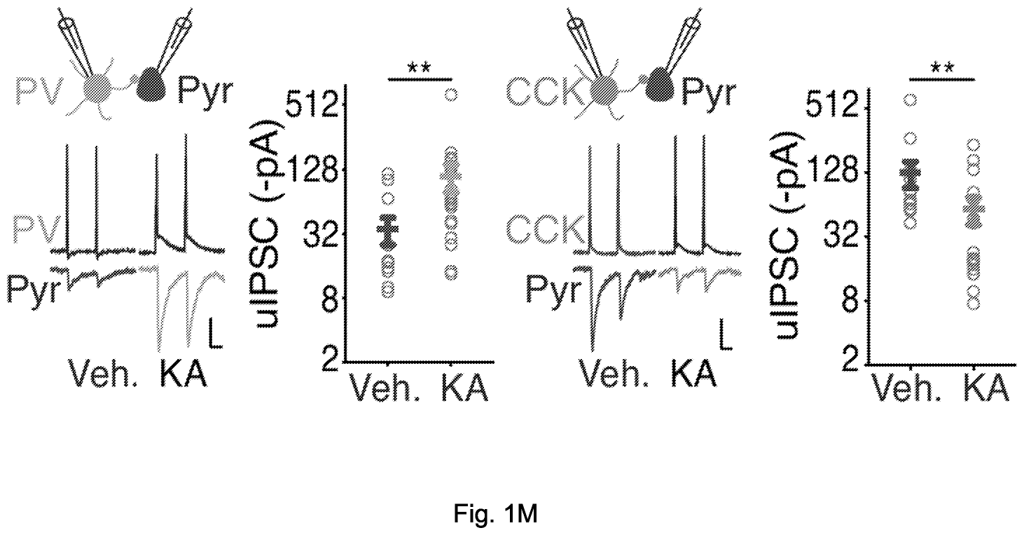

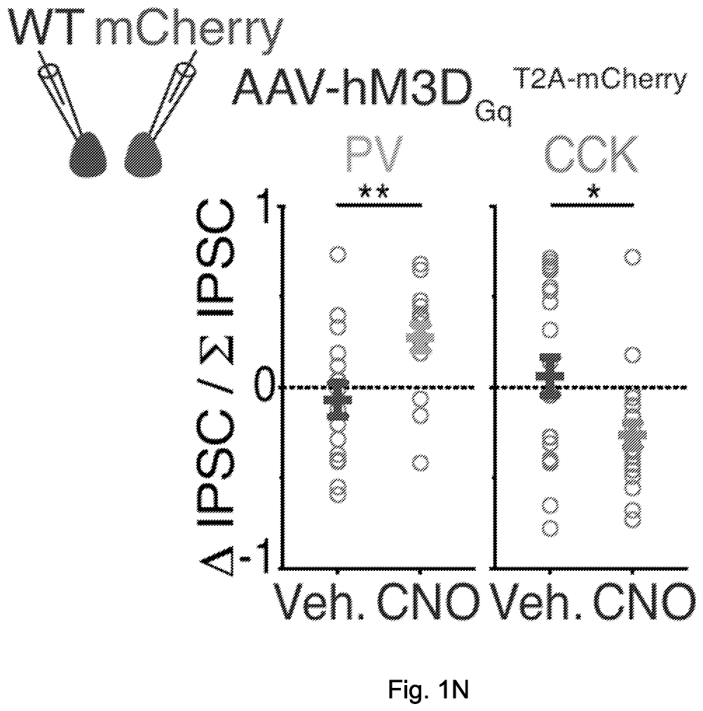

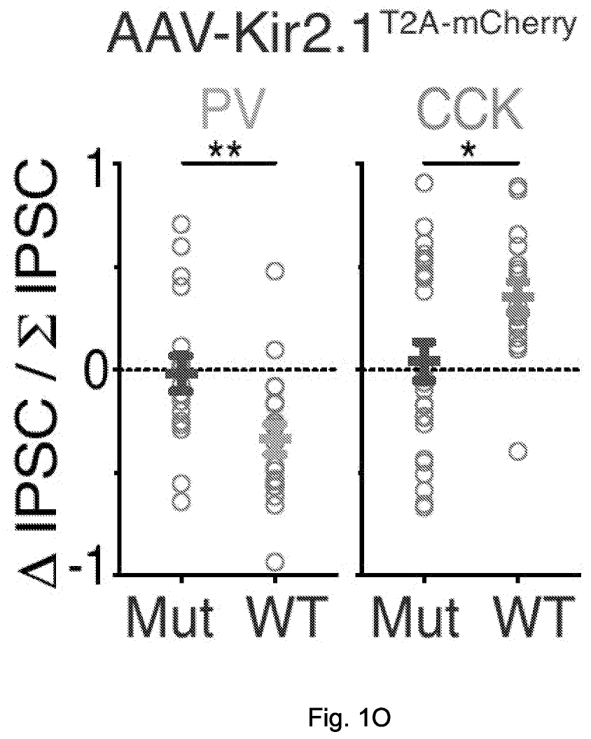

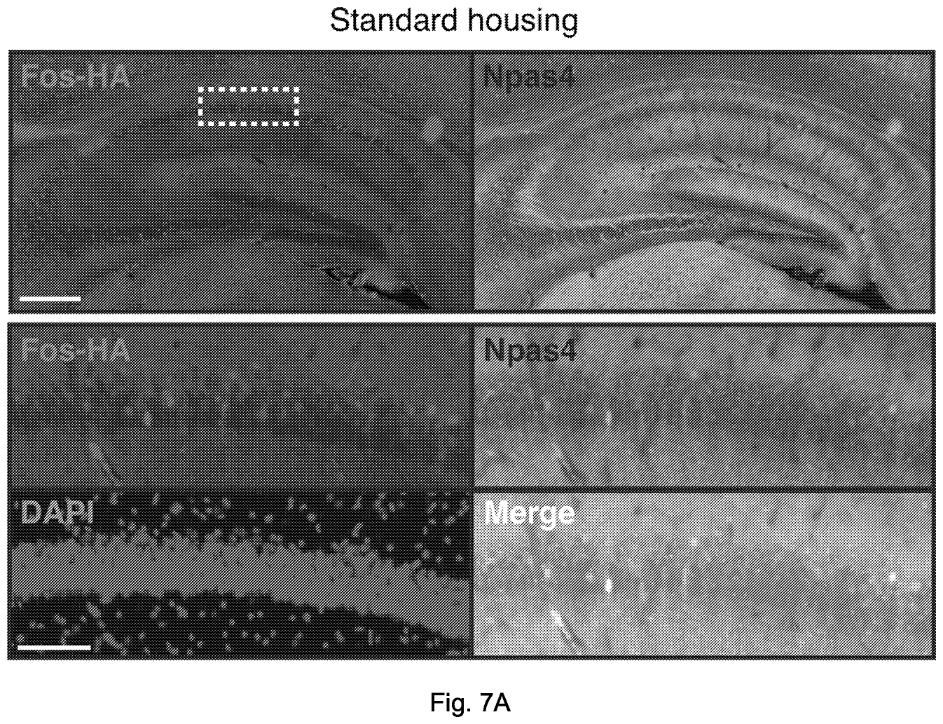

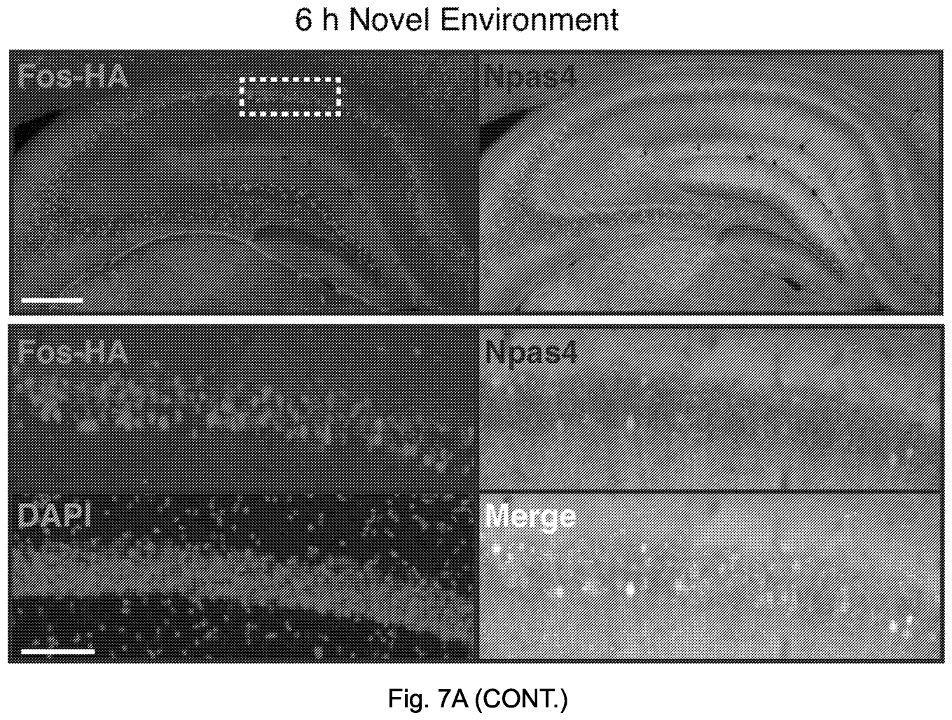

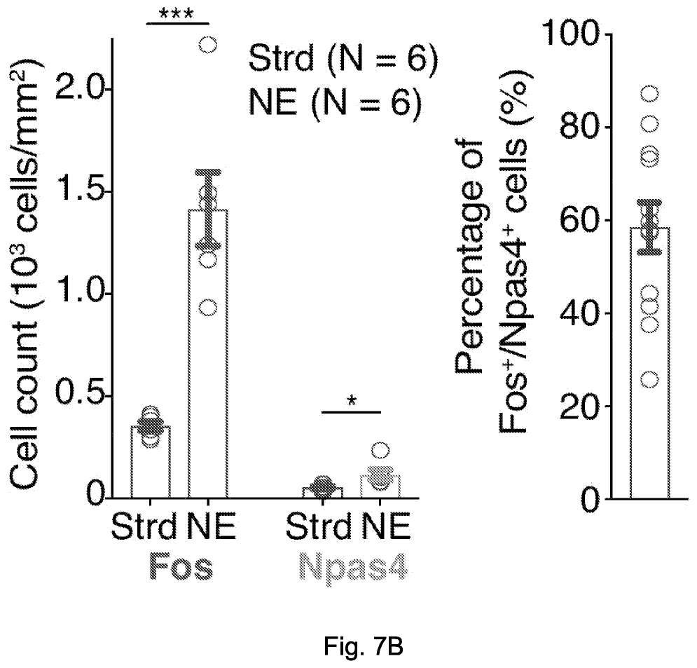

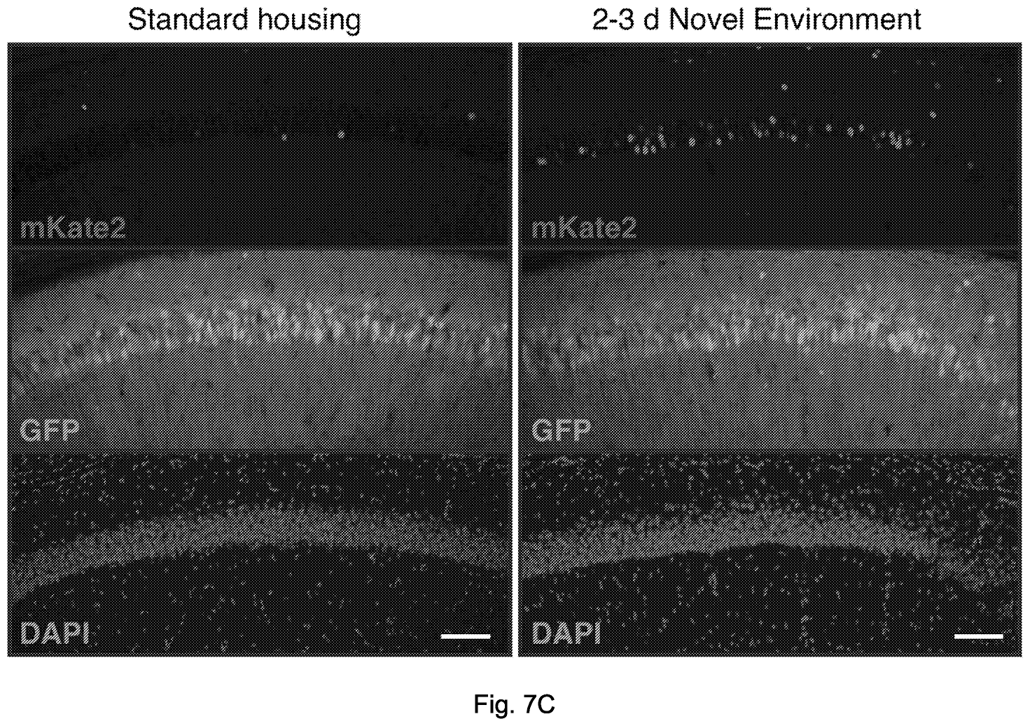

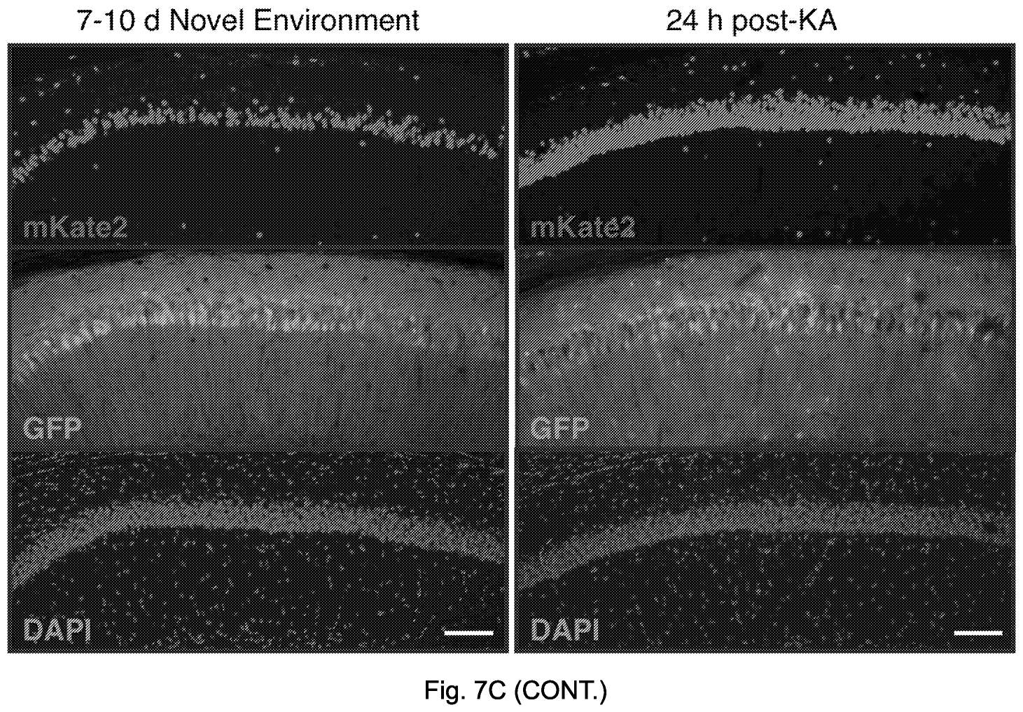

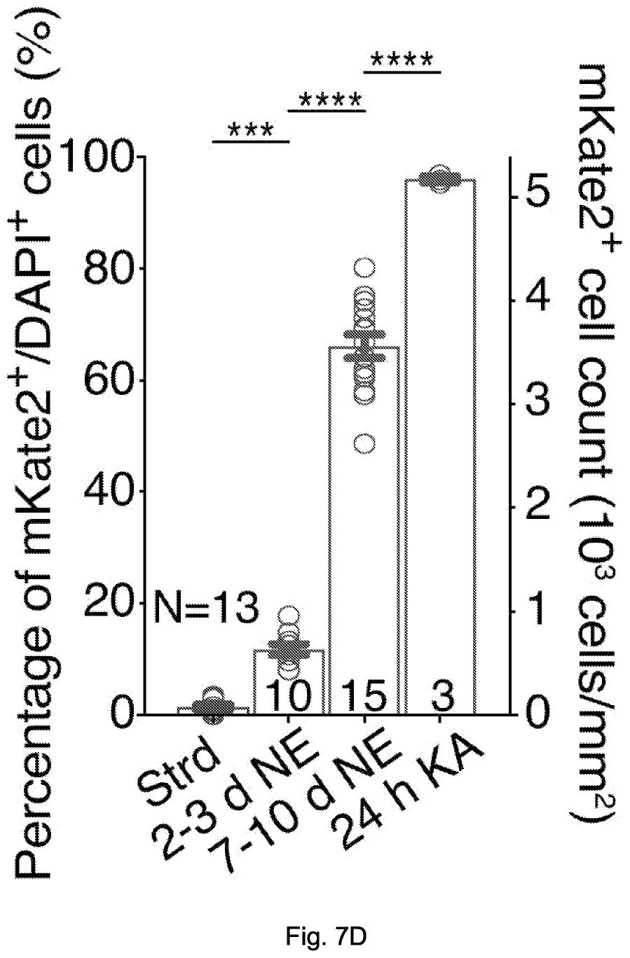

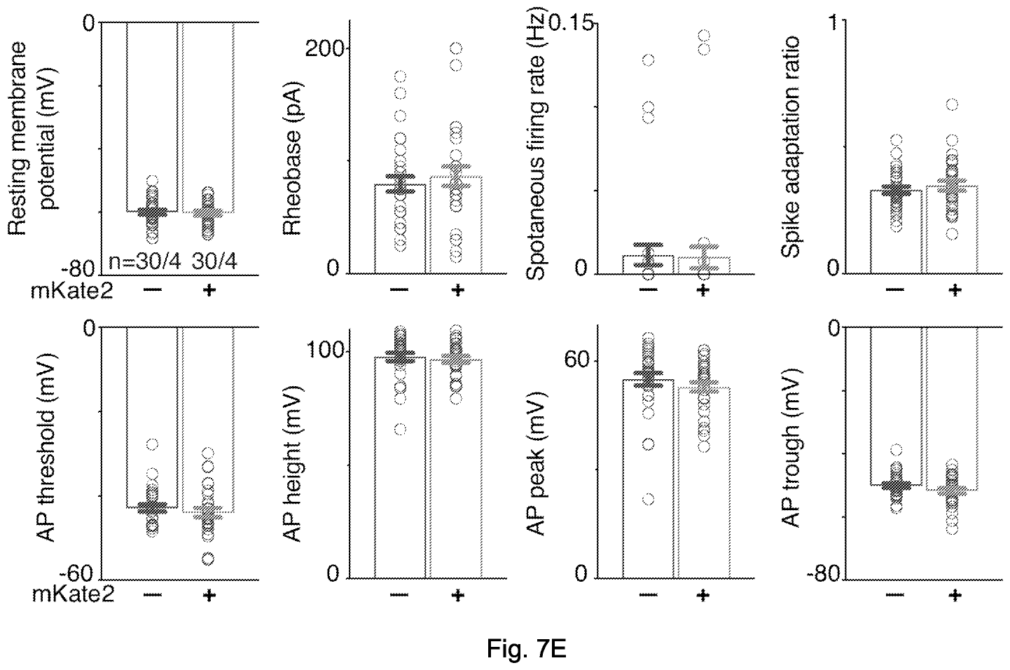

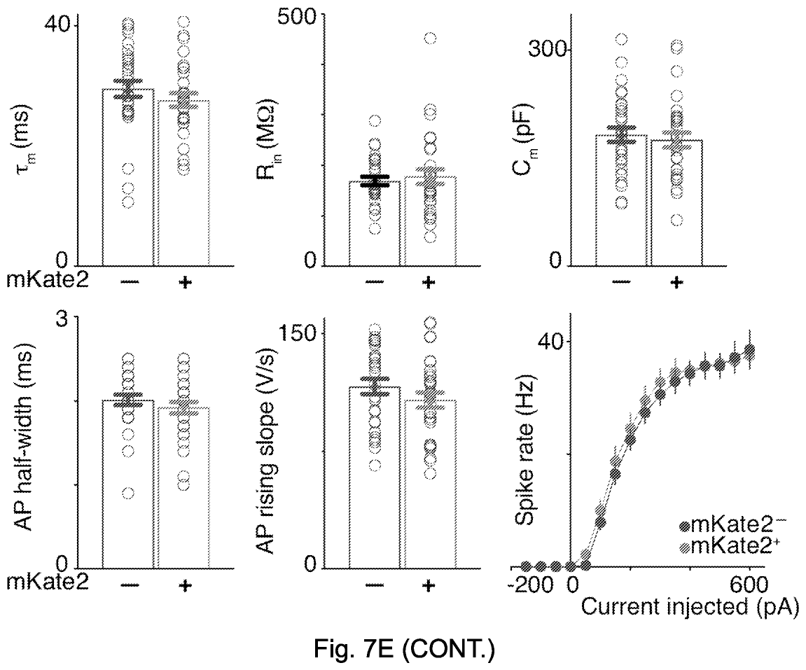

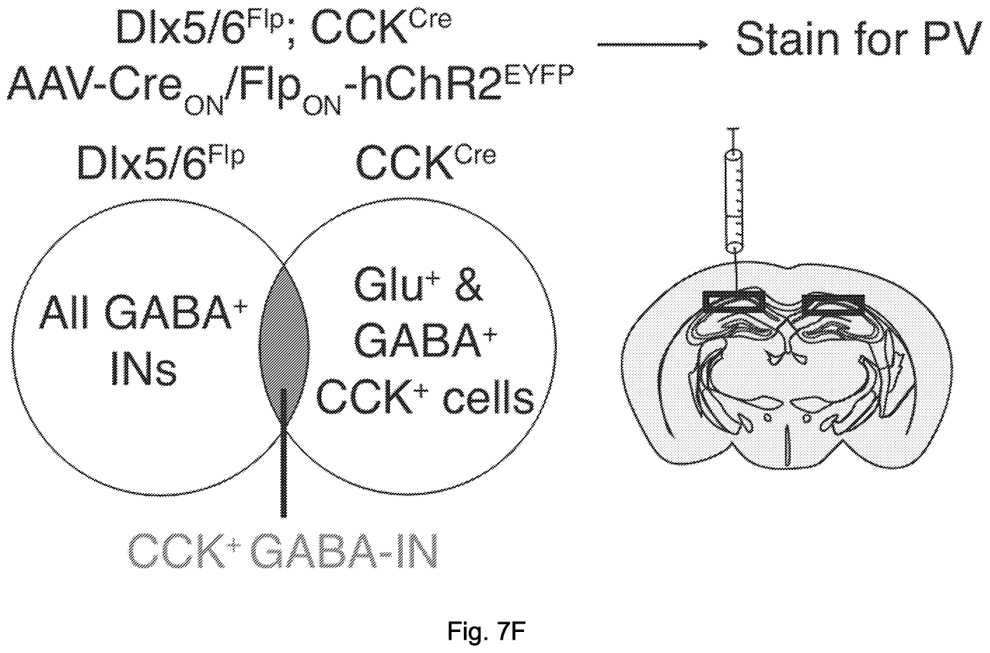

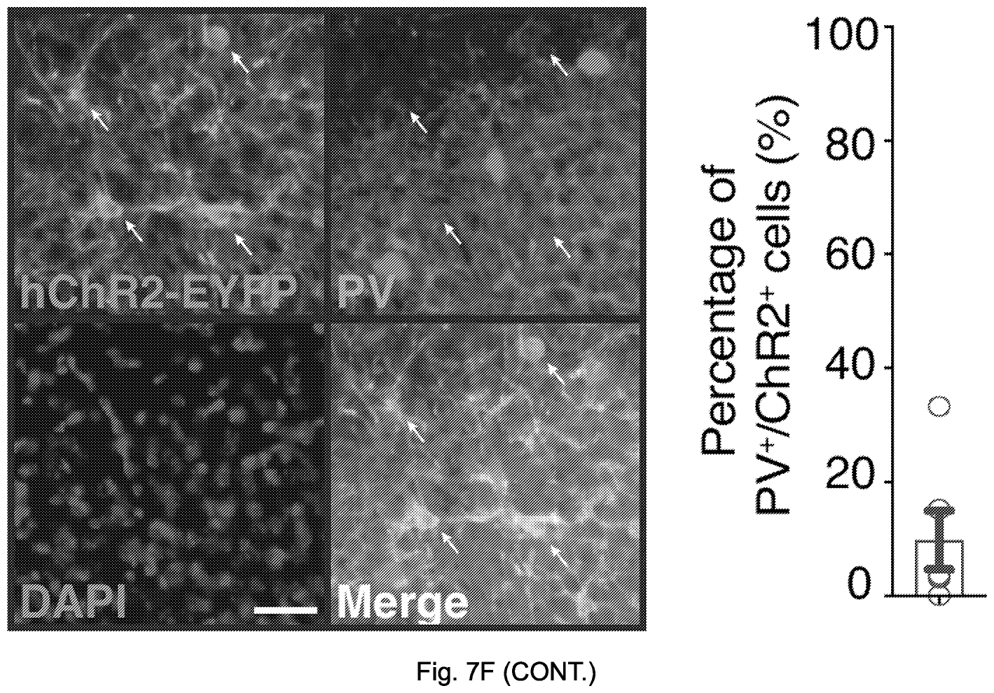

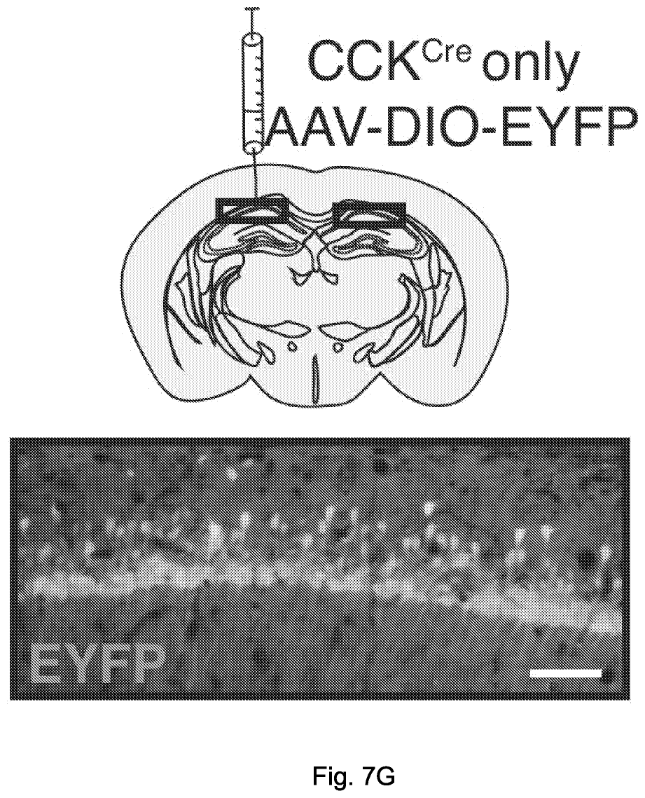

First, tests were performed to determine whether either of these forms of perisomatic inhibition (by PV-expressing INs and CCK-expressing INs) are differentially regulated onto Fos-expressing neurons compared to neighboring non-Fos-expressing neurons. Mice were exposed to a series of novel environments, which robustly activated Fos in a sparse subset of CA1 PCs (see e.g., FIG. 1A, FIG. 7A-FIG. 7D). To label these Fos-expressing neurons, an adeno-associated virus (AAV)-based reporter was used that expresses the fluorescent protein mKate2 selectively in recently activated neurons (see e.g., FIG. 1B); see e.g., Fenno et al. Nat Methods 11, 763-772, (2014), the content of which is incorporated herein by reference in its entirety. Using this reporter, a significant increase was detected in the number of recently activated neurons (mKate2⁺) in mice exposed to 2-3 days (see e.g., FIG. 1D) of novel environments (NE) compared to control mice housed under standard (Strd) conditions (see e.g., FIG. 1C). This 2-3d timepoint was therefore appropriate for assessing the long-lasting effects of Fos and its late-response target gene(s), which were usually activated within 1-12 hours (h) of stimulus onset (see e.g., FIG. 1D).

To assess PV-mediated inhibition, channelrhodopsin-2 (ChR2) was expressed via a Cre-dependent AAV in PV^{Cre} mice, which express Cre in PV-INs, permitting PV-mediated inhibitory postsynaptic currents (IPSCs) to be selectively evoked by focal photoactivation of ChR2-expressing PV-specific presynaptic boutons. PV-IPSCs were measured in CA1 PCs by performing dual whole-cell voltage-clamp recordings on pairs of recently activated (Fos⁺/mKate2⁺) and neighboring non-activated (Fos⁻/mKate2⁻) CA1 PCs in acute hippocampal slices prepared 2-3d after initial NE exposure (see e.g., FIG. 1E). The mean amplitude of PV-IPSCs in Fos⁺mKate2⁺ (311±24 pA (mean s.e.m.)) neurons was 1.7-fold higher compared with those in Fos⁻ mKate2⁻ neurons in either Strd or NE conditions (182±12 pA and 181±16 pA, respectively) (see e.g., FIG. 1F-FIG. 1H), indicating that PV-mediated inhibition was strengthened onto Fos-expressing neurons. By contrast, other electrophysiological parameters were not significantly different between the two groups (see e.g., FIG. 7E).

To assess CCK-mediated inhibition, an intersectional Flp- and Cre-dependent AAV was used in Dlx5/6^{Flp}; CCK^{Cre} mice to drive the expression of ChR2 specifically in CCK-INs, as the CCK^{Cre} driver alone labels both glutamatergic and GABAergic neurons whereas Dlx5/6Flp caused expression of Flp recombinase only in GABAergic INs (see e.g., FIG. 1I, FIG. 7F-FIG. 7G); see e.g., Taniguchi et al. Neuron 71, 995-1013 (2011); Roth, Neuron 89, 683-694 (2016); the contents of each of which are incorporated herein by reference in their entireties. When using an analogous experimental paradigm to the one described above, in contrast to the selective increase in PV-mediated inhibition onto Fos-activated CA1 PCs, the mean amplitude of CCK-IPSCs in Fos⁺CA1 PCs was significantly smaller (166±18 pA, 1.8-fold) compared with CCK-IPSCs in Fos⁻CA1 PCs (293±27 pA) (see e.g., FIG. 1J-FIG. 1L).

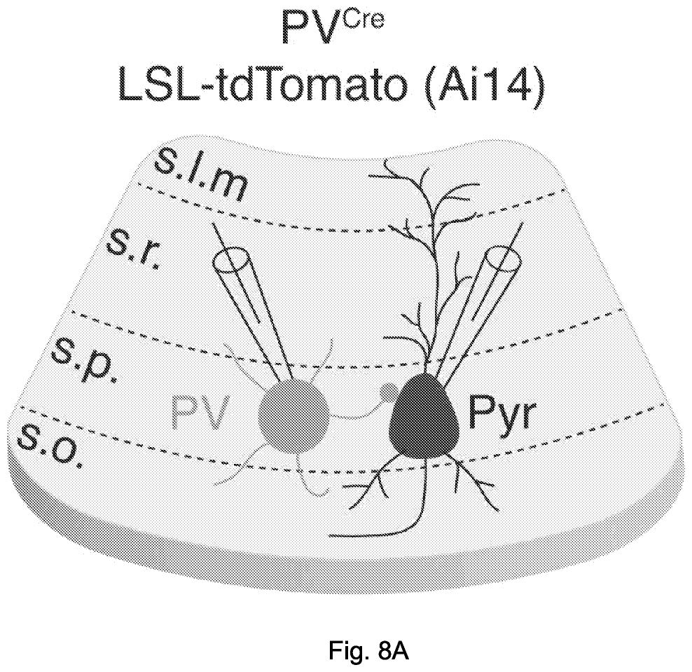

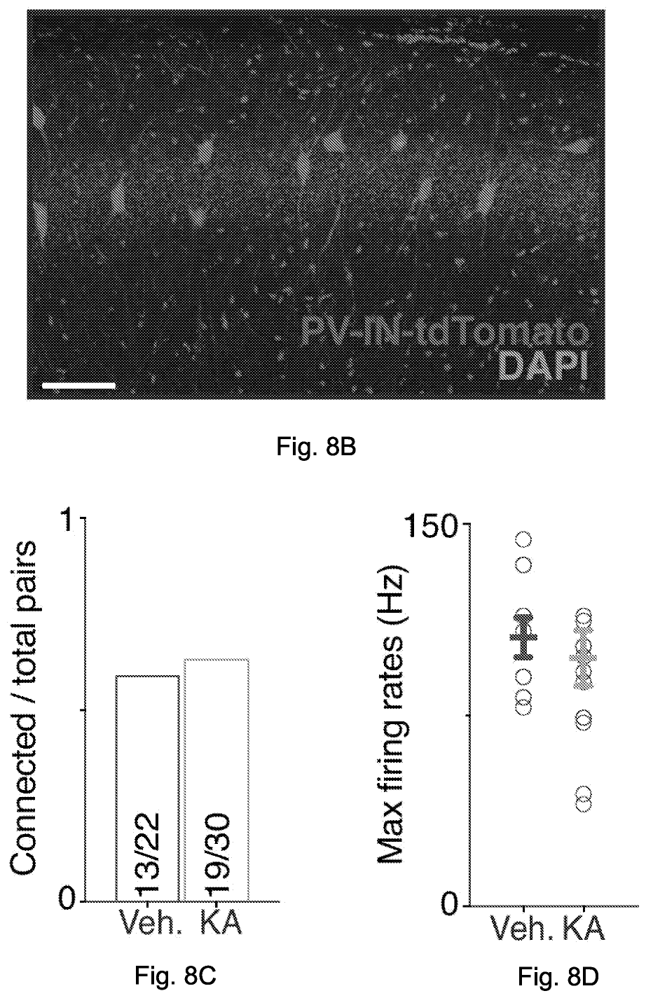

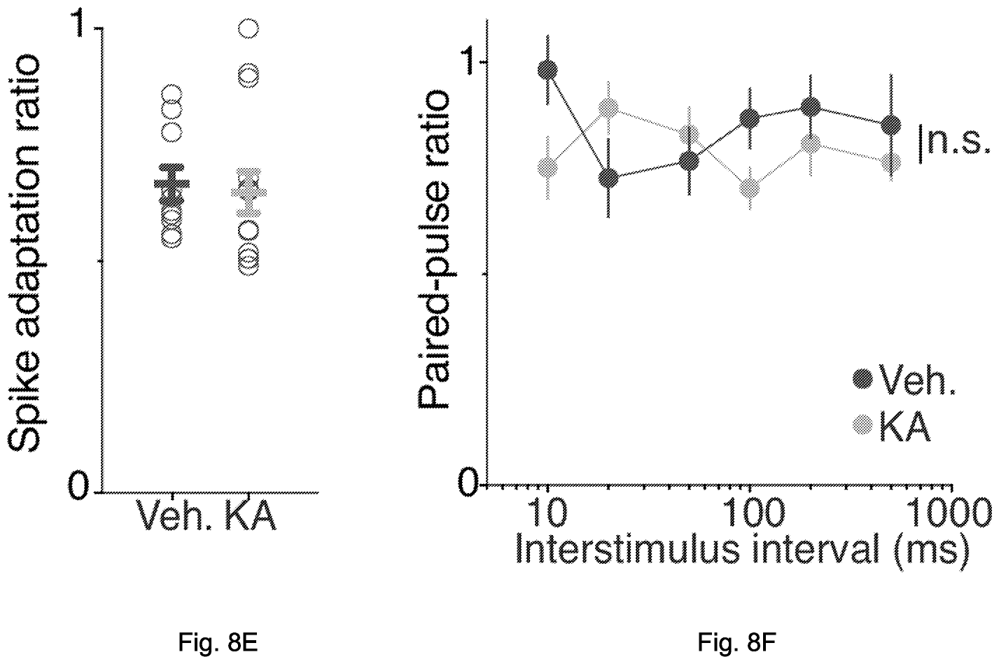

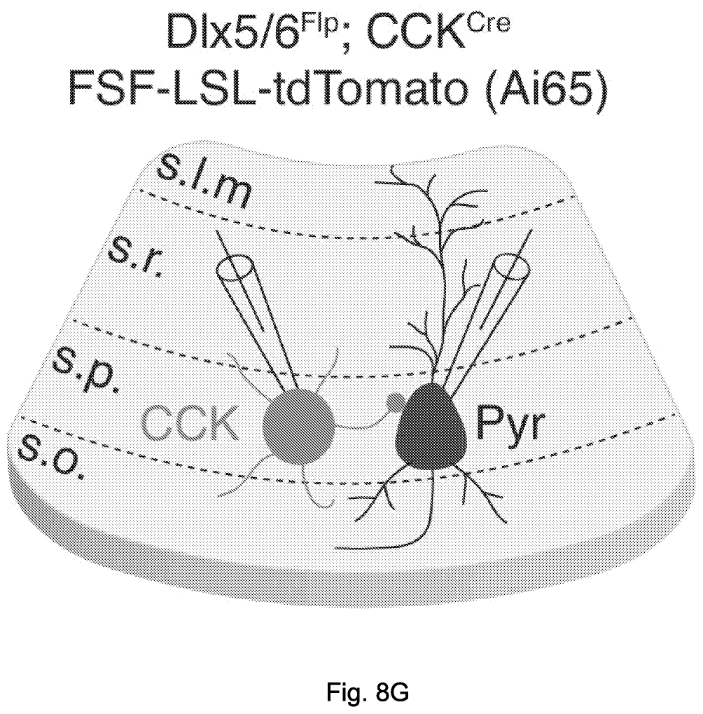

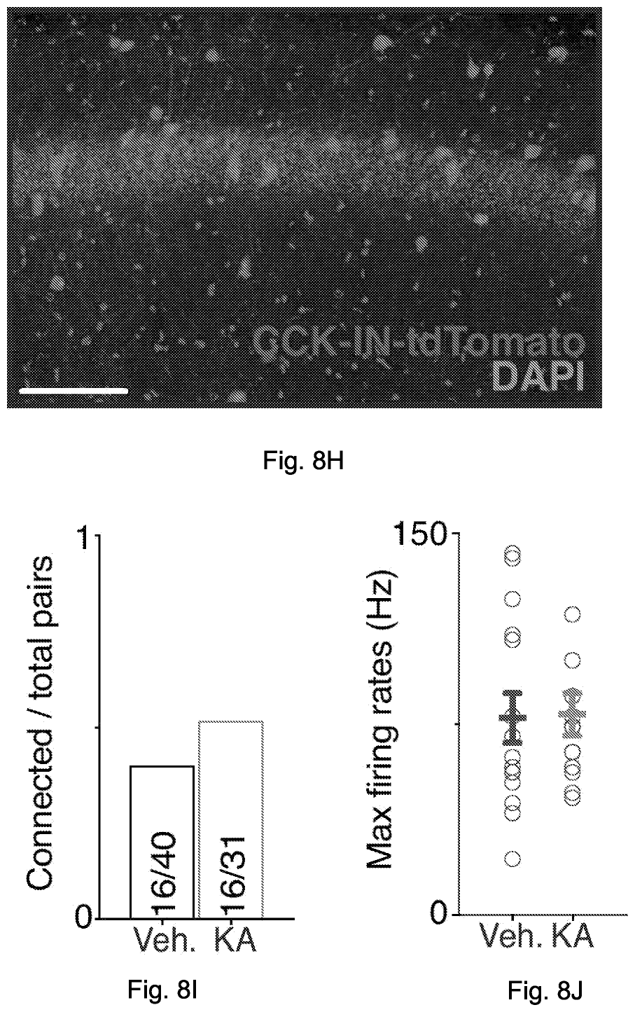

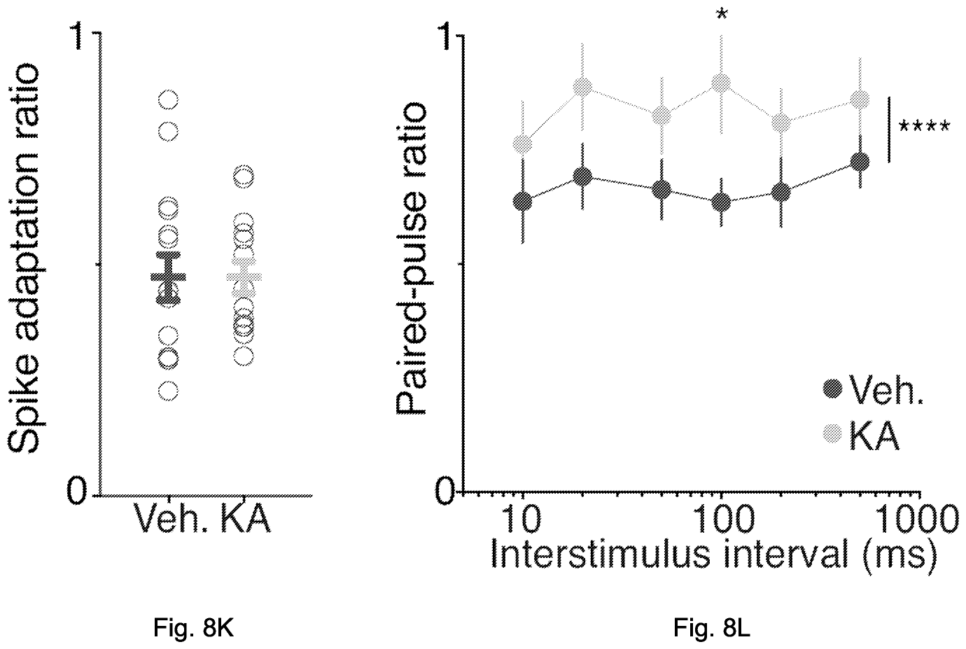

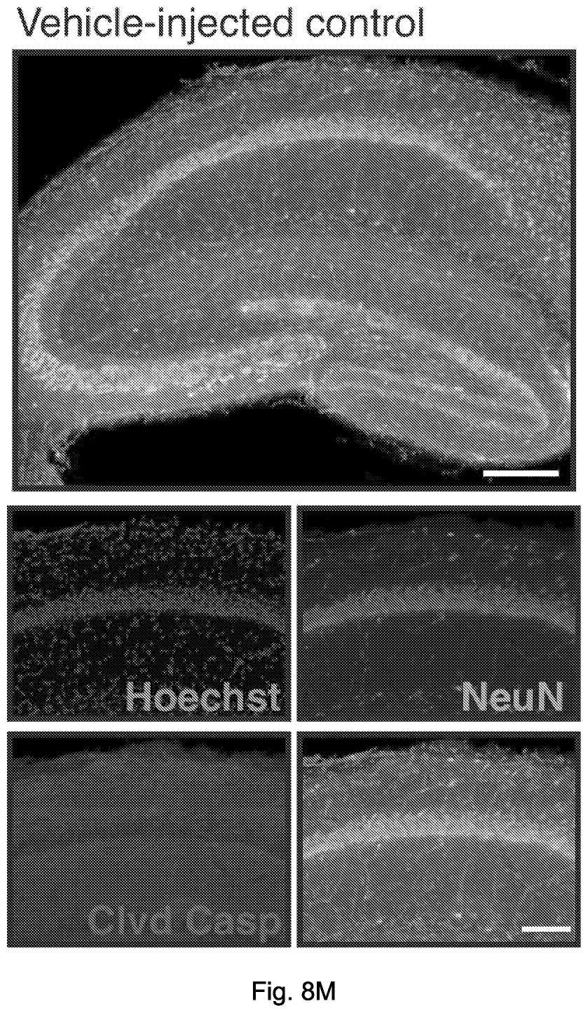

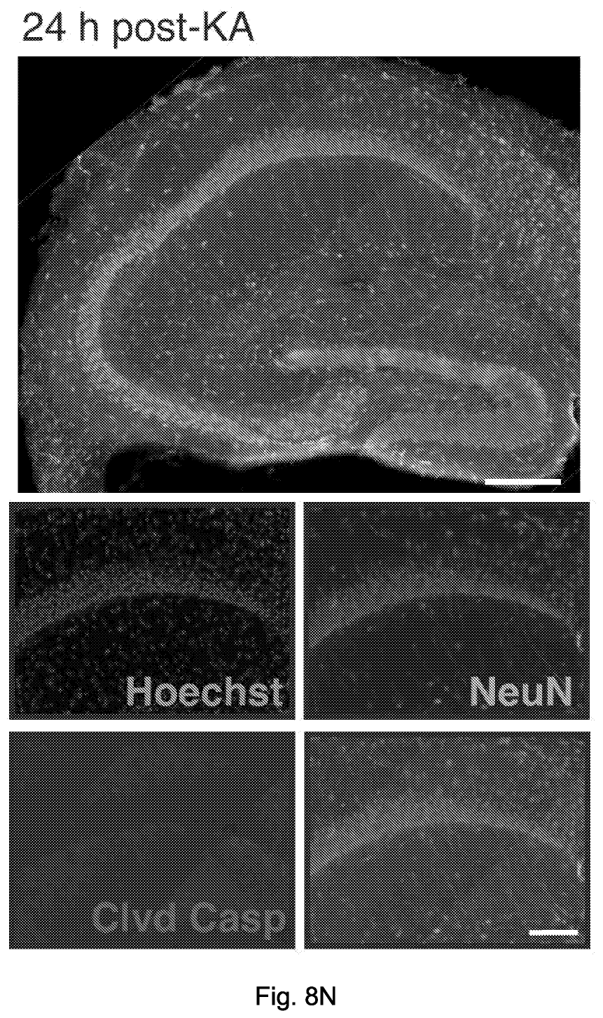

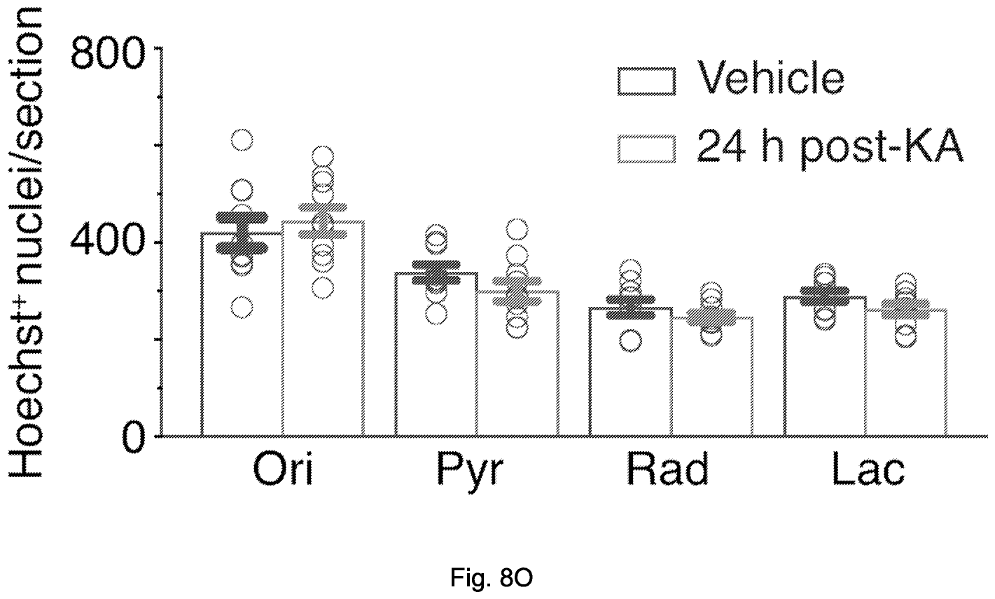

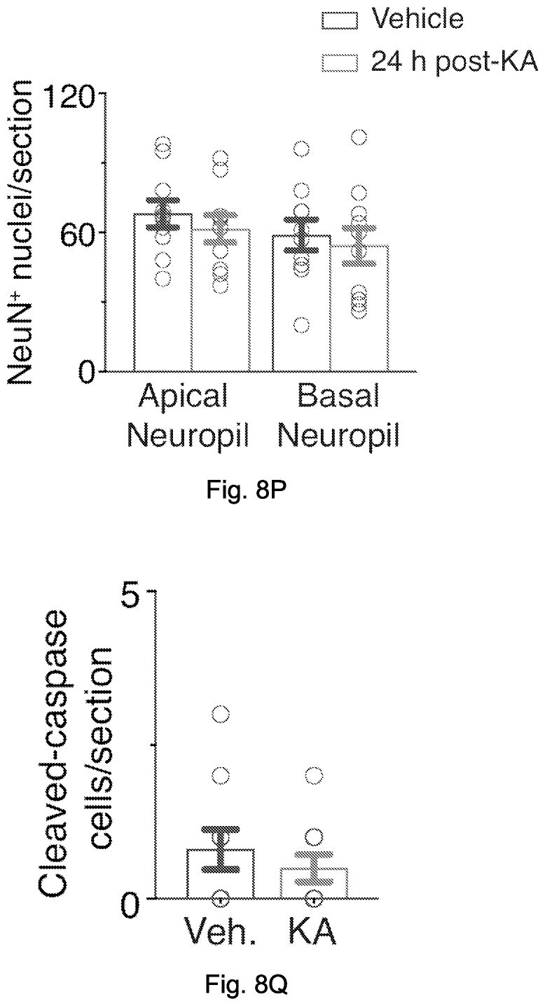

These findings were corroborated by paired recordings of IN-to-CA1 PC to measure amplitudes of unitary IPSC (uIPSC). Recordings were performed using slices prepared from PV^{Cre} or Dlx5/6^{Flp}; CCK^{Cre} tdTomato reporter mice 24 h after exposure to kainic acid (KA) to synchronously and reliably activate nearly all CA1 PCs (see e.g., FIG. 7C, FIG. 7D). Consistent with the findings using measurements of ChR2-evoked IPSCs, amplitudes of PV-uIPSC in CA1 PCs were 3.2-fold larger, whereas amplitudes of CCK-uIPSC in CA1 PCs were 2.2-fold smaller, following exposure to kainic acid (see e.g., FIG. 1M, FIG. 8A-FIG. 8Q).

These data indicate that NE exposure leads to selective, persistent bidirectional changes in perisomatic inhibition onto Fos-expressing neuronal ensembles, with PV-mediated inhibition strengthening and CCK-mediated inhibition weakening. These modifications are referred to herein as “bidirectional perisomatic inhibitory plasticity.”

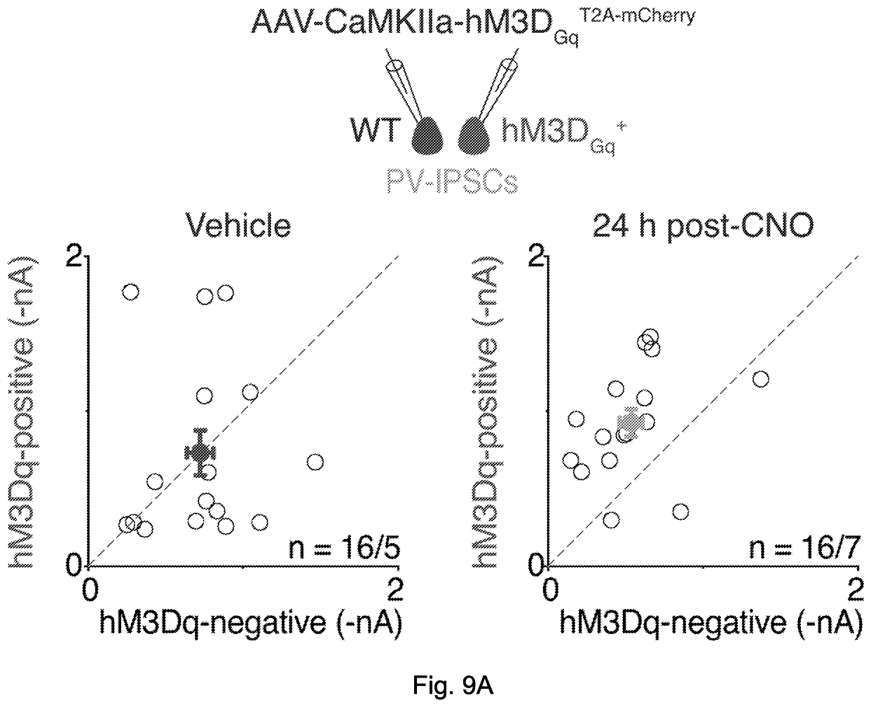

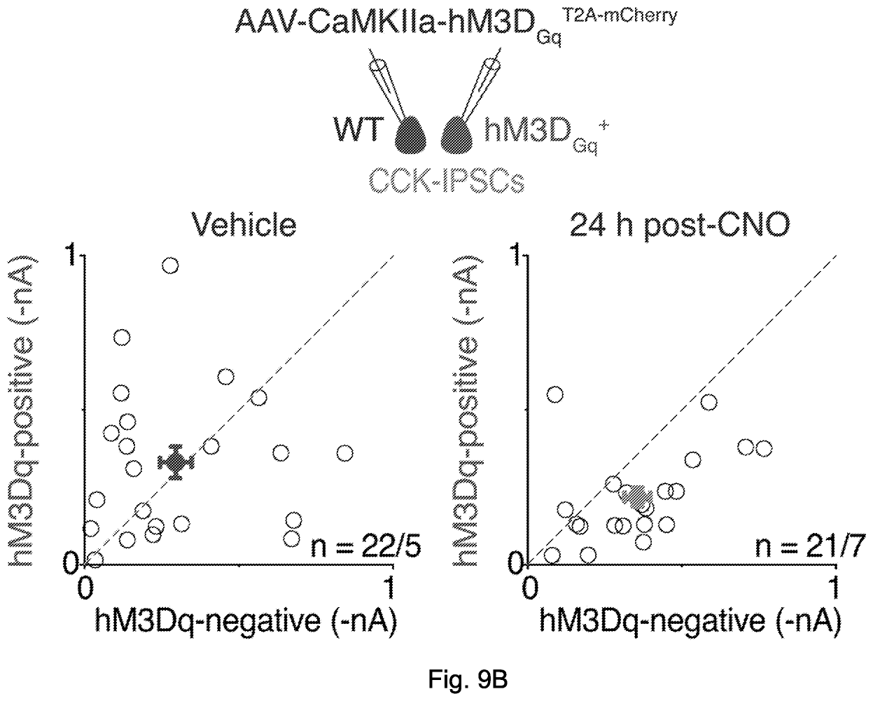

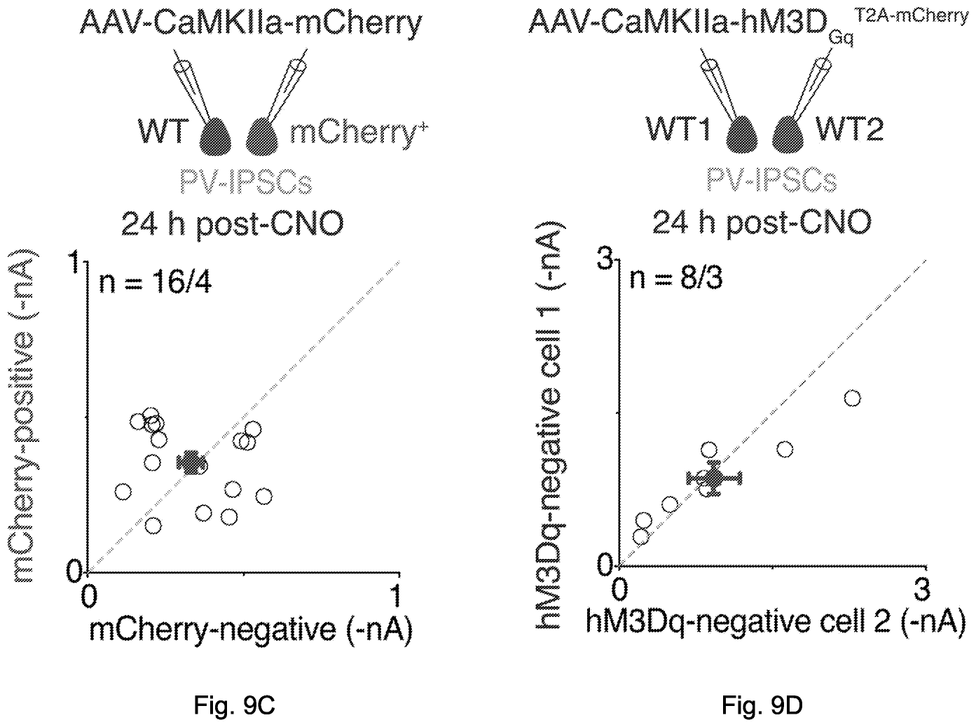

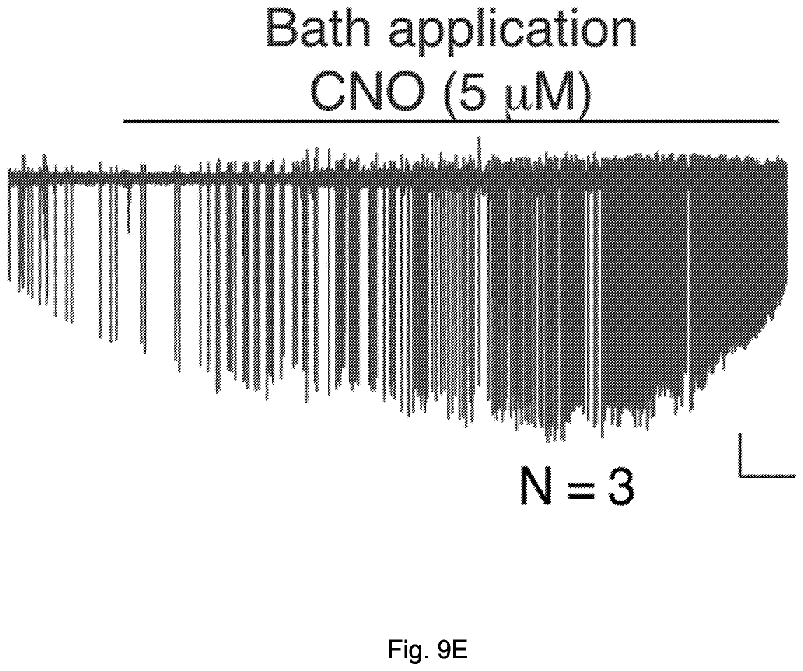

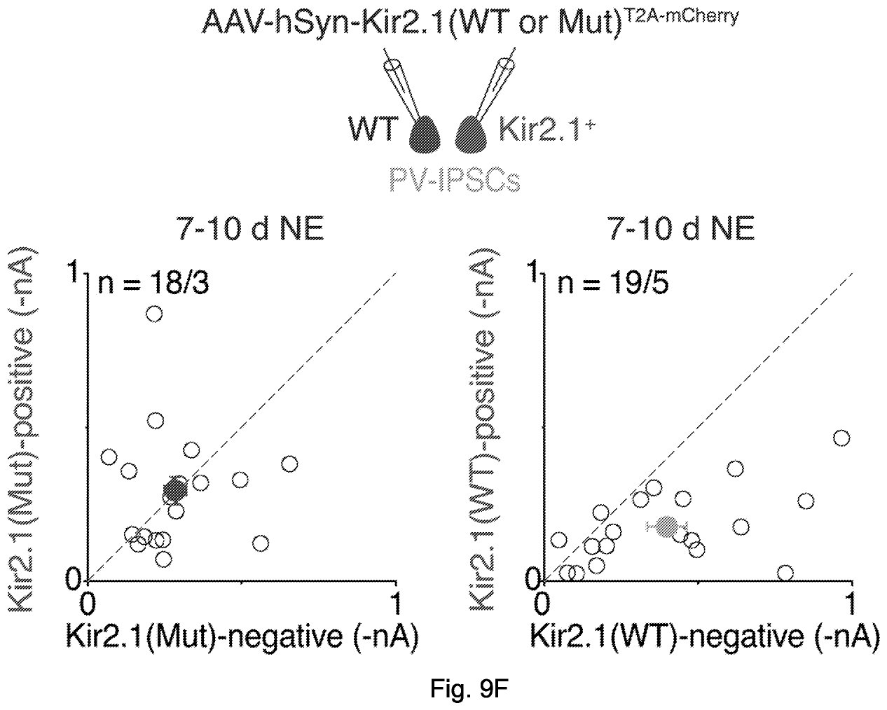

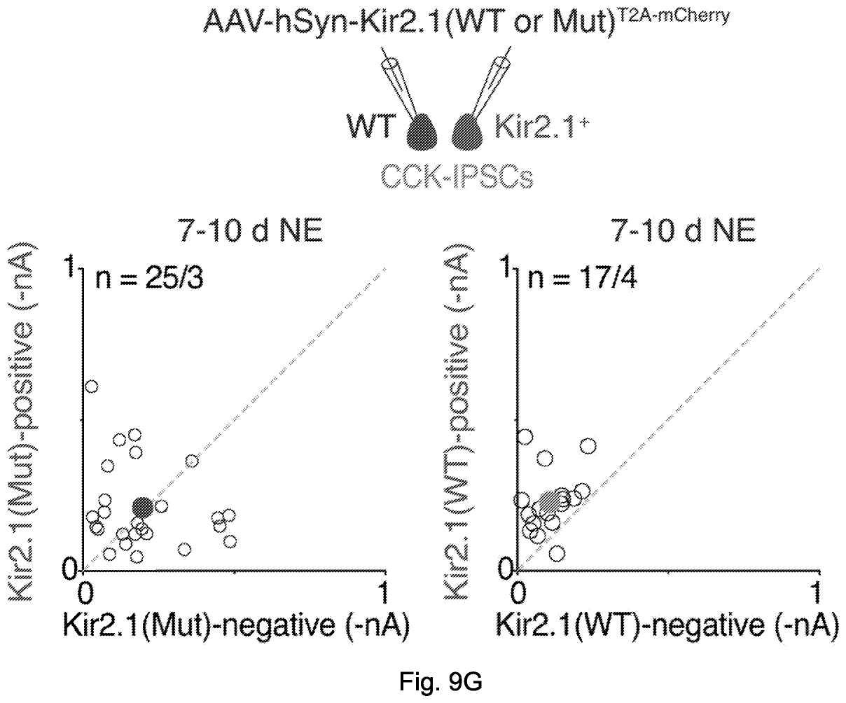

The bidirectional changes in perisomatic inhibition were a consequence of experience-driven neuronal activity, rather than a reflection of pre-existing differences between Fos⁻/mKate2⁺ and Fos⁻/mKate2⁻ CA1 PCs, insofar as they could be recapitulated by chemogenetic activation of neurons expressing the G_{q}-coupled Designer Receptors Exclusively Activated by Designer Drugs (DREADD) receptor hM3D_{Gq} (see e.g., FIG. 1N, FIG. 9A-FIG. 9E). Conversely, silencing CA1 PCs via expression of an inwardly-rectifying potassium channel Kir2.1, but not a non-conducting mutant (KirMut), led to the inverse effects (see e.g., FIG. 1O, FIG. 9F, FIG. 9G). See e.g., Roth, Neuron 89, 683-694 (2016); Xue et al. Nature 511, 596-600 (2014); the contents of each of which are incorporated herein by reference in their entireties.

### Causal Role of Fos Family Transcription Factors

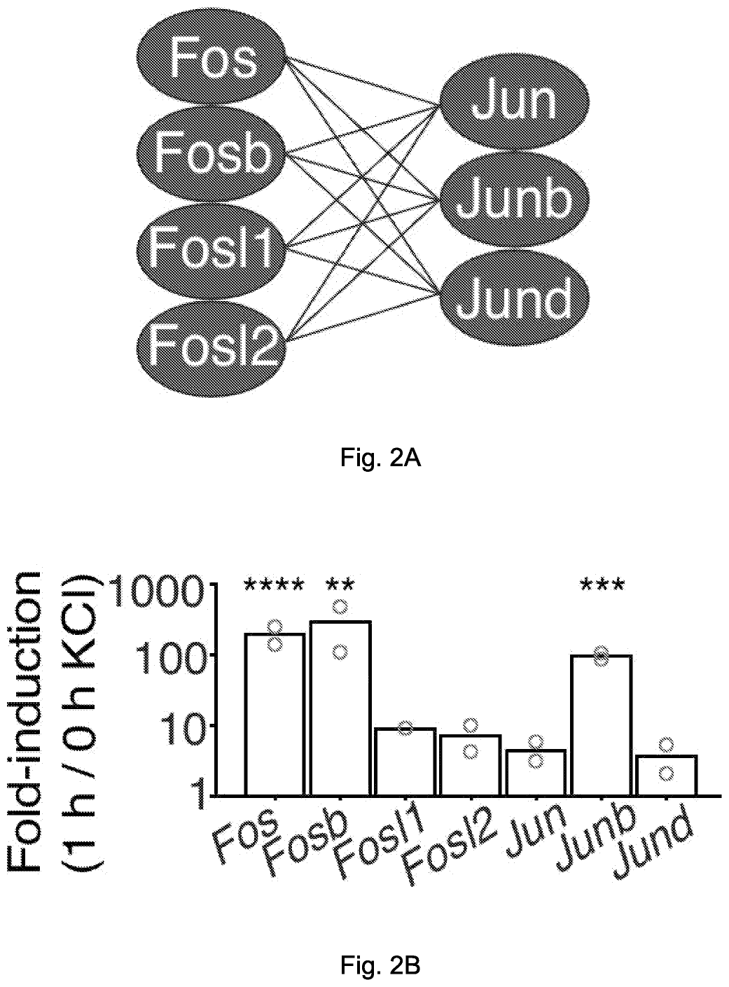

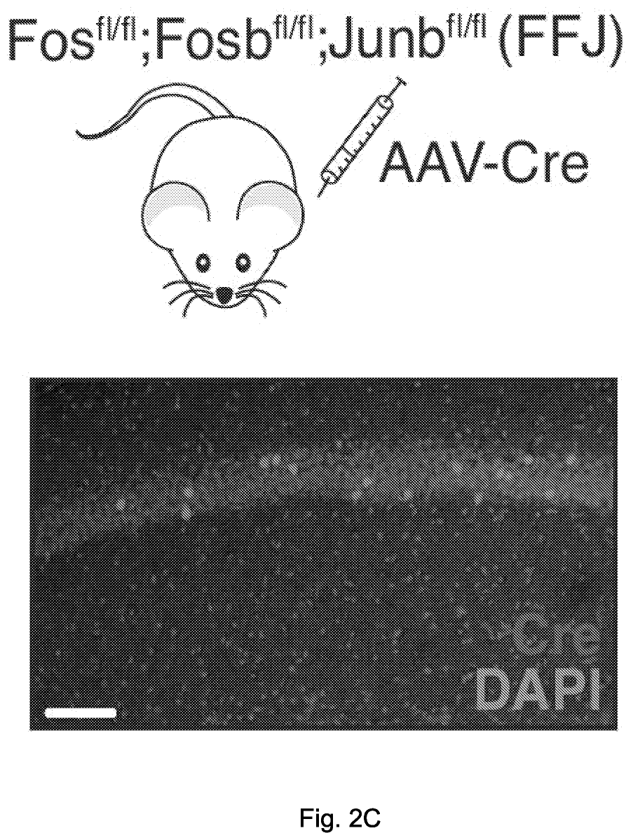

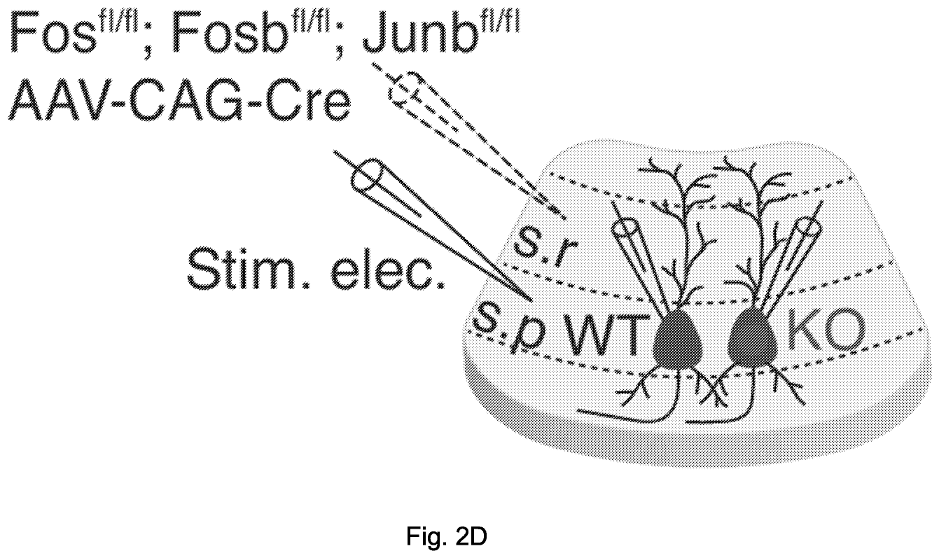

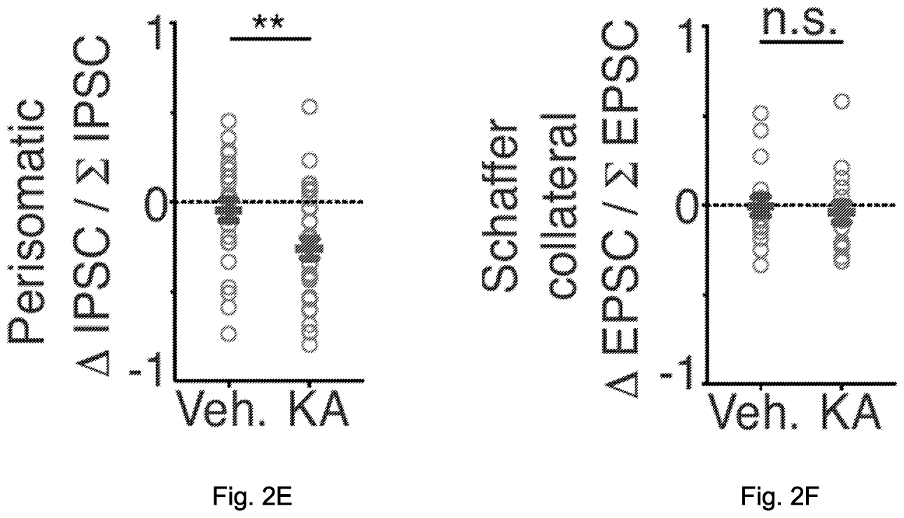

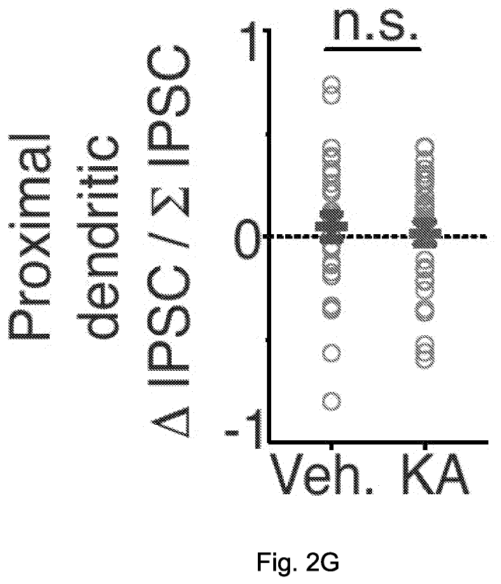

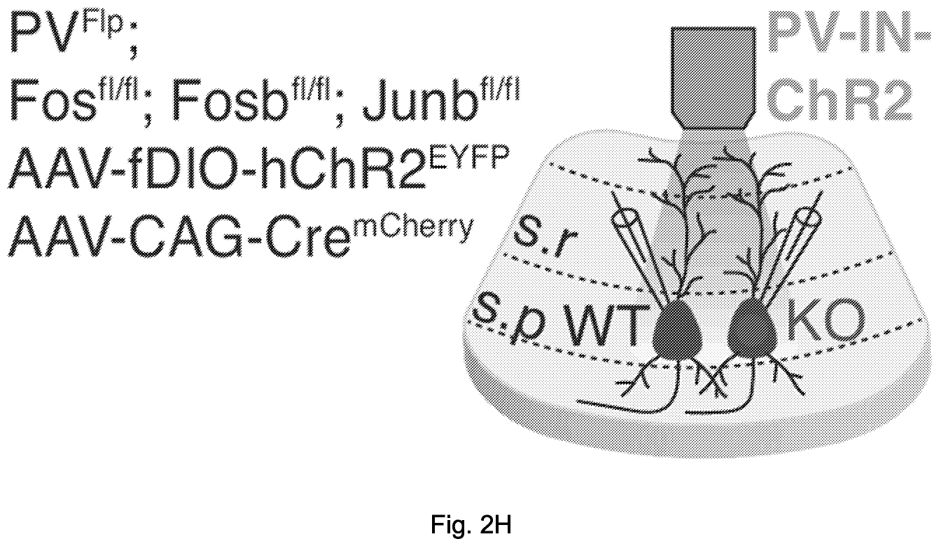

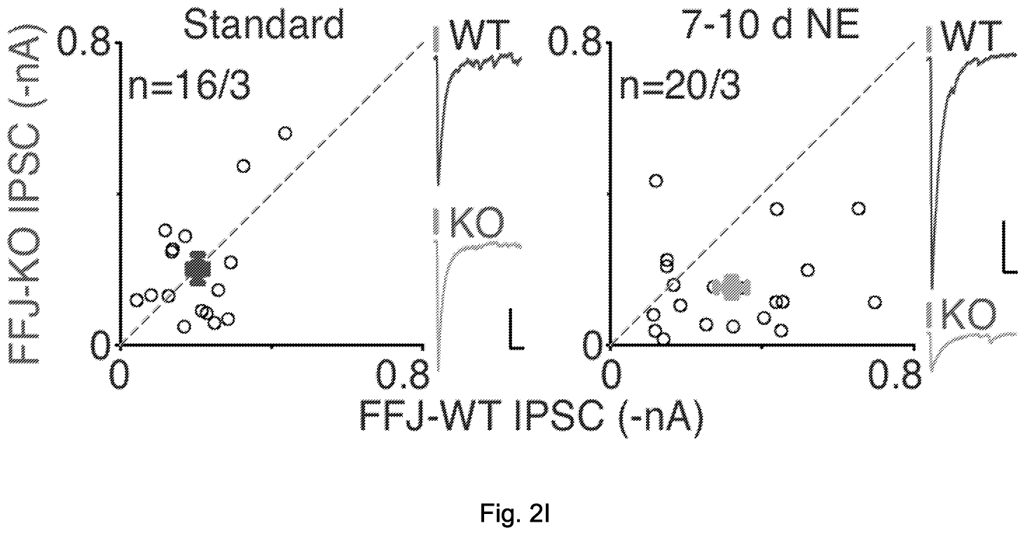

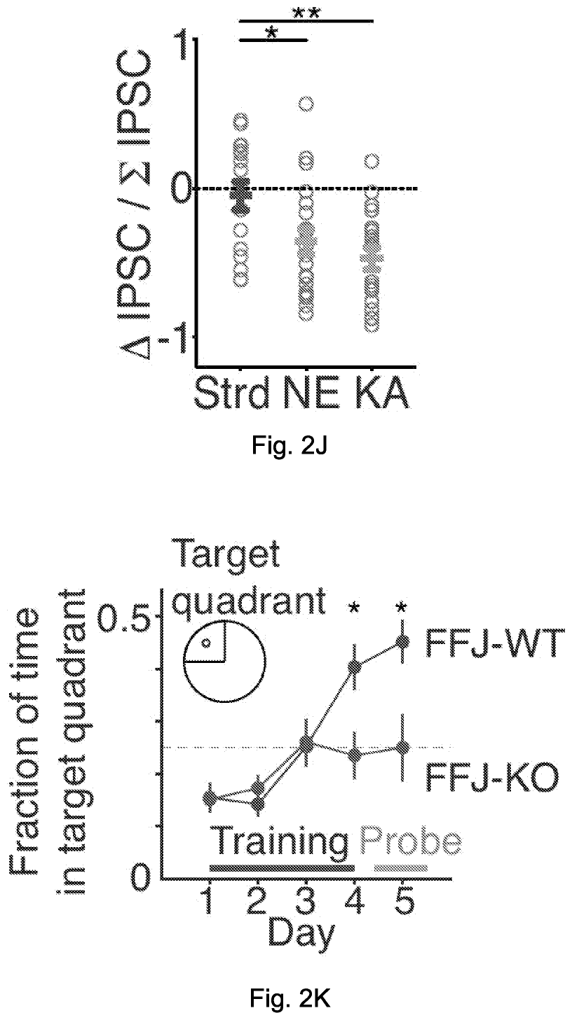

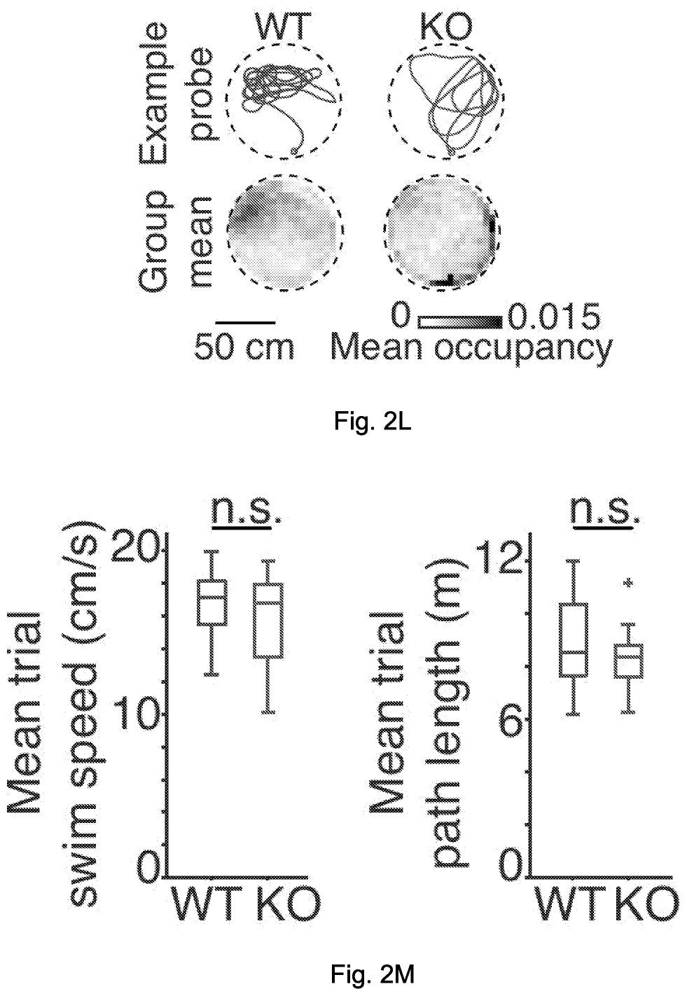

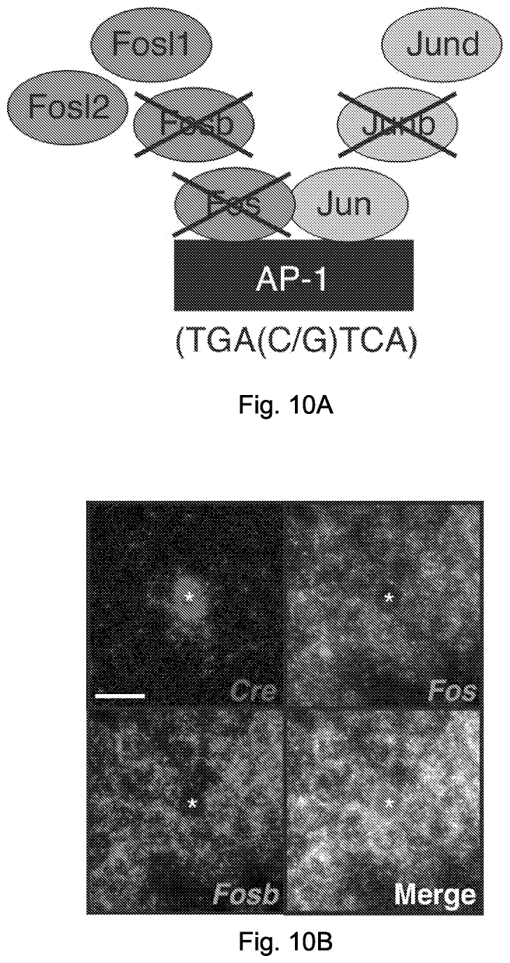

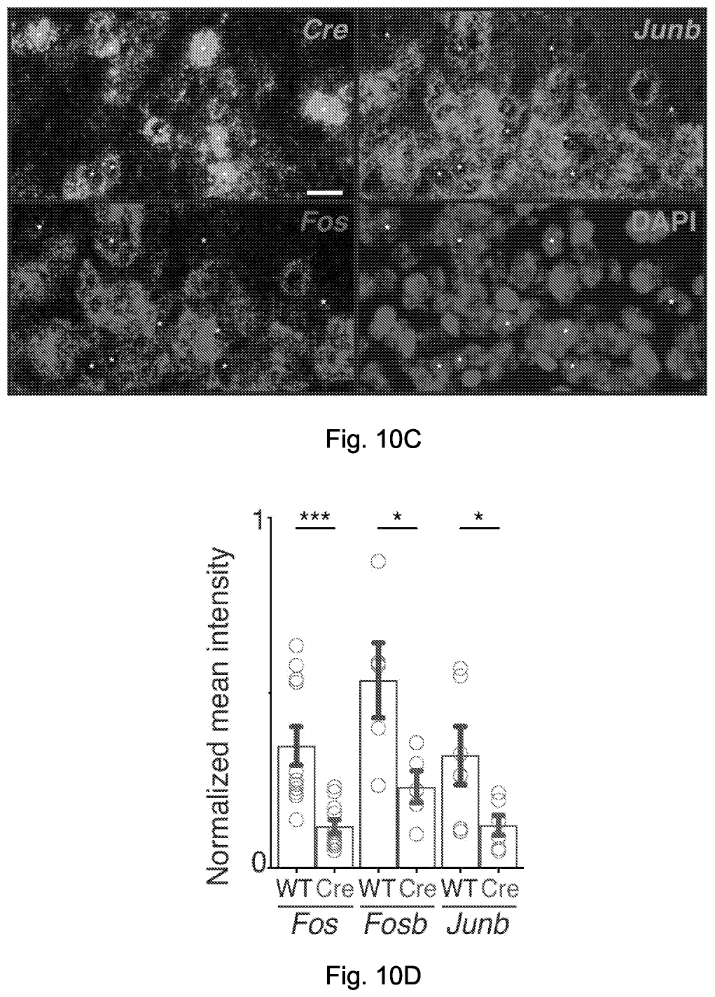

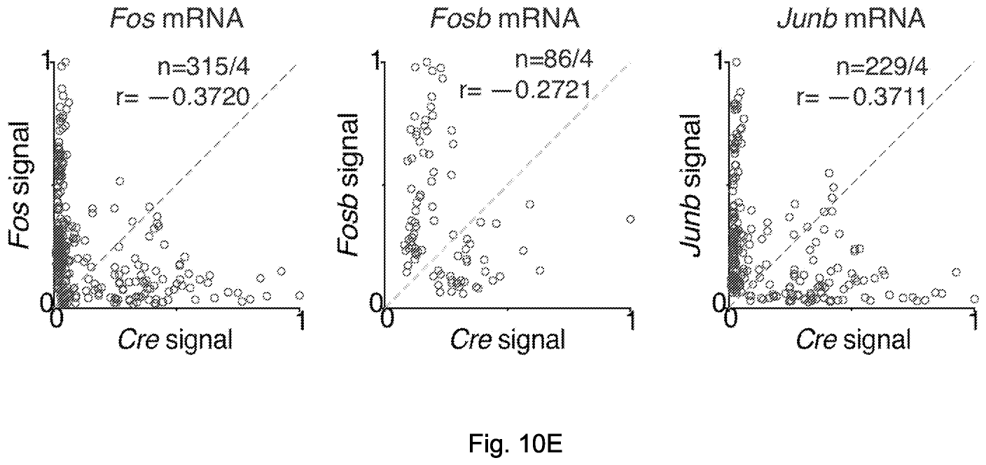

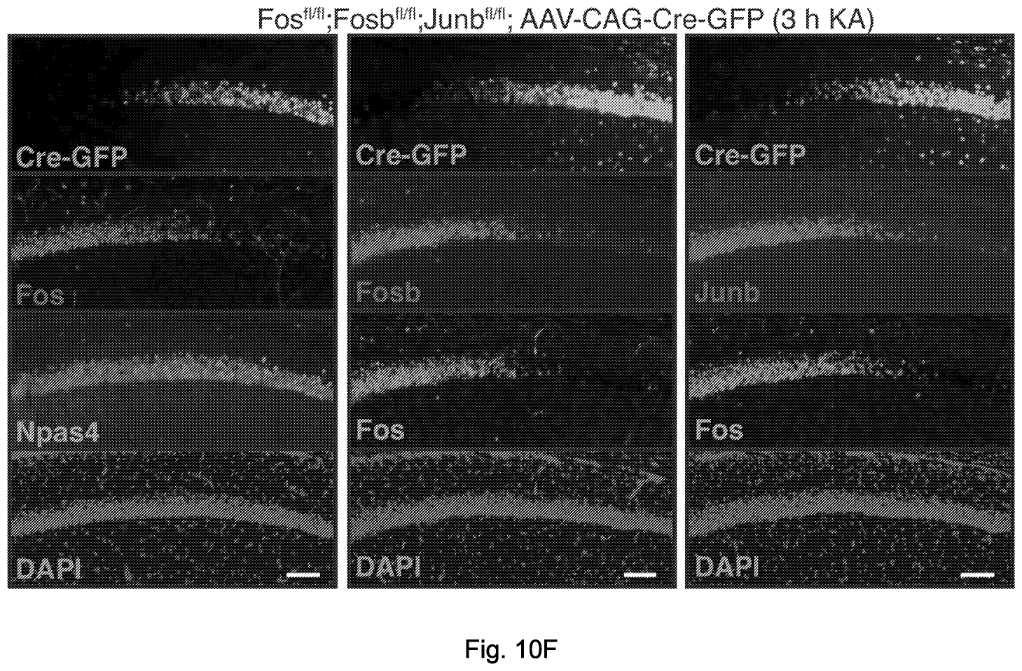

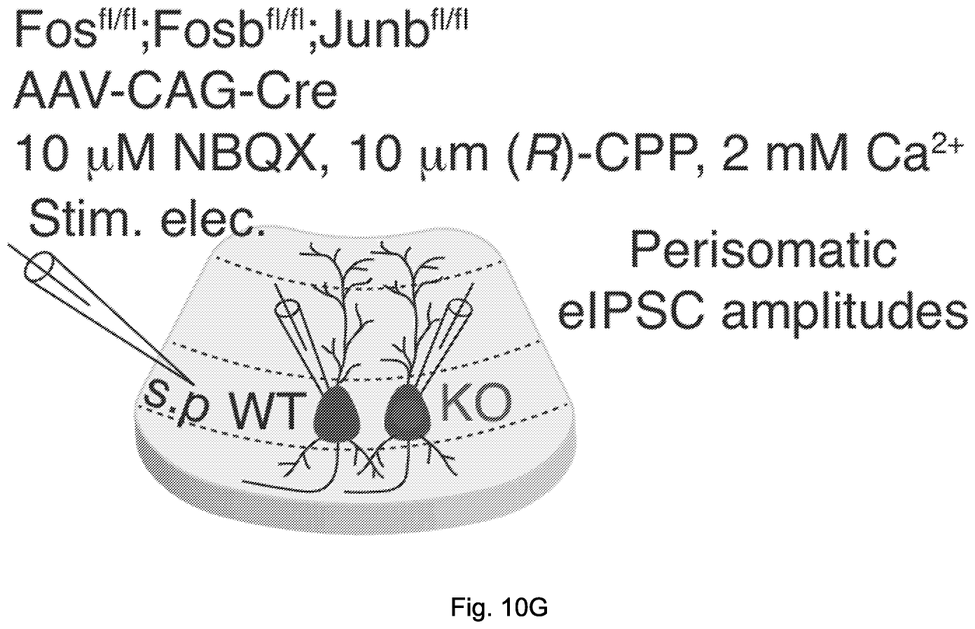

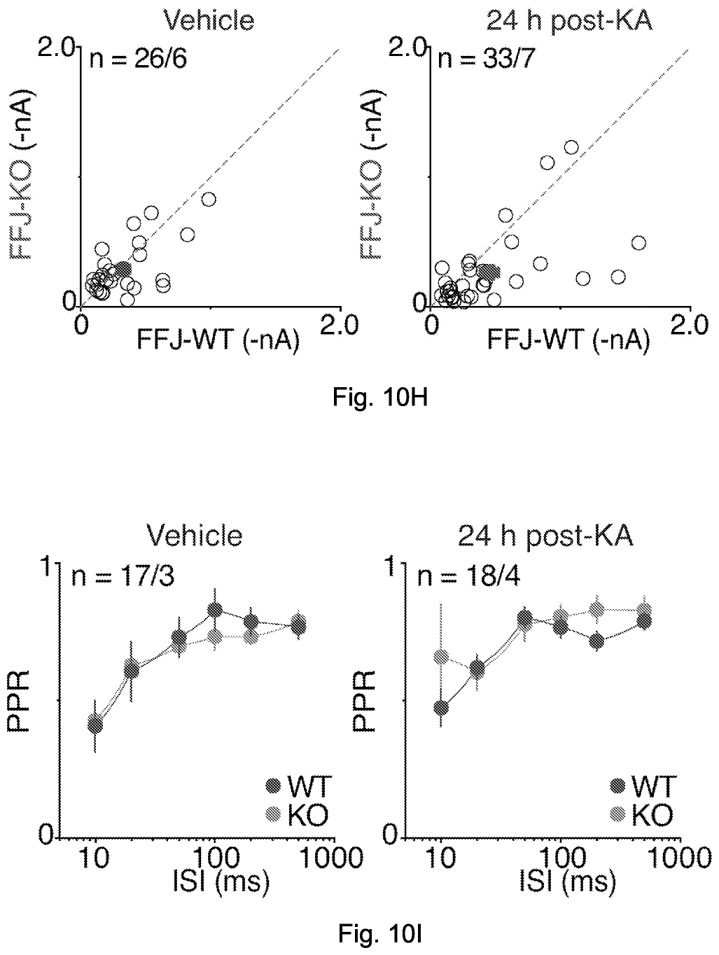

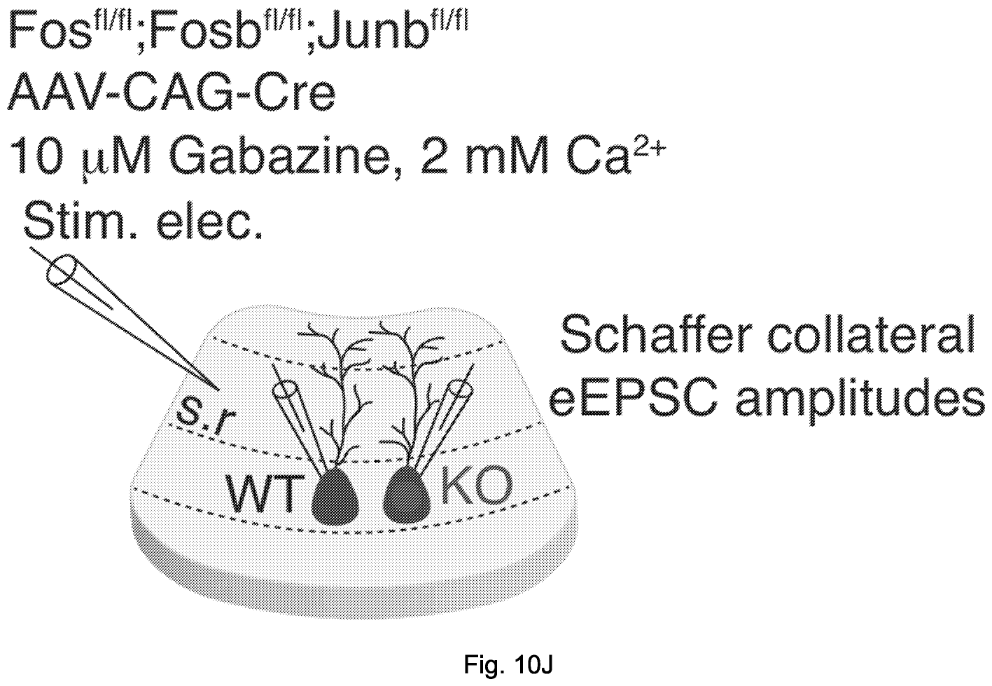

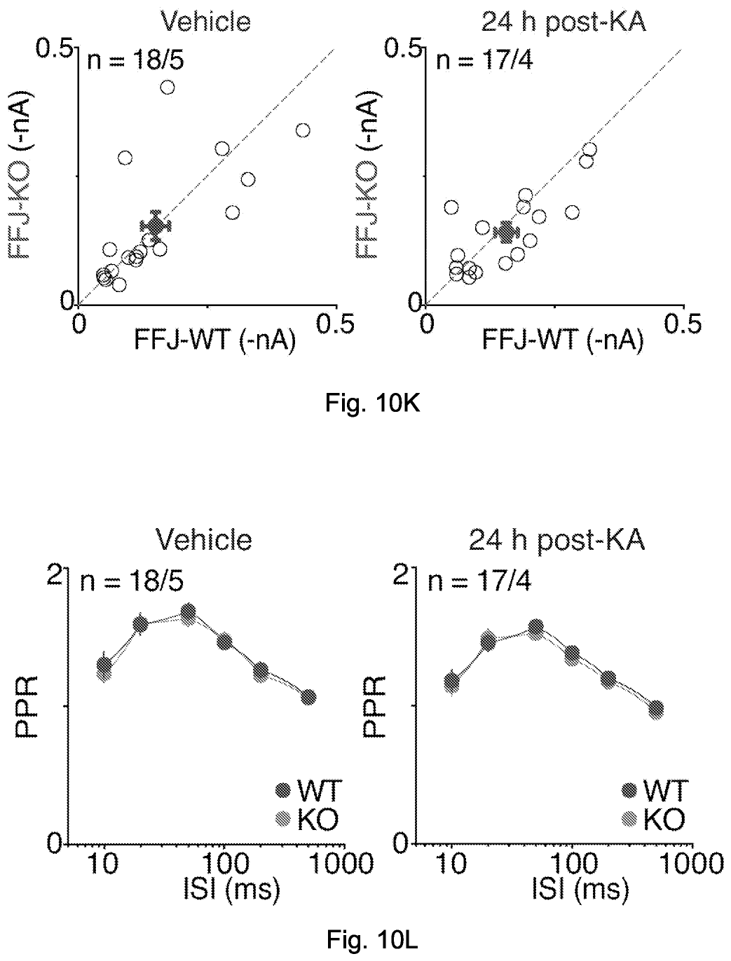

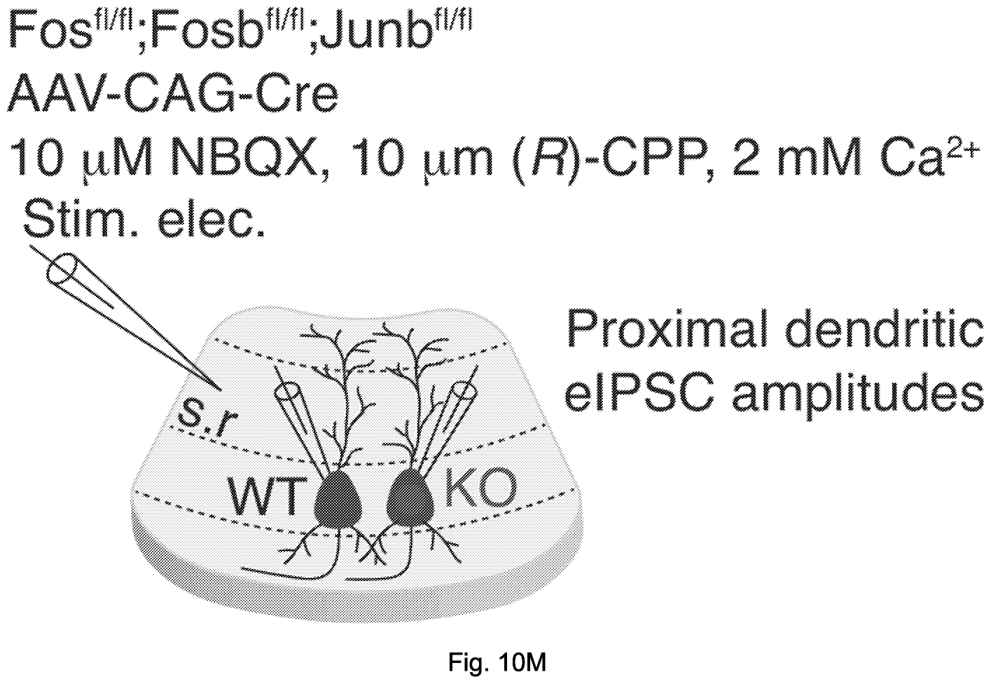

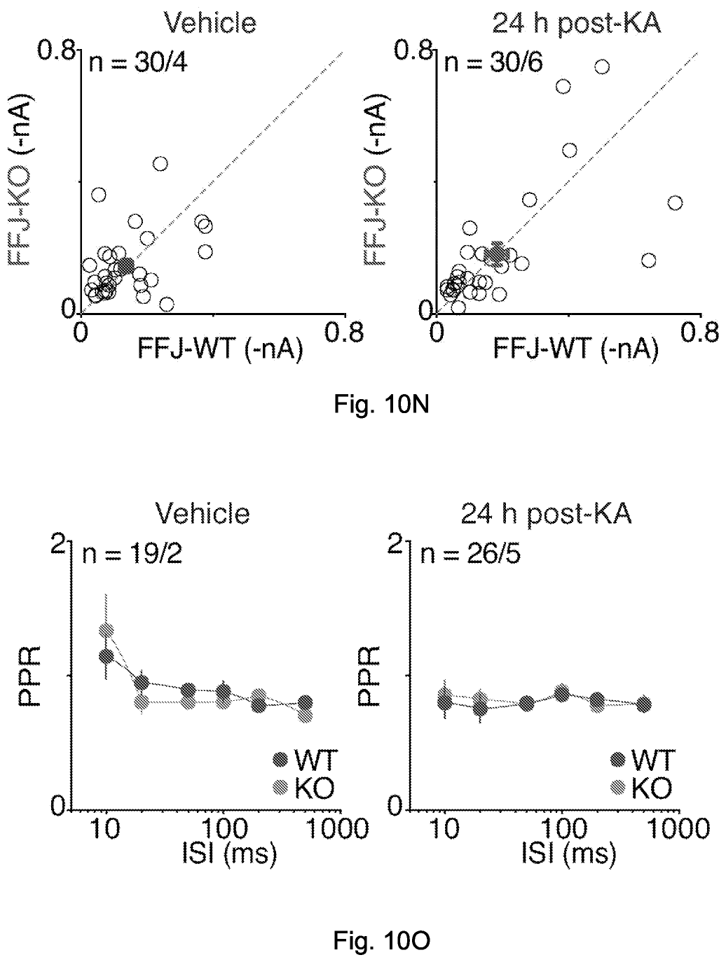

Since the induction of bidirectional perisomatic inhibitory plasticity occurs selectively onto Fos-expressing CA1 PCs, it was tested whether the Fos family of TFs, termed AP-1 factors, were mediating these changes. It was first determined which of the seven members were induced in the hippocampus by neuronal activity (see e.g., FIG. 2A). Fos, Fosb, and Junb were induced by approximately 100-fold or more in membrane-depolarized hippocampal cultured neurons, whereas the other four Fos family members were significantly less responsive (see e.g., FIG. 2B). A triple conditional knockout mouse line was therefore developed to permit the deletion of these strongly inducible AP-1 factors in a spatiotemporally-controlled manner (Fos^{fl/fl}; Fosb^{fl/fl}; Junb^{fl/fl}, also referred to herein as FFJ); the effective excision of these genes upon Cre expression in vivo was verified by single-molecule RNA fluorescence in situ hybridization (smRNA-FISH) and immunostaining for each of these three proteins (see e.g., FIG. 10A-FIG. 10F). See e.g., Vierbuchen et al. Mol Cell 68, 1067-1082 e1012 (2017), the content of which is incorporated herein by reference in its entirety.

Following sparse deletion of Fos, Fosb, and Junb mediated by AAV-expressing Cre (see e.g., FIG. 2C, FIG. 2D), dual whole-cell recordings were performed from FFJ-wildtype (FFJ-WT) and neighboring FFJ-knockout (FFJ-KO) CA1 PCs while electrically stimulating perisomatic inhibitory axons. There was a 1.7-fold decrease in pharmacologically-isolated evoked (eIPSC) amplitudes in FFJ-KO compared with FFJ-WT activated neurons (see e.g., FIG. 2E, FIG. 10G-FIG. 10I). By contrast, there was no significant differences in amplitudes of CA3 Schaffer collateral-evoked excitatory postsynaptic currents (eEPSCs) or proximal dendritic eIPSCs between FFJ-WT and FFJ-KO neurons under 24 h post-vehicle or KA conditions (see e.g., FIG. 2F, FIG. 2G, FIG. 10J-FIG. 10O). Therefore, AP-1 factors were specifically required for the regulation of perisomatic inhibition. In principle, AP-1 could also regulate Fos-activating CA1 PCs by modulating their CA3 excitatory inputs or inhibition from distinct compartments.

To directly measure PV-mediated inhibition, PV^{Flp/Flp}; FFJ mice were generated, which allowed for the expression of ChR2 specifically in PV-INs (see e.g., FIG. 2H). Simultaneous slice recordings of ChR2-evoked PV-IPSCs in FFJ-WT and neighboring FFJ-KO neurons revealed no differences in Strd housed mice (see e.g., FIG. 2I). By contrast, there was a significant decrease in PV-IPSC amplitudes onto FFJ-KO cells in mice after 7-10d NE, with 90% of FFJ-KO cells showing smaller IPSC amplitudes compared to the average for FFJ-WT cells (see e.g., FIG. 2I, FIG. 2J). These data indicate that AP-1 factors were required for the experience-dependent recruitment of PV-mediated inhibition, thus identifying their previously elusive role in long-term plasticity.

Given that loss of AP-1 leads to defects in inhibition, it was next tested whether spatial learning and memory were affected under these conditions. FFJ mice were bilaterally injected with AAV expressing Cre (FFJ-KO) or a catalytically inactive ΔCre (FFJ-WT) in the CA1 region and assessed in the Morris water maze paradigm. In contrast to FFJ-WTs, FFJ-KO mice performed significantly worse on this spatial task and were unable to learn the location of the platform in the maze (see e.g., FIG. 2K, FIG. 2L). There were no significant differences in mean swim speeds or path lengths between the two groups, indicating that there were no motor deficits in the FFJ-KOs (see e.g., FIG. 2M). These results indicate that changes in perisomatic inhibitory plasticity of Fos-activated neuronal networks can contribute to hippocampus-dependent spatial learning.

### Fos Targets in CA1 Pyramidal Neurons

Although many activity-regulated genes (ARGs) have been defined, difficulties in effectively disrupting AP-1 function in vivo have complicated the identification of genes that are specifically regulated by AP-1 factors and thus mediate the bidirectional modulation of perisomatic inhibition. The identification of AP-1 target genes has been further hampered by the pronounced neuronal cell-type-divergence of activity-dependent gene programs, and it is unclear how AP-1 factors, which are induced in nearly all cell types in the brain, contribute to this diversity. See e.g., Hrvatin et al. Nat Neurosci 21, 120-129 (2018), the content of which is incorporated herein by reference in its entirety. To address these challenges, a suite of genome-wide approaches was used to identify high-confidence AP-1 targets, focusing on CA1 PCs. The following genes were identified: 1) ARGs in CA1 PCs; 2) genes that showed reduced expression when AP-1 function was disrupted; and 3) genes that displayed activity-dependent Fos binding at nearby regulatory DNA elements. For these analyses, mice were treated with KA to strongly activate nearly all cells in CA1 and thus maximize the signal-to-noise ratio for identification of genes. AP-1 target(s) of interest identified by this method were subsequently validated under the more physiological condition of NE exposure.

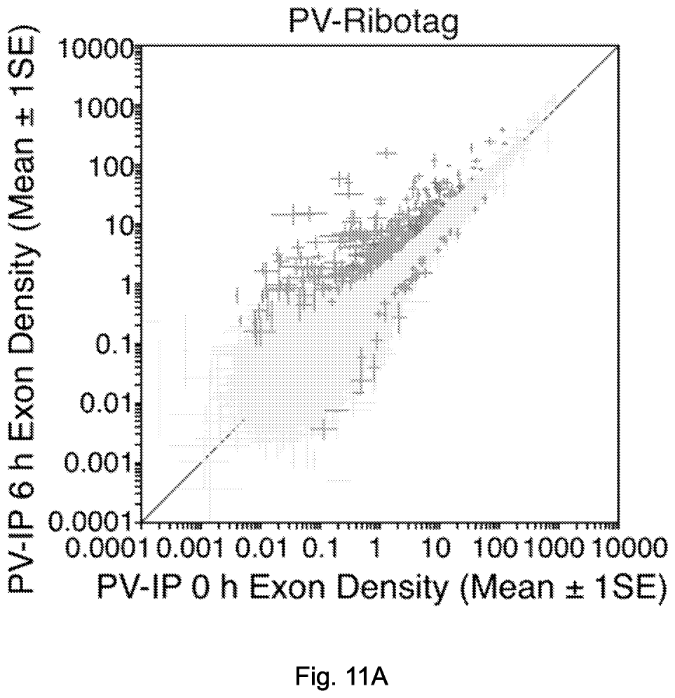

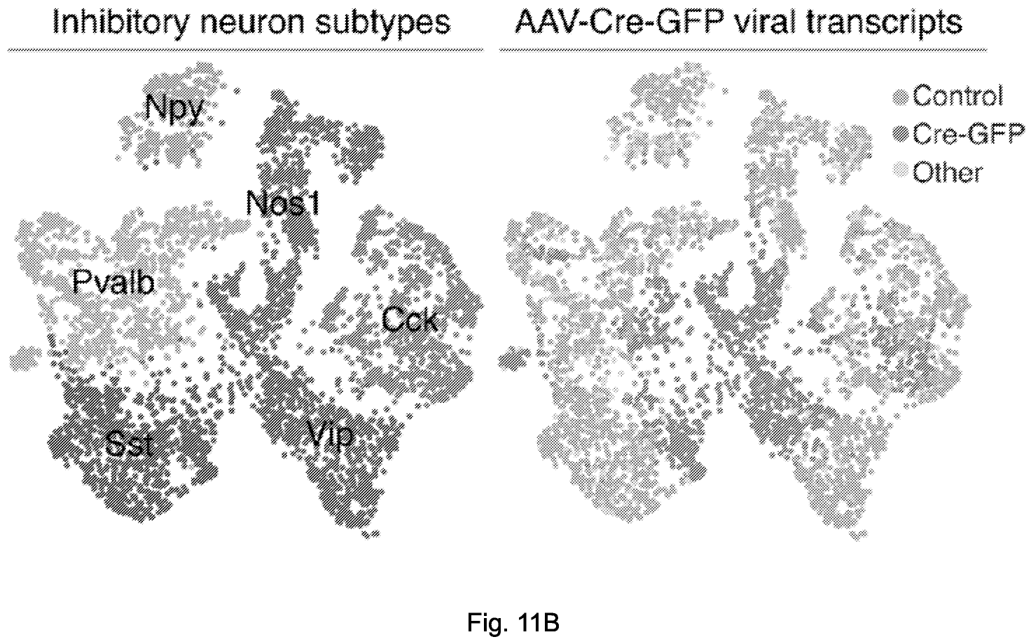

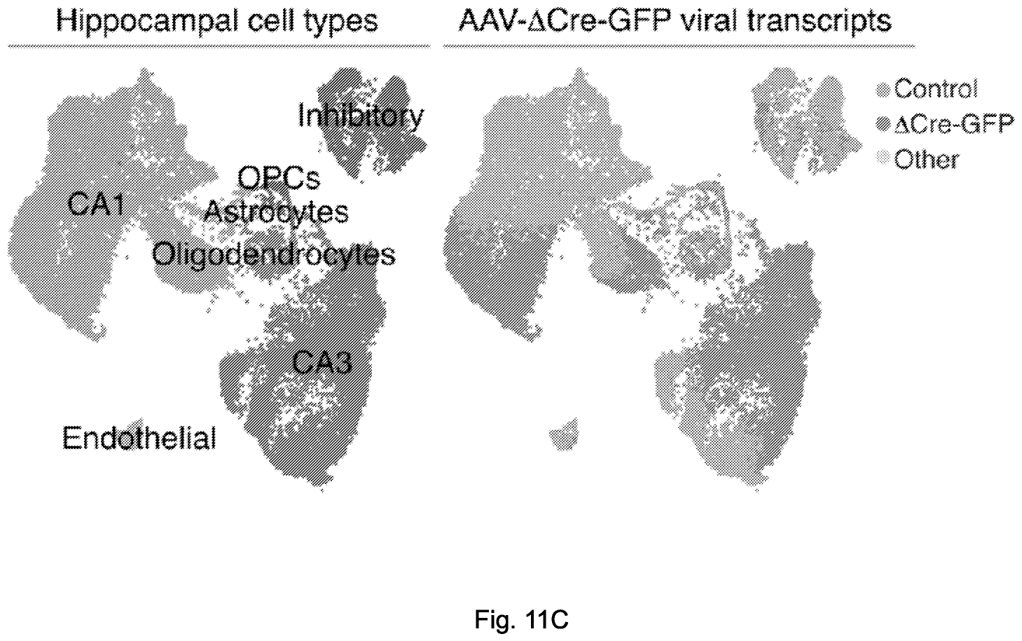

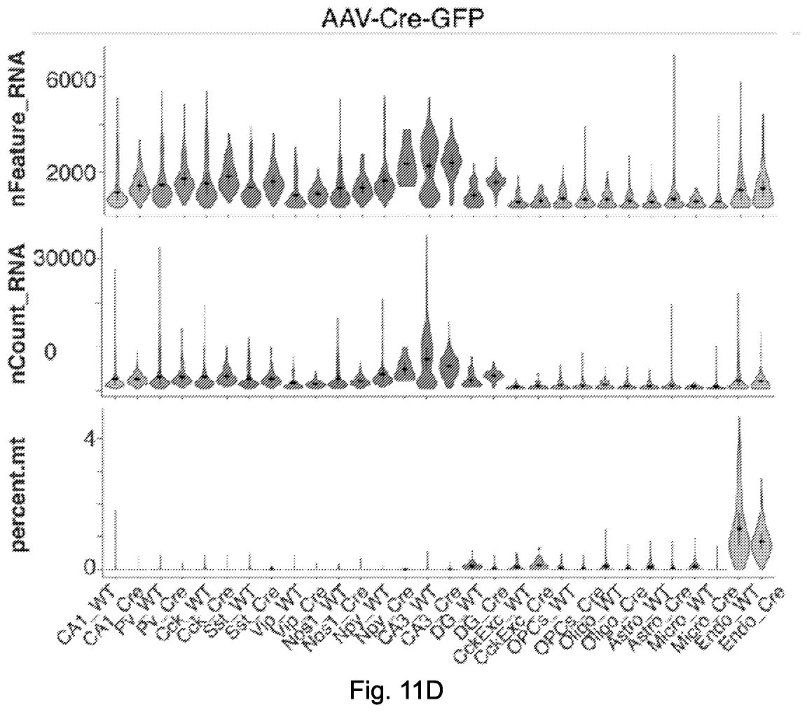

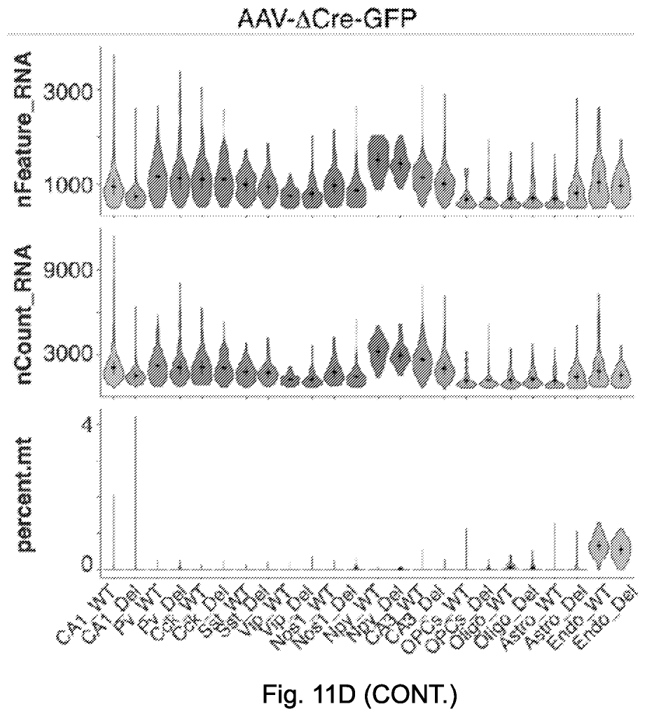

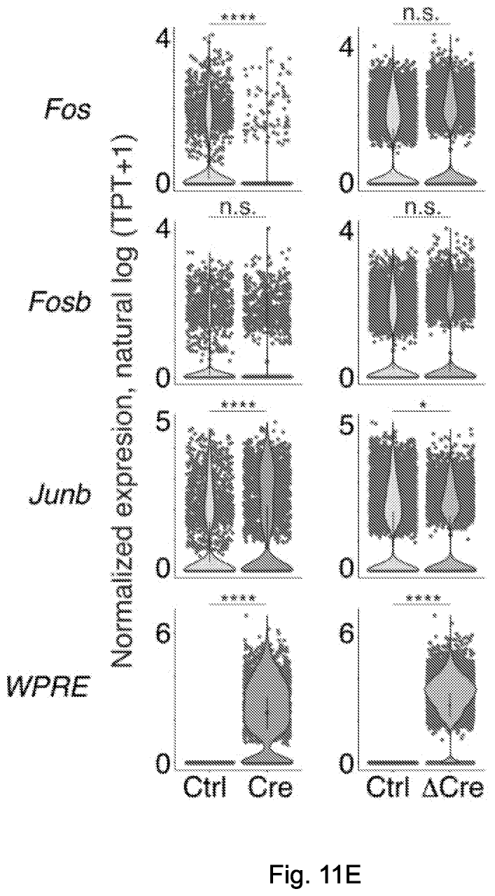

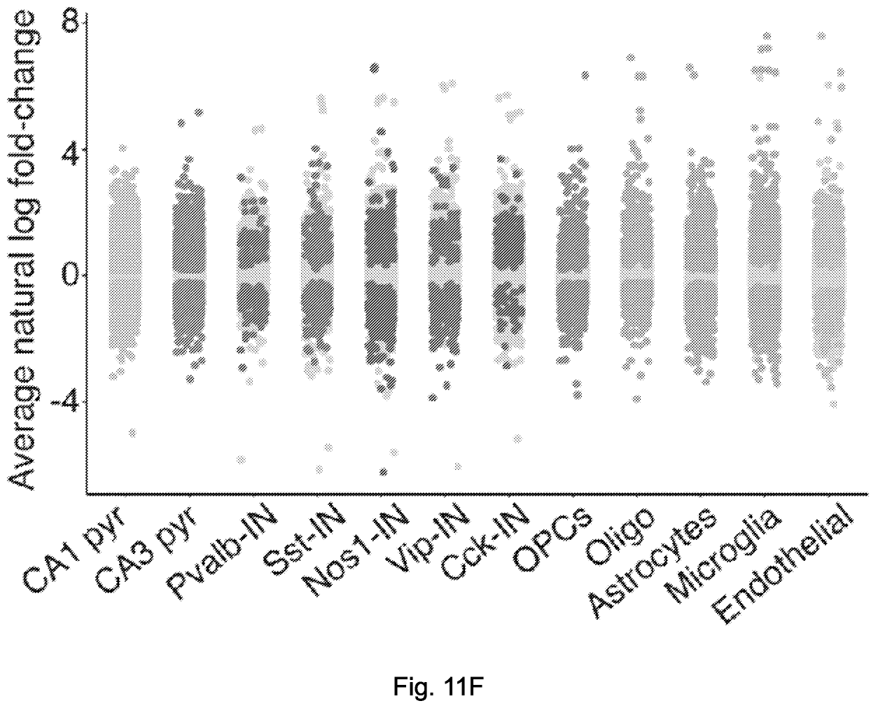

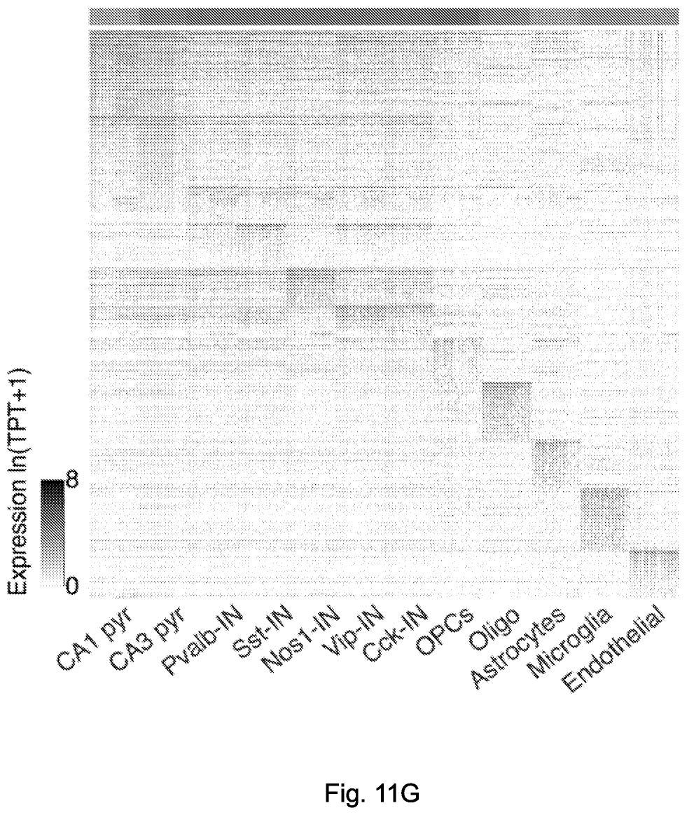

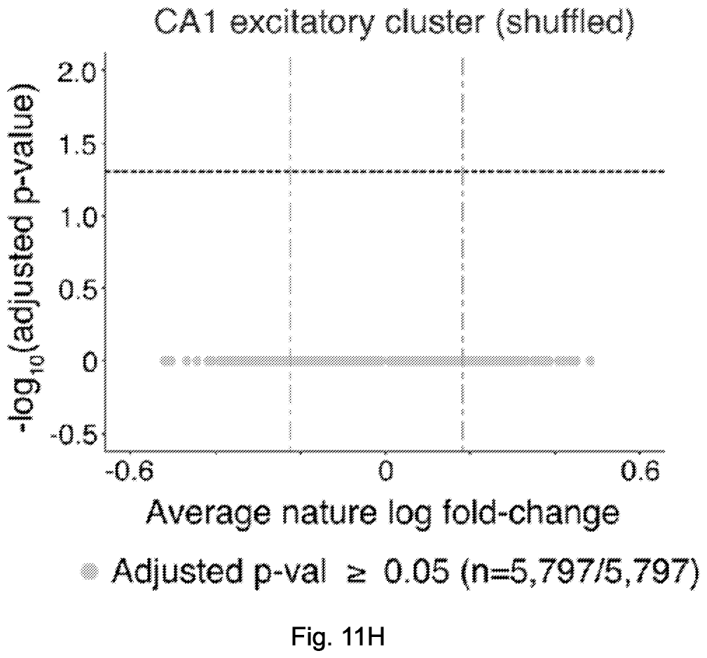

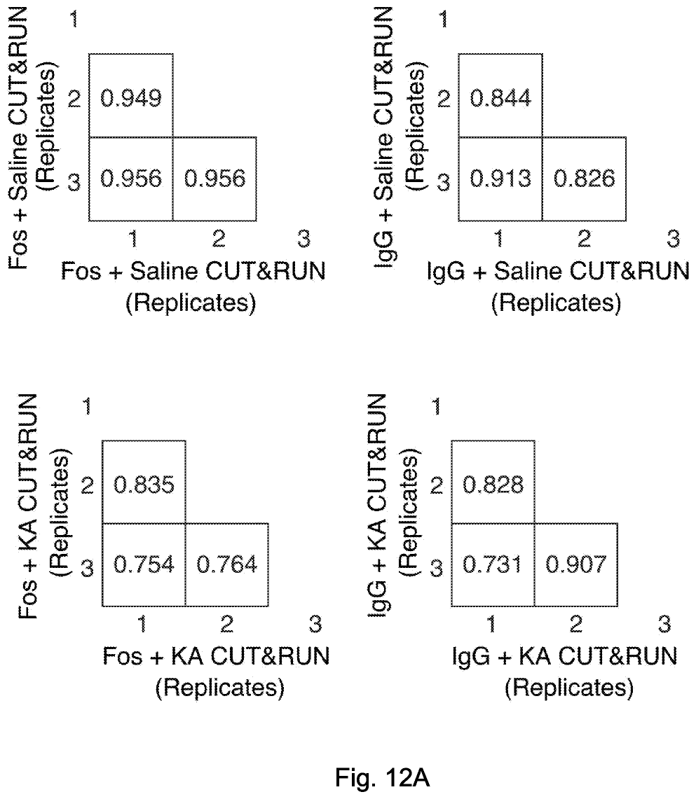

First, ARGs specific to CA1 PCs were defined by profiling ribosome-associated mRNAs (see e.g., FIG. 3A). Using CA1 tissue from CaMK2a^{Cre}; Rpl22-HA(RIBOTAG) mice treated with vehicle or KA for 6 hours, CaMK2a-specific ribosome-associated mRNAs were immunoprecipitated and sequenced; see e.g., Sanz et al. PNAS 106, 13939-13944 (2009), the content of which is incorporated herein by reference in its entirety. Analysis of differentially expressed genes (DGE) identified 795 ARGs induced by at least 2-fold (FDR 0.005), of which 111 were significantly enriched in CaMK2a-positive neurons relative to other cell types, including PV-INs (see e.g., FIG. 3B, FIG. 11A).

To determine which of these genes showed reduced expression when AP-1 function was disrupted, high-throughput single-nucleus RNA-sequencing (snRNA-seq) was performed using the FFJ mice. The mice were injected with AAV expressing Cre-GFP (Cre⁺) or ΔCre-GFP (ΔCre⁺) into one CA1 hemisphere, leaving cells in the contralateral hemisphere as untransduced controls. Mice were treated with KA for 4 hours, and CA1 nuclei were isolated and subsequently sorted using the 10× GENOMICS platform (see e.g., FIG. 3C). 83,750 single-cell transcriptomes isolated from 6 Cre⁺ and 4 ΔCre⁺ mice were sequenced (see e.g., FIG. 3D, FIG. 11B-FIG. 11E). Nuclei were clustered into 12-15 cell types using the Seurat single-cell analysis pipeline (see e.g., FIG. 3D). The presence of viral-derived transcripts was used to identify 17,027 Cre⁺ nuclei and 14,557 ΔCre⁺ nuclei. For each cell type, DGE analysis comparing Cre⁺ or ΔCre⁺ nuclei to their respective untransduced controls was used to identify AP-1-regulated genes, many of which were cell-type-specific (see e.g., FIG. 11F, FIG. 11G). These data indicate that AP-1 contributes to the cell-type-divergence of ARG expression. Specifically, within the CA1 excitatory neuron cluster, 697 genes were identified that were significantly downregulated by at least 20% in the absence of AP-1 (see e.g., FIG. 3E, FIG. 11E-FIG. 11H).

Finally, genes were identified that are direct targets of Fos in CA1 PCs using CUT&RUN, a chromatin profiling strategy in which in situ antibody-targeted controlled cleavage by micrococcal nuclease releases specific DNA complexes for sequencing (see e.g., FIG. 3F). CaMK2a-expressing CA1 nuclei from CaMK2a^{Cre}; LSL-Sun1-sfGFP-Myc mice were isolated via sorting based on Cre-dependent expression of the GFP-tagged inner nuclear membrane protein, Sun1. See e.g., Skene & Henikoff, Elife 6 (2017); Mo et al. Neuron 86, 1369-1384 (2015); the contents of each of which are incorporated herein by reference in their entireties. 3,295 Fos-bound activity-responsive loci were identified from mice exposed to 2-3 h KA as compared to vehicle treatment, with 1,109 genes containing at least one Fos-bound regulatory element within 10 kb of the transcription start site (TSS) (see e.g., FIG. 3G, FIG. 12A-FIG. 12F, FIG. 7A-FIG. 7E).

Intersection of the three datasets identified 17 genes that: (1) were inducible by activity in CA1 PCs (CaMK2a-RIBOTAG); (2) showed reduced expression with loss of AP-1 (FFJ snRNA-seq); and (3) bound Fos at nearby regulatory elements (CaMK2a-Sun1 Fos CUT&RUN). An additional 191 genes were present in two of the three datasets (see e.g., FIG. 3H, Table 1; see e.g., Yap et al., Nature, 2021, 590(7844):115-121, the content of which is incorporated herein by reference in its entirety). Further tests focused on the three high-confidence AP-1-regulated candidate genes that displayed high fold-induction and whose expression was enriched in CA1 PCs (Inhba, Bdnf, and Scg2) and three other genes shown to contribute to inhibitory plasticity that were present in two of the three genomic datasets (Rgs2, Nptx2, and Pcsk1) (see e.g., FIG. 12G-FIG. 12K, FIG. 13A); see e.g., Bloodgood et al. Nature 503, 121-125 (2013), the content of which is incorporated herein by reference in its entirety.

TABLE 1

| | FFJ snRNA | FFJ snRNA | CaMK2a-RIBOTAG |
|---|---|---|---|
| All 3 | and CaMK2a- | and CaMK2a- | and CaMK2a- |
| datasets | RIBOTAG | FosCUT&RUN | FosCUT&RUN |
| (17 genes) | (72 genes) | (46 genes) | (72 genes) |
| Inhba | 1190002N15Rik | Actr3 | Abhd2 |
| R3hdm1 | 3-Mar (Marchf3) | Atp2b1 | Ankrd55 |
| Lsm11 | 9530077C05Rik | Cadm2 | Arc |
| Scg2 | Acsl4 | Celf2 | Atf4 |
| Cgref1 | Adra1a | Ddit41 | Bag3 |
| Tmem2 | Arf2 | Dgkb | Blnk |
| Adpgk | Arid5b | Exoc5 | Blvrb |
| Itgav | Arpp19 | Fam19a1 | Brinp1 |
| Zfc3h1 | BC005561 | Gm9925 | Cap1 |
| Stmn4 | Cdh9 | Gpr85 | Conf |
| Klf6 | Cited2 | Gtpbp2 | Chml |
| Dpy19l3 | Cltb | Htr1b | Crybg3 |
| Bdnf | Dkk2 | Lrfn5 | Csrnp1 |
| Prosc | E330009J07Rik | Lrrc7 | Cyr61 |
| Lonrf1 | Fam126b | Lurap1l | Dnajb5 |
| Arpp21 | Fam65b | Mdh1 | Dusp1 |
| Spry2 | Fbxo33 | Mpp5 | Ednrb |
| | Fgfr1 | Nav3 | Efhd2 |
| | Frmd6 | Ndst3 | Fosl2 |
| | Gadd45g | Nebl | Gad1 |
| | Gmeb2 | Nectin3 | Gad2 |
| | Gne | Ociad2 | Gadd45b |
| | Gramd1b | Pkp4 | Gpr151 |
| | Hars | Plppr4 | Gpr176 |
| | Hmgcr | Rap2b | Gpx8 |
| | Hmgcs1 | Rbks | H2afz |
| | Ifrd1 | Rgs7bp | Homer1 |
| | Jarid2 | Rtn4 | Hspb6 |
| | Kitl | Serpini1 | Il1a |
| | Klf5 | Sgtb | Itpkc |
| | Lbh | Shc3 | Junb |
| | Lmo7 | Smap1 | Kdm3a |
| | Map3k5 | Sparcl1 | Kdm6b |
| | Med13 | St6galnac5 | Klf10 |
| | Mlip | Syt1 | Klf4 |
| | Mphosph10 | Thap2 | Maff |
| | Msmo1 | Tlk1 | Mapk4 |
| | Nfkbiz | Tmem38b | Myl12a |
| | Nts | Tomm70a | Nptx2 |
| | Nudt9 | Trim9 | Ovca2 |
| | Pam | Tspyl4 | Pdrg1 |
| | Pcdh8 | Tusc3 | Per2 |
| | Pcsk1 | Vamp4 | Pim1 |
| | Peli1 | Zbtb20 | Plekha2 |
| | Phlpp1 | Zfp281 | Ppp1r15b |
| | Piga | Zfr | Pvr |
| | Plat | | Pxdn |
| | Plk2 | | Rab33a |
| | Ppm1l | | Rasgef1b |
| | Prkar2a | | Rbm15 |
| | Ptgs2 | | Rcan1 |
| | Ptp4a2 | | Rgs2 |
| | Ptpn12 | | Rheb |
| | Rasa2 | | Rhoq |
| | Rbpj | | Rnf217 |
| | Rgs4 | | Rnh1 |
| | Rnd3 | | Serinc2 |
| | Rnf128 | | Sertad1 |
| | Sap30 | | Slc2a3 |
| | Sc5d | | Slc7a5 |
| | Sertm1 | | Sowahb |
| | Sgk1 | | Sox9 |
| | Snx1 | | Srxn1 |
| | Syt4 | | Sst |
| | Tmem47 | | Stk40 |
| | Tsc22d2 | | Tll1 |
| | Tspan9 | | Tnn |
| | Txnl1 | | Tpbg |
| | Uba6 | | Tra2a |
| | Uhrf2 | | Trib1 |
| | Wdfy1 | | Xirp1 |
| | Zufsp | | Zfp948 |

### Fos-Dependent Effector of Inhibition

To identify molecular effector(s) of bidirectional perisomatic inhibitory plasticity downstream of Fos activation, short hairpin RNA (shRNA)-mediated gene knockdown was used to determine if any of the six candidate genes mediate the activity-dependent strengthening of PV-mediated inhibition. After verifying the efficiency of knockdown in neurons (see e.g., FIG. 13B) and the absence of adverse effects on overall neuronal viability, individual shRNAs were cloned into a Flp-OFF AAV, allowing payload inactivation by Flp recombinase and the exclusion of shRNA expression in GABAergic INs when using Dlx5/6^{Flp} mice (see e.g., FIG. 4A, FIG. 4B, FIG. 13C, FIG. 8A).

Following sparse transduction of neurons, PV-IPSCs was simultaneously measured in neighboring pairs of shRNA-positive (mCherry⁺) and shRNA-negative (mCherry⁻) PCs by photostimulating PV-specific ChR2-expressing boutons in Dlx5/6^{Flp}; PV^{Cre} mice that had been treated with KA for 24 h (see e.g., FIG. 4B). There were no effects on amplitudes of PV-IPSC upon expression of a control scrambled shRNA or shRNAs against Inhba, Rgs2, Nptx2, or Pcsk1, and only a slight decrease with knockdown of Bdnf (see e.g., FIG. 4C, FIG. 13D); see e.g., Hensch, Cell 156, 17-19 (2014), the content of which is incorporated herein by reference in its entirety. By contrast, PV-mediated inhibition was significantly decreased by either of two independent shRNAs against Scg2 (see e.g., FIG. 4C, FIG. 4D, FIG. 13E). Similar results were observed following more the physiological condition of NE exposure (see e.g., FIG. 4E, FIG. 13F), indicating a prominent role for CA1 PC-derived Scg2 in the long-term regulation of PV-mediated inhibition.

Scg2 has been shown to be activity-regulated and to encode a neuropeptide precursor that undergoes endoproteolytic processing by Pcsk1/2 proteases to produce four distinct, non-overlapping neuropeptides: Secretoneurin, EM66, Manserin, and SgII (see e.g., FIG. 4F); however, the functions of these peptides in the brain are largely unknown. See e.g., Nedivi et al. Nature 363, 718-722 (1993); Fischer-Colbrie et al. Prog Neurobiol 46, 49-70 (1995); the contents of each of which are incorporated herein by reference in their entireties. Scg2 was highly enriched in CA1 PCs (see e.g., FIG. 4G), significantly downregulated upon AP-1 loss (see e.g., FIG. 4H), and associated with several Fos-bound regulatory elements (see e.g., FIG. 4I).

To test whether Scg2 was expressed in the CA1 in an experience-dependent manner, smRNA-FISH was performed using mice exposed to 6 h NE compared to Strd, probing for mature Fos and Scg2 RNA, as well as nascent intron-containing Scg2 transcripts (see e.g., FIG. 4J). Fos and Scg2 showed correlated expression (see e.g., FIG. 13G, FIG. 13H), with both genes significantly induced following NE (see e.g., FIG. 4K). A brief (5-min) NE exposure was sufficient to induce Fos and Scg2 in CA1 PCs when assessed by snRNA-seq 1-h or 6-h after the exposure (see e.g., FIG. 4L).

### Scg2 Regulated PV and CCK Inhibition

To investigate further the requirement of Scg2 for bidirectional perisomatic inhibitory plasticity, an Scg2 conditional knockout mouse line was generated and verified (Scg2^{fl/fl}; see e.g., FIG. 5A, FIG. 5B, FIG. 14A). These mice were crossed with PV^{Flp} mice. The resulting PV^{Flp/Flp}; Scg2^{fl/fl} mice were sparsely transduced with AAV expressing Cre and co-injected with the AAV activity reporter mKate2 (see e.g., FIG. 1B) and a separate Flp-dependent AAV to localize ChR2 expression to PV-INs (see e.g., FIG. 5C). These mice were then exposed to 2-3d NE and subsequently light-evoked PV-IPSC amplitudes were recorded simultaneously in neighboring Fos-activated neurons that were Cre-positive (Scg2-KO Cre⁺/mKate2⁺) or Cre-negative (Scg2-WT Cre⁻/mKate2⁺) (see e.g., FIG. 5C). Consistent with the data obtained by shRNA-mediated knockdown of Scg2, amplitudes of PV-IPSCs in Fos-activated Scg2-KO neurons were on average 3-fold smaller compared with those in Scg2-WT neurons (see e.g., FIG. 5D, FIG. 5E). This effect was not observed in non-Fos-activated (mKate2⁻) neurons in either Strd or NE (see e.g., FIG. 5D, FIG. 5E). Thus, Fos-activated CA1 PCs required Scg2 to induce plasticity of PV-IN synapses.

It was next investigated whether Scg2 also regulated CCK-mediated inhibition. Owing to the lack of a CCK-IN-only Flp-driver, two orthogonal approaches were used to measure CCK-IPSCs. First, a pharmacological strategy was employed in which CCK-IPSCs were specifically measured by blocking PV-IPSCs using ω-agatoxin IVA; see e.g., Freund & Katona (2007), supra; Hefft & Jonas (2005), supra. Simultaneous recordings from pairs of Scg2-WT (Cre⁻/mKate2⁺) and KO (Cre⁺/mKate2⁺) neurons after 2-3d NE exposure showed that the mean amplitude of CCK-IPSCs in Scg2-KO neurons was 2-fold larger than that in Scg2-WT neurons, specifically upon Fos activation (see e.g., FIG. 5F-FIG. 5H). Similar results were obtained with an independent approach involving an intersectional genetic strategy using Dlx5/6^{Flp}; CCK^{Cre} mice in conjunction with shRNA-mediated knockdown of Scg2 (see e.g., FIG. 14B-FIG. 14F). Thus, a single experience-regulated AP-1 target, Scg2, couples the bidirectional regulation of PV-mediated inhibition and CCK-mediated inhibition onto Fos-activated neurons.

These findings were further corroborated through a series of rescue and overexpression experiments. Notably, the defects in both PV-mediated inhibition and CCK-mediated inhibition were restored to control levels when Scg2 was exogenously expressed in shRNA-mediated knockdown or Scg2^{fl/fl} knockout conditions (see e.g., FIG. 5I, FIG. 14G, FIG. 15A-FIG. 15D). In addition, amplitudes of light-evoked PV-IPSCs or CCK-IPSC were compared in Scg2-overexpressing (Scg2-OE) and neighboring control (Scg2-WT) neurons; gain-of-function of Scg2 was sufficient to strengthen PV-mediated inhibition and weaken CCK-mediated inhibition, respectively, in the absence of neural activity (see e.g., FIG. 5J, FIG. 15E, FIG. 15F).

Cleavage of the Scg2 precursor gives rise to multiple neuropeptides with distinct functions (see e.g., FIG. 4F). Given that Scg2 cleavage is directed by a series of internal dibasic residues, a cleavage-resistant form of Scg2 was generated in which the nine dibasic sequences were mutated to alanine (9AA-Mut). It was first verified that these sequence changes do not affect Scg2 expression levels (see e.g., FIG. 15G, FIG. 8B). Expression of this cleavage-deficient Scg2 did not recapitulate the effects of overexpressing wildtype Scg2 (see e.g., FIG. 5K, FIG. 15H, FIG. 15I) or rescue the effects of loss of Scg2 (see e.g., FIG. 5I, FIG. 15C, FIG. 15D). Thus, these results indicate that the processing of Scg2 precursor protein to mature peptides can be required for experience-dependent bidirectional perisomatic inhibitory plasticity; thus, distinct Scg2-derived peptides can coordinate aspects of bidirectional plasticity.

### Scg2 was Crucial for Network Rhythms In Vivo

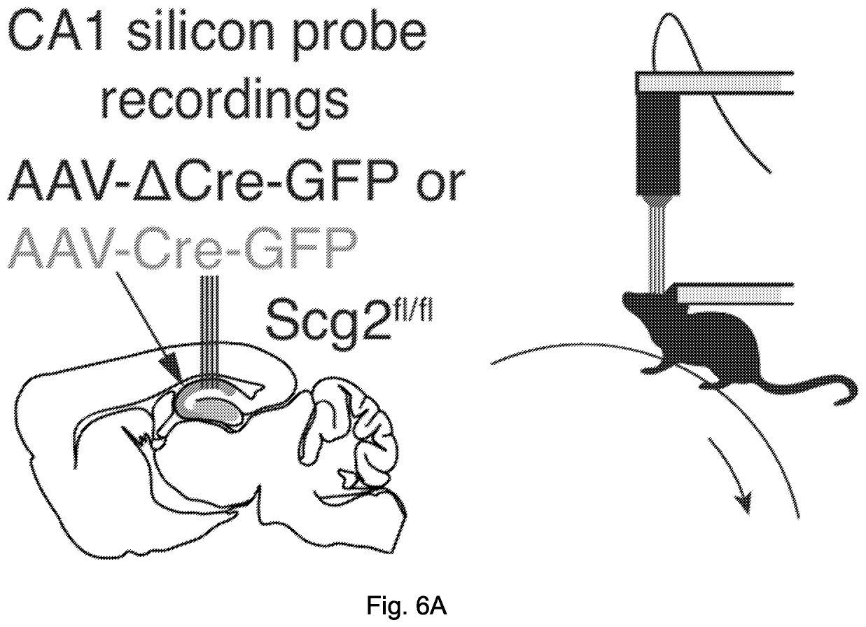

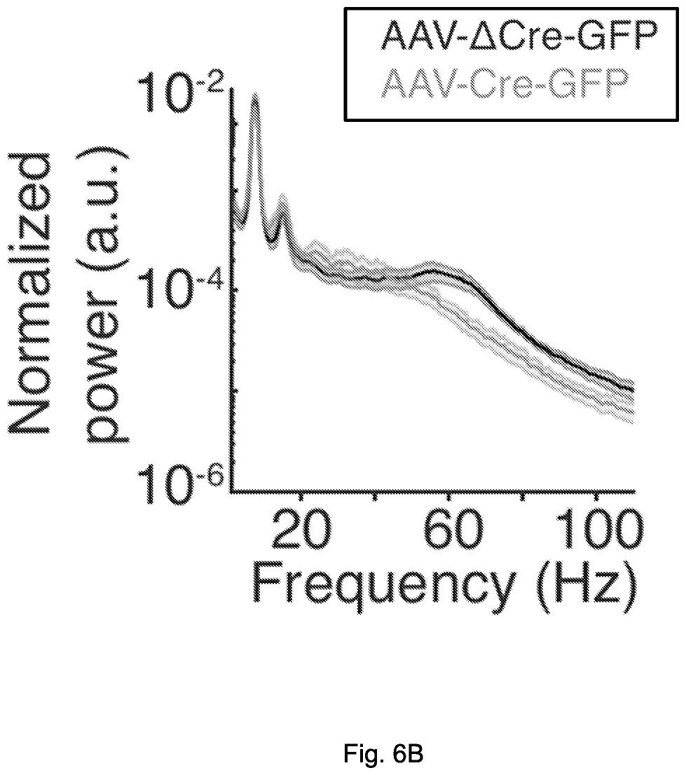

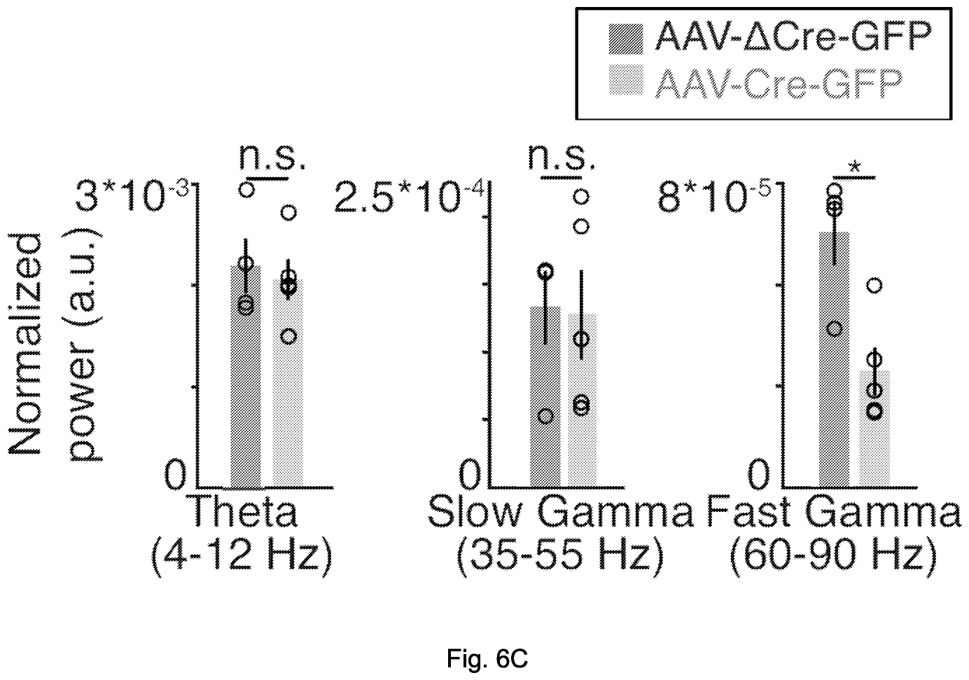

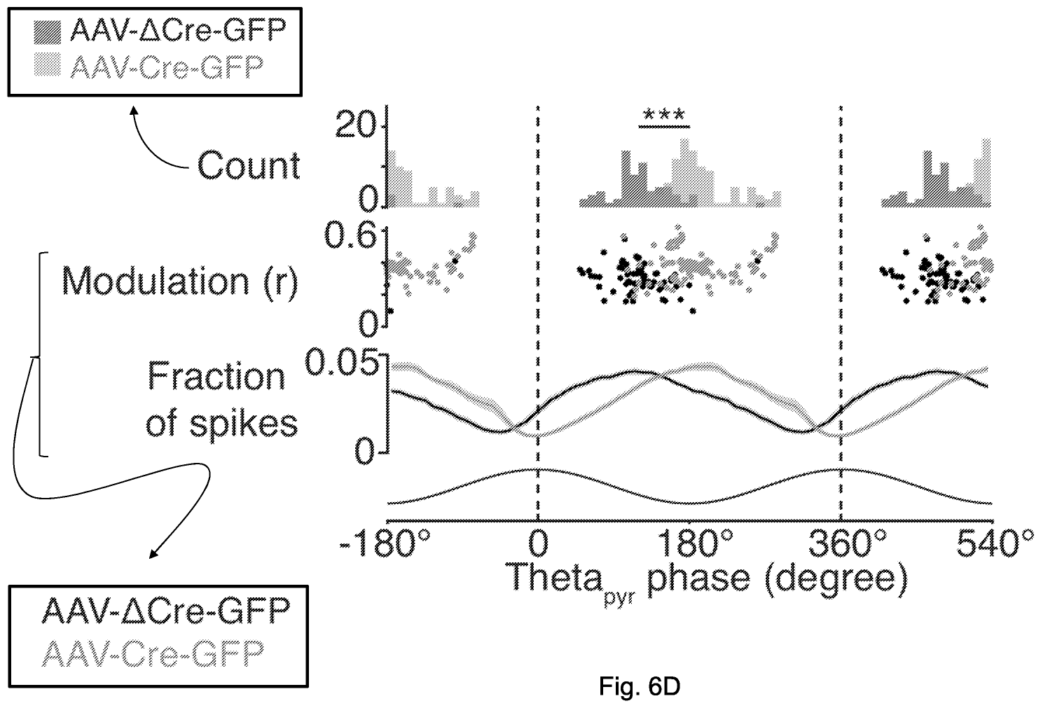

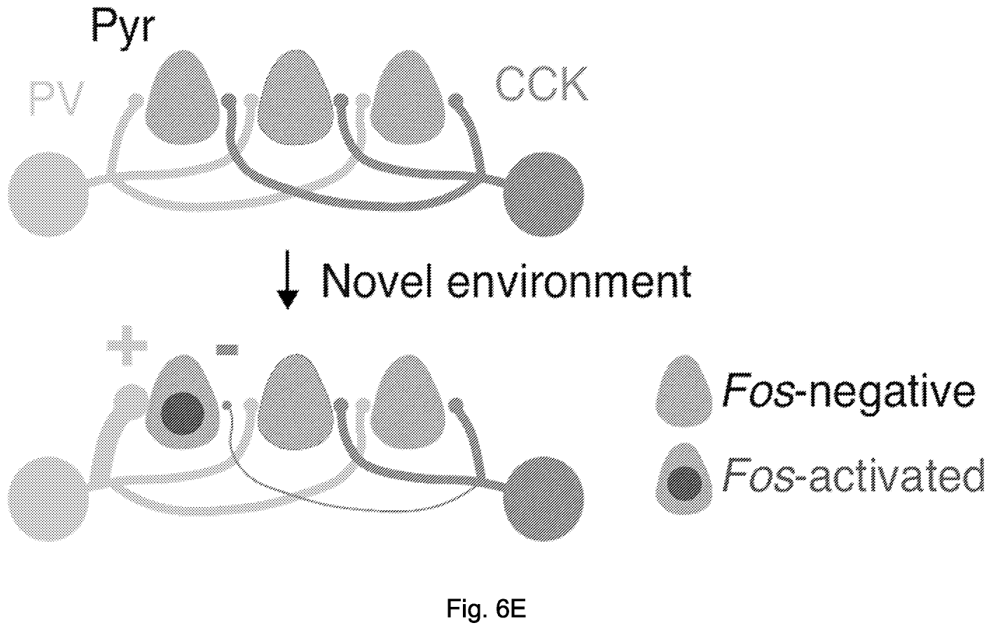

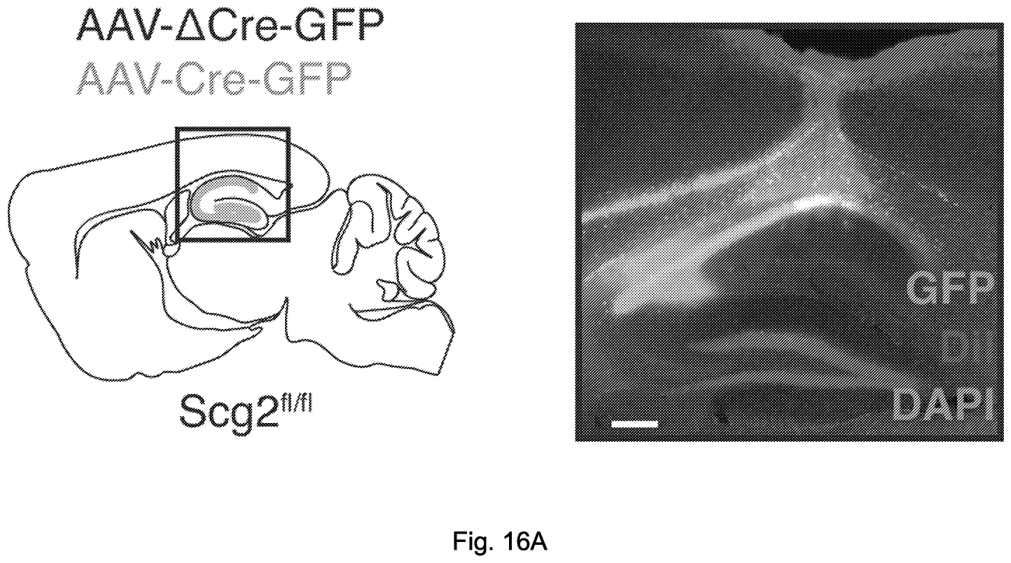

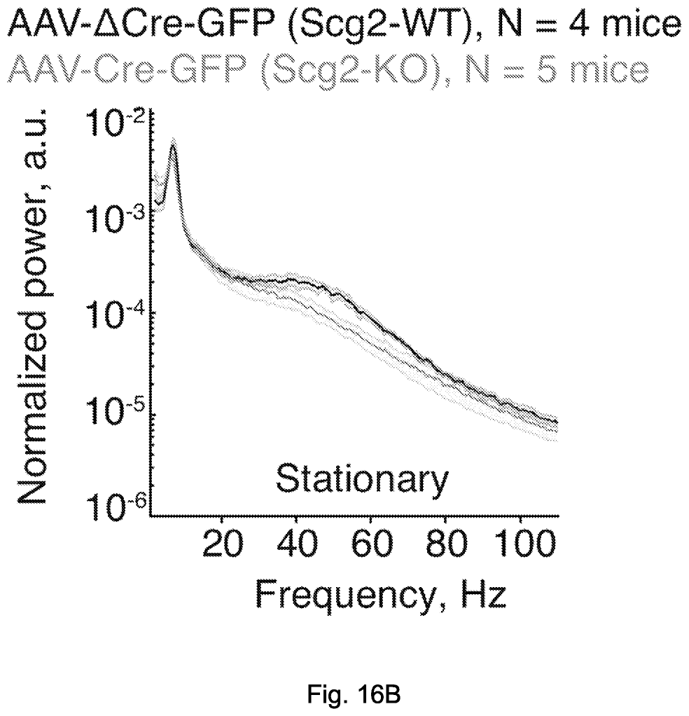

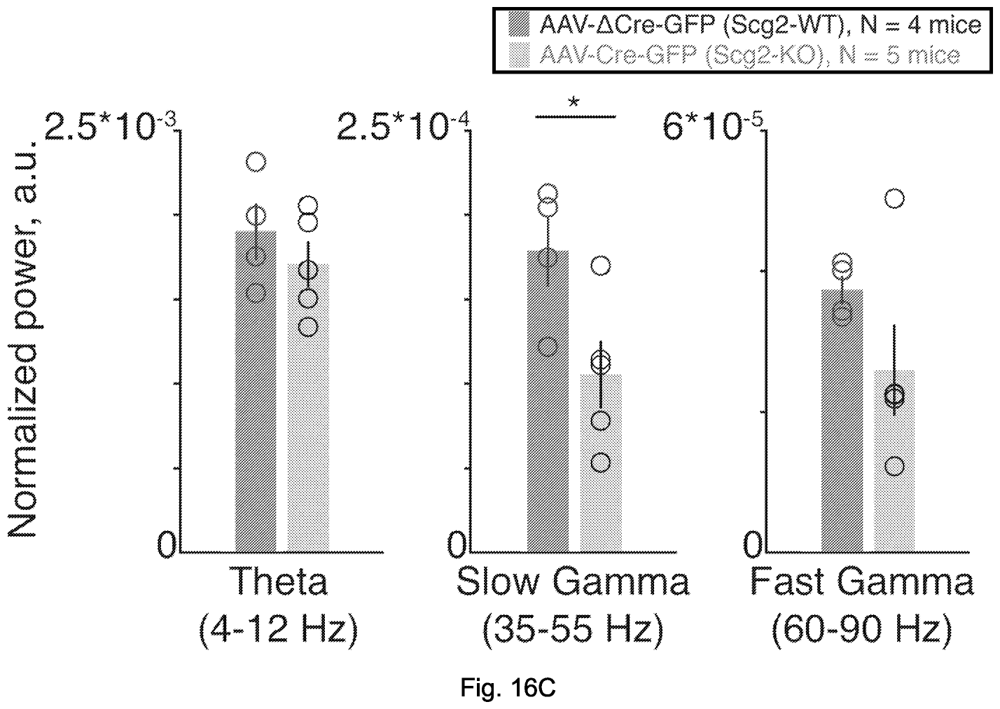

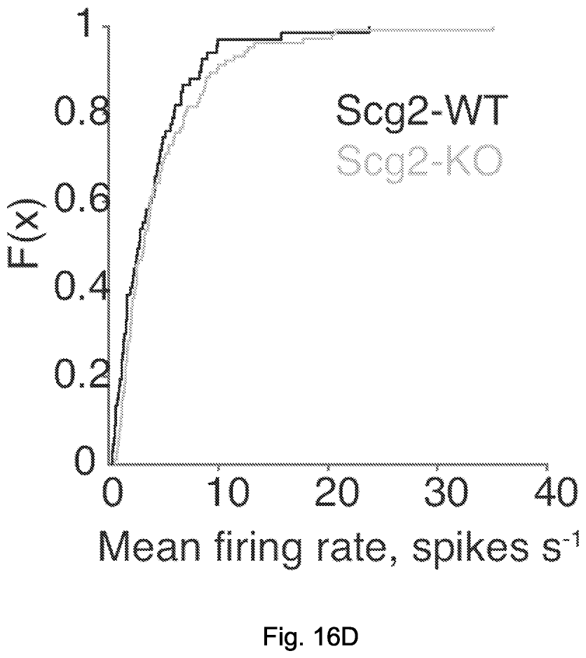

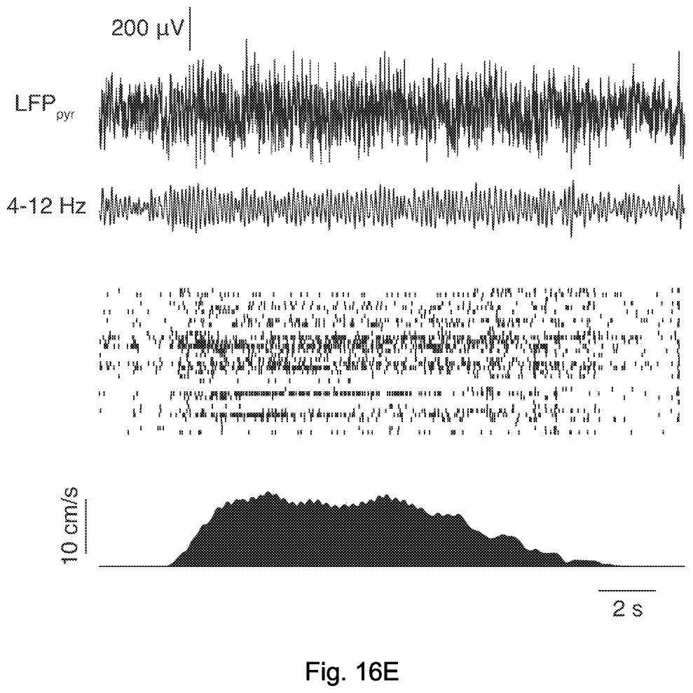

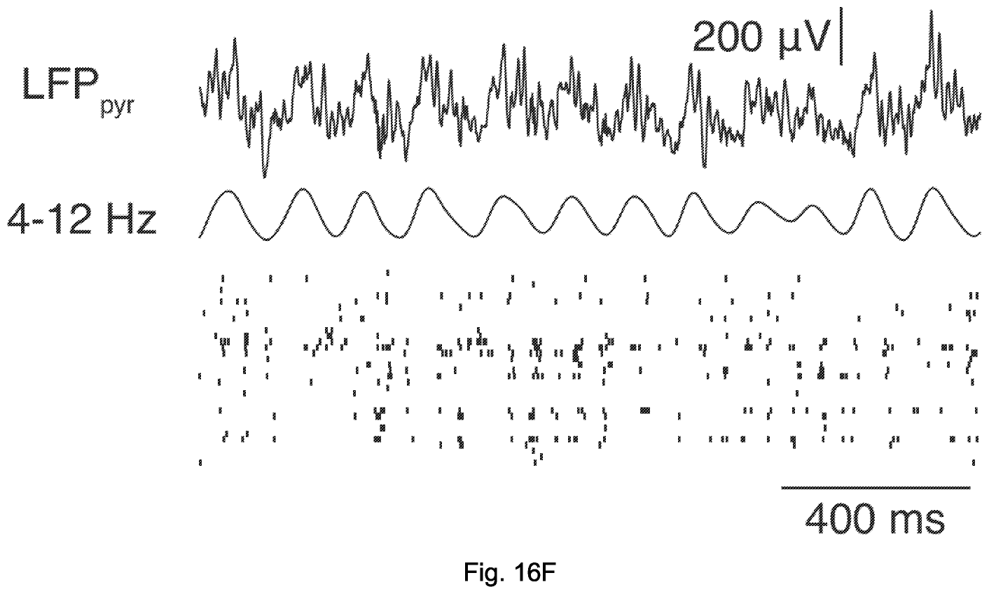

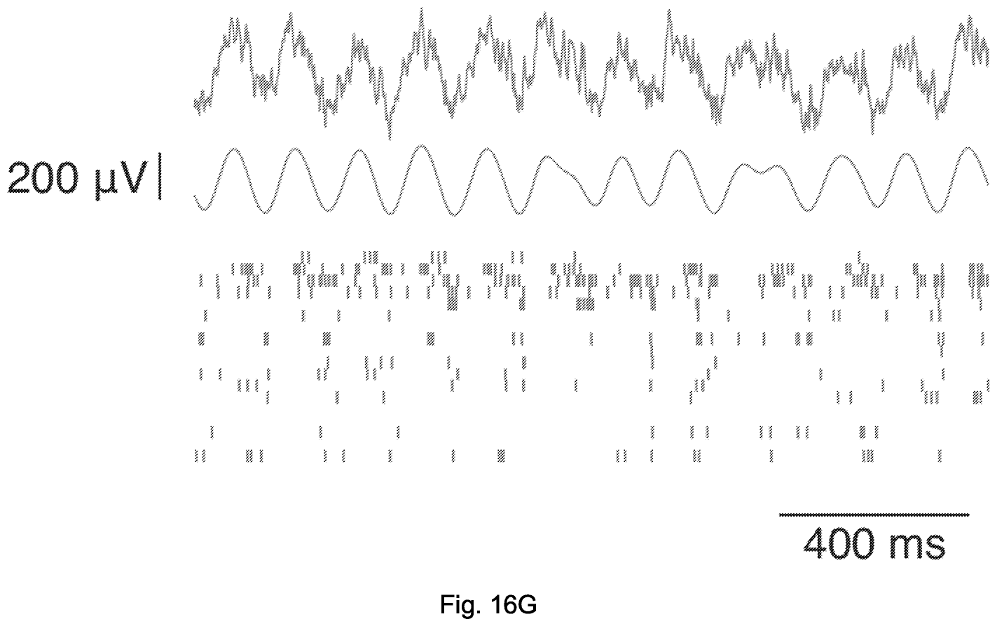

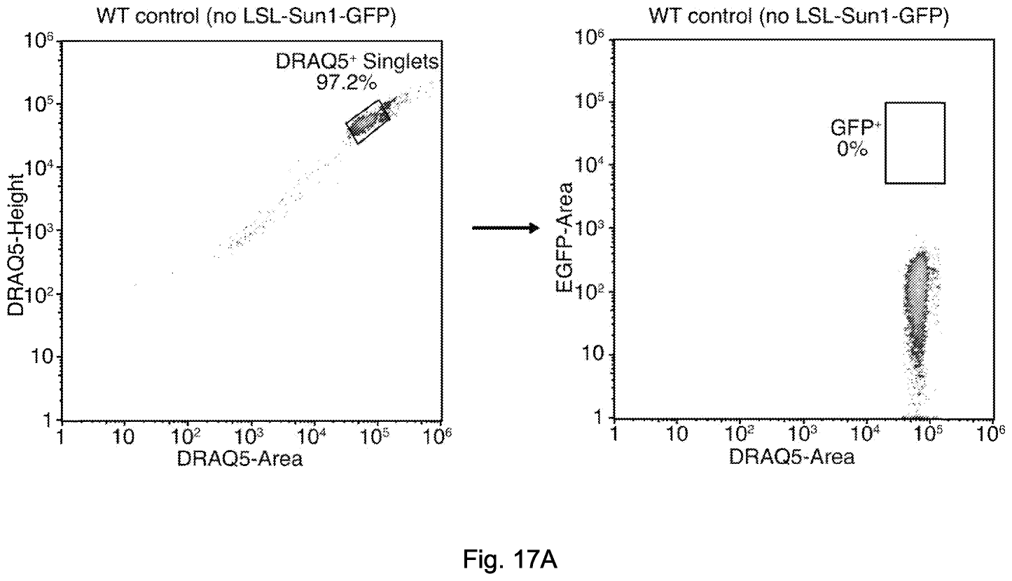

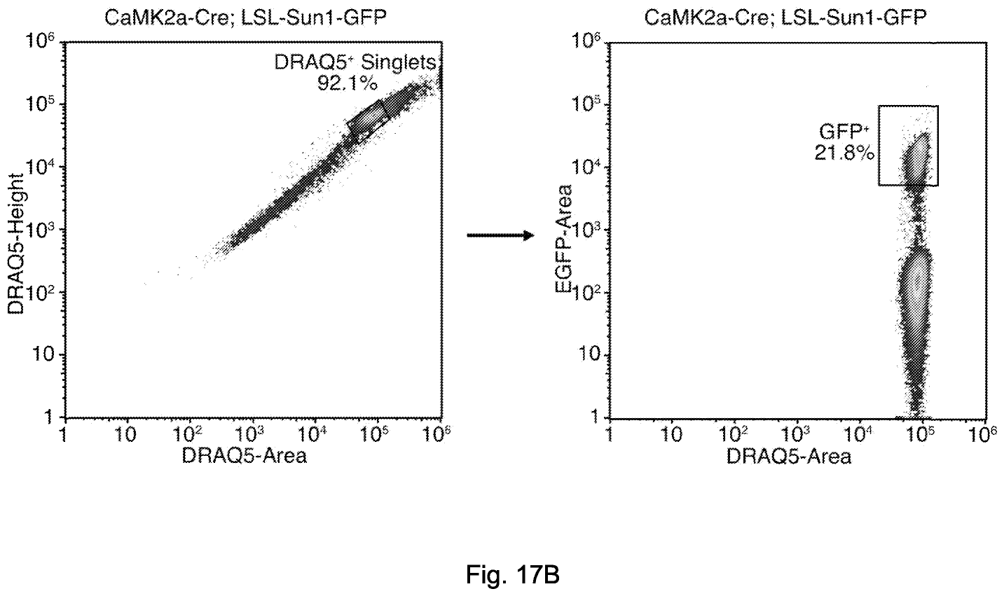

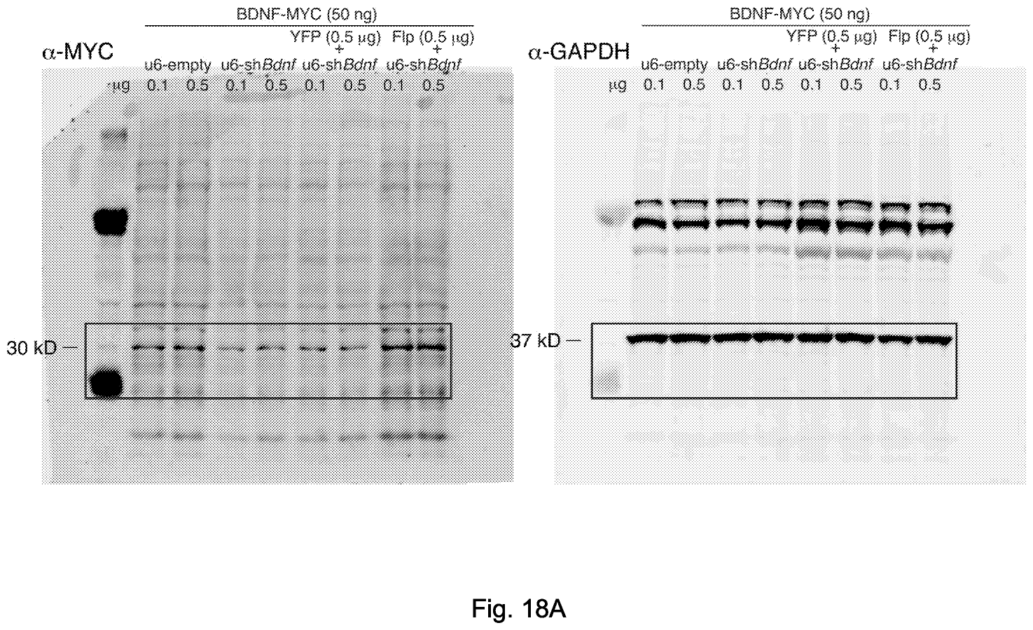

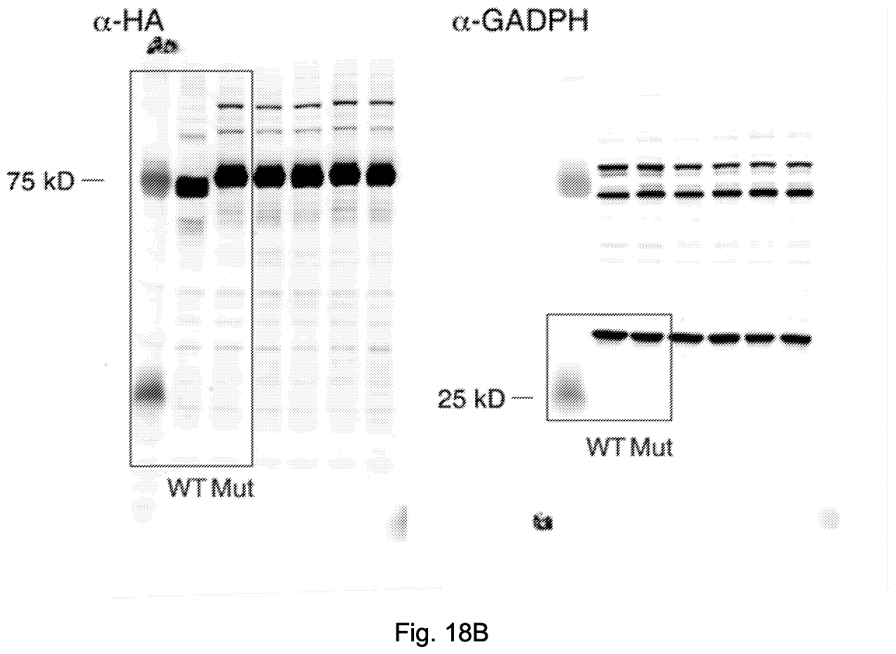

To determine whether the Fos-Scg2 pathway alters the function of hippocampal networks in vivo, the effects of disrupting Scg2 function on hippocampal network oscillations was assessed. Silicon probe recordings were performed in awake head-fixed mice running on an air-supported ball (see e.g., FIG. 6A). Scg2^{fl/fl} mice were injected with AAV expressing ΔCre- (Scg2-WT) or Cre (Scg2-KO) bilaterally into the CA1 (see e.g., FIG. 16A). The frequency spectra in the gamma range were altered, with Scg2-KO mice displaying significantly lower fast gamma (60-90 Hz) power compared to Scg2-WT mice when running (see e.g., FIG. 6B, FIG. 6C, FIG. 16B, FIG. 16C). By contrast, the power of theta rhythms (4-12 Hz) and mean spike rates were not significantly different between Scg2-WT and Scg2-KO mice (see e.g., FIG. 6B, FIG. 6C, FIG. 16B-FIG. 16G).

Additionally, PCs in Scg2-KOs fired at a significantly different preferred theta_{pyr} phase recorded in CA1 stratum pyramidale compared to Scg2-WT PCs (see e.g., FIG. 6D). Scg2-KO cells tended to fire later in the theta_{pyr} cycle, corresponding to the ascending phase of theta_{pyr}, whereas on average Scg2-WT cells fired during the descending phase of the theta_{pyr} cycle (Scg2-WT: 120.6° and Scg2-KO: 187.3° relative to the theta_{pyr} peak (0°)). These results were consistent with the observed change in the balance between PV-IN and CCK-IN inputs upon loss of Scg2, as PV-INs and CCK-INs have been observed to fire during the descending and ascending phases of theta_{pyr} oscillations, respectively; see e.g., Bartos & Elgueta (2012), supra; Yap & Greenberg (2018), supra.

### Discussion

Despite the prevalence of Fos-activated neuronal networks across many regions of the brain, there was limited understanding of the circuit and molecular mechanisms by which these networks become persistently modified to support the consolidation of experiences over time. Moreover, whether Fos has a causal role in orchestrating circuit modifications, and which of its many targets underlie these processes, was not known. Described herein is a bidirectional perisomatic inhibitory plasticity mechanism by which Fos-activated circuits were selectively reorganized in response to experience (see e.g., FIG. 6E). A Fos-to-Scg2 pathway was critical for this reorganization. Furthermore, Scg2 neuropeptidergic modulation played a role in the entrainment of PC activity relative to theta phase and the regulation of gamma rhythms. These results, together with the finding that Fos is necessary for spatial learning, indicate that Fos-dependent circuit reorganization is required to establish a network of cells for encoding and recalling memories.

Despite the broad axonal arborizations of PV-INs and CCK-INs within the CA1 pyramidal layer, distinct mechanisms specifically reorganize and establish Fos-activated microcircuits compared to non-Fos-activated networks. That PV-IN and CCK-IN synaptic strengths are oppositely regulated by novel experience indicates functional consequences for this reorganization beyond a strictly homeostatic role in which increased PC activity is balanced by increased perisomatic inhibition within the network. It is contemplated herein that this experience-dependent shift in inhibitory control can alters the temporal dynamics of network function in behaviorally adaptive ways.

For example, the peak and trough phases of theta rhythms measured in the CA1 pyramidal layer have been associated with memory encoding and recall, respectively, as the dominant source of inputs to CA1 cycles between entorhinal cortex and CA3. Fos-mediated reorganization of inputs from PV-INs and CCK-INs, which themselves fire during different phases of the theta cycle, can provide a mechanism for altering a cell's eligibility to take part in these processes. Scg2-expressing PCs fired preferentially during the descending phase of the theta cycle, which is when PV-INs also tend to fire, indicating that the Fos-dependent recruitment of PV-mediated inhibition is critical for the formation of functional PV-pyramidal cell ensembles to support the consolidation of memories. In addition, Scg2-dependent regulation of gamma rhythms can be critical for transiently synchronizing the activity of populations of neurons within and across brain regions to facilitate information processing. See e.g., Buzsaki (2002), supra; Buzsaki & Wang (2012), supra; Hasselmo & Stern (2014), supra; Bartos & Elgueta (2012), supra; Yap & Greenberg (2018), supra.

Additional distinctions in the molecular and physiological properties of PV-INs and CCK-INs can also contribute to the functional consequences of this shift. For example, it is contemplated herein that experience-dependent strengthening of PV-mediated inhibition onto PCs can increase their spike threshold and impose narrower time windows for synaptic integration, which can allow them to better synchronize their firing. It is further contemplated herein that Fos or Scg2 can contribute to endocannabinoid signaling involving presynaptic CCK-INs. See e.g., Bartos & Elgueta (2012), supra; Glickfeld & Scanziani (2006), supra; Foldy et al. Neuron 78, 498-509 (2013); Hartzell et al. Elife 7 (2018); the contents of each of which are incorporated herein by reference in their entireties.

Specific in vivo cellular and learning-related neural activity features lead to the induction of Fos during natural behaviors; see e.g., Josselyn & Tonegawa (2020), supra; Tanaka et al. (2018), supra. The findings described herein indicate that Fos expression has an instructive role in orchestrating persistent circuit modifications, beyond serving as a marker of recent neural activity. In particular, Fos coordinates neuropeptidergic networks to modulate connectivity through its regulation of Scg2. In the brain, Scg2 is mostly processed into its distinct neuropeptides, indicating that individual Scg2-derived peptides can mediate bidirectional perisomatic inhibitory plasticity. It is contemplated herein that characterization of the specific Scg2-derived peptides that are involved, their pre-synaptic or post-synaptic sites of action, and the identity of their cognate G-protein coupled receptors can be done to further assess the physiological functions of Fos-Scg2 signaling and the pathological consequences when this pathway is disrupted. See e.g., Fischer-Colbrie et al. (1995), supra; Weiler et al. Brain Res 532, 87-94 (1990); the contents of each of which are incorporated herein by reference in their entireties.

### Methods

Mice. Mice were handled according to protocols approved by a Standing Committee on Animal Care and were in accordance with federal guidelines. The following mouse lines were used: PV-Cre (JAX 017320), CCK-Cre (JAX 012706), PV-Flpo (JAX 022730), C57BL/6J (JAX 000664), Ai14 (JAX 007914), Ai65 (JAX 021875), CaMK2a-Cre (JAX 005359), Rp122/RIBOTAG (JAX 029977), LSL-Sun1-sfGFP-Myc (JAX 021039), Emx1-Cre (JAX 005628), Dlx5/6-Flpe, Fos^{fl/fl}; Fosb^{fl/fl}; Junb^{fl/fl}, Fos-FLAGHA, Npas4-FLAGHA, C57BL/6N (CHARLES RIVER LABORATORIES; for embryonic cultured neurons), and Scg2^{fl/fl} (described herein). See e.g., Vierbuchen et al. (2017), supra; Miyoshi et al. J Neurosci 30, 1582-1594 (2010); Sharma et al. Neuron 102, 390-406 e399 (2019); the contents of each of which are incorporated herein by reference in their entireties.

The conditional knockout Scg2^{fl/fl} mouse was generated with the help of a Genome Modification Facility. Briefly, LoxP sites were introduced flanking the entire coding exon of Scg2. Cas9 mRNA, two sgRNAs each targeting a site for LoxP insertion, and two 150-200 bp single-stranded oligonucleotides for repair were injected into C57BL/6J mouse zygotes. Correct cis insertion of both LoxP sites were verified by standard PCR and Sanger sequencing. A founder male was bred to C57BL/6J mice for at least three generations before experimental use.

Mice were housed in ventilated micro-isolator cages in a temperature- and humidity-controlled environment under a standard 12 h light/dark cycle, with food and water provided ad libitum. Both male and female littermate mice were used in similar proportions and divided between control and experimental groups for all experiments conducted. For in vivo silicon probe recordings and Morris water maze experiments, only male littermate mice, housed in a reverse 12 h light/dark cycle, were used.

Novel environment (NE) paradigm. Animals at weaning age and above (>P21) were placed in a large opaque cage (0.66 m×0.46 m×0.38 m) in a group with other mice, equipped with an assortment of enrichment including a running wheel, mazes, tunnels, ladders, huts, swings, and different kinds of animal bedding. Rodent pellets were hidden in mazes to encourage spatial exploration. Mice were placed in a specific environment for 12-24 h. The environments were subsequently significantly changed daily to provide novel multisensory experiences and to transcriptionally activate a larger proportion of neurons.

Intraperitoneal (i.p.) injections. For kainic acid (KA) treatment, mice were injected intraperitoneally with kainic acid (SIGMA ALDRICH, K0250) reconstituted in 0.001 N NaOH in PBS at 5-10 mg/kg for electrophysiology or 15-20 mg/kg for genomic or histological analyses. 1-1.5 h or 2-3 h KA was used as the timepoint for capturing the peak of immediate early gene (e.g., Fos) RNA or protein induction, respectively. 4 h KA was used as the timepoint for capturing the peak of nascent RNA induction for late-response genes, as nascent RNA molecules were first present in the nuclei (FFJ snRNA-seq). Subsequently, for ribosome-associated mature RNA from late-response genes, a 6 h KA timepoint was used, as more mature RNA tended to associate with ribosomes at this later timepoint (RIBOTAG). For electrophysiology, mice were sacrificed 24 h after KA injection to allow sufficient time for the expression and action of activity-dependent genes, but far in advance of any measurable seizure-related cellular toxicity (see e.g., FIG. 8M-FIG. 8Q).

For chemogenetic activation experiments, clozapine N-oxide (CNO; SIGMA C0832) reconstituted in 0.4% DMSO in PBS was injected intraperitoneally (i.p.) at 5 mg/kg in mice 24 h before electrophysiology.

Stereotaxic surgery. For acute hippocampal slice recordings, mice aged P13-15 of equal proportion male and female were anesthetized by isoflurane inhalation (2% induction, 1% maintenance) and positioned within a stereotaxic frame (KOPF MODEL 963). Animal temperature was maintained at 37° C. by a heat pad. All surgeries were performed according to protocols approved by the Standing Committee on Animal Care and were in accordance with federal guidelines. Fur around the scalp area was removed using a shaver and sterilized with three alternating washes with betadine and 70% ethanol. A burr hole was drilled through the skull above the CA1 region of hippocampus (medial/lateral, ML: ±2.9 mm; anterior/posterior, AP: −2.4 mm; dorsal/ventral, DV: −2.8 mm) to allow for specific targeting of this region with a glass pipette pulled to a tip diameter of approximately 50 μm. AAV virus (1000 nL) was injected at 150 nL/min, and the pipette was left in place for 5 min upon completion of viral infusion to allow for viral spreading. All animals were given postoperative analgesic (flunixin, 2.5 mg/kg) as well as additional injections at 12 h-intervals for the 72 h following surgery.

Viral vectors and titers. All AAVs used were prepared in a Hospital Viral Core and were of serotype AAV2/1. For sparse transductions, viruses were injected at 1×10⁸ genome copies per hippocampal hemisphere. For dense transductions, viruses were injected at 2×10⁹ genome copies (gc) per hippocampal hemisphere. The viral vectors and original titers were as follows: pAAV-EF1a-DIO-hChR2(H134R)-EYFP (ADDGENE 20298, 1.75×10¹³ gc/mL), pAAV-EF1a-fDIO-hChR2(H134R)-EYFP (ADDGENE 55639, 1.39×10¹³ gc/mL), pAAV-hSyn-Con/Fon-hChR2-EYFP (ADDGENE 55645, 2.25×10″ gc/mL), pAAV-pRAM-tTA::TRE-NLS-mKate2-WPREpA (ADDGENE 84474, 2.25×10¹³ gc/mL), pAAV-CAG-Cre-GFP (OHIO STATE UNIVERSITY, 1.75×10¹³ gc/mL), pAAV-CAG-Cre-mCherry (described herein, 9.10×10¹² gc/mL), pAAV-CAG-Cre-mTagBFP2 (described herein, 2.97×10¹² gc/mL), pAAV-CAG-deltaCre-GFP (described herein, 2.79×10¹² gc/mL), pAAV-FlpOFF-u6-shRNA-CAG-mCherry (described herein): Scrambled control shRNA (SEQ ID NO: 13, ACTTACGCTGAGTACTTCG) (5.08×10¹³ gc/mL), Inhba (SEQ ID NO: 14, CCTTCCACTCAACAGTCATT) (4.62×10¹³ gc/mL), Bdnf (SEQ ID NO: 15, GAATTGGCTGGCGATTCATA) (6.97×10¹³ gc/mL), Pcsk1 (SEQ ID NO: 16, GATAATGATCATGATCCATT) (6.02×10¹² gc/mL), Nptx2 (SEQ ID NO: 17, GAAGACATTGCCTGAGCTGT) (1.30×10¹² gc/mL), Scg2#1 (SEQ ID NO: 18, GCAGACAAGCACCTTATGAA) (8.11×10¹¹ gc/mL), Scg2#2 (SEQ ID NO: 19, CCCTTGATTCTCAGTCTATT) (2.75×10¹³ gc/mL), Rgs2 (SEQ ID NO: 20, GCTCCCAAAGAGATAAACAT) (6.14×10¹² gc/mL), pAAV-CaMKIIa-mCherry (described herein, 3.80×1012 gc/mL), pAAV-CaMKIIa-hM3D_{Gq}-T2A-mCherry (described herein, 1.20×10¹² gc/mL), pAAV-hSyn-FlpOFF-Kir2.1-T2A-mCherry (described herein, 2.26×10¹² gc/mL), pAAV-hSyn-FlpOFF-Kir.2.1(Mutant)-T2A-mCherry (described herein, 1.28×10¹² gc/mL; see e.g., Xue et al. (2014), supra), pAAV-u6(Frt)-shRNA#31-CAG-Scg2-rescue (shRNA-resistant)-1×HA-T2A-mCherry-Frt-SV40 (described herein, 1.88×10¹² gc/mL), pAAV-CAG-DIO-Scg2(WT)-3×HA-bGH polyA (described herein, 8.22×10¹³ gc/mL), pAAV-CAG-DIO-Scg2(9AA Mutant)-3×HA-bGH polyA (described herein, 6.13×10¹³ gc/mL), pAAV-CAG-Frt-Scg2(WT)-1×HA-T2A-mCherry-Frt-bGH polyA (described herein, 1.08×10¹³ gc/mL), and pAAV-CAG-Scg2(9AA Mutant)-1×HA-T2A-mCherry-Frt-bGH polyA (described herein, 3.71×10¹² gc/mL).

For lentiviral production of shRNAs, lentiviral backbone pSicoR (ADDGENE 11579) was used for cloning all shRNAs. A total of 10 mg of lentiviral plasmid was transfected into 293T cells in a 10-cm dish along with third generation packaging vectors pMD2.G (ADDGENE 12259), pRSV-rev (ADDGENE 12253) and pMDLg/pRRE (ADDGENE 12251). At 12-16 h following transfection, 293T cells were switched to NEUROBASAL media (GIBCO) containing B27 supplement (2%), penicillin (50 U/ml), streptomycin (50 U ml/L) and GLUTA-MAX (1 mM). Supernatant containing virus was collected at 36 h post-transfection, spun down to remove cellular debris at 1,000×g for 5 min, and added directly to cultured neurons.

Acute slice preparation. Transverse hippocampal slices were prepared from mice aged P23-P32. Mice were anaesthetized with ketamine/xylazine and transcardially perfused with ice-cold choline-based artificial cerebrospinal fluid (choline-ACSF) equilibrated with 95% O₂/5% CO₂ comprising (in mM): 110 choline chloride, 25 NaHCO₃, 1.25 NaH 2 PO₄, 2.5 KCl, 7 MgCl₂, 25 glucose, 0.5 CaCl₂, 11.6 sodium L-ascorbate, and 3.1 sodium pyruvate. Cerebral hemispheres were quickly removed and placed into ice-cold choline-ACSF. Tissue was rapidly blocked and transferred to a vibratome (LEICA VT1000). Dorsal hippocampal slices of 300 μm thickness were collected in a holding chamber containing ACSF comprising (in mM): 127 NaCl, 25 NaHCO₃, 1.25 NaH 2 PO₄, 2.5 KCl, 1 MgCl₂, 10 glucose, and 2 CaCl₂. For all solutions, pH was set to 7.2 and osmolarity to 300 mOsm. Slices were incubated at 32° C. for 20 min and maintained at room temperature (RT, 22° C.) for 30 min before recordings began. All recordings were performed at RT within 4-5 h of slice preparation. AAV transduction was assessed by epifluorescence. For experiments where sparse transduction of CA1 was intended, slices with 10-30% of CA1 neurons infected were used, and slices showing >30% of CA1 neurons infected were discarded from further analysis. For optogenetic stimulation experiments, slices showing channelrhodopsin-2 (ChR2) spread across the entire CA1 were used, and slices showing partial expression of ChR2 across CA1 were discarded from further analysis. For all experiments, slices were discarded if AAV transduction spread to CA3 and/or dentate gyrus regions.

Ex vivo electrophysiology. For whole-cell voltage-clamp recordings, a CsCl-based internal solution comprising (in mM): 135 CsCl, 3.3 QX314-C1, 10 HEPES (4-(2-hydroxyethyl)-1-piperazineethanesulfonic acid), 4 MgATP, 0.5 NaGTP, 8 Na₂-phosphocreatinine, 1.1 EGTA (egtazic acid), and 0.1 CaCl₂ (pH 7.2, 290 mOsm) was used for all IPSC measurements. A Cst methanesulfonate internal solution comprising (in mM): 127 CsMeSO₃, 10 CsCl, 10 HEPES, 0.5 EGTA, 2 MgCl₂, 0.16 CaCl₂), 2 MgATP, 0.4 NaGTP, 14 Na₂-phosphocreatinine, and 2 QX314-C1 (pH 7.2, 295 mOsm) was used for all EPSC measurements. A K⁺-based internal solution comprising (in mM): 142 K⁺-gluconate, 4 KCl, 10 HEPES, 4 MgATP, 0.3 NaGTP, 10 Na₂-phosphocreatinine, and 1.1 EGTA (pH 7.2, 280 mOsm) was used for all current-clamp recordings. Membrane potentials were not corrected for liquid junction potential (which were experimentally measured as −5 mV for CsCl-based internal solution, and 60 mV for K-gluconate-based internal solution). In all recordings, neurons were held at −70 mV with patch pipettes made with borosilicate glass with filament (SUTTER BF150-86-7.5) with 2-4 MΩ open pipette resistance. For all dual whole-cell recordings of pairs of CA1 pyramidal neurons, recording from neighboring neurons increased the probability that both neurons received synaptic inputs from the same population of inhibitory axons, and ensured that both neurons were exposed to an identical stimulus magnitude and intensity.

Recordings were made on an upright OLYMPUS BX51 WI microscope with an infrared CCD camera (DAGE-MTI IR-1000) and 60× water immersion objective (OLYMPUS LUMPLAN Fl/IR 60×/0.90 numerical aperture). Neurons were visualized using video-assisted infrared differential interference contrast, and fluorescence was identified by epifluorescence driven by a light-emitting diode (EXCELITAS XCITE LED120). For photostimulation of ChR2-expressing boutons, 470 nm blue light was delivered from the LED through the reflected light fluorescence illumination port and the 60× objective. Pulses were delivered at 0.4 Hz. Pulse duration (0.1-0.2 ms) and intensity (1.3-5.9 mW/mm²) were adjusted for each recording to evoke small but reliable monosynaptic IPSCs. No pharmacology was used for optogenetic stimulation experiments.

For electrical stimulation experiments, electrical current was delivered via theta glass stimulation electrode placed in the center of stratum pyramidale or stratum radiatum within 150-200 μm of the recorded neuron pair. The stimulus strength was the minimum required to generate small but reliable currents in both neurons. IPSCs were pharmacologically isolated via the addition of 10 μM NBQX (TOCRIS 1044) and 10 μM (R)-CPP (TOCRIS 0247) to the ACSF perfusion. For pharmacological isolation of CCK-IPSCs specifically, in addition to blocking excitatory currents, PV-IPSCs were blocked using 0.4 μM of ω-agatoxin IVA, a selective antagonist for P/Q-type calcium channels (PEPTIDES INTERNATIONAL, PAG-4256-s). EPSCs were pharmacologically isolated by adding 10 μM gabazine (TOCRIS 1262).

For simultaneous dual whole-cell recordings, it was determined that the IPSCs measured were monosynaptic, as the addition of NBQX and (R)-CPP in the bath did not alter the onset latency of the IPSCs. For the paired interneuron-to-CA1 pyramidal neuron recordings, the monosynaptic nature of the IPSCs was confirmed based on the expected onset latency of 1-3 ms in slice.

Data acquisition and analysis. Data were low-pass filtered at 4 kHz and sampled at 10 kHz with an AXON MULTICLAMP 700B amplifier, and digitized with an AXON DIGIDATA 1440A data acquisition system controlled using CLAMPEX 10.6 (MOLECULAR DEVICES). Experiments were discarded if holding current exceeded −500 pA, or if series resistance was greater than 30 MΩ. For the dual whole-cell recordings of CA1 pyramidal neurons, recordings were discarded if series resistance differed by more than 30% between the two neurons. The recorded traces were analyzed using CLAMPFIT 10.6 software (MOLECULAR DEVICES) or AXOGRAPH (1.7.6). All current amplitude measurements were expressed as mean±SEM, or as differences in amplitudes between a pair of neurons normalized to the total amplitudes of both neurons (ΔIPSC/EIPSC). The differences (ΔIPSC) were calculated between a fluorescently labeled (i.e., manipulated) cell minus a control (i.e., non-manipulated) cell, such that a positive number indicated a larger IPSC amplitude in the manipulated cell compared to the control cell, and vice versa.

Sample sizes were not predetermined and were similar to those previously reported; see e.g., Xue et al. (2014), supra; Bloodgood et al. (2013), supra; Sharma et al. (2019), supra. In general, approximately 15-20 pairs of neurons (n) collected from 3-5 animals (N) were sufficient for each experiment. Most data, except where specified (see e.g., FIG. 4C-FIG. 4E), were not collected blind to genotype or conditions, but all offline analyses were conducted blind. All statistical analyses were performed using PRISM 8 (GRAPHPAD). Data were tested for normality using the D'Agostino-Pearson, Shapiro-Wilk, and Kolmogorov-Smirnov normality tests. For simultaneous dual whole-cell recordings of pyramidal neurons, parametric t-tests or non-parametric Wilcoxon rank-sum tests (two-sided) were used. For recordings of unitary connections, non-parametric Mann-Whitney tests (two-sided) were used. A mixed model was used to confirm that findings were not driven by a single mouse. The numbers of cells recorded per animal were capped to ensure even sampling across mice comprising a dataset (e.g., if n=20 pairs were obtained using N=4 mice, 4-6 pairs were used per mouse).

Histology. Mice were anaesthetized with 10 mg/mL ketamine and 1 mg/mL xylazine in phosphate buffered saline (PBS) via i.p. injection. When fully anaesthetized, the animals were transcardially perfused with 5 mL ice-cold PBS followed by 20 mL of cold 4% paraformaldehyde (PFA) in PBS. Brains were dissected and post-fixed for 1 h at 4° C. in 4% PFA, followed by three washes (each for 30 min) in cold PBS. Coronal sections (40 μm thick) were subsequently cut using a LEICA VT1000 vibratome and stored in PBS in 4° C. until further use. For immunostaining, slices were permeabilized for 30 min at RT in PBS containing 0.3% TRITON X-100. Slices were blocked for 1 h at RT with PBS containing 0.3% TRITON X-100, 2% normal donkey serum and 0.1% fish gelatin. Slices were incubated in primary antibodies diluted in blocking solution at 4° C. for 48 h: rabbit anti-Fos antibody (SYNAPTIC SYSTEMS 226003, 1:3000), mouse anti-Fos (ABCAM ab208942, 1:1000), rabbit anti-Npas4 (1:1000; see e.g., Lin et al. Nature 455, 1198-1204 (2008), the content of which is incorporated herein by reference in its entirety), rabbit anti-Fosb (CELL SIGNALING TECHNOLOGY 2251S, 1:1000), rabbit anti-Junb (CELL SIGNALING TECHNOLOGY 3753S, 1:1000), rat anti-HA (SIGMA ROAHAHA, 1:500), rabbit anti-parvalbumin (SWANT PV27, 1:10,000), rabbit anti-cleaved Caspase-3 (CELL SIGNALING TECHNOLOGY 9661S, 1:1000), and mouse monoclonal anti-NeuN (MILLIPORE SIGMA, MAB377, 1:1000). Slices were then washed three times with PBS each for 10 min at RT, incubated for 2 h at RT with secondary antibodies conjugated to ALEXA FLUOR dye (LIFE TECHNOLOGIES); rat ALEXA FLUOR 555 (A21434), rabbit ALEXA FLUOR 488 (A21206), rabbit ALEXA FLUOR 555 (A31572), rabbit ALEXA FLUOR 647 (A31573), mouse ALEXA FLUOR 555 (A31570), mouse ALEXA FLUOR 647 (A31571), 1:250), and washed three times with PBS. Slices were then mounted in DAPI FLUOROMOUNT-G (SOUTHERN BIOTECH) and imaged on a virtual slide microscope (OLYMPUS VS120).

Single-molecule RNA fluorescence in situ hybridization (smRNA-FISH). For sample preparation, hippocampal hemispheres from mice were fresh- or fixed-frozen in TISSUE-TEK CRYO-OCT compound (FISHER SCIENTIFIC) on dry ice and stored in −80° C. until further use. Hippocampi were sectioned at a thickness of 15-20 μm and RNAs were detected by RNASCOPE (ADVANCED CELL DIAGNOSTICS) using the manufacturer's protocol. Probes for Fos, Fosb, and Junb were custom designed with ADVANCED CELL DIAGNOSTICS specifically to detect exons excised upon Cre recombinase expression. The following probes were used: Mm-Cre (Cat. #546951), Mm-Fos (Cat. #584741), Mm-Fosb (Cat. #584751), Mm-Junb (Cat. #584761), Mm-Scg2 (Cat. #477691), and Mm-Scg2 intron (Cat. #859141). All in situ hybridizations were imaged using a confocal microscope (ZEISS IMAGER Z2) and analyzed in IMAGEJ (FIJI v1.0).

Validation of loss of Fos, Fosb, and Junb in the Fos^{fl/fl}; Fosb^{fl/fl}; Junb^{fl/fl} (FFJ) conditional knockout mouse line. Efficient excision of Fos, Fosb, and Junb upon Cre expression was confirmed at the RNA level using smRNA-FISH and at the protein level using immunostaining for each of the three genes. The Fos conditional knockout allele allowed for deletion of three exons, including the last exon encoding the 3′ UTR, upon Cre expression, whereas the Fosb and Junb conditional knockout alleles were single-exon deletions (Exon 2 of 4 for Fosb; coding region only for Junb). As such, for smRNA-FISH, the probes were custom designed to specifically target the excised exons. Note that snRNA-seq detects the 3′ ends of transcripts, resulting in comparatively sparse coverage of full transcripts particularly at the 5′ end of genes. This approach can therefore be used to confirm the deletion of Fos but not Fosb and Junb due to the design of the conditional knockout alleles, which leaves intact the 3′ transcripts of Fosb and Junb upon Cre excision, resulting in non-trivial tags during library preparation.

Cultured hippocampal neurons and RNA isolation for RT-qPCR or bulk RNA-sequencing. Embryonic hippocampi from C57BL/6N (CHARLES RIVER LABORATORIES) or Scg2^{fl/fl} mice were dissected at age E16.5 or P0, respectively, then dissociated with papain (SIGMA ALDRICH 10108014001). Cultures were generated by combining multiple embryos of both males and females (mixed sex cultures). Papain digestion was terminated with the addition of ovomucoid (trypsin inhibitor; WORTHINGTON). Cells were gently triturated through a P1000 pipette and passed through a 40 μm filter. Neurons were plated onto cell culture dishes pre-coated overnight with poly-D-lysine (20 mg/mL) and laminin (4 mg/mL). Neurons were grown in NEUROBASAL medium (GIBCO) containing B27 supplement (2%), penicillin-streptomycin (50 U/mL penicillin and 50 U/mL streptomycin) and GLUTA-MAX (1 mM). Neurons were grown in incubators maintained at 37° C. and a CO₂ concentration of 5%. In all experiments, independent replicates were generated from dissections of mice on different days. Neurons were cultured in 6-well dishes at 1 million neurons per well. Neurons were transduced with lentiviral supernatant on days in vitro 2 (DIV2) by replacing one third of NEUROBASAL media with lentiviral supernatant. Fresh media was added at DIV4 (one fourth total volume). At DIV7, neurons were depolarized with 55 mM potassium chloride (KCl) for 1- or 6 h to assess immediate early or late-response activity-dependent genes, respectively, and RNA was subsequently harvested by gentle agitation with TRIZOL (LIFE TECHNOLOGIES 15596026) at RT for 2 min. The RNEASY MICRO KIT (QIAGEN 74004) was used according to the manufacturer's instructions to purify DNA-free RNA. For quantitative PCR with reverse transcription (RT-qPCR), RNA was converted to cDNA using 200 ng of RNA with the high-capacity cDNA reverse transcription kit (LIFE TECHNOLOGIES 4374966). qRT-PCR was performed with technical triplicates and mapped back to relative RNA concentrations using a standard curve built from a serial dilution of cDNA. Data were collected using a QUANTSTUDIO 3 qPCR machine (APPLIED BIOSYSTEMS). For bulk RNA-sequencing, 100 ng of RNA was used to generate libraries following rRNA depletion (NEBNEXT, E6310X) according to the manufacturer's instructions (NEBNEXT, E7420). The 75-bp reads were generated on the ILLUMINA NEXTSEQ 500 and subsequently analyzed using a standardized RNA-seq data analysis pipeline; see e.g., Ataman et al. Nature 539, 242-247 (2016), the content of which is incorporated herein by reference in its entirety.

Morris water maze behavioral paradigm. 8-14-week-old littermate Fos^{fl/fl}; Fosb^{fl/fl}; Junb^{fl/fl} (FFJ) mice were injected with AAV-Cre-GFP or AAV-ΔCre-GFP bilaterally into the CA1 (stereotaxic coordinates of AP −2 mm, ML ±1.5 mm, DV −1.3 mm from bregma). Mice were given injections of dexamethasone and buprenorphine SR™, and allowed to recover for 1-2 weeks before behavioral training. The maze (97 cm in diameter) was filled with RT water made opaque by the addition of tempera to a height of 40 cm. A hidden platform of 7 cm-diameter was placed 14 cm from the edge of the maze and submerged 1 cm below the water level. Distal cues were placed on all four walls of the testing room. Mice were trained in two blocks per day for four consecutive days (days 1-4). Each block consisted of four trials. In each trial, mice were placed at one of eight (randomized) start positions spaced evenly along half of the circumference of the pool opposite the half of the pool that contained the hidden platform. Mice were given 60 s to find the platform. If mice did not find the platform within this time, they were guided to the platform by the experimenter and allowed to sit for 10 s. Mice were subsequently removed from the platform and placed in a warmed cage to dry. Two 40 s probe trials were conducted one day after training (day 5) during which the platform was removed. The swim paths of the mice were recorded by a video camera suspended several feet above the center of the maze. The experimenter was blinded to the genotype of the mice. Mice that did not swim (“floaters”) were excluded from further analysis.

Analysis. All video tracking and analysis was carried out using custom MATLAB code. Swim trajectories for each trial were tracked semi-automatically and manually corrected. For one mouse in the study, due to tracking issues the trials in the second block on the first day (trials 5-8) were excluded from the analysis—therefore for that mouse only four trials were considered in the performance metric on day 1. For analyses of swim speeds and path lengths, the mean was computed for each mouse across all trials on the first two days in order to control for similar levels of exploration.

Ribosome-associated mRNA profiling. Hippocampal tissue was rapidly dissected from mice and subsequently used for isolation of ribosome-bound mRNAs. Immunopurification of ribosome-bound mRNAs was performed with 10 mM Ribonucleoside Vanadyl Complex (NEB S1402S) present in the lysis buffer and using the mouse monoclonal anti-HA antibody (SIGMA HA-7, H3663, 12 μg per immunoprecipitation); see e.g., Sanz et al. (2009), supra. A small fraction of lysate before the immunoprecipitation was used as input for each sample. All RNA samples (20 ng for CaMK2a; 2.5 ng for PV) with sufficient integrity as analyzed by 2100 BIOANALYZER were amplified using single primer isothermal amplification (SPIA) with the OVATION RNA-SEQ SYSTEM V2 (NUGEN). Subsequently, SPIA-amplified cDNA (1 μg) was fragmented to a length of approximately 400 bp using a COVARIS S2 sonicator (ACOUSTIC WAVE INSTRUMENTS). Fragmented cDNA (100 ng) was used to generate ILLUMINA-compatible sequencing libraries using the OVATION ULTRALOW SYSTEM V2 (NUGEN). Libraries were sequenced on the ILLUMINA NEXTSEQ 500 (BASESPACE) for 75 bp single-end reads to a depth of 20-40 million reads per sample.

Analysis. Analyses of RIBOTAG sequencing were performed for each sample at each stimulation time point; see e.g., Mardinly et al. Nature 531, 371-375 (2016), the content of which is incorporated herein by reference in its entirety. Briefly, raw sequencing reads ≤75 bp in length were 3′-trimmed to a uniform 70 bp (ignoring the ˜0.1% of reads that were shorter than this) and filtered for quality control. These were then mapped strand-nonspecifically to the mm10 genome (GRCm38) using the BURROWS-WHEELER ALIGNER (BWA), allowing up to 2 mismatches and no gaps. In addition to the usual assembled chromosomes, alignment targets included mitochondrial DNA and a library of ˜7 million short splice junction sequences. Typically, 75-80% of reads were mappable; nonuniquely mapped reads were discarded, as were any that mapped to loci of rRNA genes (from REPEATMASKER).

Genic features were based on exonic loci from the NCBI REFSEQ annotation for mm10. Mean expression density across a gene's exons was taken as a proxy for its expression level. (However, noncoding genes, some of which expressed quite highly and variably from one sample to the next, were excluded from these analyses.) The splice junction target sequences for each gene comprised subsequences of minimal length of all possible concatenations of two or more ordered exons such that their boundaries would be crossed by 70 bp reads. This provided an exhaustive, nonredundant set of predictable exon-junction-spanning loci which typically accounted for ˜20% of all exonic reads from mature messages.

Differential expression analyses employed EDGER in R to compare transcript levels in all biological-replicate samples at 6 h of KA stimulation to the unstimulated samples. A gene's expression level was flagged as significantly changed if (1) the Benjamini-Hochberg-corrected p value (q value) for the change, as calculated by EDGER, was consistent with a false discovery rate (FDR) of ≤0.005, and (2) it passed a modest background filter (total number of reads ≥4 across all compared samples).

Nuclei isolation. Hippocampal tissue from mice was rapidly dissected and dounce homogenized. Dounce homogenization was performed in BUFFER HB (0.25 M sucrose, 25 mM KCl, 5 mM MgCl₂, 20 mM Tricine-KOH, pH 7.8 supplemented with protease inhibitors, 1 mM dithiothreitol (DTT), 0.15 mM spermine and 0.5 mM spermidine) with a tight pestle for 20 strokes in a 1.5 mL total volume. Tissue was then supplemented with 96 uL 5% IGEPAL CA-630 and dounced an additional 5 strokes with a tight pestle. Homogenate was then filtered through a 40 μm strainer to remove large debris and collected in a 15 mL conical tube before the addition of 3.5 mL of BUFFER HB and 5 mL of working solution (50% iodixanol with 25 mM KCl, 5 mM MgCl₂, 20 mM Tricine-KOH pH 7.8 supplemented with protease inhibitors, DTT, spermine and spermidine). Homogenized tissue was underlaid with 1 mL of 30% iodixanol and 1 mL of 40% iodixanol (diluted from working solution) solutions. Samples were centrifuged at 10,000×g for 18 min. Nuclei were collected in a 70 uL or 250 uL volume at the 30/40% iodixanol interface for 10× GENOMICS and CUT&RUN protocols, respectively.

FFJ single-nucleus RNA-sequencing (snRNA-seq). FFJ snRNA-seq was performed with the 10× GENOMICS CHROMIUM SINGLE CELL KIT (v3). Approximately 7,000-10,000 nuclei were added to the reverse-transcription (RT) mix prior to loading on the microfluidic chip. Each snRNA-seq sample comprised pooled nuclei from 2 mice. All downstream steps for the cDNA synthesis, cDNA amplification and library preparation were performed according to the manufacturer's instructions (10× GENOMICS). All samples were sequenced on ILLUMINA NEXTSEQ 500 (BASESPACE) with 58 bp (read 1), 26 bp (read 2) and 8 bp (index).

Analysis. Initial FASTQ files were generated using the standard BCL2FASTQ ILLUMINA pipeline, and gene expression tables for each barcode were generated using the CELLRANGER (v3.0.0) pipeline according to instructions provided by 10× GENOMICS. AAV transduced cells were detected by the presence of mRNA species mapping to the WPRE-bGH polyA sequence present in all AAVs used herein. WPRE transcripts were PCR amplified using custom primers. Gene expression tables were then imported into R and analyzed using custom written functions as well as the SEURAT (v3) package. Exclusion criteria were as follows: nuclei were removed from the dataset if they contained fewer than 500 discovered genes or had greater than 5% of reads mapping to mitochondrial genes. General analysis parameters were as follows: raw unique molecular identifier (UMI) counts were normalized to 10⁴ UMIs per cell (i.e., tags per ten thousand, TPT). Nuclei from all Cre (or all ΔCre) mice were merged for the purposes of dimensionality reduction and clustering. Highly variable genes were identified using the FINDVARIABLEFEATURES function (selection.method=‘vst’, nFeatures=2000), which identified the 2,000 most variable genes amongst the analyzed nuclei. Principal component analysis based on the 2,000 most variable genes was performed using the RUNPCA function to reduce the dimensionality of the dataset. The top 20 principal components were retained and projected into a 2-dimensional space using the uniform manifold approximation and projection (UMAP) algorithm implemented using the RUNUMAP function (n.neighbors=50, min.dist=0.5). The following genes were used as a guide to assign cell type identities to the graph-based clusters: pan-excitatory neurons (Sid 7a7); CA1 excitatory neurons (Fibcd1, Mpped1); CA3 excitatory neurons (Spock1, Cpne4); excitatory dentate gyrus (Prox1, C1q12); pan-inhibitory interneurons (Gad2, Slc32a1); Sst⁺ interneurons (Sst); Pvalb⁺ interneurons (Pvalb); Vip⁺ interneurons (Vip); Cck⁺ interneurons (Cck); Nos1⁺ interneurons (Nos 1), Npy⁺ interneurons (Npy), Oligodendrocytes (Aspa, Opalin, Gjb1); Oligodendrocyte precursor cells (Gpr17, C1q11); Microglia (Cx3cr1, C1qc); Endothelial cells (Ly6c1, Cldn5); Astrocytes (Cldn10, Gjb6, Gfap); see e.g., Hrvatin et al. (2018), supra; Habib et al. Science 353, 925-928 (2016); Cembrowsk et al. Elife 5, e14997 (2016); the contents of each of which are incorporated herein by reference in their entireties. Differential gene expression (DGE) analysis was performed as follows: statistical significance of gene expression changes for all genes detected in greater than 5% of respective untransduced control cells for Cre or ΔCre samples was calculated using the Wilcoxon rank-sum test implemented through the FINDMARKERS function (logfc.threshold=pseudocount.use=0.001). Violin plots were generated using the VLNPLOT function with default parameters and heatmaps were generated using a custom written function in R. Heatmaps display the normalized gene expression values from 100 randomly selected cells from each indicated cell identity, and genes displayed were AP-1 targets showing reduced expression by at least 20% in the FFJ KO (Cre⁺) and whose expression was detected in at least 25% of analyzed nuclei.

CUT&RUN. Hippocampal nuclei from CaMK2a^{Cre}/+; LSL-Sun1-sfGFP-Myc/+ mice injected with saline or 2-3 h KA were isolated as described above. Isolated nuclei were diluted two-fold with CUT&RUN wash buffer supplemented with 4 mM ethylenediaminetetraacetic acid (EDTA) and stained with DRAQ5 (ABCAM ab108410) at a 1:500 dilution. CaMK2a⁺ (GFP⁺) nuclei, resulting from CaMK2a-Cre-mediated expression of Sun1-sfGFP-Myc, were isolated by flow cytometry using a SONY SH800Z CELL SORTER and subsequently analyzed using FLOWJO (10.6). Sorted nuclei were resuspended in 1 mL cold CUT&RUN wash buffer (20 mM HEPES pH 7.5, 150 mM NaCl, 0.2% TWEEN-20, 1 mg/mL bovine serum albumin (BSA), 10 mM sodium butyrate, and 0.5 mM spermidine supplemented with protease inhibitors), using 50,000 nuclei for each reaction. Nuclei were bound to magnetic Concanavalin-A (ConA) beads (BANGS LABORATORIES) that had been washed with CUT&RUN binding buffer (20 mM HEPES-KOH pH 7.9, 10 mM KCl, 1 mM CaCl₂, 1 mM MnCl₂). ConA-bead-bound nuclei were then incubated overnight in cold CUT&RUN antibody buffer (CUT&RUN wash buffer supplemented with 0.1% TRITON X-100 and 2 mM EDTA) and an in-house rabbit polyclonal anti-Fos antibody (affinity eluted #1096, 1:100) or rabbit IgG antibody (CELL SIGNALING TECHNOLOGY #2729, 1:100).

After antibody incubation, ConA-bead-bound nuclei were washed with CUT&RUN antibody buffer, resuspended in CUT&RUN TRITON-wash buffer (CUT&RUN wash buffer supplemented with 0.1% TRITON X-100), and Protein-A-MNase was added at a final concentration of 700 ng/mL. Samples were incubated at 4° C. for 1 h. The ConA-bead-bound nuclei were then washed twice with CUT&RUN TRITON-wash buffer and ultimately resuspended in 100 uL of CUT&RUN TRITON-wash buffer. 3 uL of 100 mM CaCl₂ was added to each sample and samples were incubated on ice for 30 min. The reaction was stopped by the addition of 100 uL of 2× STOP buffer (340 mM NaCl, 20 mM EDTA, 4 mM EGTA, 0.04% TRITON X-100, 20 pg/mL yeast spike-in DNA, and 0.1 μg/mL RNase A) and incubation at 37° C. for 20 min. After incubation, ConA beads were captured using a magnet and supernatants containing DNA fragments released by Protein-A-MNase were collected. Supernatants were then treated with 2 uL of 10% sodium dodecyl sulfate (SDS) and 2 uL of 20 mg/mL Proteinase-K and incubated at 65° C. with gentle shaking for 1 h. DNA was then purified using standard phenol/chloroform extraction with ethanol precipitation. DNA was resuspended in 30 uL of 0.1×TE BUFFER. CUT&RUN sequencing libraries were generated essentially as described in, e.g., Hainer & Fazzio, Curr Protoc Mol Biol 126, e85 (2019), the content of which is incorporated herein by reference in its entirety, with the following modifications: adapter ligation to end-repaired, and A-tailed DNA was performed using RAPID T4 DNA LIGASE (ENZYMATICS). PCR-amplified libraries were purified from adapter dimers using a 1.1× ratio of AMPURE XP beads, eluting in 20 uL of 10 mM Tris pH 8.0. All CUT&RUN libraries were sequenced on a NEXTSEQ 500 (BASESPACE) using paired-end 40 bp reads.

Analysis. After demultiplexing, sequencing reads were trimmed for quality and remaining adapter sequence using TRIMMOMATIC v0.36 and KSEQ. Trimmed reads were aligned to the mm10 genome using BOWTIE2 v2.2.9 with the following parameters: -local -very-sensitive-local -no-unal -dovetail -no-mixed -no-discordant -phred33 -I 10 -X 700. Trimmed reads were also aligned to the sacCer3 genome with the same parameters to recover reads corresponding to yeast spike-in DNA used in CUT&RUN. Genome-wide coverage of CUT&RUN fragments was generated using BEDTOOLS v2.27.1 GENOMECOV, normalizing to the number of yeast spike-in reads obtained for each sample. Normalized coverage tracks were visualized using IGV v2.4.10 and represent the average signal across all three biological replicates. CUT&RUN coverage over 100 bp bins genome-wide was determined using DEEPTOOLS v.3.0.2 MULTIBIGWIGSUMMARY and was used to calculate Pearson correlation between pairs of replicate samples for each antibody and stimulus condition. Peaks were identified for Fos CUT&RUN using SEACR v.1.1 using the following parameters: norm, relaxed. CUT&RUN performed using rabbit IgG was used as the negative control sample for peak calling. Peaks were subsequently filtered to identify peaks found in all three biological replicates for each condition, creating a conservative set of Fos-bound sites. Peaks within 150 bp of each other were then merged using BEDTOOLS v2.27.1 MERGE. Plots of spike-in normalized CUT&RUN coverage over peaks were generated by first centering peaks on the maximum of CUT&RUN signal within the peak. CUT&RUN coverage over 50 bp bins spanning 1,000 bp upstream and downstream of the peak center was calculated using DEEPTOOLS v.3.0.2 COMPUTEMATRIX. Coverage in each bin was averaged across all peaks, and average per-bin coverage was plotted in R using GGPLOT2.

To determine distances between transcription start sites (TSS) and Fos binding sites, positions of TSS for REFSEQ, activity-regulated (CaMK2a-RIBOTAG), and CA1 excitatory neuron-specific AP-1-regulated (FFJ snRNA-seq) genes were obtained from the UCSC table browser. Distances between Fos binding sites and the nearest TSS were calculated using BEDTOOLS v.2.27.1 CLOSEST; see e.g., Malik et al. Nat Neurosci 17, 1330-1339 (2014), the content of which is incorporated herein by reference in its entirety. Histograms of distances between Fos-bound sites and TSSs were plotted in R using GGPLOT2. The statistical significance of the differences between the distributions of distances for REFSEQ, CaMK2a-RIBOTAG, and FFJ snRNA-seq genes was determined using a Wilcoxon rank-sum test in R.

To identify enriched transcription factor motifs within Fos binding sites, genomic sequences 250 bp upstream and downstream of Fos peak centers were retrieved using BEDTOOLS v.2.27.1 GETFASTA and used as input to multiple em for motif elicitation chromatin immunoprecipitation (MEME-ChIP). Motifs were searched against the HOCOMOCO MOUSE v11 CORE database, allowing for multiple occurrences of motifs per sequence and using default settings for all other parameters. The three motifs with the lowest E-value were reported.

Novel environment single-nucleus RNA-sequencing (NE snRNA-seq). C57BL/6J mice were exposed to a brief 5-min novel environment stimulus and subsequently returned to their home cages for 1 h or 6 h before hippocampal tissue collection. Nuclei were isolated from hippocampal tissue as described above and snRNA-seq was performed using the 10× GENOMICS OR INDROPS platform; see e.g., Klein et al. Cell 161, 1187-1201 (2015), the content of which is incorporated herein by reference in its entirety. A total of 23,610 nuclei, with a range of 700-15,000 RNA molecule counts per cell and 200-2,500 unique genes per cell, were clustered into −13 cell types using the UMAP algorithm. The genes Slc17a7, Fibcd1, and Pex5l were used as a guide to assign cell type identity to the dorsal CA1 excitatory neuron cluster. Raw UMI counts for each gene were normalized to total UMI counts per cell. Differential gene expression and statistical significance were measured using the Wilcoxon rank-sum test. A down-sampled total of 1,659 CA1 excitatory nuclei were used per condition.

Immunoblotting. Whole-cell extracts from 293T cells were generated by rapid lysis of cells in boiling LAEMMLI SDS lysis buffer (4% SDS, 20% glycerol, 10% 2-mercaptoethanol, 0.004% bromophenol blue, 0.125 M Tris HCl pH 6.8). Protein extracts were resolved on 4-12% Bis-Tris gradient (see e.g., FIG. 13C) or 8% Tris-Glycine gels (see e.g., FIG. 15G) and subsequently transferred onto nitrocellulose membranes. Membranes were incubated overnight in the following primary antibodies: mouse anti-Myc (DEVELOPMENTAL STUDIES HYBRIDOMA BANK 9E10 (e.g., in FIG. 13C), 1:1000) or mouse anti-HA (SIGMA HA-7, H3663 (e.g., in FIG. 15G), 1:1000) and rabbit anti-Gapdh (SIGMA G9545, 1:2000). Following washes, membranes were incubated with secondary antibodies conjugated to IRDYE 800CW (LI-COR; mouse (926-32210), rabbit (926-32211), 1:5000) for imaging with the LI-COR ODYSSEY system.

In vivo silicon probe recordings. For all in vivo electrophysiology recordings, 8-10-week-old Scg2^{fl/fl} mice underwent two stereotaxic surgeries. In the first surgery, AAV-Cre-GFP or AAV-ΔCre-GFP was injected into the CA1, and future silicon probe sites over CA1 (stereotaxic coordinates approximately AP −2 mm and ML ±1.8 mm from bregma) were marked on the surface of the skull. METABOND (PARKELL) was used to attach a titanium headplate and cover the remaining exposed skull. Mice were given injections of dexamethasone and buprenorphine SR™, and allowed to recover for 1-2 weeks, during which they were exposed to novel environments daily and habituated to head-fixation on the air-supported STYROFOAM ball. On the day of recording, a second surgery (craniotomy) was performed at one of the marked locations on either the left or right hemisphere. The craniotomy was covered with KWIK-SIL (WORLD PRECISION INSTRUMENTS) and the mouse was allowed to recover fully from anesthesia for at least 4 h. 64-channel silicon probes (NEURONEXUS) were inserted into the cortex and slowly lowered ˜1.25-1.5 mm below the surface of the pia to the pyramidal layer of CA1. In some cases, melted agarose (2% w/v) was applied to the headplate well to stabilize the probe. Probe advancement was stopped in the pyramidal layer of CA1, as evidenced by the presence of theta oscillations and appearance of multiple units in high density across multiple channels. All data were digitized and acquired at 20 kHz (INTAN TECHNOLOGIES RHD2000 RECORDING SYSTEM).

Analysis. All data analysis was carried out with custom MATLAB scripts Channels that were outside of CA1 were excluded from analysis. Spike sorting was performed using KILOSORT2 (available on the world wide web at github.com/MouseLand/Kilosort2), followed by manual inspection and curation of clustering using PHY2 (available on the world wide web at github.com/cortex-lab/phy). Only well-isolated units were chosen for further analysis. Additionally, single units had to meet the following criteria: detected on fewer than 20 channels, half-maximum spike width of less than 1 ms, at least 1,000 spikes detected in the session, and overall firing rate of >0.01 spikes per second. Units were divided into putative excitatory and inhibitory subclasses based on the spike trough to peak duration, using a cutoff of 0.7 ms, below which units were labeled as inhibitory interneurons. Due to the low number of inhibitory interneurons recorded, these were excluded from analyses. For local field potential (LFP) analysis, data from each channel was filtered and downsampled to 1000 Hz. For theta phase-locking analysis, only periods during running were used in the analysis. A single channel within the stratum pyramidale was chosen as the reference. LFPs were filtered and the Hilbert transform was used to determine the phase. The preferred phase of each neuron was computed as the circular mean of the phase at each spike using the CIRCSTATS toolbox in MATLAB. For comparison of single cell properties in the WT and KO groups, cells were pooled across mice. Power spectra were computed between 1 and 120 Hz using the multitaper method (timebandwidth=5, tapers=3) in the CHRONUX toolbox. Power at frequencies between 58-62 Hz were excluded from all subsequent analyses due to 59-61 Hz notch filtering applied (2^{nd} order BUTTERWORTH filter) to remove noise. Power spectra were computed for each channel individually and averaged across channels. To compare across mice and sessions, individual session power spectra were normalized by the sum over all frequencies in the power spectra (1-120 Hz range). Fraction of spikes as a function of theta phase was computed on an individual unit basis by summing spikes in each 10° bin during running and then dividing by the sum of spikes across all bins. See e.g., Yatsenko et al. bioRxiv, doi:10.1101/031658 (2015); Pachitariu et al. bioRxiv, doi:10.1101/061481 (2016); Rossant et al. Nat Neurosci 19, 634-641 (2016); Bartho et al. J Neurophysiol 92, 600-608 (2004); Berens, Journal of Statistical Software 31 (2009); Bokil et al. J Neurosci Methods 192, 146-151 (2010); the contents of each of which are incorporated herein by reference in their entireties.

1. A pharmaceutical composition comprising at least one secretogranin II (scg2) neuropeptide and a pharmaceutically acceptable carrier.

2. The pharmaceutical composition of claim 1, wherein the pharmaceutical composition is formulated for delivery:
to the central nervous system (CNS);
across the blood-brain barrier (BBB),
to the brain, and/or
to pyramidal cells.

3-6. (canceled)

7. The pharmaceutical composition of claim 1, wherein the formulation of the pharmaceutical composition is selected from the group consisting of: direct injection or infusion into the CNS; formulation as a solution comprising a carrier protein; formulation as a nanoparticle; formulation as a liposome; formulation as a nucleic acid; formulation as a CNS-tropic viral vector; formulation with or linkage to an agent that is endogenously transported across the BBB; formulation with or linkage to a cell penetrating peptide (CPP); formulation with or linkage to a BBB-shuttle; formulation with or linkage to an agent that increases permeability of the BBB.

8. The pharmaceutical composition of claim 1, wherein the scg2 neuropeptide is a cleavage product of secretogranin II (scg2) polypeptide.

9. The pharmaceutical composition of claim 8, wherein the scg2 polypeptide comprises SEQ ID NO: 4.

10. The pharmaceutical composition of claim 8, wherein the scg2 neuropeptide, when present in the scg2 polypeptide, is flanked at its N-terminus and at its C-terminus by a dibasic cleavage residue.

11. The pharmaceutical composition of claim 10, wherein the dibasic cleavage residue is selected from the group consisting of:
a) arginine-lysine (RK);
b) lysine-arginine (KR); and
c) arginine-arginine (RR).

12. (canceled)

13. The pharmaceutical composition of claim 10, wherein the dibasic cleavage residue is a specific cleavage site for a Pcsk1/2 protease.

14. The pharmaceutical composition of claim 1, wherein the at least one scg2 neuropeptide is selected from the group consisting of:
a) secretoneurin comprising
(SEQ ID NO: 5)
TNEIVEEQYTPQSLATLESVFQELGKLTGPNNQ;
b) EM66 comprising
(SEQ ID NO: 6)
ERMDEEQKLYTDDEDDIYKANNIAYEDVVGGEDWNPVEEKIESQTQEE
VRDSKENIEKNEQINDEM;
c) manserin comprising
(SEQ ID NO: 7)
VPGQGSSEDDLQEEEQIEQAIKEHLNQGSSQETDKLAPVS;
and
d) SgII comprising
(SEQ ID NO: 8)
FPVGPPKNDDTPNRQYWDEDLLMKVLEYLNQEKAEKGREHIA.

15-22. (canceled)

23. The pharmaceutical composition of claim 1, wherein the scg2 neuropeptide comprises a human, mouse, rat, or chimpanzee scg2 neuropeptide or a chimera thereof.

24. The pharmaceutical composition of claim 1, wherein the scg2 neuropeptide comprises a peptidomimetic.

25. A nucleic acid comprising at least one nucleic acid sequence encoding a secretogranin II (scg2) neuropeptide.

26. The nucleic acid of claim 24, wherein the scg2 neuropeptide is selected from the group consisting of:
a) secretoneurin comprising
(SEQ ID NO: 9)
ACAAATGAAATAGTGGAGGAACAATATACTCCTCAAAGCCTTGCTACA
TTGGAATCTGTCTTCCAAGAGCTGGGGAAACTGACAGGACCAAACAAC
CAG;
b) EM66 comprising
(SEQ ID NO: 10)
GAGAGGATGGATGAGGAGCAAAAACTTTATACGGATGATGAAGATGAT
ATCTACAAGGCTAATAACATTGCCTATGAAGATGTGGTCGGGGGAGAA
GACTGGAACCCAGTAGAGGAGAAAATAGAGAGTCAAACCCAGGAAGAG
GTGAGAGACAGCAAAGAGAATATAGAAAAAAATGAACAAATCAACGAT
GAGATG;
c) manserin comprising
(SEQ ID NO: 11)
GTTCCTGGTCAAGGCTCATCTGAAGATGACCTGCAGGAAGAGGAACAA
ATTGAGCAGGCCATCAAAGAGCATTTGAATCAAGGCAGCTCTCAGGAG
ACTGACAAGCTGGCCCCGGTGAGC;
and
d) SgII comprising
(SEQ ID NO: 12)
TTCCCTGTGGGGCCCCCGAAGAATGATGATACCCCAAATAGGCAGTAC
TGGGATGAAGATCTGTTAATGAAAGTGCTGGAATACCTCAACCAAGAA
AAGGCAGAAAAGGGAAGGGAGCATATTGCT.

27-34. (canceled)

35. A vector comprising the nucleic acid of claim 25.

36-39. (canceled)

40. A viral vector comprising the nucleic acid of claim 25.

41-42. (canceled)

43. A cell comprising the pharmaceutical composition of claim 1.

44-47. (canceled)

48. A composition comprising the nucleic acid of claim 25 and a pharmaceutically acceptable carrier.

49. A method of increasing memory consolidation and/or memory retention or treating a memory-associated disorder, a learning disability, a neurodegenerative disease or disorder, or epilepsy, the method comprising administering an effective amount of the pharmaceutical composition of claim 1 to a subject in need thereof.

50-71. (canceled)

72. A method of diagnosing a memory-associated disorder, learning disability, neurodegenerative disease or disorder, or epilepsy in a subject; comprising:
a) obtaining a sample from the subject;
b) detecting the level of a memory-associated analyte in the sample; and
c) determining that the subject:
i) has or is at risk of developing a memory-associated disorder, learning disability neurodegenerative disease or disorder, or epilepsy if the analyte level is below a pre-determined level; or
ii) does not have or is not at risk of developing a memory-associated disorder, learning disability, neurodegenerative disease or disorder, or epilepsy if the analyte level is at or above a pre-determined level.

73-77. (canceled)

78. A method of increasing memory consolidation and/or memory retention in a subject in need thereof, comprising:
a) obtaining results detecting the level of a memory-associated analyte in a sample from the subject; and
b) administering to the subject:
i) the pharmaceutical composition of claim 1, if the analyte level is below a pre-determined level; or
ii) an alternative treatment, if the analyte level is at or above a pre-determined level.

79.-88. (canceled)
